(19) United States
(12) Patent Application Publication
(10) Pub. No.: US 2024/0166768 A1
(43) Pub. Date: May 23, 2024

(54) ANTI-MATRIX METALLOPROTEINASE-14 ANTIBODIES FOR THE TREATMENT OF CANCER

(71) Applicant: Yeda Research and Development Co. Ltd., Rehovot (IL)

(72) Inventors: Irit SAGI, Rehovot (IL); Roei David MAZOR, Rehovot (IL); Ziv SHULMAN, Rehovot (IL)

(73) Assignee: Yeda Research and Development Co. Ltd., Rehovot (IL)

(21) Appl. No.: 18/430,696

(22) Filed: Feb. 2, 2024

Related U.S. Application Data
(63) Continuation of application No. PCT/IL2022/050841, filed on Aug. 2, 2022.
(60) Provisional application No. 63/305,693, filed on Feb. 2, 2022.

(30) Foreign Application Priority Data
Aug. 2, 2021 (IL) 285313

(51) Int. Cl. C07K 16/40; A61K 39/00; A61K 39/395; A61P 35/00; C07K 14/725; G01N 33/573; G01N 33/574

(52) U.S. Cl. CPC C07K 16/40; A61K 39/3955; A61K 39/4613; A61P 35/00; C07K 14/7051; G01N 33/573; G01N 33/57492; C07K 2319/30; G01N 2333/96486

(57) ABSTRACT

Monoclonal antibodies for the treatment of ovarian cancer are provided also provide are uses of same. Such uses include characterizing the tumor, diagnosing it and treatment of same.

### RELATED APPLICATIONS

This application is a Continuation of PCT Patent Application No. PCT/IL2022/050841 having international date of Aug. 2, 2022 which claims the benefit of priority of U.S. Provisional Patent Application No. 63/305,693 filed on Feb. 2, 2022 and Israeli Patent Application No. 285313 filed on Aug. 2, 2021. The contents of the above applications are all incorporated by reference as if fully set forth herein in their entirety.

### SEQUENCE LISTING STATEMENT

The XML file entitled 98874SequenceListing.xml, created on Dec. 27, 2023, comprising 682,897 bytes, submitted concurrently with the filing of this application is incorporated herein by reference.

### FIELD AND BACKGROUND OF THE INVENTION

The present invention, in some embodiments thereof, relates to antibodies for the treatment of cancer.

Ovarian cancer is the most lethal gynaecological malignancy and the fifth leading cause of cancer related death in women, accounting for 5% of all cancer related deaths in this gender group. In 2004, ovarian cancer was classified into two categories according to its histological and molecular characteristics: Type I tumors consist of low grade tumors which grow in a step wise fashion, such as low grade serous ovarian carcinoma as well as ovarian carcinomas of endometroid, clear cell, mucinous and transitional histologies. These tumors comprise distinct molecular aberrations which are absent from type II tumors. For example, these include mutations involving elements of the mitogen activated protein kinase (MAPK) pathway—such as BRAF and KRAS for the serous and mucinous tumors and mutations in PTEN & β-catenin for tumors of endometroid histology. Conversely, Type II tumors consist of high grade neoplasms including high grade serous ovarian carcinoma (HGSOC), carcinosarcoma and undifferentiated ovarian carcinoma. These tumors are characterized by recurrent mutations in BRCA, BRCA2 and specifically p53—which is nearly universally mutated (96%) in HGSOC. While type I tumors arise from the ovarian surface epithelium, it is commonly accepted that type II tumors originate from the fallopian tube epithelium.

A stepwise approach to assessment, diagnosis, and treatment is vital to appropriate management of this disease process. An integrated approach with gynecologic oncologists as well as medical oncologists, pathologists, and radiologists is of paramount importance to improving outcomes. Surgical cytoreduction to R0 is the mainstay of treatment, followed by adjuvant chemotherapy. Genetic testing for gene mutations that affect treatment is the standard of care for all women with epithelial ovarian cancer. However, nearly all women will have a recurrence, and the treatment of recurrent ovarian cancer continues to be nuanced and requires extensive review of up to date modalities that balance efficacy with the patient's quality of life.

Additional background art includes: Devi et al. presented in the AACR Annual Meeting—Apr. 4-8, 2007; Los Angeles, CA, May 2007 Volume, Issue 9 Supplement a DX-2400, which is a human anti MMP14 antibody suggested for the treatment of ovarian cancer.

### SUMMARY OF THE INVENTION

According to an aspect of some embodiments of the present invention there is provided a monoclonal antibody comprising an antigen binding domain which comprises the complementarity determining regions (CDRs) CDRH1, CDRH2, CDRH3, CDRL1, CDRL2 and CDRL3 or the heavy chain and light chain of an antibody selected from the group consisting of T1, T2, T3, T4, T5, T6, T7, T8, T10, T11, T12, T13, T14, T15, T17, T18, T19, T20, T21, T22, T23, T24, T25, T26, T27, T28, T29, T30, T4-GL, T7-GL, T22-GL, T30-GL, T12 MRCA, T13 MRCA and T3-16-17 MRCA1 and T3-16-17 MRCA2.

According to an aspect of some embodiments of the present invention there is provided a monoclonal antibody comprising an antigen binding domain which binds an I-A loop of human MMP14.

According to an aspect of some embodiments of the present invention there is provided an isolated polynucleotide encoding the monoclonal antibody as described herein.

According to an aspect of some embodiments of the present invention there is provided a nucleic acid construct comprising the polynucleotide as described herein under a transcriptional control of a cis-acting regulatory element, the element being heterologous to the polynucleotide.

According to an aspect of some embodiments of the present invention there is provided a cell comprising the nucleic acid construct as described herein.

According to some embodiments of the invention, the antibody is an antibody fragment.

According to some embodiments of the invention, the antibody fragment is a single chain Fv (scFv) or a Fab.

According to some embodiments of the invention, the antibody forms a chimeric antigen receptor (CAR).

According to some embodiments of the invention, the CAR is in CAR-T or CAR-NK cells.

According to some embodiments of the invention, the antibody comprises an antibody-dependent cell mediated cytotoxicity (ADCC) or antibody-dependent cellular phagocytosis (ADCP).

According to some embodiments of the invention, the antibody, polynucleotide, construct, cell as described herein is an IgG serotype.

According to some embodiments of the invention, the antibody is humanized.

According to some embodiments of the invention, the antibody as described herein forms an antibody-drug conjugate (ADC).

According to some embodiments of the invention, the drug is a viral antigen.

According to some embodiments of the invention, the drug is mRNA.

According to some embodiments of the invention, the antibody binds the catalytic domain of MMP14.

According to some embodiments of the invention, the antibody binds OVCAR3 cells.

According to some embodiments of the invention, the antibody recruits immune cells to a tumor microenvironment.

According to an aspect of some embodiments of the present invention there is provided a method of treating cancer in a subject in need thereof, the method comprising administering to the subject a therapeutically effective amount of the antibody, polynucleotide, construct or cell or a polyclonal preparation of antibodies as described herein is from an ascites fluid of an ovarian cancer patient, thereby treating the cancer in the subject.

According to some embodiments of the invention, the antibody, polynucleotide, construct or cell as described herein or a polyclonal preparation of antibodies from an ascites fluid of an ovarian cancer patient for use in treating cancer in a subject in need thereof.

According to some embodiments of the invention, the cancer is MMP14+.

According to some embodiments of the invention, the cancer is ovarian cancer.

According to some embodiments of the invention, the ovarian cancer is high grade serous ovarian carcinoma (HGSOC).

According to some embodiments of the invention, the cancer is pancreatic cancer.

According to some embodiments of the invention, the polyclonal preparation is of the subject.

According to some embodiments of the invention, the administering is following a surgery.

According to some embodiments of the invention, the surgery is a primary debulking surgery.

According to some embodiments of the invention, the administering is by intraperitoneal administration.

According to some embodiments of the invention, the method further comprises adoptive cell therapy.

According to some embodiments of the invention, the cells of the adoptive cell therapy comprise ex vivo expanded, lymphokine-activated NK cells or Human activated NK (HaNKs) cells.

According to some embodiments of the invention, the cells of the adoptive cell therapy are autologous cells.

According to some embodiments of the invention, the cells of the adoptive cell therapy are allogeneic cells.

According to some embodiments of the invention, the method further comprises administering an ant-cancer agent different than the antibody or antibody preparation.

According to some embodiments of the invention, the anti-cancer agent is selected from the group consisting of a chemotherapy, a toxin, a radiotherapy, an immunemodulator and a toxin.

According to some embodiments of the invention, the antibody or polyclonal preparation of antibodies are formulated as an antibody drug conjugate (ADC).

According to some embodiments of the invention, the cancer is characterized by being coated with anti-MMP14 antibodies.

According to an aspect of some embodiments of the present invention there is provided a method of characterizing an MMP14+ tumor, the method comprising: determining coating of the tumor with anti MMP14 antibodies, wherein coating with the anti MMP14 antibodies indicates that the tumor is treatable with adoptive cell therapy.

According to some embodiments of the invention, the adoptive cell therapy comprises NK cells therapy.

According to some embodiments of the invention, the method further comprises treating the subject with an anti MMP 14 antibody.

According to an aspect of some embodiments of the present invention there is provided a method of diagnosing ovarian cancer in a subject in need thereof, the method comprising:
(a) providing a utero-tubal lavage of the subject; and
(b) determining in the utero-tubal lavage a presence or level of MMP14, wherein presence or level above a predetermined threshold is indicative of ovarian cancer in the subject.

According to some embodiments of the invention, the ovarian cancer is tubal carcinoma in situ.

According to some embodiments of the invention, the determining is by using anti MMP14 antibodies.

According to an aspect of some embodiments of the present invention there is provided a method of treating ovarian cancer in a subject in need thereof, the method comprising:
(a) diagnosing the ovarian cancer as described herein;
and
(b) treating the cancer.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWING(S)

The patent or application file contains at least one drawing executed in color. Copies of this patent or patent application publication with color drawing(s) will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-C show that HGSOC infiltrating ASC (Antibody secreting cells) derived monoclonal antibodies are tumor reactive. FIG. 1A. Representative images and quantification of the mean fluorescence intensity of OVCAR3 cells stained with different monoclonal antibodies.

FIG. 1B. Left panel Quantification of mean fluorescence intensity was performed on each cell separately using QuPath v0.2.0-m9. Boxes are divided by the median value and represent the interquartile range; whiskers represent the 0-90 percentiles. The dashed line represents mean background fluorescence. Scale, 50 um comparison between measurements was performed using a one-way ANOVA. FIG. 1B. Right Panel.ELISA for binding of monoclonal antibodies to various cell lines. The fluorescence intensity was normalized to DAPI staining. GD0 was used as a negative control. FIG. 1C quantification of the mean fluorescence intensity of OVCAR3 cells stained with additional monoclonal antibodies, as in FIG. 1B, left panel.

FIGS. 2A-C show that MMP14 is highly expressed in ovarian carcinoma and in other malignancies. FIG. 2A. Correlation matrix depicting the mean expression level of genes derived from previously published RNA sequencing data sets of 85 HGSOC primary tumors (GSE02073) and 35 HGSOC omental metastases (GSE7340). Each dot represents a mean Log2CPM value. Specific gene families are highlighted—MMPs in red, ADAMs in purple, LOXs in green, Kallikreins in yellow and house-keeping genes in blue. The yellow, orange and pink backgrounds represent territories corresponding to the top 0%, 5% and % genes, respectively. The dashed circle represents the most highly expressed MMPs and ADAMs in both the primary tumor and metastasis. The degree of correlation was calculated using Pearson's correlation coefficient. The black line represents a linear regression model. FIG. 2B. MMP14 immunohistochemical staining of HGSOC primary tumor and healthy controls. Scale, 50 um. FIG. 2C. Western blot for MMP14, performed on lysates from different cell lines and patient tumor samples.

FIGS. 3A-B show that Ascites derived polyclonal antibodies are MMP14 reactive. FIG. 3A. Analysis of patient-derived polyclonal antibodies binding to the indicated targets by ELISA. p53 used as positive control. BSA used as negative control. FIG. 3B. ELISA assay comparing the MMP14 reactivity of control sera to that of ascites-derived polyclonal antibodies.

FIGS. 4A-B show that HGSOC infiltrating ASC derived monoclonal antibodies are able to bind MMP14. FIG. 4A. Analysis of patient-derived monoclonal antibodies binding to the indicated targets by ELISA. The heatmap depicts the mean optical density data from two independent ELISA assays. FIG. 4B. ELISA showing the dose-dependent binding of monoclonal antibodies to MMP14 and MMP.

FIG. 4C show analysis of patient-derived monoclonal antibodies binding to the indicated targets, as determined by ELISA. The heatmap depicts the mean optical density data from two independent ELISA assays.

FIGS. 5A-C show that HGSOC derived monoclonal antibodies are not polyreactive. FIG. 5A. Polyreactivity analysis by ELISA against structurally distinct targets and HEp-2 cell line lysate. ED38 was used as a positive control and the GD0 antibody as negative control. FIG. 5B. Protein microarray reactivity analysis of T3 (left) and T5 mAbs (right). Top panel: Each dot represents a distinct protein. Normalized signal intensities are plotted against the protein array ID. Top % hits (blue) and chosen targets (red) are annotated accordingly. Bottom panel: Normalized signal intensities (F532 Median—B532, a measure of signal in a protein microarray assay)) plotted against Relative protein units (RFU) in LOGO scale. Top hits are presented.

FIGS. 6A-C show that HGSOC derived monoclonal antibodies are able to bind MMP14. FIG. 6A. Measurement of the kinetic constants governing the binding of the T3 mAb to MMP-4 by biolayer interferometry analysis using the Octet QKe platform. FIG. 6B. Western blot analysis of T3 binding to various cell line lysates. FIG. 6C. Analysis of T3 binding to K562 cells over-expressing mCherry:MMP14. Fluorescence intensity values from three independent experiments are shown. In a, formulation of the response model and determination of the goodness-of-fit parameters were obtained using Fortebio Octet Data analysis software (ForteBio). In c, comparison between measurements was performed using a one way ANOVA.

FIGS. 7A-E show epitope mapping of mAbs T2 and T3 reveals MMP14's I-A loop as their target epitope. (FIGS. 7A-B) Phage display data depicting the possible amino acid sequences in MMP14 that are bound by mAbs T2 and T3. FIG. 7A the X axis represents the amino acid sequence of the catalytic domain of MMP 4 from its N to its C terminus (amino acids 08-293). The bars are a measure of enrichment and represent the number of NGS read of different peptides that aligned to their position in the amino acid sequence. Bars in the blue spectrum depict peptide alignments to T3 and bars in the red spectrum depict peptide alignments to T2. Bar colors are coded to the peptides, as they appear in panel b. The heatmap below the x axis, labeled ACS (Average Conservation Score) represents the average level of alignment compatibility for peptides against T3 (blue heatmap) and T2 (red heatmap). The score represented by the heatmap is an average of all measurements for a given amino acid locus. A score of 3 is granted when the amino acid in the peptide is identical to that which is in the sequence of MMP14. A score of 2 indicates conservation between amino acids of strongly similar properties (scoring >0.5 in the Gonnet PAM 250 matrix). The score indicates conservation between amino acids of weakly similar properties (scoring of <0.5 in the Gonnet PAM 250 matrix). A score of 0 indicates no conservation between the amino acids. The light blue & light red regions demarcate regions of interest (ROI & ROI2 respectively), that are highly likely to represent the binding epitopes for the antibodies. The light gray region demarcates a region to which peptides did not align, as a control. FIG. 7B. Pie charts indicating the degree of enrichment of the top 5 peptide hits as averaged in three replicate experiments for both antibodies. The numbers in the center indicate the total number of NGS reads. FIG. 7C. Validation ELISA in which peptides representing the regions of interest in the MMP14 amino acid sequence were reacted in different concentrations with the two antibodies. Peptide (ROI) is bound by both antibodies. FIG. 7D. In-silico prediction of the structure of T3 using the ABpredict algorithm followed by docking prediction of the crystal structure of MMP14 to the mAb using PatchDock. MMP14 appears in green, T3 appears in magenta. CDRs appear in teal. MMP14 residues forming up to 3.5\acute{Å} interactions with the antibody appear in orange. FIG. 7E. Depiction of the phage display ROIs (in blue and red) on the docking model show a high degree of region conservation, with 7 amino acids forming interactions in the prediction and phage display experiment independently.

FIGS. 8A-C shows tumor reactive antibodies arise from autoreactive and non-functional precursors. FIG. 8A. ELISA for MMP14 binding using mutated and non-mutated monoclonal antibodies. FIG. 8B. Similar to a, with the addition of most recent common ancestors (MRCA) FIG. 8C. Quantification of OVCAR3 binding by mutated and non-mutated antibodies as measured by fluorescence microscopy. Quantification of mean fluorescence intensity was performed on each cell separately using QuPath v0.2.0-m9. Boxes are divided by the median value and represent the interquartile range; whiskers represent the 0-90 percentiles. The dashed line represents mean background fluorescence. Fold change in median fluorescence of the mutated antibody compared to the non mutated is indicated above the bars.

FIGS. 9A-D show that Tumor derived monoclonal antibodies exhibit potential anti-tumor effector functions. FIG. 9A. Flow cytometric quantification of monoclonal mAb-mediated phagocytosis of MMP14-coated beads by THP-monocytes. FIG. 9B, Longitudinal quantification of antibody dependent cell-mediated cytotoxicity (ADCC) targeting OVCAR3 cells, in the presence of NK cells together with either isotype control, Cetuximab, or monoclonal antibodies. Data were collected using the xCelligence RTCA DP platform. FIG. 9C. Phagocytosis analysis as in c by patient-derived polyclonal antibodies. FIG. 9D. OVCAR3 killing by patient-derived polyclonal antibodies as in d. Average of four reactions with standard deviation is shown. In all the panels statistical significance was determined by one way ANOVA.

FIG. 10 shows representative images and quantification of normalized fluorescence intensity of primary cultured tumors (primary tumor, omental metastasis, and ascites tumor cells) or OVCAR3 cells stained with monoclonal antibodies and DAPI. The IgG fluorescent signal was normalized to DAPI to account for potential variability in cellular density between replicates. Each colour represents an individual patient.

FIG. 11 shows images of peritoneal tumor implants of ID-8 murine ovarian carcinoma that are preferentially bound by mAbs T3 and T21.

### DESCRIPTION OF SPECIFIC EMBODIMENTS OF THE INVENTION

The present invention, in some embodiments thereof, relates to antibodies for the treatment of cancer.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

In an effort to design a novel tool for combating ovarian cancer, the present inventors focused at identifying physiological antibodies having anti-cancer activity which can be used in the clinic upon cloning with or without further modifications.

Using bait-free single cell immunoglobulin sequencing and patient-derived antibodies, it was found that somatic hypermutations (SHM) promote tumor-reactivity against surface autoantigens in high grade serous ovarian carcinoma (HGSOC). HGSOC tumor cells originating from both the primary tumor as well as from omental metastases were decorated with IgG typed antibodies and antibodies purified from the malignant ascites fluids of HGSOC patients were able to bind ovarian cancer cell lines. However, IgG typed antibodies decorating the surface of tumor cells were not exclusive to HGSOC, as tumors derived from 345 samples from 24 types of cancer were analyzed and found to be frequently and heterogeneously coated with such antibodies. Intratumoral IgG⁺ antibody secreting cells (ASCs), primarily situated at the stromal tumor microenvironment were found to be abundant in HGSOC. Single cell sequencing of these intratumoral ASCs revealed characteristic features of antigen driven selection, including highly mutated immunoglobulin genes and clonal expansion of ASCs, which were organized in complex multi-generation phylogenies. Remarkably, polyclonal antibodies purified from HGSOC ascites fluids as well as monoclonal antibodies expressed on the basis of sequenced intratumoral ASCs targeted ECM-remodeling matrix metalloproteinases (MMPs), including MMP14, a membrane tethered protease which is abundantly expressed on the tumor cell surface. These monoclonal antibodies were unable to bind structurally unrelated antigens and as such, did not show evidence of poly-reactivity. Through reversion of patient-derived monoclonal antibodies to their germline configuration, two classes of antibodies were identified: one that depends on SHM for binding to tumor autoantigens and the tumor cell surface, and a second that shows germline encoded auto-reactivity. Tumor derived monoclonal antibodies as well as ascites derived polyclonal antibodies appeared to retain their Fc mediated functions, as they were able to mediate ADCC against an HGSOC cell line and facilitated antibody dependent phagocytosis (ADCP) of MMP14 coated particles. Thus, the humoral immune response against tumors is largely intact and emerges from either non-reactive precursors in addition to pre-existing autoreactive B cells. These findings highlight the potential applicability of autoantibodies, such as anti-MMP14, for tumor targeting.

Thus, according to an aspect of the invention there is provided a monoclonal antibody comprising an antigen binding domain which comprises the complementarity determining regions (CDRs) CDRH1, CDRH2, CDRH3, CDRL1, CDRL2 and CDRL3 or the heavy chain and light chain of an antibody selected from the group consisting of T1, T2, T3, T4, T5, T6, T7, T8, T10, T11, T12, T13, T14, T15, T17, T18, T19, T20, T21, T22, T23, T24, T25, T26, T27, T28, T29, T30, T4-GL, T7-GL, T22-GL, T30-GL, T12 MRCA, T13 MRCA and T3-16-17 MRCA1 and T3-16-17 MRCA2.

According to another aspect of the invention there is provided a monoclonal antibody comprising an antigen binding domain which binds an I-A loop of human MMP14.

When referring to the antibodies in Table 1 the meaning is for heavy chain and light chain of a specific antibody e.g., T3.

TABLE 1
(The SEQ ID NO of each sequence appear on Table 1A)

| Mono- | | Heavy chain | | Light chain |
|---|---|---|---|---|
| clonal | Heavy | amino acid | Light | amino acid |
| antibody | chain nucleotide sequence | sequence | chain nucleotide sequence | sequence |
| T13 | GAGGTGCAGCTGGTGGAGTCTGGG | EVQLVESGG | GAAATAGTGATGACGCAGTCTC | EIVMTQSPAT |
| | GGAGGCTTGGTACAGCCTGGGGGG | GLVQPGGSL | CAGCCACCCTGTCTGTGTCTCCA | LSVSPGERAT |
| | TCCCTGAGACTCTCCTGTGCAGCCTC | RLSCAASGFI | GGGGAAAGGGCCACCCTCTCCT | LSCRASQNIHI |
| | TGGATTCATATTCAGTAAGCACGAC | FSKHDMYW | GCAGGGCCAGTCAGAATATTCA | NLAWYQQKP |
| | ATGTATTGGGTCCGCCAAAGACCAG | VRQRPGKGL | CATCAACTTAGCCTGGTACCAG | GQAPRLIIYA |
| | GAAAGGGTCTGGAGTGGGTCTCAC | EWVSRIGDA | CAGAAACCTGGCCAGGCTCCCA | ASTRAAGIPA |
| | GGATTGGTGATGCTGGTGACACATA | GDTYYAGSV | GGCTCATCATCTACGCTGCATCC | RFSASGSGTE |
| | CTATGCAGGCTCCGTGAAGGGCCG | KGRFTIFREN | ACCAGGGCCGCGGGTATCCCA | FTLTISSLQSE |
| | ATTCACCATCTTCAGAGAAAATGCC | AKNTLYFQM | GCCAGGTTCAGTGCCAGTGGGT | DFAVYYCQQ |
| | AAGAACACGTTGTATTTTCAAATGA | NRLTDGDTA | CTGGGACAGAGTTCACTCTCAC | FNPWSPWTF |
| | ACAGACTGACAGACGGGGACACGG | VYYCGRGMA | TATCAGCAGTCTGCAGTCTGAA | GQGTKVEVK |
| | CTGTATATTATTGTGGAAGAGGTAT | VAGFPLDVW | GATTTTGCAGTTTATTACTGTCA | |
| | GGCAGTGGCTGGATTTCCCCTGGAC | GRGTRVTVT | GCAGTTTAATCCCTGGTCTCCGT | |
| | GTGTGGGGCAGAGGGACAAGGGTC | S | GGACGTTCGGCCAAGGGACCA | |
| | ACCGTCACCTCA | | AGGTTGAAGTCAAA | |
| T12 | GAGGTGCAGCTGGTGGAGTCTGGG | EVQLVESGG | GAAATAGTGATGACGCAGTCTC | EIVMTQSPAT |
| | GGAGGCTTGGTTCAACCTGGGGGG | GLVQPGGSL | CAGCCACCCTGTCTGTGTCTCCA | LSVSPGETAT |
| | TCCCTGAGACTCTCCTGTGCAGCCTC | RLSCAASGFT | GGGGAGACAGCCACCCTCTCCT | LSCRANQTLY |
| | TGGATTCACTTTCAATAACCACGAC | FNNHDMH | GCAGGGCCAATCAGACTCTTTA | NNLAWYQQ |
| | ATGCACTGGGTCCGCCAAGTTACAG | WVRQVTGK | CAACAACTTAGCCTGGTACCAA | KPGQAPRLLI |
| | GGAAAGGTCTAGAATGGGTCTCAA | GLEWVSSIG | CAGAAACCTGGCCAGGCTCCCA | YSGSTRATGI |
| | GTATTGGTAATTTTGGTGACACATA | NFGDTYYSG | GGCTGCTCATCTATAGTGGATC | PARFSGSGSG |
| | CTATTCAGGCTCCGTGAAGGGCCGA | SVKGRFTISR | CACCAGGGCCACGGGAATCCCA | TEFSLTISSLQ |
| | TTCACCATCTCCAGACAAGATGCCA | QDAKNSLYL | GCCAGGTTCAGTGGCAGTGGG | SEDFAVYYCQ |
| | AGAACTCCTTGTATCTTCAAATGAAT | QMNRLRVG | TCTGGGACAGAGTTCAGTCTCA | HYTPWPPYT |
| | AGACTGAGAGTCGGGGACACGGCT | DTAVYYCAR | CCATCAGCAGCCTGCAGTCTGA | FGQGTKVDF |
| | GTCTATTATTGTGCAAGAGGAAGAG | GRAVAGNPL | AGACTTTGCAGTTTATTACTGTC | K |
| | CAGTGGCTGGAAATCCCCTGGACGT | DVWGKGTT | AGCACTATACTCCCTGGCCTCC | |
| | CTGGGGCAAAGGGACCACGGTCAC | VTVSS | GTATACGTTCGGCCAAGGGACC | |
| | CGTCTCCTCA | | AAGGTGGACTTCAAA | |
| T13- | GAGGTGCAGCTGGTGGAGTCTGGG | EVQLVESGG | GAAATAGTGATGACGCAGTCTC | EIVMTQSPAT |
| MRCA | GGAGGCTTGGTACAGCCTGGGGGG | GLVQPGGSL | CAGCCACCCTGTCTGTGTCTCCA | LSVSPGERAT |
| | TCCCTGAGACTCTCCTGTGCAGCCTC | RLSCAASGFT | GGGGAAAGAGCCACCCTCTCCT | LSCRASQSVN |
| | TGGATTCACCTTCAGTAACCACGAC | FSNHDMHW | GCAGGGCCAGTCAGAGTGTTA | SNLAWYQQK |
| | ATGCACTGGGTCCGCCAAGCTACAG | VRQATGKGL | ACAGCAACTTAGCCTGGTACCA | PGQAPRLLIY |
| | GAAAAGGTCTGGAATGGGTCTCAG | EWVSGIGNA | GCAGAAACCTGGCCAGGCTCCC | GASTRATGIP |
| | GTATTGGTAATGCTGGTGACACATA | GDTYYPGSV | AGGCTCCTCATCTATGGTGCAT | ARFSGSGSGT |
| | CTATCCAGGCTCCGTGAAGGGCCGA | KGRFTISREN | CCACCAGGGCCACGGGTATCCC | EFTLTISSLQS |
| | TTCACCATCTCCAGAGAAAATGCCA | AKNSLYLQM | AGCCAGGTTCAGTGGCAGTGG | EDFAVYYCQ |
| | AGAACTCCTTGTATCTTCAAATGAAC | NRLRAGDTA | GTCTGGGACAGAGTTCACTCTC | QYNPWPPW |
| | AGACTGAGAGCCGGGGACACGGCT | VYYCARGIAV | ACCATCAGCAGCCTGCAGTCTG | TFGQGTKVEI |
| | GTGTATTATTGTGCAAGAGGTATAG | AGFPLDVWG | AAGATTTTGCAGTTTATTACTGT | K |
| | CAGTGGCTGGATTTCCCCTGGACGT | KGTTVTVSS | CAGCAGTATAATCCCTGGCCTC | |
| | CTGGGGCAAAGGGACCACGGTCAC | | CGTGGACGTTCGGCCAAGGGA | |
| | CGTCTCCTCA | | CCAAGGTGGAAATCAAA | |
| T12- | GAGGTGCAGCTGGTGGAGTCTGGG | EVQLVESGG | GAAATAGTGATGACGCAGTCTC | EIVMTQSPAT |
| MRCA | GGAGGCTTGGTACAGCCTGGGGGG | GLVQPGGSL | CAGCCACCCTGTCTGTGTCTCCA | LSVSPGERAT |
| | TCCCTGAGACTCTCCTGTGCAGCCTC | RLSCAASGFT | GGGGAAAGAGCCACCCTCTCCT | LSCRASQSVN |
| | TGGATTCACCTTCAGTAACCACGAC | FSNHDMHW | GCAGGGCCAGTCAGAGTGTTA | SNLAWYQQK |
| | ATGCACTGGGTCCGCCAAGCTACAG | VRQATGKGL | ACAGCAACTTAGCCTGGTACCA | PGQAPRLLIY |
| | GAAAAGGTCTGGAATGGGTCTCAG | EWVSGIGNA | GCAGAAACCTGGCCAGGCTCCC | GASTRATGIP |
| | GTATTGGTAATGCTGGTGACACATA | GDTYYPGSV | AGGCTCCTCATCTATGGTGCAT | ARFSGSGSGT |
| | CTATCCAGGCTCCGTGAAGGGCCGA | KGRFTISREN | CCACCAGGGCCACGGGTATCCC | EFTLTISSLQS |
| | TTCACCATCTCCAGAGAAAATGCCA | AKNSLYLQM | AGCCAGGTTCAGTGGCAGTGG | EDFAVYYCQ |
| | AGAACTCCTTGTATCTTCAAATGAAC | NRLRAGDTA | GTCTGGGACAGAGTTCACTCTC | QYNPWPPW |
| | AGACTGAGAGCCGGGGACACGGCT | VYYCARGIAV | ACCATCAGCAGCCTGCAGTCTG | TFGQGTKVEI |
| | GTGTATTATTGTGCAAGAGGTATAG | AGNPLDVW | AAGATTTTGCAGTTTATTACTGT | K |
| | CAGTGGCTGGAAATCCCCTGGACGT | GKGTTVTVSS | CAGCAGTATAATCCCTGGCCTC | |
| | CTGGGGCAAAGGGACCACGGTCAC | | CGTGGACGTTCGGCCAAGGGA | |
| | CGTCTCCTCA | | CCAAGGTGGAAATCAAA | |
| T13-GL | GAGGTGCAGCTGGTGGAGTCTGGG | EVQLVESGG | GAAATAGTGATGACGCAGTCTC | EIVMTQSPAT |
| | GGAGGCTTGGTACAGCCTGGGGGG | GLVQPGGSL | CAGCCACCCTGTCTGTGTCTCCA | LSVSPGERAT |
| | TCCCTGAGACTCTCCTGTGCAGCCTC | RLSCAASGFT | GGGGAAAGAGCCACCCTCTCCT | LSCRASQSVS |
| | TGGATTCACCTTCAGTAGCTACGAC | FSSYDMHW | GCAGGGCCAGTCAGAGTGTTA | SNLAWYQQK |
| | ATGCACTGGGTCCGCCAAGCTACAG | VRQATGKGL | GCAGCAACTTAGCCTGGTACCA | PGQAPRLLIY |
| | GAAAAGGTCTGGAATGGGTCTCAG | EWVSAIGTA | GCAGAAACCTGGCCAGGCTCCC | GASTRATGIP |
| | CTATTGGTACTGCTGGTGACACATA | GDTYYPGSV | AGGCTCCTCATCTATGGTGCAT | ARFSGSGSGT |
| | CTATCCAGGCTCCGTGAAGGGCCGA | KGRFTISREN | CCACCAGGGCCACTGGTATCCC | EFTLTISSLQS |
| | TTCACCATCTCCAGAGAAAATGCCA | AKNSLYLQM | AGCCAGGTTCAGTGGCAGTGG | EDFAVYYCQ |
| | AGAACTCCTTGTATCTTCAAATGAAC | NSLRAGDTA | GTCTGGGACAGAGTTCACTCTC | QYNNWPPW |
| | AGCCTGAGAGCCGGGGACACGGCT | VYYCARGIAV | ACCATCAGCAGCCTGCAGTCTG | TFGQGTKVEI |
| | GTGTATTACTGTGCAAGAGGTATAG | AGFPLDVWG | AAGATTTTGCAGTTTATTACTGT | K |
| | CAGTGGCTGGATTTCCCCTGGACGT | KGTTVTVSS | CAGCAGTATAATAACTGGCCTC | |
| | CTGGGGCAAAGGGACCACGGTCAC | | CGTGGACGTTCGGCCAAGGGA | |
| | CGTCTCCTCA | | CCAAGGTGGAAATCAAA | |
| T12-GL | GAGGTGCAGCTGGTGGAGTCTGGG | EVQLVESGG | GAAATAGTGATGACGCAGTCTC | EIVMTQSPAT |
| | GGAGGCTTGGTACAGCCTGGGGGG | GLVQPGGSL | CAGCCACCCTGTCTGTGTCTCCA | LSVSPGERAT |
| | TCCCTGAGACTCTCCTGTGCAGCCTC | RLSCAASGFT | GGGGAAAGAGCCACCCTCTCCT | LSCRASQSVS |
| | TGGATTCACCTTCAGTAGCTACGAC | FSSYDMHW | GCAGGGCCAGTCAGAGTGTTA | SNLAWYQQK |
| | ATGCACTGGGTCCGCCAAGCTACAG | VRQATGKGL | GCAGCAACTTAGCCTGGTACCA | PGQAPRLLIY |
| | GAAAAGGTCTGGAATGGGTCTCAG | EWVSAIGTA | GCAGAAACCTGGCCAGGCTCCC | GASTRATGIP |
| | CTATTGGTACTGCTGGTGACACATA | GDTYYPGSV | AGGCTCCTCATCTATGGTGCAT | ARFSGSGSGT |
| | CTATCCAGGCTCCGTGAAGGGCCGA | KGRFTISREN | CCACCAGGGCCACTGGTATCCC | EFTLTISSLQS |
| | TTCACCATCTCCAGAGAAAATGCCA | AKNSLYLQM | AGCCAGGTTCAGTGGCAGTGG | EDFAVYYCQ |
| | AGAACTCCTTGTATCTTCAAATGAAC | NSLRAGDTA | GTCTGGGACAGAGTTCACTCTC | QYNNWPPW |
| | AGCCTGAGAGCCGGGGACACGGCT | VYYCARGIAV | ACCATCAGCAGCCTGCAGTCTG | TFGQGTKVEI |
| | GTGTATTACTGTGCAAGAGGTATAG | AGNPLDVW | AAGATTTTGCAGTTTATTACTGT | K |
| | CAGTGGCTGGAAATCCCCTGGACGT | GKGTTVTVSS | CAGCAGTATAATAACTGGCCTC | |
| | CTGGGGCAAAGGGACCACGGTCAC | | CGTGGACGTTCGGCCAAGGGA | |
| | CGTCTCCTCA | | CCAAGGTGGAAATCAAA | |
| T14 | GAGGTGCAGCTGGTGGAGTCTGGG | EVQLVESGG | GACATCGTGATGACCCAGTCTC | DIVMTQSPV |
| | GGAGGCTTGGTCCAGCCTGGGGGG | GLVQPGGSL | CAGTCTCCCTGGCTGTGTCTCTG | SLAVSLGERA |
| | TCCCTGAGACTCTCCTGTGCAGTCTC | RLSCAVSGFT | GGCGAGAGGGCCACCATCAAC | TINCKSSQSV |
| | TGGATTCACCTTTAATAACTATTGGA | FNNYWMT | TGCAAGTCCAGCCAGAGTGTTT | LYSSNNKNYL |
| | TGACCTGGGTCCGCCAGGCTCCAGG | WVRQAPGK | TATACAGCTCCAACAACAAGAA | AWYQQKPG |
| | GAAGGGGCTGGAGTGGGTGGCCAA | GLEWVANIK | CTATTTAGCTTGGTACCAGCAG | QPPKVLIYWA |
| | CATAAAGGGAGATGGGAGTGAGAA | GDGSEKTYV | AAACCAGGACAGCCTCCTAAGG | STRESGVPDR |
| | AACCTATGTGGACTCTGTGAAGGGC | DSVKGRFTVS | TACTCATTTACTGGGCTTCTACC | FSGSGSGADF |
| | CGATTCACCGTCTCCAGAGACAACG | RDNANNSLH | CGGGAATCCGGGGTCCCTGACC | TLTISSLQAED |
| | CCAACAACTCACTGCATCTGCAAAT | LQMKSLRAE | GATTCAGTGGCAGCGGGTCTG | VALYYCQQYY |
| | GAAAAGCCTGAGAGCCGAGGACAC | DTAVYYCAR | GGGCAGATTTCACTCTCACCAT | ENPTFGQGT |
| | GGCTGTCTATTACTGTGCGAGAGTT | VGGGDYYDS | CAGCAGCCTGCAGGCTGAAGAT | KVEIK |
| | GGGGGCGGTGATTATTATGACAGT | SGYYWLDT | GTGGCACTTTATTACTGTCAGC | |
| | AGTGGTTACTACTGGCTCGATACCT | WGQGTLVT | AATATTATGAGAATCCGACATT | |
| | GGGGCCAGGGAACCCTGGTCACCG | VSS | CGGCCAAGGGACCAAGGTGGA | |
| | TCTCCTCA | | AATCAAA | |
| T15 | GAGGTGCAGCTGGTGGAGTCTGGG | EVQLVESGG | GACATCGTGATGACCCAGTCTC | DIVMTQSPV |
| | GGAGGCTTGGTCCAGCCTGGGGGG | GLVQPGGSL | CAGTCTCCCTGGCTGTGTCTCTG | SLAVSLGERA |
| | TCCCTGAGACTCTCCTGTGCAGCCTC | RLSCAASGFT | GGCGAGAGGGCCACCATCAAC | TINCKSSQSV |
| | TGGATTCACCTTTAATAACTATTGGA | FNNYWMT | TGCAAGTCCAGCCAGAGTGTTT | LYNSNNKNYL |
| | TGACCTGGGTCCGCCAGGCTCCAGG | WVRQAPGK | TATACAACTCCAACAATAAGAA | AWYQQKPG |
| | GAAGGGGCTGGAGTGGGTGGCCAA | GLEWVANV | CTATTTAGCTTGGTACCAGCAG | QPPKVLIYWA |
| | CGTAAACCAAGATGGCAATGAAAA | NQDGNEKN | AAACCAGGACAGCCTCCTAAGG | STRESGVPDR |
| | AAACTATGTGGACTCTGTGAGGGG | YVDSVRGRF | TGCTCATTTACTGGGCATCTACC | FSGSGSGTDF |
| | CCGATTCACCATCTCCAGAGACAAC | TISRDNAKNS | CGGGAATCCGGGGTCCCTGACC | TLTISSLQAED |
| | GCCAAGAACTCACTGCATCTGCAAA | LHLQMKSLIS | GATTCAGTGGCAGCGGGTCTG | VAVYFCQQY |
| | TGAAAAGCCTGATATCCGACGACAC | DDTAVYYCA | GGACAGATTTCACTCTCACCATC | YDTPTFGQG |
| | GGCTGTGTATTATTGTGCGAGAGTT | RVGGGDYYD | AGCAGCCTGCAGGCTGAAGAT | TKVEIK |
| | GGGGGCGGTGATTATTATGACAGT | SSGYYWFDT | GTGGCAGTTTATTTCTGTCAGC | |
| | AGTGGTTATTACTGGTTCGATACCT | WGQGALVT | AATATTATGATACTCCGACATTC | |
| | GGGGCCAGGGAGCCCTGGTCACCG | VSS | GGCCAAGGGACCAAGGTGGAA | |
| | TCTCCTCA | | ATCAAA | |
| T1 | GAGGTGCAGCTGGTGGAGTCTGGG | EVQLVESGG | GCCATCCAGATGACCCAGTCTC | AIQMTQSPSS |
| | GGAGGCCTGGAGCAGCCAGGGCG | GLEQPGRSLR | CATCCTCCCTGTCTGCATCTGTA | LSASVGDRVT |
| | GTCCCTGAGACTCTCCTGTGCAGCT | LSCAASGFSF | GGAGACAGGGTCACCATCACTT | ITCRASQFIRN |
| | TCTGGATTCAGTTTTGGTGATAATG | GDNAMTWF | GCCGGGCAAGTCAGTTCATTAG | DLGWYQQKP |
| | CTATGACCTGGTTCCGCCAGGCTCC | RQAPGKGLE | AAATGATTTAGGCTGGTATCAG | GRAPKLLIYA |
| | AGGGAAGGGGCTGGAGTGGGTCG | WVGIIRAKG | CAGAAACCAGGGAGAGCCCCT | ASGLHSGVP |
| | GGATCATTAGAGCCAAGGGTTATG | YGGTTEYAAS | AAGCTCCTGATCTATGCTGCATC | ARFSGNGSA |
| | GTGGGACAACAGAATACGCCGCGT | VKGRFTISRD | CGGGTTACACAGTGGGGTCCCA | TDFTLTITSLQ |
| | CTGTGAAAGGCAGATTCACCATCTC | DSKSIAFLQM | GCAAGGTTCAGCGGCAATGGA | PEDFATYYCL |
| | AAGAGATGATTCCAAAAGCATCGCC | NNVKVEDTG | TCTGCCACAGATTTCACTCTCAC | QDYNFPWTF |
| | TTTCTCCAAATGAACAACGTGAAAG | VYYCAKYASG | CATCACCAGCCTGCAGCCTGAA | GQGTKVEIK |
| | TCGAGGACACAGGCGTCTATTATTG | WEVGFDPW | GATTTTGCAACTTATTACTGTCT | |
| | TGCTAAATATGCCAGTGGCTGGGA | GQGTLVTVS | ACAAGATTACAATTTCCCGTGG | |
| | GGTTGGATTCGACCCCTGGGGCCA | S | ACGTTCGGCCAAGGGACCAAG | |
| | GGGAACCCTGGTCACCGTCTCGTCA | | GTGGAAATCAAA | |
| T2 | CAGGTGCAGCTGGTGCAGTCTGGA | QVQLVQSGA | GACATCCAGATGACCCAGTCTC | DIQMTQSPS |
| | GCTGAGGTGAAGAAGCCTGGGGCC | EVKKPGASV | CTTCCACCCTGTCTGCATCTGTG | TLSASVGDRV |
| | TCAGTGAATGTCTCCTGCAAGGCTT | NVSCKASGY | GGAGACAGGGTCACCATCACTT | TITCRASQSIS |
| | CTGGTTACAATTTTAAGGCCTATGG | NFKAYGVG | GCCGGGCCAGTCAGAGTATAA | SWLAWYQQ |
| | TGTCGGCTGGGTGCGACAGGCCCCT | WVRQAPGQ | GTAGCTGGTTGGCCTGGTATCA | KAGKAPKLLIF |
| | GGACAAGGGCTTGAGTGGATGGGA | GLEWMGWI | GCAGAAAGCAGGGAAAGCCCC | DASTLQSGVP |
| | TGGATCACCCCTTACAATGGTAAAA | TPYNGKTNY | TAAGCTCCTGATCTTTGATGCCT | SRFSGSGSGT |
| | CAAACTATGCACAGAAGATCCAGG | AQKIQGRVT | CCACTTTGCAAAGTGGGGTCCC | EFSLTISSLQP |
| | GCAGAGTCACCATGACCACAGACAC | MTTDTSTTT | ATCAAGGTTCAGCGGCAGTGG | DDFATYYCQ |
| | GTCGACGACCACAGCCTACATGGAG | AYMELRSLQ | ATCTGGGACAGAATTCAGTCTC | QYYSYSTFGQ |
| | CTGAGGAGCCTGCAACCTGACGACA | PDDTAMYFC | ACCATCAGCAGCCTGCAGCCTG | GTKVEIR |
| | CGGCCATGTATTTCTGTGCAAGAAC | ARTPAALASE | ACGATTTTGCAACTTATTACTGC | |
| | CCCCGCTGCCCTGGCAAGTTTTGAC | DYWSQGTLV | CAACAGTATTATAGTTATTCGAC | |
| | TACTGGAGCCAGGGAACCCTGGTC | TVSS | GTTCGGCCAAGGGACCAAGGT | |
| | ACCGTCTCCTCA | | GGAAATCAGA | |
| T3 | GAGGTGCAGCTGGTGGAGTCTGGG | EVQLVESGG | GACATCCAGATGACCCAGTCTC | DIQMTQSPS |
| | GGAGGCTTGGTTCAGCCTGGGGGG | GLVQPGGSL | CTTCCACCCTGTCTGCATCTGTA | TLSASVGDRV |
| | TCCCTGAGACTCTCCTGTGCAGCCTC | RLSCAASGFT | GGAGACAGAGTCACCATCACTT | TITCRASQSV |
| | TGGATTCACCTATTTCAGCTATGCCA | YFSYAMTWV | GCCGGGCCAGTCAGAGTGTTTA | YRLLAWYQQ |
| | TGACCTGGGTCCGCCAGGCTCCAGG | RQAPGKGLE | TAGGTTGTTGGCCTGGTATCAG | KPGKAPKLLIY |
| | GAAGGGGCTGGAGTGGGTCTCATC | WVSSVNVRV | CAGAAACCAGGTAAAGCCCCTA | DAFKLKSGVP |
| | TGTTAATGTTCGTGTTGGTACCTCAT | GTSYYADSVK | AACTCCTGATCTATGATGCCTTC | PRFSGSGSGT |
| | ACTACGCAGACTCCGTGAAGGGCC | GRFTISSDISK | AAATTGAAAAGTGGGGTCCCAC | EFTLTISSLQT |
| | GGTTCACCATCTCCAGTGACATTTCC | NTVFLQMNS | CAAGGTTCAGCGGCAGTGGATC | DDFASYYCQ |
| | AAGAACACAGTGTTTCTGCAAATGA | LRADDTAIYY | TGGGACAGAATTCACTCTCACC | QYNSYSTFG |
| | ACAGCCTGAGAGCCGACGACACGG | CATVGATQD | ATCAGCAGCCTGCAGACTGATG | QGTKVEVK |
| | CCATATATTACTGTGCGACAGTGGG | LRYFDFWGQ | ATTTTGCAAGTTATTACTGCCAA | |
| | GGCTACCCAAGACCTTCGCTACTTT | GTLVTVSS | CAGTATAATAGTTATTCGACGTT | |
| | GACTTTTGGGGCCAGGGAACGTTG | | CGGCCAAGGGACCAAGGTGGA | |
| | GTCACCGTCTCCTCA | | AGTCAAA | |
| T4 | CAGGTTCAGCTGGTGCAGTCTGGA | QVQLVQSGG | GACATCCAGATGACCCAGTCTC | DIQMTQSPS |
| | GGTGAGGTGAAGAAGCCTGGGGCC | EVKKPGASA | CTTCCACCCTGTCTGCATCTGTG | TLSASVGDRV |
| | TCAGCGAAGGTCTCCTGCAAGACTT | KVSCKTSGFT | GGAGACCGAGTCACCATCACTT | TITCRASQIISS |
| | CTGGTTTCACCTTTACCAGTTATGGT | FTSYGISWVR | GCCGGGCCAGTCAGATTATTAG | WVAWYQQR |
| | ATCAGCTGGGTGCGACAGGCCCCT | QAPGQGLE | TAGCTGGGTGGCCTGGTATCAG | PGKAPKLLIF |
| | GGACAAGGGCTTGAGTGGATGGGG | WMGWINTY | CAGAGACCAGGGAAAGCCCCT | DASILESGVP |
| | TGGATCAACACTTACAATGGTAACA | NGNTKYAQR | AAGCTCCTGATCTTTGATGCCTC | SRFSGSATGT |
| | CAAAGTATGCACAGAGGCTCCAGG | LQGRVSMTT | CATTTTGGAAAGTGGGGTCCCA | EFSLTISGLQP |
| | GCAGAGTCTCCATGACCACAGACAC | DTSTNTAYM | TCAAGGTTCAGCGGCAGTGCAA | DDFATYYCQ |
| | ATCCACGAACACAGCCTACATGGAA | ELRSLRSDDT | CTGGGACAGAATTCAGTCTCAC | HYNDFPLSFG |
| | CTGAGGAGCCTGAGATCTGACGAC | AVYYCARGQ | CATCAGCGGTCTGCAGCCTGAT | GGTKVEIK |
| | ACGGCCGTTTATTACTGTGCGCGAG | GRYGDYIYN | GATTTTGCAACTTATTACTGCCA | |
| | GCCAAGGACGGTACGGTGACTACA | HWGQGTLVI | ACACTATAATGATTTTCCCCTCA | |
| | TTTATAATCACTGGGGCCAGGGAAC | VSS | GTTTCGGCGGAGGGACCAAGG | |
| | CCTGGTCATCGTCTCCTCA | | TGGAGATCAAA | |
| T5 | CAGGTGCAGCTGGTGGAGTCTGGG | QVQLVESGG | GACATCCAGATGACCCAGTCTC | DIQMTQSPS |
| | GGAGGCGTGGTCCAGCCTGGGAGG | GVVQPGRSL | CATCCTCCCTGTCTGCATCTGTA | SLSASVGDRV |
| | TCCCTGAGACTCTCCTGTGCAGCCTC | RLSCAASGFT | GGAGACAGAGTCACCATCACTT | TITCQASQDI |
| | TGGATTCACCTTCAGTAGCCATGCT | FSSHAMHW | GCCAGGCGAGTCAGGACATTA | SNYLNWYQQ |
| | ATGCACTGGGTCCGCCAGGCTCCAG | VRQAPGKGL | GTAACTATTTAAATTGGTATCA | KPGKAPKVLI |
| | GCAAGGGGCTGCAGTGGGTGGCAC | QWVALISYD | GCAGAAACCAGGGAAAGCCCC | YDASNLERGV |
| | TTATATCATATGATGGATACAATAA | GYNKYYAES | TAAGGTCCTGATCTACGATGCA | PSRFSGSGSG |
| | ATACTACGCAGAGTCCGTGCAGGG | VQGRFTLSR | TCCAATTTGGAAAGAGGGGTCC | TAFTFTISSLQ |
| | CCGATTCACCCTCTCCAGAGACAATT | DNSKNTLYL | CATCAAGGTTCAGTGGAAGTGG | PEDIATYYCQ |
| | CCAAGAACACGCTGTATCTGCAAAT | QMNSLRAED | ATCTGGGACAGCTTTTACTTTCA | QYDNLPSFG |
| | GAACAGCCTGAGAGCTGAGGACAC | TAVYYCARD | CCATCAGCAGCCTGCAGCCTGA | GGTKVEIK |
| | GGCTGTCTATTACTGTGCGAGAGAT | RDSSGYIFDY | AGATATTGCAACATATTACTGC | |
| | CGCGATAGTAGTGGTTATATTTTTG | WGQGTLVT | CAACAGTATGATAATCTCCCTTC | |
| | ACTACTGGGGCCAGGGAACCCTGG | VSS | TTTCGGCGGAGGGACCAAGGT | |
| | TCACCGTCTCCTCA | | GGAGATCAAA | |
| T6 | CAGGTACAGCTGCAGCAGTCAGGT | QVQLQQSGP | GAAATAGTGATGACGCAGTCTC | EIVMTQSPAT |
| | CCAGGACTGGTGAAGCCCTCGCAG | GLVKPSQTLS | CAGCCACCCTGTCTGTGTCTCCA | LSVSPGDRAT |
| | ACCCTCTCACTCACCTGTGCCATCTC | LTCAISGDSV | GGGGACAGAGCCACCCTCTCCT | LSCRASQSVS |
| | CGGGGACAGTGTCTCTAGCAACAGT | SSNSAAWN | GCAGGGCCAGTCAGAGCGTAA | NRLAWYQQK |
| | GCTGCTTGGAACTGGATCAGGCAGT | WIRQSPSRG | GCAACAGGTTGGCCTGGTACCA | PGQAPRLLIY |
| | CCCCATCGAGAGGCCTTGAGTGGCT | LEWLGRTYY | GCAGAAACCTGGCCAGGCTCCC | DASTRATDIP |
| | GGGAAGGACATATTACAGGTCCAG | RSRWYSDYA | AGGCTCCTCATCTATGATGCATC | ARFSGSGSGT |
| | GTGGTATAGTGATTATGCATTCTCT | FSVRSRIIVKA | CACCAGGGCCACTGATATCCCA | EFTLTISSLQS |
| | GTGAGAAGTCGAATAATCGTCAAG | DTSRNLFSLQ | GCCAGATTCAGTGGCAGTGGGT | EDFAIYFCHQ |
| | GCAGACACATCTAGGAACCTATTCT | LNSMTPEDT | CTGGGACAGAATTCACTCTCAC | YHNWPGFGP |
| | CCCTGCAGTTGAACTCTATGACTCCC | AIYYCARDLG | CATTAGCAGCCTGCAGTCTGAA | GTKLEIK |
| | GAGGACACGGCTATATATTACTGTG | IAAADWFDS | GACTTTGCAATTTATTTCTGTCA | |
| | CAAGAGATTTGGGTATAGCAGCGG | WGQGTLVT | CCAGTATCATAACTGGCCGGGT | |
| | CTGACTGGTTCGACTCGTGGGGCCA | VSS | TTTGGCCCGGGGACCAAGCTGG | |
| | GGGAACCCTGGTCACCGTCTCCTCA | | AGATCAAA | |
| T7 | CAGGTGCAGCTGGTGGAGTCTGGG | QVQLVESGG | GATATTGTGATGACTCAGTCTC | DIVMTQSPLS |
| | GGAGGCGTGGTCCAGCCTGGGAGG | GVVQPGRSL | CACTCTCCCTGCCCGTCACCCCT | LPVTPGEPASI |
| | TCCCTGAAACTCTCCTCTGTAGCCTC | KLSSVASGFIF | GGAGAGCCGGCCTCCATCTCCT | SCKSSQSLLLS |
| | TGGATTCATCTTCAATATGTATGGC | NMYGMHW | GCAAGTCTAGTCAGAGCCTCCT | NGYNYLDWY |
| | ATGCACTGGGTCCGCCAGGCTCCAG | VRQAPGKGL | TCTTAGTAATGGATACAACTATT | LQKPGQSPQ |
| | GCAAGGGGCTGGAATGGGTGGCTG | EWVAVISYD | TGGATTGGTACCTGCAGAAGCC | LLITLASDRAS |
| | TAATATCATATGATGGCACCAAAGA | GTKEFYADSV | AGGGCAGTCTCCACAGCTCCTG | GVPDRFSGS |
| | ATTCTACGCAGACTCCGTGAAGGGA | KGRFAISRDD | ATCACTCTGGCCTCTGATCGGG | GSGTELQLKI |
| | CGATTCGCTATCTCCAGAGACGATT | SKNTLSLQM | CCTCCGGGGTCCCTGACAGGTT | GRVEAEDVG |
| | CCAAGAACACTCTGTCTCTGCAAAT | NSLRPEDTAV | CAGTGGCAGTGGATCAGGCAC | VYYCMQGLQ |
| | GAACAGCCTGAGACCTGAAGACAC | YYSARSPSGH | AGAATTACAACTGAAAATCGGC | TPFTFGPGTK |
| | GGCTGTGTATTATTCTGCGAGGAGC | ALDLWGQG | AGAGTGGAGGCTGAGGATGTT | VEIK |
| | CCCAGTGGGCATGCTCTTGATCTCT | TVVTVSS | GGGGTTTATTACTGCATGCAAG | |
| | GGGGCCAAGGGACAGTGGTCACCG | | GTCTACAAACCCCTTTCACTTTC | |
| | TCTCTTCA | | GGCCCTGGGACCAAAGTGGAG | |
| | | | ATCAAA | |
| T8 | CAGGTGCAGCTGCAGGAGTCGGGC | QVQLQESGP | GACATCCAGATGACCCAGTCTC | DIQMTQSPS |
| | CCAGGACTGGTGAAGCCTTCGGAG | GLVKPSETLS | CATCCACCCTGTCTGCATCTGTA | TLSASVGDRV |
| | ACCCTGTCCCTCAGCTGCGCTGTCTC | LSCAVSGGSI | GGAGACAGAGTCACCATCACTT | TITCRASQSIS |
| | TGGCGGCTCCATCAGAGGTTATTCC | RGYSWSWIR | GCCGGGCCAGTCAGAGTATTAG | TWLAWYQQ |
| | TGGAGCTGGATCCGGCAGCCCCCC | QPPGKGLEW | TACCTGGTTGGCCTGGTATCAG | KPGKAPKLLIY |
| | GGGAAGGGACTGGAGTGGATTGCT | IADMLYTGTT | CAGAAACCAGGGAAAGCCCCTA | KASSLESGVP |
| | GATATGTTATATACTGGGACCACCA | TFNPSLKSRV | AACTCCTGATCTATAAGGCGTC | SRFSGSGSGT |
| | CCTTCAACCCATCCCTCAAGAGTCG | TISVDTSKNQ | TAGTTTAGAAAGTGGGGTCCCA | EFTLTISSLQP |
| | AGTCACCATATCGGTAGACACGTCC | FSLKLTSVTA | TCAAGGTTCAGCGGCAGTGGAT | DDLATYYCQ |
| | AAGAACCAGTTCTCCCTGAAGCTGA | ADTAVYYCA | CTGGGACAGAATTCACTCTCAC | QYYNYGITFG |
| | CCTCTGTGACCGCTGCGGACACGGC | RGPTVSGPIV | CATCAGCAGCCTGCAGCCTGAT | PGTKVDIQ |
| | CGTGTATTACTGTGCGAGGGGACC | VDYWGPGTL | GATCTTGCAACCTATTACTGCCA | |
| | GACTGTGTCAGGGCCCATAGTAGTT | VTVSS | ACAGTATTATAATTACGGGATC | |
| | GACTACTGGGGCCCGGGAACCCTG | | ACTTTCGGCCCTGGGACCAAAG | |
| | GTCACCGTCTCCTCA | | TGGATATCCAA | |
| T10 | CAGGTTCAGCTGGTGCAGTCTGGA | QVQLVQSGD | GACATCCAGATGACCCAGTCTC | DIQMTQSPS |
| | GATGAGGTGAAGGAGCCTGGGGCC | EVKEPGASVK | CATCCTCCCTGTCTGCATCTGTT | SLSASVGHRV |
| | TCAGTGAAGGTCTCCTGCAAGGCTT | VSCKASGYSF | GGACACAGAGTCACCATCACTT | TITCRASQTIS |
| | CTGGTTACAGCTTTACCAGGTATGG | TRYGITWVRL | GCCGGGCAAGTCAGACCATTAG | NHLNWFQQ |
| | AATCACCTGGGTGCGACTGGCCCCT | APGQGLEW | CAACCATTTAAATTGGTTTCAGC | KPGKAPKLLIY |
| | GGACAAGGGCTTGAGTGGATGGGA | MGWISAYN | AGAAGCCAGGGAAAGCCCCTA | AASRLQTGV |
| | TGGATCAGCGCTTACAATGGTGACA | GDTNLAQKF | AACTCCTGATTTATGCTGCATCC | PSRFSGSGSG |
| | CAAACTTAGCACAGAAGTTCCAGGG | QGRVTMTT | AGGTTGCAAACTGGGGTCCCAT | TDFTLTISSLQ |
| | CAGAGTCACCATGACCACAGACACA | DTSTTTAYM | CAAGGTTCAGTGGCAGTGGATC | PEDFATYFCQ |
| | TCCACGACCACAGCCTACATGGAGA | EMRNLRSDD | TGGGACAGATTTCACTCTCACC | QTYDIPPYTF |
| | TGAGGAACCTGAGATCTGACGACAC | TAVYYCGRD | ATCAGCAGTCTGCAACCTGAAG | GQGTKLEIK |
| | GGCCGTTTATTACTGTGGGAGAGAT | TRYCSGAGC | ATTTTGCGACTTACTTCTGTCAA | |
| | ACCCGATATTGTAGTGGTGCTGGCT | PRPSWYYYY | CAGACTTACGATATCCCTCCATA | |
| | GCCCCCGGCCAAGTTGGTACTACTA | MDVWGKGT | CACTTTTGGCCAGGGGACCAAG | |
| | CTACATGGACGTCTGGGGCAAAGG | TVTVSS | CTGGAGATCAAA | |
| | GACCACGGTCACCGTCTCCTCA | | | |
| T11 | CAGGTGCAGCTGGTGGAGTCGGGG | QVQLVESGG | GACATCCAGATGACCCAGTCTC | DIQMTQSPS |
| | GGAGGCGTGGTCCAGCCTGGGGGG | GVVQPGGSL | CATCCTCCCTGTCGGCATCTGTA | SLSASVGDKV |
| | TCCCTCAGACTCTCCTGTGGAGCGT | RLSCGASGFT | GGAGACAAAGTCACCATCACTT | TITCRASQDIS |
| | CCGGTTTCACCTTCAGTAGATATGG | FSRYGMHW | GCCGGGCGAGTCAGGACATTA | NYLAWFQQK |
| | CATGCACTGGGTCCGCCAGGCTCCA | VRQAPGKGL | GCAATTATTTAGCCTGGTTTCAG | PGKVPKLLIY |
| | GGCAAGGGGCTGGAGTGGGTGTCA | EWVSFIRYD | CAGAAACCAGGGAAAGTTCCCA | GASTLLSGVP |
| | TTTATACGGTATGATGGAACTGAAA | GTEKYYADSV | AGCTCCTGATCTATGGTGCATC | SRFSGSQSGT |
| | AATACTATGCGGACTCCGTGAAGGG | KGRFTISRDN | CACTTTGCTGTCAGGGGTCCCA | NFTLIISSLQP |
| | CCGATTCACCATCTCCAGAGACAAT | SKNTLYLQM | TCTCGGTTCAGTGGCAGTCAAT | EDVATYYCQK |
| | TCCAAGAACACGCTGTATCTGCAAA | NSLRADDAA | CTGGGACAAATTTCACTCTCATC | HNSAPWTFG |
| | TGAATAGCCTGAGAGCTGACGACG | VYFCANPYIT | ATCAGCAGCCTGCAGCCTGAAG | QGTKVEIK |
| | CGGCTGTATATTTCTGTGCGAATCCT | PPTNDYWG | ATGTTGCAACTTATTACTGTCAA | |
| | TATATAACGCCGCCTACTAATGACT | QGTLVTVSS | AAACATAACAGTGCCCCGTGGA | |
| | ACTGGGGCCAGGGAACCCTGGTCA | | CGTTCGGCCAAGGGACCAAGG | |
| | CCGTCTCCTCA | | TGGAAATCAAA | |
| T17 | GAGGTGCAGCTGGTGGAGTCTGGG | EVQLVESGG | GACATCCAGATGACCCAGTCTC | DIQMTQSPS |
| | GGAGGCTTGGTGCAGCCGGGGGG | GLVQPGGSL | CTTCCACCCTGTCTGCTTCTGTA | TLSASVGDRV |
| | GTCCCTGACACTCTCCTGTACAGCCT | TLSCTASGFT | GGAGACAGAGTCACCATCACTT | TITCRASQSV |
| | CTGGATTCACCTACTTCGGCTTTGCC | YFGFAMTW | GCCGGGCCAGTCAGAGTGTTTA | YRWLAWYQ |
| | ATGACCTGGGTCCGCCAACCTCCAG | VRQPPGKGL | TAGGTGGTTGGCCTGGTATCAG | QKPGEAPKLL |
| | GGAAGGGGCTGGAGTGGATCTCAT | EWISSINLLS | CAGAAACCAGGTGAAGCCCCTA | VYGAFSLQSG |
| | CTATTAATCTTCTTTCTGGTACCACA | GTTYYVDSVK | AACTCCTGGTCTATGGTGCCTTC | VPPRFSGSGY |
| | TACTATGTGGACTCGGTGAAGGGCC | GRFTISRDNS | AGTTTGCAGAGTGGGGTCCCAC | GTEFTLTISSL |
| | GCTTCACCATCTCCAGAGACAATTCC | KNTVFLQMK | CGAGGTTCAGCGGCAGTGGAT | QPDDFATYY |
| | AAGAATACAGTGTTTCTGCAAATGA | GLTAEDTAVY | ATGGGACAGAATTCACTCTCAC | CQQYNSHST |
| | AAGGACTGACAGCCGAGGACACGG | YCAKVGATQ | CATCAGCAGCCTGCAGCCTGAT | FGQGTKVEV |
| | CCGTTTATTACTGTGCGAAAGTTGG | DLHYFDFWG | GATTTTGCAACTTATTATTGCCA | K |
| | AGCTACCCAGGACCTTCACTACTTT | QGTLVTVSS | ACAATATAATAGTCATTCGACG | |
| | GACTTCTGGGGCCAGGGAACCCTG | | TTCGGCCAAGGGACCAAGGTG | |
| | GTCACCGTCTCCTCA | | GAAGTCAAA | |
| T3-16-17 | GAGGTGCAGCTGGTGGAGTCTGGG | EVQLVESGG | GACATCCAGATGACCCAGTCTC | DIQMTQSPS |
| MRCA1 | GGAGGCTTGGTACAGCCTGGGGGG | GLVQPGGSL | CTTCCACCCTGTCTGCATCTGTA | TLSASVGDRV |
| | TCCCTGAGACTCTCCTGTGCAGCCTC | RLSCAASGFT | GGAGACAGAGTCACCATCACTT | TITCRASQSV |
| | TGGATTCACCTTTTTCAGCTATGCCA | FFSYAMTWV | GCCGGGCCAGTCAGAGTGTTA | SRWLAWYQ |
| | TGACCTGGGTCCGCCAGGCTCCAGG | RQAPGKGLE | GTAGGTGGTTGGCCTGGTATCA | QKPGKAPKLL |
| | GAAGGGGCTGGAGTGGGTCTCATC | WVSSINVRG | GCAGAAACCAGGTAAAGCCCCT | IYDAFSLESGV |
| | TATTAATGTTCGTGGTGGTACCACA | GTTYYADSVK | AAACTCCTGATCTATGATGCCTT | PPRFSGSGSG |
| | TACTACGCAGACTCCGTGAAGGGCC | GRFTISRDNS | CAGTTTGGAAAGTGGGGTCCCA | TEFTLTISSLQ |
| | GGTTCACCATCTCCAGAGACAATTC | KNTVFLQMN | CCAAGGTTCAGCGGCAGTGGAT | PDDFATYYC |
| | CAAGAACACAGTGTTTCTGCAAATG | SLRAEDTAVY | CTGGGACAGAATTCACTCTCAC | QQYNSYSTF |
| | AACAGCCTGAGAGCCGAGGACACG | YCAKVGATQ | CATCAGCAGCCTGCAGCCTGAT | GQGTKVEVK |
| | GCCGTATATTACTGTGCGAAAGTGG | DLRYFDYWG | GATTTTGCAACTTATTACTGCCA | |
| | GGGCTACCCAAGACCTTCGCTACTT | QGTLVTVSS | ACAGTATAATAGTTATTCGACG | |
| | TGACTACTGGGGCCAGGGAACCCT | | TTCGGCCAAGGGACCAAGGTG | |
| | GGTCACCGTCTCCTCA | | GAAGTCAAA | |
| T3-16-17 | GAGGTGCAGCTGGTGGAGTCTGGG | EVQLVESGG | GACATCCAGATGACCCAGTCTC | DIQMTQSPS |
| MRCA2 | GGAGGCTTGGTACAGCCTGGGGGG | GLVQPGGSL | CTTCCACCCTGTCTGCATCTGTA | TLSASVGDRV |
| | TCCCTGAGACTCTCCTGTGCAGCCTC | RLSCAASGFT | GGAGACAGAGTCACCATCACTT | TITCRASQSV |
| | TGGATTCACCTTTTTCAGCTATGCCA | FFSYAMTWV | GCCGGGCCAGTCAGAGTGTTA | SRWLAWYQ |
| | TGACCTGGGTCCGCCAGGCTCCAGG | RQAPGKGLE | GTAGGTGGTTGGCCTGGTATCA | QKPGKAPKLL |
| | GAAGGGGCTGGAGTGGGTCTCATC | WVSSINVLG | GCAGAAACCAGGTAAAGCCCCT | IYDAFSLESGV |
| | TATTAATGTTCTTGGTGGTACCACAT | GTTYYADSVK | AAACTCCTGATCTATGATGCCTT | PPRFSGSGSG |
| | ACTACGCAGACTCCGTGAAGGGCC | GRFTISRDNS | CAGTTTGGAAAGTGGGGTCCCA | TEFTLTISSLQ |
| | GGTTCACCATCTCCAGAGACAATTC | KNTVFLQMN | CCAAGGTTCAGCGGCAGTGGAT | PDDFATYYC |
| | CAAGAACACAGTGTTTCTGCAAATG | SLRAEDTAVY | CTGGGACAGAATTCACTCTCAC | QQYNSYSTF |
| | AACAGCCTGAGAGCCGAGGACACG | YCAKVGATQ | CATCAGCAGCCTGCAGCCTGAT | GQGTKVEVK |
| | GCCGTTTATTACTGTGCGAAAGTAG | DLHYFDYWG | GATTTTGCAACTTATTACTGCCA | |
| | GGGCTACCCAAGACCTTCACTACTT | QGTLVTVSS | ACAGTATAATAGTTATTCGACG | |
| | TGACTACTGGGGCCAAGGAACCCT | | TTCGGCCAAGGGACCAAGGTG | |
| | GGTCACCGTCTCCTCA | | GAAGTCAAA | |
| T3-16-17 | GAGGTGCAGCTGGTGGAGTCTGGG | EVQLVESGG | GACATCCAGATGACCCAGTCTC | DIQMTQSPS |
| GL | GGAGGCTTGGTACAGCCTGGGGGG | GLVQPGGSL | CTTCCACCCTGTCTGCATCTGTA | TLSASVGDRV |
| | TCCCTGAGACTCTCCTGTGCAGCCTC | RLSCAASGFT | GGAGACAGAGTCACCATCACTT | TITCRASQSIS |
| | TGGATTCACCTTTAGCAGCTATGCC | FSSYAMSWV | GCCGGGCCAGTCAGAGTATTAG | SWLAWYQQ |
| | ATGAGCTGGGTCCGCCAGGCTCCA | RQAPGKGLE | TAGCTGGTTGGCCTGGTATCAG | KPGKAPKLLIY |
| | GGGAAGGGGCTGGAGTGGGTCTCA | WVSAISGSG | CAGAAACCAGGGAAAGCCCCTA | DASSLESGVP |
| | GCTATTAGTGGTAGTGGTGGTAGCA | GSTYYADSVK | AGCTCCTGATCTATGATGCCTCC | SRFSGSGSGT |
| | CATACTACGCAGACTCCGTGAAGGG | GRFTISRDNS | AGTTTGGAAAGTGGGGTCCCAT | EFTLTISSLQP |
| | CCGGTTCACCATCTCCAGAGACAAT | KNTLYLQMN | CAAGGTTCAGCGGCAGTGGATC | DDFATYYCQ |
| | TCCAAGAACACGCTGTATCTGCAAA | SLRAEDTAVY | TGGGACAGAATTCACTCTCACC | QYNSYSTFG |
| | TGAACAGCCTGAGAGCCGAGGACA | YCAKVGATQ | ATCAGCAGCCTGCAGCCTGATG | QGTKVEIK |
| | CGGCCGTATATTACTGTGCGAAAGT | DLRYFDYWG | ATTTTGCAACTTATTACTGCCAA | |
| | GGGAGCTACCCAAGACCTTCGCTAC | QGTLVTVSS | CAGTATAATAGTTATTCGACGTT | |
| | TTTGACTACTGGGGCCAGGGAACCC | | CGGCCAAGGGACCAAGGTGGA | |
| | TGGTCACCGTCTCCTCA | | AATCAAA | |
| T18 | GAGGTGCAGCTGGTGGAGTCTGGG | EVQLVESGG | GACATCCAGATGACCCAGTCTC | DIQMTQSPS |
| | GGAGGCTTGGTCCAGCCTGGGGGG | GLVQPGGSL | CATCCTCCCTGTCTGCATCTGTA | SLSASVGDRV |
| | TCCCTGAGACTCTCCTGTGCAGCCTC | RLSCAASGFT | GGAGACAGAGTCACCATCACTT | TITCRASQSIA |
| | TGGATTCACCTTTAGTAGCTATTGG | FSSYWMTW | GCCGGGCTAGTCAGAGCATTGC | SYLNWYQQK |
| | ATGACCTGGGTCCGCCAGGCTCCAG | VRQAPGKGL | CAGCTATTTAAATTGGTATCAG | PGRAPKLLIY |
| | GGAAGGGGCTGGAGTGGGTGGCC | EWVANIKPD | CAGAAACCAGGGAGAGCCCCT | AASTSGVPSR |
| | AATATAAAGCCAGATGGAAGTGAG | GSEKNYVDS | AAGCTCCTGATCTATGCTGCATC | FSGSGSGTDF |
| | AAAAACTATGTGGACTCTGTGAAGG | VKGRFTISRD | CACAAGTGGGGTCCCATCAAGG | TLTISSLQPED |
| | GCCGATTCACCATCTCCAGAGACAA | NAENSLYLQ | TTCAGTGGCAGTGGCTCTGGTA | FATYYCQQSY |
| | CGCCGAGAACTCACTGTATCTGCAA | MNSLRAEDT | CAGATTTCACTCTCACCATCAGC | TTPRTFGQGT |
| | ATGAACAGCCTGAGAGCCGAGGAC | AVFYCARDLR | AGTCTGCAACCTGAAGATTTTG | KVEIK |
| | ACGGCTGTATTTTACTGTGCGAGAG | YCSSTSCSPA | CAACTTACTACTGTCAACAGAG | |
| | ACCTAAGATATTGTAGTAGTACTAG | LDYWGQGTL | TTACACTACCCCCCGGACGTTC | |
| | CTGTTCACCAGCTCTTGACTACTGG | LTVSS | GGCCAAGGGACCAAGGTGGAA | |
| | GGCCAGGGAACCCTGCTCACCGTCT | | ATCAAA | |
| | CCTCA | | | |
| T19 | GAGGTGCAGCTGTTGGAGTCTGGG | EVQLLESGG | GAAATTGTGTTGACACAGTCTC | EIVLTQSPATL |
| | GGAGGCTTGGTACAGCCGGGGGGG | GLVQPGGSL | CAGCCACCCTGTCTTTGTCTCCA | SLSPGDRATL |
| | TCCCTGAGACTCTCCTGTGCAGCCTC | RLSCAASGFT | GGGGACAGAGCCACCCTCTCCT | SCRASQSVNT |
| | TGGATTCACCTTTGACAATTATGGC | FDNYGMSW | GCAGGGCCAGTCAGAGTGTTA | YLAWYQHKP |
| | ATGAGTTGGGTCCGCCAGGCTCCAG | VRQAPGKGL | ACACCTACTTAGCCTGGTACCA | GQAPRLLIYD |
| | GGAAGGGGCTGGAGTGGGTCTCAG | EWVSVISSG | ACACAAACCTGGCCAGGCTCCC | ASKRATGIPA |
| | TTATTAGCAGTGGTGGTGTTGGCAC | GVGTYYADS | AGGCTCCTCATCTATGATGCATC | RFSGSGSGTD |
| | ATACTACGCAGACTCCGTGAAGGGC | VKGRFIISRD | CAAGAGGGCCACTGGCATCCCA | FTLTISSLEPE |
| | CGGTTCATCATCTCCAGAGACAATG | NAKNTLYLQ | GCCAGGTTCAGTGGCAGTGGG | DFAVYYCQQ |
| | CCAAGAACACACTGTATCTGCAAAT | MNSLRAEDT | TCTGGGACAGACTTCACTCTCA | RANRPPLTFG |
| | GAACAGCCTGAGAGCCGAGGACAC | AVYYCAKDLL | CCATCAGCAGCCTAGAGCCTGA | GGTKVEIK |
| | GGCCGTATATTATTGTGCGAAAGAT | RYESSGYSP | AGATTTTGCAGTTTATTACTGTC | |
| | TTATTACGTTATGAGAGTAGTGGCT | WGQGTLVT | AGCAGCGTGCCAACAGGCCTCC | |
| | ATAGCCCGTGGGGCCAGGGAACCC | VSS | GCTCACTTTCGGCGGAGGGACG | |
| | TGGTCACCGTCTCCTCC | | AAGGTGGAGATCAAA | |
| T20 | CAGGTGCAGCTGCAGGAGTCGGGC | QVQLQESGP | GAAATTGTGTTGACGCAGTCTC | EIVLTQSPGTL |
| | CCAGGACTACTGAAGCCTTCACAGA | GLLKPSQTLS | CAGGCACCCTGTCTTTGTCTCCA | SLSPGERATL |
| | CCCTGTCTCTCACCTGCACTGTCTCT | LTCTVSGAFL | GGGGAAAGAGCCACCCTCTCCT | SCRASQSVTS |
| | GGTGCCTTCCTCAACAGTGGTGGTT | NSGGYYWT | GCAGGGCCAGTCAGAGTGTTAC | NFVGWYQQ |
| | ACTACTGGACCTGGATCCGCCAGCA | WIRQHPGKG | TAGCAACTTCGTAGGCTGGTAT | KPGQAPRLLI |
| | CCCAGGAAAGGGCCTGGAGTGGAT | LEWIGYIYYS | CAGCAGAAACCTGGCCAGGCTC | YAASSRPTGI |
| | TGGGTACATCTATTACAGTGGGACT | GTTYYNPSLK | CCAGGCTCCTCATCTATGCTGCA | PERFSGSGSG |
| | ACCTACTACAACCCGTCCCTCAAGA | SRVTISVDTS | TCCAGCAGGCCCACTGGCATCC | TDFTLTISRLE |
| | GTCGAGTTACCATTTCAGTGGACAC | KNQFSLNVN | CAGAGAGATTCAGTGGCAGTG | PEDFAVYYCE |
| | GTCTAAGAACCAGTTCTCCCTCAAC | SVIAADTAVY | GGTCTGGGACAGACTTCACTCT | QYGSSPRTFG |
| | GTGAACTCTGTGATTGCCGCGGACA | YCARAPRIPF | CACCATCAGCAGACTGGAGCCT | PGTKVEIK |
| | CGGCCGTGTATTACTGTGCGAGAGC | GEVIGGAAF | GAAGATTTTGCAGTGTATTACT | |
| | CCCGCGGATTCCTTTTGGGGAAGTT | DVWGQGTT | GTGAACAATATGGTAGCTCACC | |
| | ATAGGGGGTGCTGCTTTCGACGTCT | VIVSS | TCGGACGTTCGGCCCAGGGACC | |
| | GGGGCCAAGGGACAACGGTCATTG | | AAGGTGGAAATCAAA | |
| | TCTCTTCA | | | |
| T21 | CAGGTGCAGCTGCAGGAGTCGGGC | QVQLQESGP | GACATCCAGATGACCCAGTCTC | DIQMTQSPS |
| | CCAGGACTGGTGAAGCCTTCACAGA | GLVKPSQTLS | CATCCTCCCTGTCTGCATCTGTA | SLSASVGDRV |
| | CCCTGTCCCTCACCTGCACTGTCTCT | LTCTVSGGSF | GGGGACAGAGTCACCATCACTT | TITCRASQSIN |
| | GGTGGCTCCTTCAGTAATAGTAATT | SNSNYYWS | GCCGGGCAAGTCAGAGCATTA | NFLNWYQQK |
| | ATTACTGGAGTTGGATCCGGCAGCC | WIRQPAGKG | ACAACTTTTTAAATTGGTATCAA | PGKAPKLLIY |
| | CGCCGGGAAGGGACTGGAGTGGAT | LEWIGHIYAS | CAGAAGCCAGGGAAAGCCCCT | GASSLQSGVP |
| | AGGGCATATCTATGCCAGTGCGATC | AITHYNPSLK | AAGCTCCTGATCTATGGAGCAT | SRFSASGSGT |
| | ACCCACTACAATCCCTCCCTTAAGAG | SRVTISKDTS | CCAGTTTGCAGAGTGGGGTCCC | DFSLTISGLIP |
| | TCGAGTCACCATATCAAAAGACACG | KNQFSLNLRS | ATCACGGTTCAGTGCCAGTGGG | EDFAIYYCQQ |
| | TCCAAGAATCAGTTCTCCCTGAATCT | VTAADTAVY | TCTGGGACAGATTTCAGTCTCA | SDTTPWTFG |
| | GAGGTCTGTGACCGCCGCAGACAC | FCARGFRLAA | CCATCAGCGGTCTGATTCCTGA | QGTKVDIK |
| | GGCCGTGTATTTTTGTGCGAGAGGA | EAYYHGMDV | GGATTTTGCAATTTACTACTGTC | |
| | TTCCGTTTGGCAGCAGAAGCCTACT | WGQGTTVT | AACAGAGTGACACTACCCCGTG | |
| | ACCACGGCATGGACGTCTGGGGCC | VSS | GACGTTCGGCCAAGGGACCAA | |
| | AAGGGACCACCGTCACCGTCTCCTC | | GGTGGACATCAAG | |
| | A | | | |
| T22 | CAGGTGCAGCTGGTGCAGTCTGGG | QVQLVQSGA | GACATCGTGATGACCCAGTCTC | DIVMTQSPD |
| | GCTGAGGTGAAGAAGCCTGGGTCC | EVKKPGSSVK | CAGACTCCCTGGCTGTGTCTCT | SLAVSLGERA |
| | TCGGTGAAGGTCGCCTGCAAGGCTT | VACKASGGT | GGGCGAGAGGGCCACCATCAA | TIKCKSSQTIL |
| | CTGGAGGCACCTTCAGCAGGTCTGC | FSRSAISWVR | GTGCAAGTCCAGCCAGACTATT | YSSNNNNYL |
| | AATCAGCTGGGTGCGACAGGCCCCT | QAPGQGLE | TTATACAGCTCCAACAATAACA | AWYQQKPG |
| | GGACAAGGGCTTGAGTGGATGGGA | WMGGIIRIF | ACTACTTAGCTTGGTACCAGCA | QPPKLLIYWA |
| | GGGATCATCCGTATTTTTAATACAG | NTADYAQKF | GAAACCAGGACAGCCTCCTAAG | STRESGVPDR |
| | CGGACTACGCACAGAAGTTCCAGG | QGRVTMSA | CTGCTCATTTACTGGGCGTCTAC | FSGSGSGTDF |
| | GCAGAGTCACAATGTCCGCGGACG | DESTSTAYM | CCGGGAATCCGGGGTCCCTGAC | TLTISSLQAED |
| | AATCCACGAGCACAGCCTACATGGA | ELSSLRSEDT | CGATTCAGTGGCAGTGGGTCTG | VAVYYCQQY |
| | ATTGAGCAGCCTGAGATCTGAGGAC | AVYYCATSSL | GGACAGATTTCACTCTCACCATC | YSSPLTFGGG |
| | ACGGCCGTGTATTACTGTGCGACGT | SDIVVAEGAF | AGCAGCCTGCAGGCTGAAGAT | TKVEIK |
| | CCTCACTCAGCGATATTGTAGTGGC | VDHYFGMD | GTGGCAGTTTATTACTGTCAAC | |
| | GGAAGGTGCCTTTGTCGACCACTAC | VWGQGTTV | AATATTATAGTTCTCCCCTCACT | |
| | TTTGGTATGGACGTCTGGGGCCAAG | TVSS | TTCGGCGGAGGGACCAAAGTG | |
| | GGACCACGGTCACCGTCTCCTCA | | GAGATCAAA | |
| T23 | GAGGTGCAGCTGGTGGAGTCTGGG | EVQLVESGG | GACATCCAGATGACCCAGTCTC | DIQMTQSPS |
| | GGAGGCTTGGTACAGCCTGGAGGG | GLVQPGGSL | CATCCTCCCTGTCTGCATCTGTA | SLSASVGDRV |
| | TCCCTGAGACTCTCGTGTGTAGGCT | RLSCVGSAFT | GGAGACAGAGTCACCATCACTT | TITCRASQSIS |
| | CTGCATTCACCTTGAGTAGATATGA | LSRYEINWVR | GCCGGGCAAGTCAGAGCATTA | SYLNWYQQK |
| | AATAAACTGGGTCCGCCAGGCTCCA | QAPGKGLE | GCAGTTATTTAAATTGGTATCA | PGKAPKLLIY |
| | GGGAAGGGACTGGAGTGGATTTCA | WISHISSSGD | GCAGAAACCAGGGAAAGCCCC | AASTLQSGVP |
| | CACATAAGTAGTAGTGGGGATACCA | TINYADSVKG | TAAACTCCTGATCTATGCTGCAT | SRFSGSGSGT |
| | TAAATTACGCAGACTCTGTGAAGGG | RFTISRDNSK | CTACTTTGCAAAGTGGGGTCCC | DFTLTISSLQS |
| | CCGATTCACCATCTCCAGAGACAAC | NSNFLQMN | ATCAAGGTTCAGTGGCAGTGGA | EDFATYYCQ |
| | TCCAAGAACTCAAATTTTCTGCAAAT | RLRAEDTAVY | TCTGGGACAGATTTCACTCTCAC | QSFNTPRTFG |
| | GAACAGGCTGAGAGCCGAGGACAC | YCATWGLGY | CATCAGCAGTCTACAATCTGAA | QGTKVEIK |
| | GGCTGTTTATTACTGTGCGACATGG | CNSTGCYITD | GATTTTGCTACCTACTACTGTCA | |
| | GGACTGGGATATTGTAATAGTACCG | GMDVWGQ | ACAGAGCTTCAATACCCCTCGG | |
| | GCTGCTATATCACTGACGGTATGGA | GTTVTVSS | ACGTTCGGCCAAGGGACCAAG | |
| | CGTCTGGGGGCAAGGGACCACGGT | | GTGGAAATCAAA | |
| | CACCGTCTCCTCA | | | |
| T24 | CAGCTGCAGCTGCAGGAGTCCGGC | QLQLQESGS | TCCTATGAGCTGACACAGCCAA | SYELTQPTSV |
| | TCAGGACTGGTGAAGCCTTCACAGA | GLVKPSQTLS | CCTCGGTGTCAGTGTCCCCAGG | SVSPGQTARI |
| | CCCTGTCCCTCACCTGCGCTGTGTCT | LTCAVSGGSI | ACAGACGGCGAGGATCACCTG | TCSGDALPSQ |
| | GGTGGCTCCATCAGCAGTAGTGATT | SSSDYSWSW | CTCTGGAGATGCATTGCCGAGC | YAYWYQQKP |
| | ACTCCTGGAGCTGGATCCGGCAGCC | IRQPPGKGLE | CAATATGCTTATTGGTACCAGC | GQAPILIIYKD |
| | ACCAGGGAAGGGCCTGGAGTGGAT | WIGYIYHSG | AGAAGCCAGGCCAGGCCCCTAT | NKRPSGIPER |
| | TGGGTACATCTATCATAGTGGGAAC | NTYYNPSVKS | CTTAATAATATATAAAGACAAT | FSGSSSGTRV |
| | ACCTACTACAATCCGTCCGTCAAGA | RVTISLDSSK | AAGAGGCCCTCAGGGATCCCTG | TLTISGVQPE |
| | GTCGAGTCACCATTTCACTAGACAG | NHFSLTLTSV | AGCGATTCTCTGGCTCCAGCTC | DEADYYCQS |
| | CTCCAAGAACCACTTCTCCCTGACCC | TAADTAIYYC | AGGGACAAGAGTCACCTTGACC | ADRSGRYVF |
| | TGACCTCTGTGACTGCCGCAGACAC | ARDPGGNSG | ATCAGTGGAGTCCAGCCAGAG | GTGTRVPVL |
| | GGCCATATATTATTGTGCCAGGGAT | WFDPWGQG | GACGAGGCTGACTATTACTGTC | |
| | CCCGGTGGTAACTCCGGCTGGTTCG | ALVTVSS | AATCAGCAGACCGCAGTGGTCG | |
| | ACCCCTGGGGCCAGGGAGCCCTGG | | TTATGTCTTCGGGACTGGAACC | |
| | TCACCGTCTCCTCA | | AGGGTCCCCGTCCTA | |
| T25 | GAGGTGCAGCTGGTGGAGTCTGGG | EVQLVESGG | CAGTCTGTGCTGACTCAGCCAC | QSVLTQPPSA |
| | GGAGGCTTGGTAAAGCCTGGGGGG | GLVKPGGSLR | CCTCAGCGTCTGGGACCCCCGG | SGTPGQRVTI |
| | TCCCTTAGACTCTCCTGTGCACCCTC | LSCAPSGFIFS | GCAGAGGGTCACCATCTCTTGT | SCSGSTSNVG |
| | TGGATTCATTTTCAGTAATACATGG | NTWMSWVR | TCTGGAAGCACCTCCAACGTCG | TNYVYWCQ |
| | ATGAGCTGGGTCCGCCAGGCTCCA | QAPGKGLE | GAACTAATTATGTGTACTGGTG | QFPGAAPKLL |
| | GGGAAGGGGCTGGAGTGGGTTGG | WVGHIKSKA | CCAGCAGTTCCCAGGAGCGGCC | MYRNDRRPS |
| | CCACATTAAAAGCAAAGCTGATGGT | DGGTTDYAA | CCCAAACTCCTCATGTATAGGA | GVPDRFSGSK |
| | GGGACAACAGACTACGCTGCACCC | PVKGRFTISR | ATGATCGCCGGCCCTCAGGGGT | SGTSASLAISG |
| | GTGAAAGGCAGATTCACCATCTCCA | DDSRNTLYL | CCCTGACCGATTCTCTGGCTCCA | LRSEDEADYY |
| | GAGATGATTCAAGAAATACGCTATA | QMDSLKTED | AGTCTGGCACCTCAGCCTCCCT | CAAWDDSLS |
| | TCTACAAATGGACAGCCTGAAAACC | TGVYYCTTG | GGCCATCAGTGGGCTCCGGTCC | SWVFGGGTK |
| | GAGGACACAGGCGTGTATTACTGTA | WYSTLDYW | GAGGATGAGGCTGATTATTATT | LTVL |
| | CCACAGGCTGGTATTCCACCCTTGA | GQGTLVTVS | GTGCAGCATGGGATGACAGCCT | |
| | CTACTGGGGCCAGGGAACCCTGGT | S | GAGTAGTTGGGTGTTCGGCGG | |
| | CACCGTCTCCTCA | | AGGGACCAAGCTGACCGTCCTA | |
| T26 | CAGGTTCAGCTGGTGCAGTCTGGAT | QVQLVQSGS | CAGTCTGTGCTGACGCAGCCGC | QSVLTQPPSV |
| | CTGAGGTGAAGAAGCCTGGGGCCT | EVKKPGASV | CCTCAGTGTCTGGGGCCCCAGG | SGAPGQRVTI |
| | CAGTGAAGGTCTCCTGCAAGGCTTC | KVSCKASGYT | GCAGAGGGTCACCATCTCCTGC | SCTGSSSNIG |
| | TGGCTACACCTTTACCAGTAATGGT | FTSNGISWV | ACTGGGAGTAGTTCCAACATCG | AGYDVHWY |
| | ATCAGTTGGGTGCGACAGGCCCCTG | RQAPGQGLE | GGGCAGGTTATGATGTACACTG | QQLPGTAPKL |
| | GACAAGGGCTTGAGTGGATGGGAT | WMGWISAY | GTACCAGCAACTTCCAGGGACA | LIHGNSNRPS |
| | GGATCAGCGCTTACAATGGTAACAC | NGNTNYAQK | GCCCCCAAACTCCTCATCCATG | GVPDRFSGSK |
| | AAACTATGCACAGAAGTTCCAGGGC | FQGRVSLTTD | GTAACAGCAATCGGCCCTCAGG | SGTSASLAIT |
| | AGAGTCTCCTTGACCACAGACACAT | TSTSTAYMEL | GGTCCCTGACCGATTTTCTGGCT | GLQAEDEAD |
| | CCACGAGCACAGCCTACATGGAGCT | RNLTSDDTA | CCAAGTCTGGCACCTCAGCCTC | YYCQSFDSSL |
| | GCGGAACCTGACATCTGACGACACG | RYYCARSRG | CCTGGCCATCACTGGGCTCCAG | SGSVVFGGG |
| | GCCAGATATTACTGTGCGAGAAGCC | HYGDYLYGY | GCTGAGGATGAGGCTGATTATT | TKLTVL |
| | GAGGTCACTACGGTGACTACCTTTA | WGQGTLVT | ACTGCCAGTCCTTTGACAGCAG | |
| | TGGCTACTGGGGCCAGGGAACCCT | VSS | CCTGAGTGGTTCTGTGGTGTTC | |
| | GGTCACCGTCTCCTCA | | GGCGGAGGGACCAAACTGACC | |
| | | | GTCCTA | |
| T27 | GAAGTGCAGCTGGTGGAGTCTGGG | EVQLVESGG | CAGTCTGCCCTGACTCAGCCTG | QSALTQPASV |
| | GGAGGCTTGGTACAGCCTGGCAGG | GLVQPGRSL | CCTCCGTGTCTCGGTCTCCCGG | SRSPGQSITIS |
| | TCCCTGAGACTCTCCTGTGCAGCCTC | RLSCAASGFT | ACAGTCGATCACCATCTCCTGC | CTGTKNDVG |
| | TGGATTCACATTTGAAACTTATGCC | FETYAMHW | ACGGGAACCAAAAATGATGTTG | SYNHVSWYQ |
| | ATGCACTGGGTCCGACAAGCTCCAG | VRQAPGKGL | GAAGTTATAACCATGTCTCCTG | QHPGKGPKLI |
| | GGAAGGGCCTGGAGTGGGTCTCTG | EWVSGISFNS | GTACCAACAGCACCCAGGCAAG | IYEVNKRPSG |
| | GTATCAGTTTCAATAGTCGTAGTAG | RSRGYADAV | GGCCCCAAACTCATAATTTATG | VSDRFSGSKS |
| | AGGCTATGCGGACGCTGTGAGGGG | RGRFTISRDN | AGGTCAATAAGCGGCCCTCAGG | GNTASLTISG |
| | CCGATTCACCATCTCCAGAGACAAC | SKNSLFLEM | AGTTTCTGATCGCTTCTCTGGCT | LQAEDEAYYS |
| | TCCAAGAACTCCCTCTTTCTGGAAAT | NSLRPEDTAL | CCAAGTCTGGCAACACGGCCTC | CSSYAPMTTL |
| | GAATAGTCTGAGACCTGAGGACAC | YYCARDEKW | CCTGACAATCTCTGGGCTCCAG | VFGGGTKLTV |
| | GGCCTTGTATTATTGTGCAAGAGAT | GTPSDWGQ | GCTGAGGACGAGGCTTATTATT | L |
| | GAGAAATGGGGGACTCCGAGTGAC | GVLVTVSS | CCTGCTCCTCATATGCACCTATG | |
| | TGGGGCCAGGGAGTCCTGGTCACC | | ACCACTTTAGTGTTCGGCGGAG | |
| | GTCTCCTCA | | GGACCAAGCTGACCGTCCTA | |
| T28 | CAGGTGCAGCTGCAGGAGTCGGGC | QVQLQESGP | AATTTTATGCTGACTCAGCCCCA | NFMLTQPHS |
| | CCAGGACTGGTGAAGCCTTCGGAG | GLVKPSETLS | CTCTGTGTCGGAGTCTCCGGGG | VSESPGKTVTI |
| | ACCCTGTCCCTCAGCTGCGCTGTCTC | LSCAVSGGSI | AAGACGGTAACCATCTCCTGCA | SCTGSSGSIAS |
| | TGGCGGCTCCATCAGAGGTTATTCC | RGYSWSWIR | CCGGCAGCAGTGGCAGCATTGC | NYVQWYQQ |
| | TGGAGCTGGATCCGGCAGCCCCCC | QPPGKGLEW | CAGCAACTATGTGCAGTGGTAC | RPGSAPTTLIY |
| | GGGAAGGGACTGGAGTGGATTGCT | IADMLYTGTT | CAGCAGCGCCCGGGCAGTGCC | EDKQRPSGV |
| | GATATGTTATATACTGGGACCACCA | TFNPSLKSRV | CCCACCACTTTGATCTATGAGG | PDRFSGSIDS |
| | CCTTCAACCCATCCCTCAAGAGTCG | TISVDTSKNQ | ATAAGCAAAGACCCTCTGGGGT | STNSASLTISG |
| | AGTCACCATATCGGTAGACACGTCC | FSLKLTSVTA | CCCTGATCGGTTCTCTGGCTCCA | LKTEDEADYY |
| | AAGAACCAGTTCTCCCTGAAGCTGA | ADTAVYYCA | TCGACAGCTCCACCAACTCTGC | CQSFDSSNR |
| | CCTCTGTGACCGCTGCGGACACGGC | RGPTVSGPIV | CTCCCTCACCATCTCTGGACTGA | WVFGGGTKV |
| | CGTGTATTACTGTGCGAGGGGACC | VDYWGPGTL | AGACTGAGGACGAGGCTGACT | TVL |
| | GACTGTGTCAGGGCCCATAGTAGTT | VTVSS | ACTACTGTCAGTCTTTTGATAGC | |
| | GACTACTGGGGCCCGGGAACCCTG | | AGTAATCGTTGGGTGTTCGGCG | |
| | GTCACCGTCTCCTCA | | GAGGGACCAAAGTGACCGTCCT | |
| | | | A | |
| T29 | CAGCTGCAGCTGCAGGAGTCGGGC | QLQLQESGP | CAGTCTGTGTTGACGCAGCCGC | QSVLTQPPSV |
| | CCAGGACTGGTGAAGCCTTCGGAG | GLVKPSETLS | CCTCAGTGTCTGCGGCCCCAGG | SAAPGQKVTI |
| | ACCCTGTCCCTCACCTGCACTGTCTC | LTCTVSGGSI | ACAGAAGGTCACCATCTCCTGC | SCSGSSSNIG |
| | TGGTGGCTCCATCAGCAGTAGTAGT | SSSSYYWGW | TCTGGAAGCAGCTCCAACATTG | NNYVSWYQ |
| | TACTACTGGGGCTGGATCCGCCAGC | IRQPPGKGLE | GGAATAATTATGTATCCTGGTA | QLPGTAPKLLI |
| | CCCCAGGGAAGGGGCTGGAGTGGA | WIGSLSYTGS | CCAGCAGCTCCCAGGAACAGCC | YDNNKRPSGI |
| | TTGGGAGTCTCTCTTATACTGGGAG | TYYNPSLKSR | CCCAAACTCCTCATTTATGACAA | PDRFSGSKSG |
| | CACCTACTACAACCCGTCCCTCAAG | VTISLDTSKN | TAATAAGCGACCCTCAGGGATT | TSATLGISGL |
| | AGTCGAGTCACCATATCACTAGACA | QFSLKLSSVT | CCTGACCGATTCTCTGGCTCCAA | QTGDEADYY |
| | CGTCCAAGAACCAATTCTCCCTGAA | AADTAVYYC | GTCTGGCACGTCAGCCACCCTG | CGTWDSSLS |
| | GCTGAGCTCTGTGACCGCCGCGGAC | ARESGSGGT | GGCATCAGCGGACTCCAGACTG | AWVFGGGTK |
| | ACGGCCGTGTATTACTGTGCGAGAG | HTDSWGQG | GGGACGAGGCCGATTATTACTG | LTVL |
| | AGAGTGGTAGTGGTGGTACCCATA | TLVSVSS | CGGAACATGGGATAGCAGCCT | |
| | CTGACTCCTGGGGCCAGGGAACCCT | | GAGTGCTTGGGTTTTCGGCGGA | |
| | GGTCTCCGTCTCCTCA | | GGGACCAAGCTGACCGTCCTA | |
| T30 | GAGGTGCAGCTGGTGGAGTCTGGG | EVQLVESGG | CAGGCTGTGGTGACTCAGGAG | QAVVTQEPSL |
| | GGAGGTGTACTACGGCCTGGGGAA | GVLRPGESLR | CCCTCACTGACTGTGTCCCCAG | TVSPGGTVTL |
| | TCCCTGAGACTCTCCTGTGTAGCCTC | LSCVASGFTF | GAGGGACAGTCACTCTCACCTG | TCGSSTGAVT |
| | TGGATTCACCTTTGAGGATTATGGC | EDYGMSWV | TGGCTCCAGCACTGGAGCTGTC | SGHYPYWFQ |
| | ATGAGTTGGGTCCGCCAAGTTCCAG | RQVPGKGLE | ACCAGTGGTCATTATCCCTACTG | QKPGQAPRT |
| | GGAAGGGGCTGGAGTGGGTCTCTG | WVSGINWN | GTTCCAGCAGAAGCCTGGCCAA | LIYDTSDRHS |
| | GAATTAATTGGAATGGTGGTAGCAC | GGSTRYADS | GCCCCCCGGACACTGATTTATG | WTPARFSGS |
| | ACGATATGCAGACTCTGTGAAGGGC | VKGRFTISRD | ATACCAGCGACAGACACTCCTG | LRGGKAALTL |
| | CGATTCACCATCTCCAGAGACAACG | NANNSLYLQ | GACACCTGCCCGGTTTTCAGGC | SGAQPEDEA |
| | CCAACAATTCCCTGTATCTGCAAATG | MNSLRAEDT | TCCCTTCGTGGGGGCAAAGCAG | DYYCFLSYNG |
| | AACAGTCTGAGAGCCGAAGACACG | ALYHCARDK | CCCTGACCCTTTCGGGTGCGCA | ARVFGGGTK |
| | GCCTTGTATCACTGTGCGAGAGATA | AIQGALMVY | GCCTGAAGATGAGGCTGACTAT | LTVL |
| | AGGCCATCCAGGGTGCACTAATGGT | AMRGRWFD | TATTGCTTCCTCTCCTATAATGG | |
| | GTATGCTATGAGGGGTCGGTGGTTC | PWGQGTLVT | TGCTCGGGTATTCGGCGGAGG | |
| | GACCCCTGGGGCCAGGGAACCCTG | VSS | GACCAAGCTGACCGTCCTG | |
| | GTCACCGTCTCCTCA | | | |
| T1-GL | GAGGTGCAGCTGGTGGAGTCTGGG | EVQLVESGG | GCCATCCAGATGACCCAGTCTC | AIQMTQSPSS |
| | GGAGGCTTGGTACAGCCAGGGCGG | GLVQPGRSL | CATCCTCCCTGTCTGCATCTGTA | LSASVGDRVT |
| | TCCCTGAGACTCTCCTGTACAGCTTC | RLSCTASGFT | GGAGACAGAGTCACCATCACTT | ITCRASQGIR |
| | TGGATTCACCTTTGGTGATTATGCTA | FGDYAMSW | GCCGGGCAAGTCAGGGCATTA | NDLGWYQQ |
| | TGAGCTGGTTCCGCCAGGCTCCAGG | FRQAPGKGL | GAAATGATTTAGGCTGGTATCA | KPGKAPKLLIY |
| | GAAGGGGCTGGAGTGGGTAGGTTT | EWVGFIRSK | GCAGAAACCAGGGAAAGCCCC | AASSLQSGVP |
| | CATTAGAAGCAAAGCTTATGGTGGG | AYGGTTEYA | TAAGCTCCTGATCTATGCTGCAT | SRFSGSGSGT |
| | ACAACAGAATACGCCGCGTCTGTGA | ASVKGRFTIS | CCAGTTTACAAAGTGGGGTCCC | DFTLTISSLQP |
| | AAGGCAGATTCACCATCTCAAGAGA | RDDSKSIAYL | ATCAAGGTTCAGCGGCAGTGG | EDFATYYCLQ |
| | TGATTCCAAAAGCATCGCCTATCTG | QMNSLKTED | ATCTGGCACAGATTTCACTCTCA | DYNYPWTFG |
| | CAAATGAACAGCCTGAAAACCGAG | TAVYYCTKYA | CCATCAGCAGCCTGCAGCCTGA | QGTKVEIK |
| | GACACAGCCGTGTATTACTGTACTA | SGWEVGFDP | AGATTTTGCAACTTATTACTGTC | |
| | AATATGCCAGTGGCTGGGAGGTTG | WGQGTLVT | TACAAGATTACAATTACCCGTG | |
| | GATTCGACCCCTGGGGCCAGGGAA | VSS | GACGTTCGGCCAAGGGACCAA | |
| | CCCTGGTCACCGTCTCCTCA | | GGTGGAAATCAAA | |
| T2-GL | CAGGTTCAGCTGGTGCAGTCTGGA | QVQLVQSGA | GACATCCAGATGACCCAGTCTC | DIQMTQSPS |
| | GCTGAGGTGAAGAAGCCTGGGGCC | EVKKPGASV | CTTCCACCCTGTCTGCATCTGTA | TLSASVGDRV |
| | TCAGTGAAGGTCTCCTGCAAGGCTT | KVSCKASGYT | GGAGACAGAGTCACCATCACTT | TITCRASQSIS |
| | CTGGTTACACCTTTACCAGCTATGGT | FTSYGISWVR | GCCGGGCCAGTCAGAGTATTAG | SWLAWYQQ |
| | ATCAGCTGGGTGCGACAGGCCCCT | QAPGQGLE | TAGCTGGTTGGCCTGGTATCAG | KPGKAPKLLIY |
| | GGACAAGGGCTTGAGTGGATGGGA | WMGWISAY | CAGAAACCAGGGAAAGCCCCTA | DASSLESGVP |
| | TGGATCAGCGCTTACAATGGTAACA | NGNTNYAQK | AGCTCCTGATCTATGATGCCTCC | SRFSGSGSGT |
| | CAAACTATGCACAGAAGCTCCAGGG | LQGRVTMTT | AGTTTGGAAAGTGGGGTCCCAT | EFTLTISSLQP |
| | CAGAGTCACCATGACCACAGACACA | DTSTSTAYM | CAAGGTTCAGCGGCAGTGGATC | DDFATYYCQ |
| | TCCACGAGCACAGCCTACATGGAGC | ELRSLRSDDT | TGGGACAGAATTCACTCTCACC | QYNSYSTFG |
| | TGAGGAGCCTGAGATCTGACGACA | AVYYCARTP | ATCAGCAGCCTGCAGCCTGATG | QGTKVEIR |
| | CGGCCGTGTATTACTGTGCGAGAAC | AALASFDYW | ATTTTGCAACTTATTACTGCCAA | |
| | CCCCGCTGCCCTGGCAAGTTTTGAC | GQGTLVTVS | CAGTATAATAGTTATTCGACGTT | |
| | TACTGGGGCCAGGGAACCCTGGTC | S | CGGCCAAGGGACCAAGGTGGA | |
| | ACCGTCTCCTCA | | AATCAGA | |
| T4-GL | CAGGTTCAGCTGGTGCAGTCTGGA | QVQLVQSGA | GACATCCAGATGACCCAGTCTC | DIQMTQSPS |
| | GCTGAGGTGAAGAAGCCTGGGGCC | EVKKPGASV | CTTCCACCCTGTCTGCATCTGTA | TLSASVGDRV |
| | TCAGTGAAGGTCTCCTGCAAGGCTT | KVSCKASGYT | GGAGACAGAGTCACCATCACTT | TITCRASQSIS |
| | CTGGTTACACCTTTACCAGCTATGGT | FTSYGISWVR | GCCGGGCCAGTCAGAGTATTAG | SWLAWYQQ |
| | ATCAGCTGGGTGCGACAGGCCCCT | QAPGQGLE | TAGCTGGTTGGCCTGGTATCAG | KPGKAPKLLIY |
| | GGACAAGGGCTTGAGTGGATGGGA | WMGWISAY | CAGAAACCAGGGAAAGCCCCTA | DASSLESGVP |
| | TGGATCAGCGCTTACAATGGTAACA | NGNTNYAQK | AGCTCCTGATCTATGATGCCTCC | SRFSGSGSGT |
| | CAAACTATGCACAGAAGCTCCAGGG | LQGRVTMTT | AGTTTGGAAAGTGGGGTCCCAT | EFTLTISSLQP |
| | CAGAGTCACCATGACCACAGACACA | DTSTSTAYM | CAAGGTTCAGCGGCAGTGGATC | DDFATYYCQ |
| | TCCACGAGCACAGCCTACATGGAGC | ELRSLRSDDT | TGGGACAGAATTCACTCTCACC | QYNDFPLTFG |
| | TGAGGAGCCTGAGATCTGACGACA | AVYYCARGQ | ATCAGCAGCCTGCAGCCTGATG | GGTKVEIK |
| | CGGCCGTGTATTACTGTGCGAGAG | GRYGDYIYN | ATTTTGCAACTTATTACTGCCAA | |
| | GCCAAGGACGGTACGGTGACTACA | HWGQGTLV | CAGTATAATGATTTTCCCCTCAC | |
| | TTTATAATCACTGGGGCCAGGGAAC | TVSS | TTTCGGCGGAGGGACCAAGGT | |
| | CCTGGTCACCGTCTCCTCA | | GGAGATCAAA | |
| T5-GL | CAGGTGCAGCTGGTGGAGTCTGGG | QVQLVESGG | GACATCCAGATGACCCAGTCTC | DIQMTQSPS |
| | GGAGGCGTGGTCCAGCCTGGGAGG | GVVQPGRSL | CATCCTCCCTGTCTGCATCTGTA | SLSASVGDRV |
| | TCCCTGAGACTCTCCTGTGCAGCCTC | RLSCAASGFT | GGAGACAGAGTCACCATCACTT | TITCQASQDI |
| | TGGATTCACCTTCAGTAGCTATGCT | FSSYAMHW | GCCAGGCGAGTCAGGACATTA | SNYLNWYQQ |
| | ATGCACTGGGTCCGCCAGGCTCCAG | VRQAPGKGL | GCAACTATTTAAATTGGTATCA | KPGKAPKLLIY |
| | GCAAGGGGCTGGAGTGGGTGGCA | EWVAVISYD | GCAGAAACCAGGGAAAGCCCC | DASNLETGVP |
| | GTTATATCATATGATGGAAGCAATA | GSNKYYADS | TAAGCTCCTGATCTACGATGCA | SRFSGSGSGT |
| | AATACTACGCAGACTCCGTGAAGGG | VKGRFTISRD | TCCAATTTGGAAACAGGGGTCC | DFTFTISSLQP |
| | CCGATTCACCATCTCCAGAGACAAT | NSKNTLYLQ | CATCAAGGTTCAGTGGAAGTGG | EDIATYYCQQ |
| | TCCAAGAACACGCTGTATCTGCAAA | MNSLRAEDT | ATCTGGGACAGATTTTACTTTCA | YDNLPSFGG |
| | TGAACAGCCTGAGAGCTGAGGACA | AVYYCARDR | CCATCAGCAGCCTGCAGCCTGA | GTKVEIK |
| | CGGCTGTGTATTACTGTGCGAGAGA | DSSGYIFDY | AGATATTGCAACATATTACTGTC | |
| | TCGCGATAGTAGTGGTTATATTTTT | WGQGTLVT | AACAGTATGATAATCTCCCTTCT | |
| | GACTACTGGGGCCAGGGAACCCTG | VSS | TTCGGCGGAGGGACCAAGGTG | |
| | GTCACCGTCTCCTCA | | GAGATCAAA | |
| T6-GL | CAGGTACAGCTGCAGCAGTCAGGT | QVQLQQSGP | GAAATAGTGATGACGCAGTCTC | EIVMTQSPAT |
| | CCAGGACTGGTGAAGCCCTCGCAG | GLVKPSQTLS | CAGCCACCCTGTCTGTGTCTCCA | LSVSPGERAT |
| | ACCCTCTCACTCACCTGTGCCATCTC | LTCAISGDSV | GGGGAAAGAGCCACCCTCTCCT | LSCRASQSVS |
| | CGGGGACAGTGTCTCTAGCAACAGT | SSNSAAWN | GCAGGGCCAGTCAGAGTGTTA | SNLAWYQQK |
| | GCTGCTTGGAACTGGATCAGGCAGT | WIRQSPSRG | GCAGCAACTTAGCCTGGTACCA | PGQAPRLLIY |
| | CCCCATCGAGAGGCCTTGAGTGGCT | LEWLGRTYY | GCAGAAACCTGGCCAGGCTCCC | GASTRATGIP |
| | GGGAAGGACATACTACAGGTCCAA | RSKWYNDYA | AGGCTCCTCATCTATGGTGCAT | ARFSGSGSGT |
| | GTGGTATAATGATTATGCAGTATCT | VSVKSRITINP | CCACCAGGGCCACTGGTATCCC | EFTLTISSLQS |
| | GTGAAAAGTCGAATAACCATCAACC | DTSKNQFSL | AGCCAGGTTCAGTGGCAGTGG | EDFAVYYCQ |
| | CAGACACATCCAAGAACCAGTTCTC | QLNSVTPED | GTCTGGGACAGAGTTCACTCTC | QYNNWPGF |
| | CCTGCAGCTGAACTCTGTGACTCCC | TAVYYCARDL | ACCATCAGCAGCCTGCAGTCTG | GQGTKLEIK |
| | GAGGACACGGCTGTGTATTACTGTG | GIAAADWFD | AAGATTTTGCAGTTTATTACTGT | |
| | CAAGAGATTTGGGTATAGCAGCGG | SWGQGTLVT | CAGCAGTATAATAACTGGCCGG | |
| | CTGACTGGTTCGACTCCTGGGGCCA | VSS | GTTTTGGCCAGGGGACCAAGCT | |
| | AGGAACCCTGGTCACCGTCTCCTCA | | GGAGATCAAA | |
| T7-GL | CAGGTGCAGCTGGTGGAGTCTGGG | QVQLVESGG | GATATTGTGATGACTCAGTCTC | DIVMTQSPLS |
| | GGAGGCGTGGTCCAGCCTGGGAGG | GVVQPGRSL | CACTCTCCCTGCCCGTCACCCCT | LPVTPGEPASI |
| | TCCCTGAGACTCTCCTGTGCAGCCTC | RLSCAASGFT | GGAGAGCCGGCCTCCATCTCCT | SCRSSQSLLH |
| | TGGATTCACCTTCAGTAGCTATGGC | FSSYGMHW | GCAGGTCTAGTCAGAGCCTCCT | SNGYNYLDW |
| | ATGCACTGGGTCCGCCAGGCTCCAG | VRQAPGKGL | GCATAGTAATGGATACAACTAT | YLQKPGQSP |
| | GCAAGGGGCTGGAGTGGGTGGCA | EWVAVISYD | TTGGATTGGTACCTGCAGAAGC | QLLIYLGSNR |
| | GTTATATCATATGATGGAAGTAATA | GSNKYYADS | CAGGGCAGTCTCCACAGCTCCT | ASGVPDRFS |
| | AATACTATGCAGACTCCGTGAAGGG | VKGRFTISRD | GATCTATTTGGGTTCTAATCGG | GSGSGTDFTL |
| | CCGATTCACCATCTCCAGAGACAAT | NSKNTLYLQ | GCCTCCGGGGTCCCTGACAGGT | KISRVEAEDV |
| | TCCAAGAACACGCTGTATCTGCAAA | MNSLRAEDT | TCAGTGGCAGTGGATCAGGCAC | GVYYCMQAL |
| | TGAACAGCCTGAGAGCTGAGGACA | AVYYCARSPS | AGATTTTACACTGAAAATCAGC | QTPFTFGPGT |
| | CGGCTGTGTATTACTGTGCGAGGAG | GHAFDVWG | AGAGTGGAGGCTGAGGATGTT | KVDIK |
| | CCCCAGTGGGCATGCTTTTGATGTC | QGTMVTVSS | GGGGTTTATTACTGCATGCAAG | |
| | TGGGGCCAAGGGACAATGGTCACC | | CTCTACAAACTCCTTTCACTTTC | |
| | GTCTCTTCA | | GGCCCTGGGACCAAAGTGGAT | |
| | | | ATCAAA | |
| T8-GL | CAGGTGCAGCTGCAGGAGTCGGGC | QVQLQESGP | GACATCCAGATGACCCAGTCTC | DIQMTQSPS |
| | CCAGGACTGGTGAAGCCTTCGGAG | GLVKPSETLS | CTTCCACCCTGTCTGCATCTGTA | TLSASVGDRV |
| | ACCCTGTCCCTCACCTGCACTGTCTC | LTCTVSGGSI | GGAGACAGAGTCACCATCACTT | TITCRASQSIS |
| | TGGTGGCTCCATCAGTAGTTACTAC | SSYYWSWIR | GCCGGGCCAGTCAGAGTATTAG | SWLAWYQQ |
| | TGGAGCTGGATCCGGCAGCCCCCA | QPPGKGLEW | TAGCTGGTTGGCCTGGTATCAG | KPGKAPKLLIY |
| | GGGAAGGGACTGGAGTGGATTGG | IGYIYYSGSTN | CAGAAACCAGGGAAAGCCCCTA | KASSLESGVP |
| | GTATATCTATTACAGTGGGAGCACC | YNPSLKSRVT | AGCTCCTGATCTATAAGGCGTC | SRFSGSGSGT |
| | AACTACAACCCCTCCCTCAAGAGTC | ISVDTSKNQF | TAGTTTAGAAAGTGGGGTCCCA | EFTLTISSLQP |
| | GAGTCACCATATCAGTAGACACGTC | SLKLSSVTAA | TCAAGGTTCAGCGGCAGTGGAT | DDFATYYCQ |
| | CAAGAACCAGTTCTCCCTGAAGCTG | DTAVYYCAR | CTGGGACAGAATTCACTCTCAC | QYNSYGITFG |
| | AGCTCTGTGACCGCTGCGGACACG | GPTVSGPIVV | CATCAGCAGCCTGCAGCCTGAT | PGTKVDIK |
| | GCCGTGTATTACTGTGCGAGGGGA | DYWGQGTL | GATTTTGCAACTTATTACTGCCA | |
| | CCGACTGTGTCAGGGCCCATAGTAG | VTVSS | ACAGTATAATAGTTACGGGATC | |
| | TTGACTACTGGGGCCAGGGAACCCT | | ACTTTCGGCCCTGGGACCAAAG | |
| | GGTCACCGTCTCCTCA | | TGGATATCAAA | |
| T10-GL | CAGGTTCAGCTGGTGCAGTCTGGA | QVQLVQSGA | GACATCCAGATGACCCAGTCTC | DIQMTQSPS |
| | GCTGAGGTGAAGAAGCCTGGGGCC | EVKKPGASV | CATCCTCCCTGTCTGCATCTGTA | SLSASVGDRV |
| | TCAGTGAAGGTCTCCTGCAAGGCTT | KVSCKASGYT | GGAGACAGAGTCACCATCACTT | TITCRASQSIS |
| | CTGGTTACACCTTTACCAGCTATGGT | FTSYGISWVR | GCCGGGCAAGTCAGAGCATTA | SYLNWYQQK |
| | ATCAGCTGGGTGCGACAGGCCCCT | QAPGQGLE | GCAGCTATTTAAATTGGTATCA | PGKAPKLLIY |
| | GGACAAGGGCTTGAGTGGATGGGA | WMGWISAY | GCAGAAACCAGGGAAAGCCCC | AASSLQSGVP |
| | TGGATCAGCGCTTACAATGGTAACA | NGNTNYAQK | TAAGCTCCTGATCTATGCTGCAT | SRFSGSGSGT |
| | CAAACTATGCACAGAAGCTCCAGGG | LQGRVTMTT | CCAGTTTGCAAAGTGGGGTCCC | DFTLTISSLQP |
| | CAGAGTCACCATGACCACAGACACA | DTSTSTAYM | ATCAAGGTTCAGTGGCAGTGGA | EDFATYYCQ |
| | TCCACGAGCACAGCCTACATGGAGC | ELRSLRSDDT | TCTGGGACAGATTTCACTCTCAC | QSYSTPPYTF |
| | TGAGGAGCCTGAGATCTGACGACA | AVYYCARDT | CATCAGCAGTCTGCAACCTGAA | GQGTKLEIK |
| | CGGCCGTGTATTACTGTGCGAGAGA | RYCSGGSCPR | GATTTTGCAACTTACTACTGTCA | |
| | TACCCGATATTGTAGTGGTGGTAGC | PSWYYYYMD | ACAGAGTTACAGTACCCCTCCA | |
| | TGCCCCCGGCCAAGTTGGTACTACT | VWGKGTTVT | TACACTTTTGGCCAGGGGACCA | |
| | ACTACATGGACGTCTGGGGCAAAG | VSS | AGCTGGAGATCAAA | |
| | GGACCACGGTCACCGTCTCCTCA | | | |
| T11-GL | CAGGTGCAGCTGGTGGAGTCTGGG | QVQLVESGG | GACATCCAGATGACCCAGTCTC | DIQMTQSPS |
| | GGAGGCGTGGTCCAGCCTGGGGGG | GVVQPGGSL | CATCCTCCCTGTCTGCATCTGTA | SLSASVGDRV |
| | TCCCTGAGACTCTCCTGTGCAGCGT | RLSCAASGFT | GGAGACAGAGTCACCATCACTT | TITCRASQGIS |
| | CTGGATTCACCTTCAGTAGCTATGG | FSSYGMHW | GCCGGGCGAGTCAGGGCATTA | NYLAWYQQK |
| | CATGCACTGGGTCCGCCAGGCTCCA | VRQAPGKGL | GCAATTATTTAGCCTGGTATCA | PGKVPKLLIYA |
| | GGCAAGGGGCTGGAGTGGGTGGC | EWVAFIRYD | GCAGAAACCAGGGAAAGTTCCT | ASTLQSGVPS |
| | ATTTATACGGTATGATGGAAGTAAT | GSNKYYADS | AAGCTCCTGATCTATGCTGCATC | RFSGSGSGTD |
| | AAATACTATGCAGACTCCGTGAAGG | VKGRFTISRD | CACTTTGCAATCAGGGGTCCCA | FTLTISSLQPE |
| | GCCGATTCACCATCTCCAGAGACAA | NSKNTLYLQ | TCTCGGTTCAGTGGCAGTGGAT | DVATYYCQKY |
| | TTCCAAGAACACGCTGTATCTGCAA | MNSLRAEDT | CTGGGACAGATTTCACTCTCAC | NSAPWTFGQ |
| | ATGAACAGCCTGAGAGCTGAGGAC | AVYYCANPYI | CATCAGCAGCCTGCAGCCTGAA | GTKVEIK |
| | ACGGCTGTGTATTACTGTGCGAATC | TPPTNDYWG | GATGTTGCAACTTATTACTGTCA | |
| | CTTATATAACGCCGCCTACTAATGAC | QGTLVTVSS | AAAGTATAACAGTGCCCCGTGG | |
| | TACTGGGGCCAGGGAACCCTGGTC | | ACGTTCGGCCAAGGGACCAAG | |
| | ACCGTCTCCTCA | | GTGGAAATCAAA | |
| T18-GL | GAGGTGCAGCTGGTGGAGTCTGGG | EVQLVESGG | GACATCCAGATGACCCAGTCTC | DIQMTQSPS |
| | GGAGGCTTGGTCCAGCCTGGGGGG | GLVQPGGSL | CATCCTCCCTGTCTGCATCTGTA | SLSASVGDRV |
| | TCCCTGAGACTCTCCTGTGCAGCCTC | RLSCAASGFT | GGAGACAGAGTCACCATCACTT | TITCRASQSIS |
| | TGGATTCACCTTTAGTAGCTATTGG | FSSYWMSW | GCCGGGCAAGTCAGAGCATTA | SYLNWYQQK |
| | ATGAGCTGGGTCCGCCAGGCTCCA | VRQAPGKGL | GCAGCTATTTAAATTGGTATCA | PGKAPKLLIY |
| | GGGAAGGGGCTGGAGTGGGTGGC | EWVANIKQD | GCAGAAACCAGGGAAAGCCCC | AASSLQSGVP |
| | CAACATAAAGCAAGATGGAAGTGA | GSEKYYVDSV | TAAGCTCCTGATCTATGCTGCAT | SRFSGSGSGT |
| | GAAATACTATGTGGACTCTGTGAAG | KGRFTISRDN | CCAGTTTGCAAAGTGGGGTCCC | DFTLTISSLQP |
| | GGCCGATTCACCATCTCCAGAGACA | AKNSLYLQM | ATCAAGGTTCAGTGGCAGTGGA | EDFATYYCQ |
| | ACGCCAAGAACTCACTGTATCTGCA | NSLRAEDTA | TCTGGGACAGATTTCACTCTCAC | QSYSTPRTFG |
| | AATGAACAGCCTGAGAGCCGAGGA | VYYCARDLRY | CATCAGCAGTCTGCAACCTGAA | QGTKVEIK |
| | CACGGCTGTGTATTACTGTGCGAGA | CSSTSCSPAL | GATTTTGCAACTTACTACTGTCA | |
| | GACCTAAGATATTGTAGTAGTACCA | DYWGQGTL | ACAGAGTTACAGTACCCCCCGG | |
| | GCTGTTCACCAGCTCTTGACTACTG | VTVSS | ACGTTCGGCCAAGGGACCAAG | |
| | GGGCCAGGGAACCCTGGTCACCGT | | GTGGAAATCAAA | |
| | CTCCTCA | | | |
| T19-GL | GAGGTGCAGCTGTTGGAGTCTGGG | EVQLLESGG | GAAATTGTGTTGACACAGTCTC | EIVLTQSPATL |
| | GGAGGCTTGGTACAGCCTGGGGGG | GLVQPGGSL | CAGCCACCCTGTCTTTGTCTCCA | SLSPGERATL |
| | TCCCTGAGACTCTCCTGTGCAGCCTC | RLSCAASGFT | GGGGAAAGAGCCACCCTCTCCT | SCRASQSVSS |
| | TGGATTCACCTTTAGCAGCTATGCC | FSSYAMSWV | GCAGGGCCAGTCAGAGTGTTA | YLAWYQQKP |
| | ATGAGCTGGGTCCGCCAGGCTCCA | RQAPGKGLE | GCAGCTACTTAGCCTGGTACCA | GQAPRLLIYD |
| | GGGAAGGGGCTGGAGTGGGTCTCA | WVSAISGSG | ACAGAAACCTGGCCAGGCTCCC | ASNRATGIPA |
| | GCTATTAGTGGTAGTGGTGGTAGCA | GSTYYADSVK | AGGCTCCTCATCTATGATGCATC | RFSGSGSGTD |
| | CATACTACGCAGACTCCGTGAAGGG | GRFTISRDNS | CAACAGGGCCACTGGCATCCCA | FTLTISSLEPE |
| | CCGGTTCACCATCTCCAGAGACAAT | KNTLYLQMN | GCCAGGTTCAGTGGCAGTGGG | DFAVYYCQQ |
| | TCCAAGAACACGCTGTATCTGCAAA | SLRAEDTAVY | TCTGGGACAGACTTCACTCTCA | RSNWPPLTF |
| | TGAACAGCCTGAGAGCCGAGGACA | YCAKDLLRYD | CCATCAGCAGCCTAGAGCCTGA | GGGTKVEIK |
| | CGGCCGTATATTACTGTGCGAAAGA | SSGYSPWGQ | AGATTTTGCAGTTTATTACTGTC | |
| | TTTATTACGTTATGATAGTAGTGGTT | GTLVTVSS | AGCAGCGTAGCAACTGGCCTCC | |
| | ATAGCCCCTGGGGCCAGGGAACCC | | GCTCACTTTCGGCGGAGGGACC | |
| | TGGTCACCGTCTCCTCC | | AAGGTGGAGATCAAA | |
| T20-GL | CAGGTGCAGCTGCAGGAGTCGGGC | QVQLQESGP | GAAATTGTGTTGACGCAGTCTC | EIVLTQSPGTL |
| | CCAGGACTGGTGAAGCCTTCACAGA | GLVKPSQTLS | CAGGCACCCTGTCTTTGTCTCCA | SLSPGERATL |
| | CCCTGTCCCTCACCTGCACTGTCTCT | LTCTVSGGSI | GGGGAAAGAGCCACCCTCTCCT | SCRASQSVSS |
| | GGTGGCTCCATCAGCAGTGGTGGTT | SSGGYYWS | GCAGGGCCAGTCAGAGTGTTA | SYLAWYQQK |
| | ACTACTGGAGCTGGATCCGCCAGCA | WIRQHPGKG | GCAGCAGCTACTTAGCCTGGTA | PGQAPRLLIY |
| | CCCAGGGAAGGGCCTGGAGTGGAT | LEWIGYIYYS | CCAGCAGAAACCTGGCCAGGCT | GASSRATGIP |
| | TGGGTACATCTATTACAGTGGGAGC | GSTYYNPSLK | CCCAGGCTCCTCATCTATGGTG | DRFSGSGSGT |
| | ACCTACTACAACCCGTCCCTCAAGA | SRVTISVDTS | CATCCAGCAGGGCCACTGGCAT | DFTLTISRLEP |
| | GTCGAGTTACCATATCAGTAGACAC | KNQFSLKLSS | CCCAGACAGGTTCAGTGGCAGT | EDFAVYYCQ |
| | GTCTAAGAACCAGTTCTCCCTGAAG | VTAADTAVY | GGGTCTGGGACAGACTTCACTC | QYGSSPRTFG |
| | CTGAGCTCTGTGACTGCCGCGGACA | YCARAPRITF | TCACCATCAGCAGACTGGAGCC | QGTKVEIK |
| | CGGCCGTGTATTACTGTGCGAGAGC | GGVIGGAAF | TGAAGATTTTGCAGTGTATTACT | |
| | CCCGCGGATTACGTTTGGGGGAGTT | DVWGQGT | GTCAGCAGTATGGTAGCTCACC | |
| | ATAGGGGGTGCTGCTTTTGATGTCT | MVTVSS | TCGGACGTTCGGCCAAGGGACC | |
| | GGGGCCAAGGGACAATGGTCACCG | | AAGGTGGAAATCAAA | |
| | TCTCTTCA | | | |
| T21-GL | CAGGTGCAGCTGCAGGAGTCGGGC | QVQLQESGP | GACATCCAGATGACCCAGTCTC | DIQMTQSPS |
| | CCAGGACTGGTGAAGCCTTCACAGA | GLVKPSQTLS | CATCCTCCCTGTCTGCATCTGTA | SLSASVGDRV |
| | CCCTGTCCCTCACCTGCACTGTCTCT | LTCTVSGGSI | GGAGACAGAGTCACCATCACTT | TITCRASQSIS |
| | GGTGGCTCCATCAGCAGTGGTAGTT | SSGSYYWSW | GCCGGGCAAGTCAGAGCATTA | SYLNWYQQK |
| | ACTACTGGAGCTGGATCCGGCAGCC | IRQPAGKGLE | GCAGCTATTTAAATTGGTATCA | PGKAPKLLIY |
| | CGCCGGGAAGGGACTGGAGTGGAT | WIGRIYTSGS | GCAGAAACCAGGGAAAGCCCC | AASSLQSGVP |
| | TGGGCGTATCTATACCAGTGGGAGC | TNYNPSLKSR | TAAGCTCCTGATCTATGCTGCAT | SRFSGSGSGT |
| | ACCAACTACAACCCCTCCCTCAAGA | VTISVDTSKN | CCAGTTTGCAAAGTGGGGTCCC | DFTLTISSLQP |
| | GTCGAGTCACCATATCAGTAGACAC | QFSLKLSSVT | ATCAAGGTTCAGTGGCAGTGGA | EDFATYYCQ |
| | GTCCAAGAACCAGTTCTCCCTGAAG | AADTAVYYC | TCTGGGACAGATTTCACTCTCAC | QSYSTPWTF |
| | CTGAGCTCTGTGACCGCCGCAGACA | ARGLRLAAE | CATCAGCAGTCTGCAACCTGAA | GQGTKVEIK |
| | CGGCCGTGTATTACTGTGCGAGAG | AYYYGMDV | GATTTTGCAACTTACTACTGTCA | |
| | GATTACGTTTGGCAGCAGAAGCCTA | WGQGTTVT | ACAGAGTTACAGTACCCCGTGG | |
| | CTACTACGGTATGGACGTCTGGGGC | VSS | ACGTTCGGCCAAGGGACCAAG | |
| | CAAGGGACCACGGTCACCGTCTCCT | | GTGGAAATCAAG | |
| | CA | | | |
| T22-GL | CAGGTGCAGCTGGTGCAGTCTGGG | QVQLVQSGA | GACATCGTGATGACCCAGTCTC | DIVMTQSPD |
| | GCTGAGGTGAAGAAGCCTGGGTCC | EVKKPGSSVK | CAGACTCCCTGGCTGTGTCTCT | SLAVSLGERA |
| | TCGGTGAAGGTCTCCTGCAAGGCTT | VSCKASGGTF | GGGCGAGAGGGCCACCATCAA | TINCKSSQSV |
| | CTGGAGGCACCTTCAGCAGCTATGC | SSYAISWVR | CTGCAAGTCCAGCCAGAGTGTT | LYSSNNKNYL |
| | TATCAGCTGGGTGCGACAGGCCCCT | QAPGQGLE | TTATACAGCTCCAACAATAAGA | AWYQQKPG |
| | GGACAAGGGCTTGAGTGGATGGGA | WMGGIIPIF | ACTACTTAGCTTGGTACCAGCA | QPPKLLIYWA |
| | GGGATCATCCCTATCTTTGGTACAG | GTANYAQKF | GAAACCAGGACAGCCTCCTAAG | STRESGVPDR |
| | CAAACTACGCACAGAAGTTCCAGGG | QGRVTITADE | CTGCTCATTTACTGGGCATCTAC | FSGSGSGTDF |
| | CAGAGTCACGATTACCGCGGACGA | STSTAYMELS | CCGGGAATCCGGGGTCCCTGAC | TLTISSLQAED |
| | ATCCACGAGCACAGCCTACATGGAG | SLRSEDTAVY | CGATTCAGTGGCAGCGGGTCTG | VAVYYCQQY |
| | CTGAGCAGCCTGAGATCTGAGGAC | YCATSSLSDIV | GGACAGATTTCACTCTCACCATC | YSTPLTFGGG |
| | ACGGCCGTGTATTACTGTGCGACGT | VVVAAFVDH | AGCAGCCTGCAGGCTGAAGAT | TKVEIK |
| | CCTCACTCAGCGATATTGTAGTGGT | YYGMDVWG | GTGGCAGTTTATTACTGTCAGC | |
| | GGTAGCTGCCTTTGTCGACCACTAC | QGTTVTVSS | AATATTATAGTACTCCCCTCACT | |
| | TACGGTATGGACGTCTGGGGCCAA | | TTCGGCGGAGGGACCAAGGTG | |
| | GGGACCACGGTCACCGTCTCCTCA | | GAGATCAAA | |
| T23-GL | GAGGTGCAGCTGGTGGAGTCTGGG | EVQLVESGG | GACATCCAGATGACCCAGTCTC | DIQMTQSPS |
| | GGAGGCTTGGTACAGCCTGGAGGG | GLVQPGGSL | CATCCTCCCTGTCTGCATCTGTA | SLSASVGDRV |
| | TCCCTGAGACTCTCCTGTGCAGCCTC | RLSCAASGFT | GGAGACAGAGTCACCATCACTT | TITCRASQSIS |
| | TGGATTCACCTTCAGTAGTTATGAA | FSSYEMNWV | GCCGGGCAAGTCAGAGCATTA | SYLNWYQQK |
| | ATGAACTGGGTCCGCCAGGCTCCAG | RQAPGKGLE | GCAGCTATTTAAATTGGTATCA | PGKAPKLLIY |
| | GGAAGGGGCTGGAGTGGGTTTCAT | WVSYISSSGS | GCAGAAACCAGGGAAAGCCCC | AASSLQSGVP |
| | ACATTAGTAGTAGTGGTAGTACCAT | TIYYADSVKG | TAAGCTCCTGATCTATGCTGCAT | SRFSGSGSGT |
| | ATACTACGCAGACTCTGTGAAGGGC | RFTISRDNAK | CCAGTTTGCAAAGTGGGGTCCC | DFTLTISSLQP |
| | CGATTCACCATCTCCAGAGACAACG | NSLYLQMNS | ATCAAGGTTCAGTGGCAGTGGA | EDFATYYCQ |
| | CCAAGAACTCACTGTATCTGCAAAT | LRAEDTAVYY | TCTGGGACAGATTTCACTCTCAC | QSYSTPRTFG |
| | GAACAGCCTGAGAGCCGAGGACAC | CATWGLGYC | CATCAGCAGTCTGCAACCTGAA | QGTKVEIK |
| | GGCTGTTTATTACTGTGCGACATGG | SSTSCYITDG | GATTTTGCAACTTACTACTGTCA | |
| | GGACTGGGATATTGTAGTAGTACCA | MDVWGQG | ACAGAGTTACAGTACCCCTCGG | |
| | GCTGCTATATCACTGACGGTATGGA | TTVTVSS | ACGTTCGGCCAAGGGACCAAG | |
| | CGTCTGGGGGCAAGGGACCACGGT | | GTGGAAATCAAA | |
| | CACCGTCTCCTCA | | | |
| T25-GL | GAGGTGCAGCTGGTGGAGTCTGGG | EVQLVESGG | CAGTCTGTGCTGACTCAGCCAC | QSVLTQPPSA |
| | GGAGGCTTGGTAAAGCCTGGGGGG | GLVKPGGSLR | CCTCAGCGTCTGGGACCCCCGG | SGTPGQRVTI |
| | TCCCTTAGACTCTCCTGTGCAGCCTC | LSCAASGFTF | GCAGAGGGTCACCATCTCTTGT | SCSGSSSNIG |
| | TGGATTCACTTTCAGTAACGCCTGG | SNAWMSWV | TCTGGAAGCAGCTCCAACATCG | SNYVYWYQQ |
| | ATGAGCTGGGTCCGCCAGGCTCCA | RQAPGKGLE | GAAGTAATTATGTATACTGGTA | LPGTAPKLLIY |
| | GGGAAGGGGCTGGAGTGGGTTGG | WVGRIKSKT | CCAGCAGCTCCCAGGAACGGCC | RNNQRPSGV |
| | CCGTATTAAAAGCAAAACTGATGGT | DGGTTDYAA | CCCAAACTCCTCATCTATAGGA | PDRFSGSKSG |
| | GGGACAACAGACTACGCTGCACCC | PVKGRFTISR | ATAATCAGCGGCCCTCAGGGGT | TSASLAISGLR |
| | GTGAAAGGCAGATTCACCATCTCAA | DDSKNTLYLQ | CCCTGACCGATTCTCTGGCTCCA | SEDEADYYCA |
| | GAGATGATTCAAAAAACACGCTGTA | MNSLKTEDT | AGTCTGGCACCTCAGCCTCCCT | AWDDSLSG |
| | TCTGCAAATGAACAGCCTGAAAACC | AVYYCTTGCY | GGCCATCAGTGGGCTCCGGTCC | WVFGGGTKL |
| | GAGGACACAGCCGTGTATTACTGTA | STLDYWGQG | GAGGATGAGGCTGATTATTACT | TVL |
| | CCACAGGCTGCTATTCCACCCTTGA | TLVTVSS | GTGCAGCATGGGATGACAGCCT | |
| | CTACTGGGGCCAGGGAACCCTGGT | | GAGTGGTTGGGTGTTCGGCGG | |
| | CACCGTCTCCTCA | | AGGGACCAAGCTGACCGTCCTA | |
| T27-GL | GAAGTGCAGCTGGTGGAGTCTGGG | EVQLVESGG | CAGTCTGCCCTGACTCAGCCTG | QSALTQPASV |
| | GGAGGCTTGGTACAGCCTGGCAGG | GLVQPGRSL | CCTCCGTGTCTGGGTCTCCTGG | SGSPGQSITIS |
| | TCCCTGAGACTCTCCTGTGCAGCCTC | RLSCAASGFT | ACAGTCGATCACCATCTCCTGC | CTGTSSDVGS |
| | TGGATTCACCTTTGATGATTATGCCA | FDDYAMHW | ACTGGAACCAGCAGTGATGTTG | YNLVSWYQQ |
| | TGCACTGGGTCCGGCAAGCTCCAG | VRQAPGKGL | GGAGTTATAACCTTGTCTCCTG | HPGKAPKLMI |
| | GGAAGGGCCTGGAGTGGGTCTCAG | EWVSGISWN | GTACCAACAGCACCCAGGCAAA | YEVSKRPSGV |
| | GTATTAGTTGGAATAGTGGTAGCAT | SGSIGYADSV | GCCCCCAAACTCATGATTTATG | SNRFSGSKSG |
| | AGGCTATGCGGACTCTGTGAAGGG | KGRFTISRDN | AGGTCAGTAAGCGGCCCTCAG | NTASLTISGL |
| | CCGATTCACCATCTCCAGAGACAAC | AKNSLYLQM | GGGTTTCTAATCGCTTCTCTGGC | QAEDEADYY |
| | GCCAAGAACTCCCTGTATCTGCAAA | NSLRAEDTAL | TCCAAGTCTGGCAACACGGCCT | CCSYAGSSTL |
| | TGAACAGTCTGAGAGCTGAGGACA | YYCAKDEKW | CCCTGACAATCTCTGGGCTCCA | VFGGGTKLTV |
| | CGGCCTTGTATTACTGTGCAAAAGA | GTPSDWGQ | GGCTGAGGACGAGGCTGATTA | L |
| | TGAGAAATGGGGGACTCCGAGTGA | GTLVTVSS | TTACTGCTGCTCATATGCAGGT | |
| | CTGGGGCCAGGGAACCCTGGTCAC | | AGTAGCACTTTAGTGTTCGGCG | |
| | CGTCTCCTCA | | GAGGGACCAAGCTGACCGTCCT | |
| | | | A | |
| T28-GL | CAGGTGCAGCTGCAGGAGTCGGGC | QVQLQESGP | AATTTTATGCTGACTCAGCCCCA | NFMLTQPHS |
| | CCAGGACTGGTGAAGCCTTCGGAG | GLVKPSETLS | CTCTGTGTCGGAGTCTCCGGGG | VSESPGKTVTI |
| | ACCCTGTCCCTCACCTGCACTGTCTC | LTCTVSGGSI | AAGACGGTAACCATCTCCTGCA | SCTGSSGSIAS |
| | TGGTGGCTCCATCAGTAGTTACTAC | SSYYWSWIR | CCGGCAGCAGTGGCAGCATTGC | NYVQWYQQ |
| | TGGAGCTGGATCCGGCAGCCCCCA | QPPGKGLEW | CAGCAACTATGTGCAGTGGTAC | RPGSAPTTVI |
| | GGGAAGGGACTGGAGTGGATTGG | IGYIYYSGSTN | CAGCAGCGCCCGGGCAGTGCC | YEDNQRPSG |
| | GTATATCTATTACAGTGGGAGCACC | YNPSLKSRVT | CCCACCACTGTGATCTATGAGG | VPDRFSGSID |
| | AACTACAACCCCTCCCTCAAGAGTC | ISVDTSKNQF | ATAACCAAAGACCCTCTGGGGT | SSSNSASLTIS |
| | GAGTCACCATATCAGTAGACACGTC | SLKLSSVTAA | CCCTGATCGGTTCTCTGGCTCCA | GLKTEDEADY |
| | CAAGAACCAGTTCTCCCTGAAGCTG | DTAVYYCAR | TCGACAGCTCCTCCAACTCTGCC | YCQSYDSSNR |
| | AGCTCTGTGACCGCTGCGGACACG | GPTVSGPIVV | TCCCTCACCATCTCTGGACTGAA | WVFGGGTKL |
| | GCCGTGTATTACTGTGCGAGGGGA | DYWGQGTL | GACTGAGGACGAGGCTGACTA | TVL |
| | CCGACTGTGTCAGGGCCCATAGTAG | VTVSS | CTACTGTCAGTCTTATGATAGCA | |
| | TTGACTACTGGGGCCAGGGAACCCT | | GCAATCGTTGGGTGTTCGGCGG | |
| | GGTCACCGTCTCCTCA | | AGGGACCAAGCTGACCGTCCTA | |
| T30-GL | GAGGTGCAGCTGGTGGAGTCTGGG | EVQLVESGG | CAGGCTGTGGTGACTCAGGAG | QAVVTQEPSL |
| | GGAGGTGTGGTACGGCCTGGGGGG | GVVRPGGSL | CCCTCACTGACTGTGTCCCCAG | TVSPGGTVTL |
| | TCCCTGAGACTCTCCTGTGCAGCCTC | RLSCAASGFT | GAGGGACAGTCACTCTCACCTG | TCGSSTGAVT |
| | TGGATTCACCTTTGATGATTATGGC | FDDYGMSW | TGGCTCCAGCACTGGAGCTGTC | SGHYPYWFQ |
| | ATGAGCTGGGTCCGCCAAGCTCCAG | VRQAPGKGL | ACCAGTGGTCATTATCCCTACTG | QKPGQAPRT |
| | GGAAGGGGCTGGAGTGGGTCTCTG | EWVSGINW | GTTCCAGCAGAAGCCTGGCCAA | LIYDTSNKHS |
| | GTATTAATTGGAATGGTGGTAGCAC | NGGSTGYAD | GCCCCCAGGACACTGATTTATG | WTPARFSGS |
| | AGGTTATGCAGACTCTGTGAAGGG | SVKGRFTISR | ATACAAGCAACAAACACTCCTG | LLGGKAALTL |
| | CCGATTCACCATCTCCAGAGACAAC | DNAKNSLYL | GACACCTGCCCGGTTCTCAGGC | SGAQPEDEA |
| | GCCAAGAACTCCCTGTATCTGCAAA | QMNSLRAED | TCCCTCCTTGGGGGCAAAGCTG | EYYCLLSYSG |
| | TGAACAGTCTGAGAGCCGAGGACA | TALYHCARD | CCCTGACCCTTTCGGGTGCGCA | ARVFGGGTK |
| | CGGCCTTGTATCACTGTGCGAGAGA | KAIQGALMV | GCCTGAGGATGAGGCTGAGTA | LTVL |
| | TAAGGCCATCCAGGGTGCACTAATG | YAMRGRWF | TTACTGCTTGCTCTCCTATAGTG | |
| | GTGTATGCTATGAGGGGTCGGTGG | DPWGQGTL | GTGCTCGGGTATTCGGCGGAG | |
| | TTCGACCCCTGGGGCCAGGGAACCC | VTVSS | GGACCAAGCTGACCGTCCTG | |
| | TGGTCACCGTCTCCTCA | | | |

*GL-All germline antibodies are entitled as : T##-GL.The germline versions used in the study: T1-2, T4-8, T10-11, T18-23, T25, T27-28, T30.

TABLE 1A

| Monoclonal | SEQ ID NO Heavy chain | |
|---|---|---|
| antibody | nucleotide sequence | |
| T13 | 1 | |
| T12 | 2 | |
| T13-MRCA | 3 | |
| T12-MRCA | 4 | |
| T13-GL | 5 | |
| T12-GL | 6 | |
| T14 | 7 | |
| T15 | 8 | |
| T1 | 9 | |
| T2 | 10 | |
| T3 | 11 | |
| T4 | 12 | |
| T5 | 13 | |
| T6 | 14 | |
| T7 | 15 | |
| T8 | 16 | |
| T10 | 17 | |
| T11 | 18 | |
| T17 | 19 | |
| T3-16-17 MRCA1 | 20 | |
| T3-16-17 MRCA2 | 21 | |
| T3-16-17 GL | 22 | |
| | SEQ ID NO: Heavy chain | |
| | amino acid sequence | |
| T13 | 23 | |
| T12 | 24 | |
| T13-MRCA | 25 | |
| T12-MRCA | 26 | |
| T13-GL | 27 | |
| T12-GL | 28 | |
| T14 | 29 | |
| T15 | 30 | |
| T1 | 31 | |
| T2 | 32 | |
| T3 | 33 | |
| T4 | 34 | |
| T5 | 35 | |
| T6 | 36 | |
| T7 | 37 | |
| T8 | 38 | |
| T10 | 39 | |
| T11 | 40 | |
| T17 | 41 | |
| T3-16-17 MRCA1 | 42 | |
| T3-16-17 MRCA2 | 43 | |
| T3-16-17 GL | 44 | |
| | SEQ ID NO: Light chain | |
| | nucleotide sequence | |
| T13 | 45 | |
| T12 | 46 | |
| T13-MRCA | 47 | |
| T12-MRCA | 48 | |
| T13-GL | 49 | |
| T12-GL | 50 | |
| T14 | 51 | |
| T15 | 52 | |
| T1 | 53 | |
| T2 | 54 | |
| T3 | 55 | |
| T4 | 56 | |
| T5 | 57 | |
| T6 | 58 | |
| T7 | 59 | |
| T8 | 60 | |
| T10 | 61 | |
| T11 | 62 | |
| T17 | 63 | |
| T3-16-17 MRCA1 | 64 | |
| T3-16-17 MRCA2 | 65 | |
| T3-16-17 GL | 66 | |
| | SEQ ID NO: Light chain | |
| | amino acid sequence | |
| T13 | 67 | |
| T12 | 68 | |
| T13-MRCA | 69 | |
| T12-MRCA | 70 | |
| T13-GL | 71 | |
| T12-GL | 72 | |
| T14 | 73 | |
| T15 | 74 | |
| T1 | 75 | |
| T2 | 76 | |
| T3 | 77 | |
| T4 | 78 | |
| T5 | 79 | |
| T6 | 80 | |
| T7 | 81 | |
| T8 | 82 | |
| T10 | 83 | |
| T11 | 84 | |
| T17 | 85 | |
| T3-16-17 MRCA1 | 86 | |
| T3-16-17 MRCA2 | 87 | |
| T3-16-17 GL | 88 | |
| T18 | | |
| T19 | | |
| T20 | | |
| T21 | | |
| T22 | | |
| T23 | | |
| T24 | | |
| T25 | | |
| T26 | | |
| T27 | | |
| T28 | | |
| T29 | | |
| T30Z | | |
| T4-G1 | | |
| T7-GL | | |
| T22-GL | | |
| T30-GL | | |
| | CDR sequences SEQ ID NOs: | |
| T13 | 89 | GFIFSKHDD143D95:D148D95:DD95:D211 |
| | 90 | IGDAGDT |
| | 91 | GRGMAVAGFPLDV |
| | 92 | QNIHIN |
| | 93 | AAS |
| | 94 | QQFNPWSPWT |
| T12 | 95 | GFTFNNHD |
| | 96 | IGNFGDT |
| | 97 | ARGRAVAGNPLD |
| | 98 | QTLYNN |
| | 99 | SGS |
| | 100 | QHYTPWPPYT |
| T13-MRCA | 101 | GFTFSNHD |
| | 102 | IGNAGDT |
| | 103 | ARGIAVAGFPLDV |
| | 104 | QSVNSN |
| | 105 | GAS |
| | 106 | QQYNPWPPWT |
| T12-MRCA | 107 | GFTFSNHD |
| | 108 | IGNAGDT |
| | 109 | ARGIAVAGNPLDV |
| | 110 | QSVNSN |
| | 111 | GAS |
| | 112 | QQYNPWPPWT |
| T13-GL | 113 | GFTFSSYD |
| | 114 | IGTAGDT |
| | 115 | ARGIAVAGFPLDV |
| | 116 | QSVSSN |
| | 117 | GAS |
| | 118 | QQYNNWPPWT |
| T12-GL | 119 | GFTFSSYD |
| | 120 | IGTAGDT |
| | 121 | ARGIAVAGNPLDV |
| | 122 | QSVSSN |
| | 123 | GAS |
| | 124 | QQYNNWPPWT |
| T14 | 125 | GFTFNNYW |
| | 126 | IKGDGSEK |
| | 127 | ARVGGGDYYDSSGYYWLDT |
| | 128 | QSVLYSSNNKNY |
| | 129 | WAS |
| | 130 | QQYYENPT |
| T15 | 131 | GFTFNNYW |
| | 132 | VNQDGNEK |
| | 133 | ARVGGGDYYDSSGYYWFDT |
| | 134 | QSVLYNSNNKNY |
| | 135 | WAS |
| | 136 | QQYYDTPT |
| T1 | 137 | GFSFGDNA |
| | 138 | IRAKGYGGTT |
| | 139 | AKYASGWEVGFDP |
| | 140 | QFIRND |
| | 141 | AAS |
| | 142 | LODYNFPWT |
| T2 | 143 | GYNFKAYG |
| | 144 | ITPYNGKT |
| | 145 | ARTPAALASFDY |
| | 146 | QSISSW |
| | 147 | DAS |
| | 148 | QQYYSYST |
| T3 | 149 | GFTYFSYA |
| | 150 | VNVRVGTS |
| | 151 | ATVGATQDLRYFDF |
| | 152 | QSVYRL |
| | 153 | DAF |
| | 154 | QQYNSYST |
| T4 | 155 | GFTFTSYG |
| | 156 | INTYNGNT |
| | 157 | ARGQGRYGDYIYNH |
| | 158 | QIISSW |
| | 159 | DAS |
| | 160 | QHYNDFPLS |
| T5 | 161 | GFTFSSHA |
| | 162 | ISYDGYNK |
| | 163 | ARDRDSSGYIFDY |
| | 164 | QDISNY |
| | 165 | DAS |
| | 166 | QQYDNLPS |
| T6 | 167 | GDSVSSNSAA |
| | 168 | TYYRSRWYS |
| | 169 | ARDLGIAAADWEDS |
| | 170 | QSVSNR |
| | 171 | DAS |
| | 172 | HQYHNWPG |
| T7 | 173 | GFIFNMYG |
| | 174 | ISYDGTKE |
| | 175 | ARSPSGHALDL |
| | 176 | QSLLLSNGYNY |
| | 177 | LAS |
| | 178 | MQGLQTPFT |
| T8 | 179 | GGSIRGYS |
| | 180 | MLYTGTT |
| | 181 | ARGPTVSGPIVVDY |
| | 182 | QSISTW |
| | 183 | KAS |
| | 184 | QQYYNYGIT |
| T10 | 185 | GYSFTRYG |
| | 186 | ISAYNGDT |
| | 187 | GRDTRYCSGAGCPRPSWYYYYMDV |
| | 188 | QTISNH |
| | 189 | AAS |
| | 190 | QQTYDIPPYT |
| T11 | 191 | GFTFSRYG |
| | 192 | IRYDGTEK |
| | 193 | ANPYITPPTNDY |
| | 194 | QDISNY |
| | 195 | GAS |
| | 196 | QKHNSAPWT |
| T17 | 197 | GFTYFGFA |
| | 198 | INLLSGTT |
| | 199 | AKVGATQDLHYFDF |
| | 200 | QSVYRW |
| | 201 | GAF |
| | 202 | QQYNSHST |
| T3-16-17 MRCA1 | 203 | GFTFFSYA |
| | 204 | INVRGGTT |
| | 205 | AKVGATQDLRYFDY |
| | 206 | QSVSRW |
| | 207 | DAF |
| | 208 | QQYNSYST |
| T3-16-17 MRCA2 | 209 | GFTFFSYA |
| | 210 | INVLGGTT |
| | 211 | AKVGATQDLHYFDY |
| | 212 | QSVSRW |
| | 213 | DAF |
| | 214 | QQYNSYST |
| T3-16-17 GL | 215 | GFTFSSYA |
| | 216 | ISGSGGST |
| | 217 | AKVGATQDLRYFDY |
| | 218 | QSISSW |
| | 219 | DAS |
| | 220 | QQYNSYST |
| T18 | 221 | |
| T19 | 222 | |
| T20 | 223 | |
| T21 | 224 | |
| T22 | 225 | |
| T23 | 226 | |
| T24 | 227 | |
| T25 | 228 | |
| T26 | 229 | |
| T27 | 230 | |
| T28 | 231 | |
| T29 | 232 | |
| T30 | 233 | |
| T1-GL | 234 | |
| T2-GL | 235 | |
| T4-GL | 236 | |
| T5-GL | 237 | |
| T6-GL | 238 | |
| T7-GL | 239 | |
| T8-GL | 240 | |
| T10-GL | 241 | |
| T11-GL | 242 | |
| T18-GL | 243 | |
| T19-GL | 244 | |
| T20-GL | 245 | |
| T21-GL | 246 | |
| T22-GL | 247 | |
| T23-GL | 248 | |
| T25-GL | 249 | |
| T27-GL | 250 | |
| T28-GL | 251 | |
| T30-GL | 252 | |
| Heavy chain | | |
| amino acid | | |
| sequence | | |
| T18 | 253 | |
| T19 | 254 | |
| T20 | 255 | |
| T21 | 256 | |
| T22 | 257 | |
| T23 | 258 | |
| T24 | 259 | |
| T25 | 260 | |
| T26 | 261 | |
| T27 | 262 | |
| T28 | 263 | |
| T29 | 264 | |
| T30 | 265 | |
| T1-GL | 266 | |
| T2-GL | 267 | |
| T4-GL | 268 | |
| T5-GL | 269 | |
| T6-GL | 270 | |
| T7-GL | 271 | |
| T8-GL | 272 | |
| T10-GL | 273 | |
| T11-GL | 274 | |
| T18-GL | 275 | |
| T19-GL | 276 | |
| T20-GL | 277 | |
| T21-GL | 278 | |
| T22-GL | 279 | |
| T23-GL | 280 | |
| T25-GL | 281 | |
| T27-GL | 282 | |
| T28-GL | 283 | |
| T30-GL | 284 | |
| Light chain | | |
| nucleotide | | |
| sequence | | |
| T18 | 285 | |
| T19 | 286 | |
| T20 | 287 | |
| T21 | 288 | |
| T22 | 289 | |
| T23 | 290 | |
| T24 | 291 | |
| T25 | 292 | |
| T26 | 293 | |
| T27 | 294 | |
| T28 | 295 | |
| T29 | 296 | |
| T30 | 297 | |
| T1-GL | 298 | |
| T2-GL | 299 | |
| T4-GL | 300 | |
| T5-GL | 301 | |
| T6-GL | 302 | |
| T7-GL | 303 | |
| T8-GL | 304 | |
| T10-GL | 305 | |
| T11-GL | 306 | |
| T18-GL | 307 | |
| T19-GL | 308 | |
| T20-GL | 309 | |
| T21-GL | 310 | |
| T22-GL | 311 | |
| T23-GL | 312 | |
| T25-GL | 313 | |
| T27-GL | 314 | |
| T28-GL | 315 | |
| T30-GL | 316 | |
| Light chain | | |
| amino acid | | |
| sequence | | |
| T18 | 317 | |
| T19 | 318 | |
| T20 | 319 | |
| T21 | 320 | |
| T22 | 321 | |
| T23 | 322 | |
| T24 | 323 | |
| T25 | 324 | |
| T26 | 325 | |
| T27 | 326 | |
| T28 | 327 | |
| T29 | 328 | |
| T30 | 329 | |
| T1-GL | 330 | |
| T2-GL | 331 | |
| T4-GL | 332 | |
| T5-GL | 333 | |
| T6-GL | 334 | |
| T7-GL | 335 | |
| T8-GL | 336 | |
| T10-GL | 337 | |
| T11-GL | 338 | |
| T18-GL | 339 | |
| T19-GL | 340 | |
| T20-GL | 341 | |
| T21-GL | 342 | |
| T22-GL | 343 | |
| T23-GL | 344 | |
| T25-GL | 345 | |
| T27-GL | 346 | |
| T28-GL | 347 | |
| T30-GL | 348 | |
| CDRs | | |
| T18 | 349 | GFTFSSYW |
| | 350 | IKPDGSEK |
| | 351 | ARDLRYCSSTSCSPALDY |
| | 352 | QSIASY |
| | 353 | AAS |
| | 354 | QQSYTTPRT |
| T19 | 355 | GFTFDNYG |
| | 356 | ISSGGVGT |
| | 357 | AKDLLRYESSGYSP |
| | 358 | QSVNTY |
| | 359 | DAS |
| | 360 | QQRANRPPLT |
| T20 | 361 | GAFLNSGGYY |
| | 362 | IYYSGTT |
| | 363 | RAPRIPFGEVIGGAAFDV |
| | 364 | QSVTSNF |
| | 365 | AAS |
| | 366 | EQYGSSPRT |
| T21 | 367 | GAFLNSGGYY |
| | 368 | GGSFSNSNYY |
| | 369 | IYASAIT |
| | 370 | ARGFRLAAEAYYHGMDV |
| | 371 | QSINNF |
| | 372 | GAS |
| | 373 | QQSDTTPWT |
| T22 | 374 | GGTFSRSA |
| | 375 | IIRIENTA |
| | 376 | ATSSLSDIVVAEGAFVDHYFGMDV |
| | 377 | QTILYSSNNNNY |
| | 378 | WAS |
| | 379 | QQYYSSPLT |
| T23 | 380 | AFTLSRYE |
| | 381 | ISSSGDTI |
| | 382 | ATWGLGYCNSTGCYITDGMDV |
| | 383 | QSISSY |
| | 384 | AAS |
| | 385 | QQSFNTPR |
| T24 | 386 | GGSISSSDYS |
| | 387 | IYHSGNT |
| | 388 | ARDPGGNSGWFDP |
| | 389 | ALPSQY |
| | 390 | KDN |
| | 391 | QSADRSGRYV |
| T25 | 392 | GFIFSNTW |
| | 393 | IKSKADGGTT |
| | 394 | TTGWYSTLDY |
| | 395 | TSNVGTNY |
| | 396 | RND |
| | 397 | AAWDDSLSSWV |
| T26 | 398 | GYTFTSNG |
| | 399 | ISAYNGNT |
| | 400 | ARSRGHYGDYLYGY |
| | 401 | SSNIGAGYD |
| | 402 | GNS |
| | 403 | QSFDSSLSGSVV |
| T27 | 404 | GFTFETYA |
| | 405 | ISFNSRSR |
| | 406 | ARDEKWGTPSD |
| | 407 | KNDVGSYNH |
| | 408 | EVN |
| | 409 | SSYAPMTTLV |
| T28 | 410 | GGSIRGYS |
| | 411 | MLYTGTT |
| | 412 | ARGPTVSGPIVVDY |
| | 413 | SGSIASNY |
| | 414 | EDK |
| | 415 | QSFDSSNRWV |
| T29 | 416 | GGSISSSSYY |
| | 417 | LSYTGST |
| | 418 | ARESGSGGTHTDS |
| | 419 | SSNIGNNY |
| | 420 | DNN |
| | 421 | GTWDSSLSAWV |
| T30 | 422 | GFTFEDYG |
| | 423 | NWNGGST |
| | 424 | ARDKAIQGALMVYAMRGRWFDP |
| | 425 | TGAVTSGHY |
| | 426 | DTS |
| | 427 | FLSYNGARV |
| T1-GL | 428 | GFTFGDYA |
| | 429 | IRSKAYGGTT |
| | 430 | TKYASGWEVGFDP |
| | 431 | QGIRND |
| | 432 | AAS |
| | 433 | LODYNYPWT |
| T2-GL | 434 | GYTFTSYG |
| | 435 | ISAYNGNT |
| | 436 | ARTPAALASFDY |
| | 437 | QSISSW |
| | 438 | DAS |
| | 439 | QQYNSYST |
| T4-GL | 440 | GYTFTSYG |
| T4-GL | 441 | ISAYNGNT |
| T4-GL | 442 | ARGQGRYGDYIYNH |
| T4-GL | 443 | QSISSW |
| T4-GL | 444 | DAS |
| T4-GL | 445 | QQYNDFPLT |
| T5-GL | 446 | GFTFSSYA |
| T5-GL | 447 | ISYDGSNK |
| T5-GL | 448 | ARDRDSSGYIFDY |
| T5-GL | 449 | QDISNY |
| T5-GL | 450 | DAS |
| T5-GL | 451 | QQYDNLPS |
| T6-GL | 452 | GDSVSSNSAA |
| T6-GL | 453 | TYYRSKWYN |
| T6-GL | 454 | ARDLGIAAADWFDS |
| T6-GL | 455 | QSVSSN |
| T6-GL | 456 | GAS |
| T6-GL | 457 | QQYNNWPG |
| T7-GL | 458 | GFTFSSYG |
| T7-GL | 459 | ISYDGSNK |
| T7-GL | 460 | ARSPSGHAFDV |
| T7-GL | 461 | QSLLHSNGYNY |
| T7-GL | 462 | LGS |
| T7-GL | 463 | MQALQTPFT |
| T8-GL | 464 | GGSISSYY |
| T8-GL | 465 | IYYSGST |
| T8-GL | 466 | ARGPTVSGPIVVDY |
| T8-GL | 467 | QSISSW |
| T8-GL | 468 | KAS |
| T8-GL | 469 | QQYNSYGIT |
| T10-GL | 470 | GYTFTSYG |
| T10-GL | 471 | ISAYNGNT |
| T10-GL | 472 | ARDTRYCSGGSCPRPSWYYYYMDV |
| T10-GL | 473 | QSISSY |
| T10-GL | 474 | AAS |
| T10-GL | 475 | QQSYSTPPYT |
| T11-GL | 476 | GFTFSSYG |
| T11-GL | 477 | IRYDGSNK |
| T11-GL | 478 | ANPYITPPTNDY |
| T11-GL | 479 | QGISNY |
| T11-GL | 480 | AAS |
| T11-GL | 481 | QKYNSAPWT |
| T18-GL | 482 | GFTFSSYW |
| T18-GL | 483 | IKQDGSEK |
| T18-GL | 484 | ARDLRYCSSTSCSPALDY |
| T18-GL | 485 | QSISSY |
| T18-GL | 486 | AAS |
| T18-GL | 487 | QQSYSTPRT |
| T19-GL | 488 | GFTFSSYA |
| T19-GL | 489 | ISGSGGST |
| T19-GL | 490 | AKDLLRYDSSGYSP |
| T19-GL | 491 | QSVSSY |
| T19-GL | 492 | DAS |
| T19-GL | 493 | QQRSNWPPLT |
| T20-GL | 494 | GGSISSGGYY |
| T20-GL | 495 | IYYSGST |
| T20-GL | 496 | ARAPRITFGGVIGGAAFDV |
| T20-GL | 497 | QSVSSSY |
| T20-GL | 498 | GAS |
| T20-GL | 499 | QQYGSSPRT |
| T21-GL | 500 | GGSISSGSYY |
| T21-GL | 501 | IYTSGST |
| T21-GL | 502 | ARGLRLAAEAYYYGMDV |
| T21-GL | 503 | QSISSY |
| T21-GL | 504 | AAS |
| T21-GL | 505 | QQSYSTPWT |
| T22-GL | 506 | GGTFSSYA |
| T22-GL | 507 | IIPIFGTA |
| T22-GL | 508 | ATSSLSDIVVVVAAFVDHYYGMDV |
| T22-GL | 509 | QSVLYSSNNKNY |
| T22-GL | 510 | WAS |
| T22-GL | 511 | QQYYSTPLT |
| T23-GL | 512 | GFTFSSYE |
| T23-GL | 513 | ISSSGSTI |
| T23-GL | 514 | ATWGLGYCSSTSCYITDGMDV |
| T23-GL | 515 | QSISSY |
| T23-GL | 516 | AAS |
| T23-GL | 517 | QQSYSTPRT |
| T25-GL | 518 | GFTFSNAW |
| T25-GL | 519 | IKSKTDGGT |
| T25-GL | 520 | TTGCYSTLDY |
| T25-GL | 521 | SSNIGSNY |
| T25-GL | 522 | RNN |
| T25-GL | 523 | AAWDDSLSGWV |
| T27-GL | 524 | GFTFDDYA |
| T27-GL | 525 | ISWNSGSI |
| T27-GL | 526 | AKDEKWGTPSD |
| T27-GL | 527 | SSDVGSYNL |
| T27-GL | 528 | EVS |
| T27-GL | 529 | CSYAGSSTLV |
| T28-GL | 530 | GGSISSYY |
| T28-GL | 531 | IYYSGST |
| T28-GL | 532 | ARGPTVSGPIVVDY |
| T28-GL | 533 | SGSIASNY |
| T28-GL | 534 | EDN |
| T28-GL | 535 | QSYDSSNRWV |
| T30-GL | 536 | GFTFDDYG |
| T30-GL | 537 | INWNGGST |
| T30-GL | 538 | ARDKAIQGALMVYAMRGRWFDP |
| T30-GL | 539 | TGAVTSGHY |
| T30-GL | 540 | DTS |
| T30-GL | 541 | LLSYSGARV |

The term “antibody” as used in this invention includes intact molecules as well as functional fragments thereof (such as Fab, F(ab′)2, Fv, scFv, dsFv, or single domain molecules such as VH and VL) that are capable of binding to an epitope of an antigen, in this case PstS.

According to specific embodiments, the antibody is a whole or intact antibody.

According to specific embodiments, the antibody is an antibody fragment.

Suitable antibody fragments for practicing some embodiments of the invention include a complementarity-determining region (CDR) of an immunoglobulin light chain (referred to herein as “light chain”), a complementarity-determining region of an immunoglobulin heavy chain (referred to herein as “heavy chain”), a variable region of a light chain, a variable region of a heavy chain, a light chain, a heavy chain, an Fd fragment, and antibody fragments comprising essentially whole variable regions of both light and heavy chains such as an Fv, a single chain Fv Fv (scFv), a disulfide-stabilized Fv (dsFv), an Fab, an Fab′, and an F(ab′)2.

As used herein, the terms “complementarity-determining region” or “CDR” are used interchangeably to refer to the antigen binding regions found within the variable region of the heavy and light chain polypeptides. Generally, antibodies comprise three CDRs in each of the VH (CDRH1 or H1; CDRH2 or H2; and CDRH3 or H3) and three in each of the VL (CDRL1 or L1; CDRL2 or L2; and CDR L3 or L3).

The identity of the amino acid residues in a particular antibody that make up a variable region or a CDR can be determined using methods well known in the art and include methods such as sequence variability as defined by Kabat et al. (See, e.g., Kabat et al., 992, Sequences of Proteins of Immunological Interest, 5th ed., Public Health Service, NIH, Washington D.C.), location of the structural loop regions as defined by Chothia et al. (see, e.g., Chothia et al., Nature 342:877-883, 989.), a compromise between Kabat and Chothia using Oxford Molecular's AbM antibody modeling software (now Accelrys®, see, Martin et al., 989, Proc. Natl Acad Sci USA. 86:9268; and world wide web site www(dot)bioinf-org(dot)uk/abs), available complex crystal structures as defined by the contact definition (see MacCallum et al., J. Mol. Biol. 262:732-745, 996) and the “conformational definition” (see, e.g., Makabe et al., Journal of Biological Chemistry, 283:56-66, 2008).

CDRs shown in Table 1 were determined as follows: Antibody nucleotide sequences were identified using IgBlast, based on the human IMGT database. CDR sequences are derived based on IgBlast. Amino acid sequences were obtained using the Expasy Translate tool.

As used herein, the “variable regions” and “CDRs” may refer to variable regions and CDRs defined by any approach known in the art, including combinations of approaches.

Functional antibody fragments comprising whole or essentially whole variable regions of both light and heavy chains are defined as follows:
(i) Fv, defined as a genetically engineered fragment consisting of the variable region of the light chain (VL) and the variable region of the heavy chain (VH) expressed as two chains;
(ii) single chain Fv (“scFv”), a genetically engineered single chain molecule including the variable region of the light chain and the variable region of the heavy chain, linked by a suitable polypeptide linker as a genetically fused single chain molecule.
(iii) disulfide-stabilized Fv (“dsFv”), a genetically engineered antibody including the variable region of the light chain and the variable region of the heavy chain, linked by a genetically engineered disulfide bond.
(iv) Fab, a fragment of an antibody molecule containing a monovalent antigen-binding portion of an antibody molecule which can be obtained by treating whole antibody with the enzyme papain to yield the intact light chain and the Fd fragment of the heavy chain which consists of the variable and CH domains thereof;
(v) Fab′, a fragment of an antibody molecule containing a monovalent antigen-binding portion of an antibody molecule which can be obtained by treating whole antibody with the enzyme pepsin, followed by reduction (two Fab′ fragments are obtained per antibody molecule);
(vi) F(ab′)2, a fragment of an antibody molecule containing a monovalent antigen-binding portion of an antibody molecule which can be obtained by treating whole antibody with the enzyme pepsin (i.e., a dimer of Fab′ fragments held together by two disulfide bonds); and
(vii) Single domain antibodies or nanobodies are composed of a single VH or VL domains which exhibit sufficient affinity to the antigen.

According to specific embodiments the antibody heavy chain constant region is chosen from, e.g., IgG1, IgG2, IgG3, IgG4, IgM, IgA, IgA2, IgD, and IgE.

According to specific embodiments, the antibody is an IgG antibody, e.g., IgG1.

According to a specific embodiment the antibody isotype is IgG1 or IgG3.

According to a specific embodiment the antibody isotype is IgG1 or IgG4.

The choice of antibody type will depend on the immune effector function that the antibody is designed to elicit.

According to specific embodiments, the antibody comprises an Fc domain.

According to specific embodiments, the antibody is a naked antibody.

As used herein, the term “naked antibody” refers to an antibody which does not comprise a heterologous effector moiety e.g. therapeutic moiety, detectable moiety.

As used herein “heterologous” means not occurring in nature in conjunction with the antibody.

According to specific embodiments, the antibody comprises a heterologous effector moiety e.g. e.g. therapeutic moiety, detectable moiety. The effector moiety can be proteinaceous or non-proteinaceous; the latter generally being generated using functional groups on the antibody and on the conjugate partner. The effector moiety may be any molecule, including small molecule chemical compounds and polypeptides. For example the effector moiety can be a known drug to cancer.

According to specific embodiments, the antibody is a monoclonal antibody.

Antibody fragments according to some embodiments of the invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 433,647, and references contained therein, which patents are hereby incorporated by reference in their entirety. See also Porter, R. R. [Biochem. J. 73: 9-26 (959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (9720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-05 (99); Bird et al., Science 242:423-426 (988); Pack et al., Bio/Technology: 27-77 (993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 06-0 (99)].

It will be appreciated that for human therapy or diagnostics, humanized antibodies and human antibodies are preferably used.

When referring to humanized antibodies the meaning is to implant the CDRs of the human antibodies on a backbone of a human antibody e.g., human constant region.

According to specific embodiments, the antibody is a humanized antibody. Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 32:522-525 (986); Riechmann et al., Nature, 332:323-329 (988); and Presta, Curr. Op. Struct. Biol., 2:593-596 (992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 32:522-525 (986); Riechmann et al., Nature 332:323-327 (988); Verhoeyen et al., Science, 239:534-536 (988)], by substituting rodent CDRs or CDR sequences for the corresponding sequences of a human antibody. Accordingly, such humanized antibodies are chimeric antibodies, wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

According to preferred embodiments, the antibody is a human antibody, such as that derived from the ascites fluid of ovarian cancer patients.

According to a specific embodiment, the human antibody carries human Vh, Dh, Jh, Vl, J, gene segments such as in germ line antibodies or natural variants thereof. Although synthetic antibodies are also contemplated.

According to a specific embodiment, the antibody is a homolog of a human antibody comprising an amino acid sequence at least 90%, 91%, 92%, 93%, 94%, 95%, 96%, 97% 98% or 99% identical to VH chain of T1, T2, T3, T4, T5, T6, T7, T8, T10, T11, T12, T13, T14, T15, T17, T18, T19, T20, T21, T22, T23, T24, T25, T26, T27, T28, T29, T30, T4-GL, T7-GL, T22-GL, T30-GL, T12 MRCA, T13 MRCA and T3-16-17 MRCA1 and T3-16-17 MRCA2, as long as it is capable of binding ovarian cancer cells.

According to a specific embodiment, the antibody is a homolog of a human antibody comprising an amino acid sequence at least 90%, 91%, 92%, 93%, 94%, 95%, 96%, 97% 98% or 99% identical to VL chain of T1, T2, T3, T4, T5, T6, T7, T8, T10, T11, T12, T13, T14, T15, T17, T18, T19, T20, T21, T22, T23, T24, T25, T26, T27, T28, T29, T30, T4-GL, T7-GL, T22-GL, T30-GL, T12 MRCA, T13 MRCA and T3-16-17 MRCA1 and T3-16-17 MRCA2 as long as it is capable of binding ovarian cancer cells.

As used herein, “sequence identity” or “identity” in the context of two nucleic acid or polypeptide sequences includes reference to the residues in the two sequences which are the same when aligned. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are considered to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of and a non-conservative substitution is given a score of zero, a conservative substitution is given a score between zero and 1. The scoring of conservative substitutions is calculated, e.g., according to the algorithm of Henikoff S and Henikoff J G. [Amino acid substitution matrices from protein blocks. Proc. Natl. Acad. Sci. U.S.A. 992, 89(22): 095-9].

Identity (e.g., percent homology) can be determined using any homology comparison software, including for example, the BlastN or BlastP software of the National Center of Biotechnology Information (NCBI) such as by using default parameters.

When referring to “at least 90% identity” the claimed invention also refer to at least 91%, at least 92%, at least 93%, at least 94%, at least 95%, at least 96%, at least 97%, at least 98%, at least 98% or 100% identity where each represents a different embodiment.

According to a specific embodiment, the level of identity is at least 90% over the entire sequence (any of the VH and/or VL chains described herein) such as determined as described herein.

According to a specific embodiment, the level of identity is at least 90%, 91%, 92%, 93%, 94%, 95%, 96%, 97%, 98%, 99% over at least one (or at least 2, 3, 4 or 5) of the CDR sequences of an antibody of Table 1 as described herein.

Exemplary CDR sequences and complete light and heavy chains of human antibodies are provided in Table 1 above.

Antibody fragments according to some embodiments of the invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945, and references contained therein, which patents are hereby incorporated by reference in their entirety]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (9720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (99); Bird et al., Science 242:423-426 (988); Pack et al., Bio/Technology: 271-77 (993); and U.S. Pat. No. 4,946,778, which is hereby incorporated by reference in its entirety.

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells.

It will be appreciated that for human therapy or diagnostics, humanized antibodies are preferably used.

According to specific embodiments, the antibody is a humanized antibody. Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 32:522-525 (986); Riechmann et al., Nature, 332:323-329 (988); and Presta, Curr. Op. Struct. Biol., 2:593-596 (992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 32:522-525 (986); Riechmann et al., Nature 332:323-327 (988); Verhoeyen et al., Science, 239:534-536 (988)], by substituting rodent CDRs or CDR sequences for the corresponding sequences of a human antibody. Accordingly, such humanized antibodies are chimeric antibodies (U.S. Pat. No. 4,865), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

According to preferred embodiments, the antibody is a human antibody, such as that derived from the ascites fluid of ovarian cancer patients.

According to a specific embodiment, the human antibody carries human Vh, Dh, Jh, Vl, J, gene segments such as in germ line antibodies or natural variants thereof. Although synthetic antibodies are also contemplated, where for example, the CDRs are implanted on human scaffolds of interest.

According to a specific embodiment, the antibody is a homolog of a human antibody comprising an amino acid sequence at least 90%, 91%, 92%, 93%, 94%, 95%, 96%, 97% 98% or 99% identical to VH chain of T1, T2, T3, T4, T5, T6, T7, T8, T10, T11, T12, T13, T14, T15, T17, T18, T19, T20, T21, T22, T23, T24, T25, T26, T27, T28, T29, T30, T4-GL, T7-GL, T22-GL, T30-GL, T12 MRCA or T13 MRCA as long as it is capable of binding ovarian cancer cells.

According to a specific embodiment, the antibody is a homolog of a human antibody comprising an amino acid sequence at least 90%, 91%, 92%, 93%, 94%, 95%, 96%, 97% 98% or 99% identical to VL chain of T1, T2, T3, T4, T5, T6, T7, T8, T10, T11, T12, T13, T14, T15, T17, T18, T19, T20, T21, T22, T23, T24, T25, T26, T27, T28, T29, T30, T4-GL, T7-GL, T22-GL, T30-GL, T12 MRCA or T13 MRCA as long as it is capable of binding ovarian cancer cells.

As used herein, “sequence identity” or “identity” in the context of two nucleic acid or polypeptide sequences includes reference to the residues in the two sequences which are the same when aligned. When percentage of sequence identity is used in reference to proteins it is recognized that residue positions which are not identical often differ by conservative amino acid substitutions, where amino acid residues are substituted for other amino acid residues with similar chemical properties (e.g. charge or hydrophobicity) and therefore do not change the functional properties of the molecule. Where sequences differ in conservative substitutions, the percent sequence identity may be adjusted upwards to correct for the conservative nature of the substitution. Sequences which differ by such conservative substitutions are considered to have “sequence similarity” or “similarity”. Means for making this adjustment are well-known to those of skill in the art. Typically this involves scoring a conservative substitution as a partial rather than a full mismatch, thereby increasing the percentage sequence identity. Thus, for example, where an identical amino acid is given a score of and a non-conservative substitution is given a score of zero, a conservative substitution is given a score between zero and. The scoring of conservative substitutions is calculated, e.g., according to the algorithm of Henikoff S and Henikoff J G. [Amino acid substitution matrices from protein blocks. Proc. Natl. Acad. Sci. U.S.A. 992, 89(22): 095-9].

Identity (e.g., percent homology) can be determined using any homology comparison software, including for example, the BlastN or BlastP software of the National Center of Biotechnology Information (NCBI) such as by using default parameters.

When referring to “at least 90% identity” the claimed invention also refer to at least 91%, at least 92%, at least 93%, at least 94%, at least 95%, at least 96%, at least 97%, at least 98%, at least 98% or 100% identity where each represents a different embodiment.

According to a specific embodiment, the level of identity is at least 90% over the entire sequence (any of the VH and/or VL chains described herein) such as determined as described herein.

According to a specific embodiment, the level of identity is at least 90%, 91%, 92%, 93%, 94%, 95%, 96%, 97%, 98%, 99% over at least one (or at least 2, 3, 4 or 5) of the CDR sequences of an antibody of Table 1 as described herein.

Exemplary CDR sequences and complete light and heavy chains of human antibodies are provided in Table above.

According to an aspect of the invention there is provided a method of producing an antibody, the method comprising:
(a) expressing in a host cell a heterologous polynucleotide encoding the antibody as described herein; and optionally
(b) recovering the antibody from the host cell.

Thus, a polynucleotide encoding an antibody of some embodiments of the invention is cloned into an expression construct selected according to the expression system used. Exemplary polynucleotide sequences are provided in SEQ ID NOs: which appear on Table 1A.

A variety of prokaryotic or eukaryotic cells can be used as host-expression systems to express the antibody of some embodiments of the invention. These include, but are not limited to, microorganisms, such as bacteria transformed with a recombinant bacteriophage DNA, plasmid DNA or cosmid DNA expression vector containing the coding sequence; yeast transformed with recombinant yeast expression vectors containing the coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the coding sequence. Mammalian expression systems can also be used to express the antibodies of some embodiments of the invention.

Examples for mammalian expression vectors include, but are not limited to, pcDNA3, pcDNA3.(+/−), pGL3, pZeoSV2(+/−), pSecTag2, pDisplay, pEF/myc/cyto, pCMV/myc/cyto, pCR3, pSinRep5, DH26S, DHBB, pNMT, pNMT4, pNMT8, which are available from Invitrogen, pCI which is available from Promega, pMbac, pPbac, pBK-RSV and pBK-CMV which are available from Strategene, pTRES which is available from Clontech, and their derivatives. As shown in the Examples section which follows, heavy chains were cloned and expressed on the basis of the AbVec2.0-IGHG1 vector (see: Addgene: AbVec2.0-IGHG1). Light (Kappa) chains were cloned and expressed on the basis of AbVec1.1-IGKC (See: Addgene: AbVec1.1-IGKC), each of which is contemplated herein.

Expression vectors containing regulatory elements from eukaryotic viruses such as retroviruses can be also used. SV40 vectors include pSVT7 and pMT2. Vectors derived from bovine papilloma virus include pBV-MTHA, and vectors derived from Epstein Bar virus include pHEBO, and p2O5. Other exemplary vectors include pMSG, pAV009/A⁺, pMTO0/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

Examples of bacterial constructs include the pET series of E. coli expression vectors [Studier et al. (990) Methods in Enzymol. 85:60-89).

In yeast, a number of vectors containing constitutive or inducible promoters can be used, as disclosed in U.S. Pat. No. 5,932,447. Alternatively, vectors can be used which promote integration of foreign DNA sequences into the yeast chromosome.

In cases where plant expression vectors are used, the expression of the coding sequence can be driven by a number of promoters. For example, viral promoters such as the 35S RNA and 9S RNA promoters of CaMV [Brisson et al. (984) Nature 30:5-54], or the coat protein promoter to TMV [Takamatsu et al. (987) EMBO J. 6:307-3] can be used. Alternatively, plant promoters such as the small subunit of RUBISCO [Coruzzi et al. (984) EMBO J. 3:-680 and Brogli et al., (984) Science 224:838-843] or heat shock promoters, e.g., soybean hsp7.5-E or hsp7.3-B [Gurley et al. (986) Mol. Cell. Biol. 6:559-565] can be used. These constructs can be introduced into plant cells using Ti plasmid, Ri plasmid, plant viral vectors, direct DNA transformation, microinjection, electroporation and other techniques well known to the skilled artisan. See, for example, Weissbach & Weissbach, 988, Methods for Plant Molecular Biology, Academic Press, NY, Section VIII, pp 42-463.

Other expression systems such as insects and mammalian host cell systems which are well known in the art and are further described hereinbelow can also be used by some embodiments of the invention.

According to a specific embodiment, antibodies are expressed in HEK293T cells such as by using polyethyleneimine as the transfection reagent.

It will be appreciated that antibodies can also be produced in in-vivo systems such as in mammals, e.g., goats, rabbits etc.

Recovery of the recombinant antibody is effected following an appropriate time (in culture). The phrase “recovering the antibody” refers to collecting the whole fermentation medium containing the antibody and need not imply additional steps of separation or purification. Notwithstanding the above, antibodies of some embodiments of the invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

Once antibodies are obtained, they may be tested for activity.

Thus, antibodies described herein may be tested and/or characterized using a variety of methods. Such methods may be used to determine a variety of characteristics that may include, but are not limited to, antibody affinity; specificity; and activity (e.g., activation or inhibition of cellular signaling pathways, target binding, cell killing or other cellular or biological activities). Antibody testing may further include testing in vivo (e.g., in animal and/or human studies) for one or more of toxicity, therapeutic effect, pharmacodynamics, pharmacokinetics, absorption, deposition, metabolism, and excretion. Testing in animals may include, but is not limited to, testing in mice, rats, rabbits, guinea pigs, pigs, primates (e.g., cynomolgus monkeys), sheep, goats, horses, and cattle.

In some embodiments, antibodies of the present invention may be tested or characterized through the use of one or more cell-based assays. Such cell-based assays may be carried out in vitro with cells in culture. In some cases, cell-based assays may be carried out in vivo. Examples of cell-based in vivo assays include tumor models in which tumor cells are injected or otherwise introduced into a host.

In some cases, cell-based assays used herein may include the use of cancer cells. Many cancer cell lines are available for experiments to test antibodies of the invention. Such cells preferably express the target antigen e.g., MMP14 and/or MMP1. Additionally, cancer cell lines may be used to test antibodies of the invention, where the cancer cell lines are representative of cancer stem cells. Cancer stem cell (CSC) cell lines may be isolated or differentiated from cancer cells grown in culture (e.g., through sorting based on markers specific for cancer stem cells). Cell lines used in cell-based assays may include, but are not limited to ovary and pancreas.

In some embodiments, ovarian cancer cell lines may be used. Such cell lines may include, but are not limited to SKOV3, OVCAR3, OV90 and A2870 cell lines.

OVCAR3 cells were first established using malignant ascites obtained from a patient suffering from progressive ovarian adenocarcinoma (Hamilton, T. C. et al., 983. Cancer Res. 43: 5379-89). Cancer stem cell populations may be isolated from OVCAR3 cell cultures through selection based on specific cell surface markers such as CD44 (involved in cell adhesion and migration), CD33 and CD7 (Liang, D. et al., 202. BMC Cancer. 2: 20, the contents of which are herein incorporated by reference in their entirety). OV90 cells are epithelial ovarian cancer cells that were similarly derived from human ascites (see U.S. Pat. No. 570,038). OV-90 cells may also express CD44 when activated (Meunier, L. et al., 200. Transl Oncol. 3(4): 230-8).

According to some embodiments, the antibody binds MMP14.

As used herein “MMP14” refers to matrix metalloproteinase-14, an enzyme that in humans is encoded by the MMP14 gene.

Also referred to as MMP-14, MMP-X, MT1-MMP, MT-MMP, MTMMP, MTMMP, WNCHRS, matrix metallopeptidase 14.

According to an additional or an alternative embodiment, the antibody binds MMP1.

As used herein “MMP1” also known as “interstitial collagenase” and “fibroblast collagenase” is an enzyme that in humans is encoded by the MMP gene.

According to a specific embodiment, the antibody binds, in addition to MMP14, also MMP9 and MMP13, albeit with a lower affinity.

According to a specific embodiment, the antibody comprises the CDR sequences of T2, T3, T21, T27 or T30 or CDR sequences being at least 90% identical to the CDRs of T2, T3, T21, T27 or T30.

Assays for determining binding of an antibody to a target antigen include, but are not limited to, ELISA and surface plasmon resonance (SPR).

As used herein “binding” or “binds” refers to an antibody-antigen mode of binding, which is generally, in the range of K_{D} below 500 nM, such as determined by ELISA.

According to another specific embodiment, the affinity of the antibody to its antigen is determined by Surface Plasmon Resonance (SPR).

According to a specific embodiment, the kinetic constants of the antibody is determined using biolayer interferometry (e.g., such as with T13).

Specific examples for determining antibody binding are provided in the Examples section which follows.

As used herein the term “K_{D}” refers to the equilibrium dissociation constant between the antigen binding domain and its respective antigen.

According to a specific embodiment, the K_{D} for binding the target (e.g., MMP14) is typically in the range of 0.1-500 nM For example between 1-10 nM, 1-50 nM, 0.1-10 nM, 0.1-50 nM, 0.1-100 nM.

High binders which are specifically contemplated herein include, but are not limited to, T3, T12, T13, T10, T11, T8, T27, T17 or T30.

The antibody may be soluble or non-soluble.

Non-soluble antibodies may be a part of a particle (synthetic or non-synthetic, e.g., liposome) or a cell (e.g., CAR-T cells, in which the antibody is part of a chimeric antigen receptor (CAR) typically as an scFv fragment).

Increasing the cytotoxic activity of an antibody where necessary can also be achieved such as by using an antibody-drug conjugate (ADC) concept. In such a configuration the antibody is attached to a heterologous effector moiety that can be used to increase its toxicity or to render it detectable.

In some embodiments, antibodies of the invention may be developed for antibody drug conjugate (ADC) therapeutic applications. ADCs are antibodies in which one or more cargo (e.g., therapeutic agents) are attached [e.g. directly or via linker (e.g. a cleavable linker or a non-cleavable linker)]. ADCs are useful for delivery of therapeutic agents (e.g., drugs or cytotoxic agents) to one or more target cells or tissues (Panowski, S. et al., 204. mAbs 6: 34-45). In some cases, ADCs may be designed to bind to a surface antigen on a targeted cell. Upon binding, the entire antibody-antigen complex may be internalized and directed to a cellular lysosome. ADCs may then be degraded, releasing the bound cargo.

It will be appreciated that also polyclonal antibodies can be formulated as ADCs and as such are envisaged herein.

The therapeutic agent may be a small molecule drug, a proteinaceous agent, a nucleic acid agent, radio-isotopes and carbohydrate and the like. These can serve as cytotoxic agents, e.g., chemotherapy.

According to a specific embodiment, the therapeutic agent is a nucleic acid sequence (e.g., DNA or RNA, e.g., mRNA) which codes for a viral antigen, in order to elicit an anti viral immune response against the tumor. Examples of viral antigens include, but are not limited to CMV antigens, EBV antigens, Coronavirus antigens and the like. Generally, any mRNA for stimulating an immune response can be used.

Where the cargo is a cytotoxic agent, the target cell will be killed or otherwise disabled. Cytotoxic agents may include, but are not limited to cytoskeletal inhibitors [e.g., tubulin polymerization inhibitors, and kinesin spindle protein (KSP) inhibitors], DNA damaging agents (e.g., calicheamicins, duocarmycins, and pyrrolobenzodiazepine dimers such as talirine and tesirine), topoisomerase inhibitors [e.g., camptothecin compounds or derivatives such as 7-ethyl-0-hydroxycamptothecin (SN-38) and exatecan derivative DXd], transcription inhibitors (e.g., RNA polymerase inhibitors such as amanitin), and kinase inhibitors [e.g., phosphoinositide 3-kinase (PI3K) inhibitors or mitogen-activated protein kinase kinase (MEK) inhibitors].

Tubulin polymerization inhibitors may include, but are not limited to, maytansines (e.g., emtansine [DM] and ravtansine [DM4]), auristatins, tubulysins, and vinca alkaloids or derivatives thereof. Exemplary auristatins include auristatin E (also known as a derivative of dolastatin-0), auristatin EB (AEB), auristatin EFP (AEFP), monomethyl auristatin E (MMAE), monomethyl auristatin F (MMAF), auristatin F and dolastatin. Exemplary tubulysin compounds include naturally occurring tubulysins A, B, C, D, E, F, G, H, I, U, and V, and tubulysin analogs such as pretubulysin D (PTb-D43) and N.sup.4-desacetoxytubulysin H (Tbl). Exemplary vinca alkaloids include vincristine, vinblastine, vindesine, and navelbine (vinorelbine). In some embodiments, cytotoxic agents may include auristatin derivatives [e.g. -aminopropan-2-yl-auristatin F, auristatin F-hydroxypropylamide, auristatin F-propylamide, auristatin F phenylenediamine (AFP)]; tubulysin derivatives; vinca alkaloid derivatives [e.g. N-(3-hydroxypropyl)vindesine (HPV)], and any of those described in U.S. Pat. Nos. 8,524,24; 8,685,383; 8,808,9; and 9,254,339; US Patent Application Publications US205034008A, US2060220696A and US2060022829A; the contents of each of which are herein incorporated by reference in their entirety.

Examples of Gold-standard chemotherapy useful for the treatment of ovarian cancer include, but are not limited to, single or combination therapy such as with a platinum compound (usually cisplatin or carboplatin), and a taxane, such as paclitaxel (Taxol®) or docetaxel (Taxotere®) or Albumin bound paclitaxel (nab-paclitaxel, Abraxane®), Altretamine (Hexalen®), Capecitabine (Xeloda®), Cyclophosphamide (Cytoxan®), Etoposide (VP-6), Gemcitabine (Gemzar®), Ifosfamide (Ifex®), Irinotecan (CPT-, Camptosar®), Liposomal doxorubicin (Doxil®), Melphalan, Pemetrexed (Alimta®), Topotecan, Vinorelbine (Navelbine®).

Examples of Gold-standard chemotherapy useful for the treatment of pancreatic cancer include, but are not limited to Gemcitabine (Gemzar), 5-fluorouracil (5-FU), Oxaliplatin (Eloxatin), Albumin-bound paclitaxel (Abraxane), Capecitabine (Xeloda), Cisplatin, Irinotecan (Camptosar).

In some embodiments, antibody-drug conjugates (ADCs) of the invention may further comprise one or more polymeric carrier connecting the antibody and the therapeutic agents (e.g., antibody-polymer-drug conjugates). As used herein, the term “polymeric carrier” refers to a polymer or a modified polymer, which may be covalently attached to one or more therapeutic agents and/or antibodies. Polymeric carriers may provide additional conjugation sites for therapeutic agents, increasing the drug-to-antibody ratio and enhancing therapeutic effects of ADCs. In some embodiments, polymeric carriers used in this invention may be water soluble and/or biodegradable. Such polymeric carriers may include, but are not limited to poly(ethylene glycol) (PEG), poly(N-(2-hydroxypropyl)methacrylamide) (polyHPMA), poly(.alpha.-amino acids) [e.g., poly(L-lysine), poly(L-glutamic acid), and poly((N-hydroxyalky)glutamine)], carbohydrate polymers [e.g., dextrins, hydroxyethylstarch (HES), and polysialic acid], glycopolysaccharides (e.g., homopolysaccharide such as cellulose, amylose, dextran, levan, fucoidan, carraginan, inulin, pectin, amylopectin, glycogen and lixenan; or homopolysaccharide such as agarose, hyluronan, chondroitinsulfate, dermatansulfate, keratansulfate, alginic acid and heparin), glycolipids, glycoconjugates, polyglycerols, polyvinyl alcohols, poly(acrylic acid), polyketal and polyacetal [e.g., poly(1-hydroxymethylethylene hydroxymethylformal), also known as PHF or FLEXIMER®, described in U.S. Pat. Nos. 5,811,501; 5,863,990; and 5,958,398; the contents of each of which are herein incorporated by reference in their entirety], and derivatives, dendrimers, copolymers and mixtures thereof. For example, the polymeric carrier may include a copolymer of a polyacetal/polyketal (e.g., PHF) and a hydrophilic polymer such as polyacrylates, polyvinyl polymers, polyesters, polyorthoesters, polyamides, polypeptides, and derivatives thereof.

In some embodiments, therapeutic agents are attached (e.g., covalently bonded) to antibodies of the invention directly or via linkers. In some embodiments, therapeutic agents are attached to polymeric carriers directly or via linkers, and the polymeric carriers are attached to the antibodies directly or via linkers. In some embodiments, linkers may comprise an oxalic, malonic, succinic, glutaric, adipic, pimelic, suberic, azelaic, sebacic, phthalic, isophthalic, terephthalic, diglycolic acid, tartaric, glutamic, fumaric, or aspartic moiety, including amide, imide, or cyclic-imide derivatives of each thereof, and each optionally substituted. Exemplary linkers may include any of those disclosed in U.S. Pat. Nos. 8,524,241; 8,685,383; 8,808,911; 9,254,339; and/or 95,552 the contents of each of which are herein incorporated by reference in their entirety.

In some embodiments, linkers may be cleavable linkers. Cleavable linkers may break down under certain conditions (such as changes in pH, temperature, or reduction) or cleaved by enzymes (e.g., proteases and glucuronidases) to allow release of therapeutic agents from ADCs. Such linkers may include a labile bond such as an ester bond, amide bond, or disulfide bond. Non-limiting cleavable linkers may include pH-sensitive linkers (e.g., hydrazone, semicarbazone, thiosemicarbazone, cis-aconitic amide, thioether, orthoester, acetal, or ketal); reduction-sensitive linkers [e.g., N-succinimidyl 3-(2-pyridyldithio)propionate (SPDP), N-succinimidyl 4-(2-pyridyldithio)butanoate (SPDB), N-succinimidyl 4-(2-pyridyldithio)pentanoate (SPP), N-succinimidyl-S-acetylthioacetate (SATA) and N-succinimidyl-oxycarbonyl-alpha-methyl-alpha-(2-pyridyl-dithio)toluene or 2,5-dioxopyrrolidin-1-yl 4-(-(pyridin-2-yldisulfanyl)ethyl)benzoate (SMPT)]; photosensitive linkers; and enzymatically cleavable linkers [e.g., peptide linkers such as valine-citrulline, valine-citrulline-p-aminobenzoyloxycarbonyl (vc-PAB), maleimidocaproyl-valine-citrulline-p-aminobenzoyloxycarbonyl (MC-vc-PAB), linkers cleavable by glucuronidases, such as glucuronide-MABC, or linkers cleavable by esterases].

In other embodiments, linkers may be non-cleavable linkers. Non-cleavable linkers may increase plasma stability of the ADCs compared to cleavable linkers. Exemplary non-cleavable linkers include maleimide alkane and maleimide cyclohexane (MCC).

Antibody-drug conjugates (ADCs) of the invention may be prepared using any method known in the art. For example, therapeutic agents may be modified to contain a functional group that can react with a functional group on the antibody. Antibody-drug conjugates (ADCs) may be prepared by reacting the two functional groups to form a conjugate. In some cases, polymeric carriers may be modified to contain functional groups that can react with the functional group on the therapeutic agents and the functional group on the antibody under different chemical conditions. Antibodies, polymeric carriers, and therapeutic agents may be linked to form the antibody-polymer-drug conjugates through sequential chemical reactions. Conjugation to antibodies may employ a lysine or a cysteine residue as the conjugation site. In some embodiments, antibodies may be engineered to have additional lysine or cysteine residues. Such approaches may avoid disruption of antibody structure (e.g., interchain disulfide bonds) and maintain antibody stability and/or activity.

In some embodiments, antibodies of the invention may be tested for their ability to promote cell death per se or when developed as ADCs.

In some embodiments, antibody sequences of the invention may be used to develop a chimeric antigen receptor (CAR). CARs are transmembrane receptors expressed on immune cells that facilitate recognition and killing of target cells (e.g. tumor cells). CARs typically include three basic parts. These include an ectodomain (also known as the recognition domain), a transmembrane domain and an intracellular (signaling) domain. Ectodomains facilitate binding to cellular antigens on target cells, while intracellular domains typically include cell signaling functions to promote the killing of bound target cells. Further, they may have an extracellular domain with one or more of the antibody variable domains described herein or fragments thereof. CARs of the invention also include a transmembrane domain and cytoplasmic tail. CARs may be designed to include one or more segments of an antibody, antibody variable domain and/or antibody CDR, such that when such CARs are expressed on immune effector cells, the immune effector cells bind and clear any cells that are recognized by the antibody portions of the CARs.

Characteristics of CARs include their ability to redirect T-cell specificity and reactivity toward a selected target in a non-MHC-restricted manner, exploiting the antigen-binding properties of monoclonal antibodies. The non-MHC-restricted antigen recognition gives T cells expressing CARs the ability to recognize antigen independent of antigen processing, thus bypassing a major mechanism of tumor escape. Moreover, when expressed in T-cells, CARs advantageously do not dimerize with endogenous T cell receptor (TCR) alpha and beta chains.

CARs engineered to target tumors may have specificity for MMP14 and/or MMP according to some embodiments of the invention. In some embodiments, ectodomains of these CARs may include one or more antibody variable domains or a fragment thereof. In some embodiments, CARs are expressed in T cells, and may be referred to as “CAR-engineered T cells” or “CAR-Ts”. CAR-Ts may be engineered with CAR ectodomains having one or more antibody variable domains.

Thus, in some embodiments of the present disclosure, antibody sequences of the invention may be used to develop a chimeric antigen receptor (CAR). In some embodiments, CARs are transmembrane receptors expressed on immune cells that facilitate recognition and killing of target cells (e.g. tumor cells).

In some embodiments, antibodies of the present invention may bind more than one epitope. As used herein, the terms “multibody” or “multispecific antibody” refer to an antibody wherein two or more variable regions bind to different epitopes. The epitopes may be on the same or different targets. In certain embodiments, a multi-specific antibody is a “bispecific antibody,” which recognizes two different epitopes on the same or different antigens.

Bispecific antibodies are capable of binding two different antigens. Such antibodies typically comprise antigen-binding regions from at least two different antibodies. For example, a bispecific monoclonal antibody (BsMAb, BsAb) is an artificial protein composed of fragments of two different monoclonal antibodies, thus allowing the BsAb to bind to two different types of antigen. One common application for this technology is in cancer immunotherapy, where BsMAbs are engineered to simultaneously bind to a cytotoxic cell (using a receptor like CD3) and a target like a tumor cell to be destroyed.

Bispecific antibodies may include any of those described in Riethmuller, G., 202. Cancer Immunity. 2:2-8; Marvin, J. S. et al., 2005. Acta Pharmacologica Sinica. 26(6):649-58; and Schaefer, W. et al., 20. PNAS. 08(27):87-92, the contents of each of which are herein incorporated by reference in their entirety.

New generations of BsMAb, called “trifunctional bispecific” antibodies, have been developed. These consist of two heavy and two light chains, one each from two different antibodies, where the two Fab regions (the arms) are directed against two antigens, and the Fc region (the foot) comprises the two heavy chains and forms the third binding site.

Other types of bispecific antibodies have been designed to overcome certain problems, such as short half-life, immunogenicity and side-effects caused by cytokine liberation and are contemplated herein. They include chemically linked Fabs, consisting only of the Fab regions, and various types of bivalent and trivalent single-chain variable fragments (scFvs), fusion proteins mimicking the variable domains of two antibodies. The furthest developed of these newer formats are the bi-specific T-cell engagers (BiTEs) and mAb2's, antibodies engineered to contain an Fcab antigen-binding fragment instead of the Fc constant region.

In some embodiments, antibodies of the present invention may be diabodies. Diabodies are functional bispecific single-chain antibodies (bscAb). These bivalent antigen-binding molecules are composed of non-covalent dimers of scFvs, and can be produced in mammalian cells using recombinant methods. (See, e.g., Mack et al, Proc. Natl. Acad. Sci., 92: 702-7025, 995). Few diabodies have entered clinical development. An iodine-23-labeled diabody version of the anti-CEA chimeric antibody cT84.66 has been evaluated for pre-surgical immunoscintigraphic detection of colorectal cancer in a study sponsored by the Beckman Research Institute of the City of Hope (Clinicaltrials(dot)gov NCT0064753) (Nelson, A. L., MAbs. 200. January-February; 2( ):77-83).

Also included are maxibodies (bivalent scFV fused to the amino terminus of the Fc (CH2-CH3 domains) of IgG.

Bispecific T-cell-engager (BiTE) antibodies are designed to transiently engage cytotoxic T-cells for lysis of selected target cells. These typically include two scFvs (one binding to CD3 on Tcells and one binding to a target antigen on the surface of a cell being targeted for destruction). In some embodiments, the two scFvs are joined by a linker. In other embodiments, the two scFvs are different regions on an antibody. The clinical activity of BiTE antibodies corroborates findings that ex vivo expanded, autologous T-cells derived from tumor tissue, or transfected with specific T-cell receptors, have shown therapeutic potential in the treatment of solid tumors. While these personalized approaches prove that T-cells alone can have considerable therapeutic activity, even in late-stage cancer, they are cumbersome to perform on a broad basis. This is different for cytotoxic T-lymphocyte antigen 4 (CTLA-4) antibodies, which facilitate generation of tumor-specific T-cell clones, and also for bi- and tri-specific antibodies that directly engage a large proportion of patients' T-cells for cancer cell lysis. The potential of global T-cell engagement for human cancer therapy by T-cell-engaging antibodies is under active investigation (Baeuerle P A, et al., Current Opinion in Molecular Therapeutics. 2009, ( ):22-30 and Baeuerle P A and Reinhardt C, Cancer Res. 2009, 69(2): 494-4, the contents of each of which are herein incorporated by reference in their entirety).

In a whole antibody, a therapeutic activity is intrinsic to the molecule since the Fc domain activates antibody-dependent cell-mediated cytotoxicity (ADCC). ADCC is a mechanism of cell-mediated immune defense whereby an effector cell of the immune system actively lyses a target cell, whose membrane-surface antigens have been bound by specific antibodies. It is one of the mechanisms through which antibodies, as part of the humoral immune response, can act to limit and contain infection. Classical ADCC is mediated by natural killer (NK) cells; macrophages, neutrophils and eosinophils can also mediate ADCC. For example, eosinophils can kill certain parasitic worms known as helminths through ADCC mediated by IgE. ADCC is part of the adaptive immune response due to its dependence on a prior antibody response.

The term “Fc domain” or “Fc region” herein is used to define a C-terminal region of an immunoglobulin heavy chain that contains at least a portion of the constant region. The term includes native sequence Fc regions and variant Fc regions. Unless otherwise specified herein, numbering of amino acid residues in the Fc region or constant region is according to the EU numbering system, also called the EU index, as described in Kabat et al, Sequences of Proteins of Immunological Interest, 5th Ed. Public Health Service, National Institutes of Health, Bethesda, Md., 99. An “Fc polypeptide” of a dimeric Fc as used herein refers to one of the two polypeptides forming the dimeric Fc domain, i.e. a polypeptide comprising C-terminal constant regions of an immunoglobulin heavy chain, capable of stable self-association. For example, an Fc polypeptide of a dimeric IgG Fc comprises an IgG CH2 and an IgG CH3 constant domain sequence. An Fc can be of the class IgA, IgD, IgE, IgG, and IgM, and several of these may be further divided into subclasses (isotypes), e.g., IgG, IgG2, IgG3, IgG4, IgA, and IgA2.

The terms “Fc receptor” and “FcR” are used to describe a receptor that binds to the Fc region of an antibody. For example, an FcR can be a native sequence human FcR. Generally, an FcR is one which binds an IgG antibody (a gamma receptor) and includes receptors of the Fc.gamma.RI, Fc.gamma.RII, and Fc.gamma.RIII subclasses, including allelic variants and alternatively spliced forms of these receptors. Fc.gamma.RII receptors include Fc.gamma.RIIA (an “activating receptor”) and Fc.gamma.RIIB (an “inhibiting receptor”), which have similar amino acid sequences that differ primarily in the cytoplasmic domains thereof. Immunoglobulins of other isotypes can also be bound by certain FcRs (see, e.g., Janeway et al., Immuno Biology: the immune system in health and disease, (Elsevier Science Ltd., NY) (4th ed., 999)). Activating receptor Fc.gamma.RIIA contains an immunoreceptor tyrosine-based activation motif (ITAM) in its cytoplasmic domain. Inhibiting receptor Fc.gamma.RIIB contains an immunoreceptor tyrosine-based inhibition motif (ITIM) in its cytoplasmic domain (reviewed in Daeron, Annu. Rev. Immunol. 5:203-234 (997)). FcRs are reviewed in Ravetch and Kinet, Annu. Rev. Immunol 9:457-92 (99); Capel et al., Immunomethods 4:25-34 (994); and de Haas et al., J. Lab. Clin. Med. 26:330-4 (995). Other FcRs, including those to be identified in the future, are encompassed by the term “FcR” herein. The term also includes the neonatal receptor, FcRn, which is responsible for the transfer of maternal IgGs to the fetus (Guyer et al., J. Immunol. 7:587 (976); and Kim et al., J. Immunol. 24:249 (994)).

Modifications in the CH2 domain can affect the binding of FcRs to the Fc. A number of amino acid modifications in the Fc region are known in the art for selectively altering the affinity of the Fc for different Fcgamma receptors. In some aspects, the Fc comprises one or more modifications to promote selective binding of Fc-gamma receptors.

Exemplary mutations that alter the binding of FcRs to the Fc are listed below:

S298A/E333A/K334A, S298A/E333A/K334A/K326A (Lu Y, Vernes J M, Chiang N, et al. J Immunol Methods. 20 February 28; 365(-2): 32-4);

F243L/R292P/Y300L/V305/P396L, F243L/R292P/Y300L/L235V/P396L (Stavenhagen J B, Gorlatov S, Tuaillon N, et al. Cancer Res. 2007 Sep. 5; (8):8882-90; Nordstrom J L, Gorlatov S, Zhang W, et al. Breast Cancer Res. 20 November 30; 3(6):R23); F243L (Stewart R, Thom G, Levens M, et al. Protein Eng Des Sel. 20 September; 24(9):-8.), S298A/E333A/K334A (Shields R L, Namenuk A K, Hong K, et al. J Biol Chem. 200 March 2; 276(9):659-604);

S239D/I332E/A330L, S239D/I332E (Lazar G A, Dang W, Karki S, et al. Proc Natl Acad Sci USA. 2006 Mar. 4; 03( ):4005-0); S239D/S2E, S2E/L328F (Chu S Y, Vostiar I, Karki S, et al. Mol Immunol. 2008 September; 45(5):3926-33);

S239D/D265S/S298A/I332E, S239E/S298A/K326A/A327H, G237F/S298A/A330L/I332E, S239D/I332E/S298A, S239D/K326E/A330L/I332E/S298A, G236A/S239D/D270L/I332E, S239E/S2E/H268D, L 234F/S2E/N325L, G237F/V266L/S2D and other mutations listed in WO20/2034 and WO20/2035, herein incorporated by reference. Therapeutic Antibody Engineering (by William R. Strohl and Lila M. Strohl, Woodhead Publishing series in Biomedicine No, ISBN 907568 37 9, October 202) lists mutations on page 283.

In some embodiments an antibody described herein includes modifications to improve its ability to mediate effector function. Such modifications are known in the art and include afucosylation, or engineering of the affinity of the Fc towards an activating receptor, mainly FCGR3a for ADCC, and towards Cq for CDC.

Methods of producing antibodies with little or no fucose on the Fc glycosylation site (Asn 297 EU numbering) without altering the amino acid sequence are well known in the art.

In some embodiments, an antibody has antibody-dependent cellular phagocytosis (ADCP) activity. ADCP can occur when antibodies bind to antigens on the surface of pathogenic or tumorigenic target-cells. Phagocytic cells bearing Fc receptors on their cell surface, including monocytes and macrophages, recognize and bind the Fc region of antibodies bound to target-cells. Upon binding of the Fc receptor to the antibody-bound target cell, phagocytosis of the target cell can be initiated. ADCP can be considered a form of ADCC.

Antibodies of some embodiments may be useful in the clinic: diagnosis (e.g., predicting survival and rate of progression of HGSOC) and treatment.

Thus, according to an aspect of the invention there is provided a method of prognosing ovarian cancer. The method comprising determining a level of MMP14 using the antibodies of the invention, where a level above a predetermined threshold relative to a healthy control sample (normal sample of the same tissue, e.g., adjacent) is indicative of poor prognosis.

As used herein “prognosing” or “providing prognosis” refers to a predetermined years survival and or rate of progression, where higher expression relative to normal tissue of the same type (control) is indicative of lower survival and/or higher rate of progression. It will be appreciated that high expression of MMP14 (RNA or protein) is indicative of poor prognosis. It will be appreciated that correlation of expression of MMP14 to prognosis according to the Human Protein Atlas is as follows:

High expression of MMP14 entails an overall 5 year survival of 22%

Low expression of MMP14 entails an overall 5 year survival of 35%

In the case of high grade serous ovarian cancer, it was found that antibody coating of above 10% of the tumor cells correlate with superior progression free survival (Median progression free survival: IgG<10%—12.51 months, IgG>10%—35.84 months) and superior overall survival (Median survival: IgG<10%—30.21 months, IgG>10%—93.47 months).

It will be appreciated that MMP14 expression and tumor coating are not necessarily correlated. High expression of MMP14 may confer a worse prognosis for patients, whereas tumor coating may confer better prognosis.

According to a further aspect of the invention there is provided a method of characterizing an MMP14+ tumor, the method comprising: determining coating of the tumor with anti MMP14 antibodies, wherein coating with said anti MMP14 antibodies indicates that the tumor is treatable with adoptive cell therapy.

Coating should be determined using a label, which can be directly conjugated to the antibody (or antibodies) or by the use of an indirect label such as attached to a secondary antibody, however the skilled artisan is aware of various types of labels.

Tumor coating assay is provided in the Examples section which follows.

Tumor coating can be used as a selection parameter for treatment with adoptive cell therapy, as further described hereinbelow.

According to a specific embodiment, the adoptive cell therapy comprises NK cells therapy.

The NK cells can be activated with lymphokines as already known to the skilled artisans, such as with IL-2 and/or IL-15.

Alternatively, genetically modified NK cells can be used such as the NK 92 cell line which is genetically modified with IL2 and CD16a, such cells are also termed as HaNKs and are readily available from ImmunityBio®.

Such a treatment modality can be augmented by further treating the subject with an anti MMP 14 antibody (or antibodies) as described herein.

Also provided is a method of diagnosing ovarian cancer in a subject in need thereof, the method comprising:
(a) providing a utero-tubal lavage of the subject; and
(b) determining in said utero-tubal lavage a presence or level of MMP14, wherein presence or level above a predetermined threshold is indicative of ovarian cancer in the subject.

According to a specific embodiment, determining is by using anti MMP14 antibodies.

The use of uterine liquid biopsies is known in the art but it wasn't known that it includes MMP14.

Barnabas et al. Molecular and Cellular Proteomics 2019 18:865-875, describes methods for obtaining utero-tubal lavage (liquid biopsy) for early detection of ovarian cancer and is hereby incorporated by reference in its entirety.

Once the sample is obtained it can be subjected to various protein detection assays such as ELISA using the antibodies to MMP14 to detect cancer excretions (i.e., MMP14) in the fluid.

As used herein the term “diagnosing” refers to determining presence or absence of a pathology (e.g., a disease, disorder, condition or syndrome), classifying a pathology or a symptom, determining a severity of the pathology, monitoring pathology progression, forecasting an outcome of a pathology and/or prospects of recovery and screening of a subject for a specific disease.

According to some embodiments of the invention, screening of the subject for a specific disease is followed by substantiation of the screen results using gold standard methods such as imaging e.g., PET-CT, which can employ the labeled antibodies (e.g., radio labeled) of the invention.

Such a diagnostic modality, allows early detection of s tubal carcinoma in situ, when any other symptoms are absent.

Once diagnosis is made, the subject can be treated with an anti cancer agent, such as described herein.

Thus, there is provided a method of treating ovarian cancer in a subject in need thereof, the method comprising:
(a) diagnosing the ovarian cancer as described herein;
and
(b) treating the cancer.

Diagnosis/prognosis may be corroborated using Gold-standard methods such as by the use of imaging (as mentioned hereinabove) and molecular markers.

Once diagnosis/prognosis is made the subject is directed to treatment at the discretion of the physiologist.

According to some embodiments, a preparation comprising the antibodies (polyclonal) derived from the tumor such as containing the above-mentioned sequences can be used in the treatment of cancer as described herein.

According to another aspect of the invention, there is provided a method of treating cancer in a subject in need thereof, the method comprising administering to the subject a therapeutically effective amount of the antibody, polynucleotide, construct or cell as described herein, or a polyclonal preparation of antibodies from an ascites fluid of an ovarian cancer patient, thereby treating the cancer in the subject.

Although the present embodiments are especially relevant to ovarian cancer, any other ascites forming cancer is contemplated herein, e.g., ovarian, breast, colon, stomach, pancreas.

According to an embodiment a protein G/A affinity chromatography is used to isolate the ascites borne antibodies. These in the majority of cases are tumor reactive. In the absence of the immune-inhibitory environment of the ascites (e.g., IL-8, TGFb) and in the presence of ADCC competent cytolytic cells such as lymphokine activated NKs, these antibodies can generate an anti-tumor response.

In the case of ovarian cancer, such treatment is preferably effected s following the first surgery—when tumor burden is at its lowest and lacks a robust protective microenvironment.

Alternatively, there is provided the antibody, polynucleotide, construct or cell or a polyclonal preparation of antibodies from an ascites fluid of an ovarian cancer patient as described herein for use in treating cancer in a subject in need thereof.

According to a specific embodiment, the polyclonal preparation of the subject i.e., retrieved from the subject (autologous) and returned

For example, when the patient presents with ascites, the patient undergoes paracentesis (drainage) of the fluids. Diagnosis of cancer (e.g., ovarian) is made such as described herein and/or using gold standard methods. Up until surgery, NK cells are harvested from the peripheral blood of the patient and are activated ex-vivo (e.g., using IL-2 and/or IL-15). In other embodiments universal NK cells (allogeneic cells) are used such as HaNKs as described above. The ascites fluids recovered, undergo protein G/A affinity chromatography-based separation. Immediately following surgery, the patient receives intraperitoneally: (A) a preparation of her autologous antibodies, purified from her ascites fluids. (B) lymphokine activated natural killers.

It will be appreciated that monoclonal antibodies may also be used.

According to another embodiment, the preparation is allogeneic to the subject.

According to a specific embodiment, the cancer is a primary tumor.

According to a specific embodiment, the cancer is metastatic.

According to a specific embodiment, the cancer is drug-resistant.

According to a specific embodiment, the treatment as described herein with the antibody is a first line treatment.

According to a specific embodiment, the treatment is following surgery, e.g., in ovarian cancer typically, bilateral salpingo-oophorectomy or BSO and in pancreatic cancer typically, the Whipple procedure.

According to a specific embodiment, the cancer is MMP14+ and/or MMP1+.

This means that cancer cells overexpress or have elevated above normal levels of MMP14 and/or MMP1. Methods of detecting MMP expression are well known in the art and can be determined at the RNA and/or protein level.

The phrase “elevated above normal”, as used herein, refers to expression of MMP14 and/or MMP1 that is detected at a level significantly greater than the level expected for the same type of diagnostic sample taken from a non-diseased subject or patient (i.e., one who does not have cancer, such as ovarian cancer) of the same gender and of similar age. As further used herein, “significantly greater” refers to a statistically significant difference between the level of MMP14 and/or MMP1 expression elevated above normal and the expected (normal) level of MMP14 and/or MMP1. Preferably, MMP14 and/or MMP1 expression that is elevated above normal is expression of MMP14 and/or MMP1 at a level that is at least 0% greater than the level of MMP14 and/or MMP expression otherwise expected. Where MMP14 and/or MMP1 expression is expected to be absent from a particular diagnostic sample taken from a particular subject or patient, the normal level of MMP14 and/or MMP1 expression for that subject or patient is nil. Where a particular diagnostic sample taken from a particular subject or patient is expected to have a low level of constitutive MMP14 and/or MMP1 expression, that low level is the normal level of MMP14 and/or MMP1 expression for that subject or patient.

A “reference sample” or “control sample”, as discussed herein, is a biological sample provided from a reference or control group of apparently healthy individuals for the purpose of evaluation in vitro. Similarly, the expressions “reference concentration”, “reference value”, and “reference level”, as used herein, refer to a value established in a reference or control group of apparently healthy individuals. Determination of the reference concentration of MMP14 and/or MMP1 or MMP14 and/or MMP expression can be made based on an amount or concentration which best distinguishes patient and healthy populations. By way of example, the value for MMP14 and/or MMP as determined in a control group or a control population establishes a “cut-off value” or a “reference range”. A value above such cut-off or threshold, or outside the reference range at its higher end, is considered to be “elevated above normal” or “diagnostic of ovarian cancer”. The reference level can be a single number, equally applicable to every subject, or the reference level can vary, according to specific subpopulations of subjects. For example, post-menopausal subjects can have a different reference level for ovarian cancer than pre-menopausal subjects. In addition, a subject with more advanced ovarian cancer (e.g., stages II-IV) can have a different reference value than one who has early stage ovarian cancer (e.g., stage I).

TABLE 2
MMP14+ disorders (adapted from Genecards and references therein)
Disorder

fibrosarcoma
cervical squamous cell
carcinoma
larynx cancer
brain cancer
colorectal cancer
ductal carcinoma in situ
ovarian cancer
breast cancer
serous cystadenocarcinoma
tongue squamous cell carcinoma
gallbladder cancer
keratoconus
aortic aneurysm, familial
abdominal,
melanoma in congenital
melanocytic nevus
gastric cancer
esophageal cancer
pancreatic cancer
prostate cancer
endometrial cancer
melanoma
lung cancer
squamous cell carcinoma, head
and neck
rhabdomyosarcoma
hepatocellular carcinoma

MMP1+ disorders include but are not limited to ovarian cancer, gastric cancer. esophageal squamous cell carcinoma, Cervical Squamous Cell Carcinoma, head and neck cancer, cervical cancer, liver, or renal cancer.

According to a specific embodiment, the cancer is ovarian cancer.

Ovarian cancer is classified into two categories according to its histological and molecular characteristics, both of which should be considered as combined or separate embodiments of the present teachings. Type I tumors consist of low grade tumors which grow in a step wise fashion, such as low grade serous ovarian carcinoma as well as ovarian carcinomas of endometroid, clear cell, mucinous and transitional histologies. These tumors comprise distinct molecular aberrations which are absent from type II tumors. For example, these include mutations involving elements of the mitogen activated protein kinase (MAPK) pathway—such as BRAF and KRAS for the serous and mucinous tumors and mutations in PTEN and β-catenin for tumors of endometroid histology. Conversely, Type II tumors consist of high grade neoplasms including high grade serous ovarian carcinoma (HGSOC), carcinosarcoma and undifferentiated ovarian carcinoma. These tumors are characterized by recurrent mutations in BRCA, BRCA2 and specifically p53—which is nearly universally mutated (96%) in HGSOC. While type I tumors arise from the ovarian surface epithelium, it is commonly accepted that type II tumors originate from the fallopian tube epithelium.

According to a specific embodiment, the ovarian cancer is high grade serous ovarian carcinoma (HGSOC).

According to another specific embodiment, the cancer is pancreatic cancer.

Pancreatic cancer is typically divided into two general groups both of which should be considered as combined or separate embodiments of the present teachings. The vast majority of cases (about 95%) occur in the part of the pancreas that produces digestive enzymes, known as the exocrine component. Several subtypes of exocrine pancreatic cancers are described, but their diagnosis and treatment have much in common. The small minority of cancers that arise in the hormone-producing (endocrine) tissue of the pancreas have different clinical characteristics and are called pancreatic neuroendocrine tumors, sometimes abbreviated as “PanNETs”.

The term “treating” refers to inhibiting, preventing or arresting the development of a pathology (e.g., cancer) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “preventing” refers to keeping a disease, disorder or condition from occurring in a subject who may be at risk for the disease, but has not yet been diagnosed as having the disease.

As used herein, the term “subject” includes mammals, preferably human beings at any age which suffer from the pathology. Preferably, this term encompasses individuals who are at risk to develop the pathology. According to a specific embodiment, the subject is a female suffering from ovarian cancer.

The antibodies of some embodiments of the invention can be administered to an organism per se, or in a pharmaceutical composition where it is mixed with suitable carriers or excipients.

Thus, according to an aspect of the invention there is provided a pharmaceutical composition comprising the antibody, cell, polynucleotide, construct as described herein.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the antibody, cell, polynucleotide, construct accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, PA, latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, intraperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

Pharmaceutical compositions of some embodiments of the invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with some embodiments of the invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to some embodiments of the invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of some embodiments of the invention may also be formulated in rectal compositions such as suppositories or retention enemas, using, e.g., conventional suppository bases such as cocoa butter or other glycerides.

Pharmaceutical compositions suitable for use in context of some embodiments of the invention include compositions wherein the active ingredients are contained in an amount effective to achieve the intended purpose. More specifically, a therapeutically effective amount means an amount of active ingredients (antibody, cell, polynucleotide, construct) effective to prevent, alleviate or ameliorate symptoms of a disorder (e.g., cancer) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 975, in “The Pharmacological Basis of Therapeutics”, Ch. p.).

Dosage amount and interval may be adjusted individually to provide effective tissue levels of the active ingredient are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of some embodiments of the invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

Treatment may be augmented by the use of other treatment modules such as chemotherapy, radiotherapy, biological therapy (other than the claimed antibodies) or surgery.

As used herein the term “about” refers to ±0%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from to 6 should be considered to have specifically disclosed subranges such as from to 3, from to 4, from to 5, from 2 to 4, from 2 to 6, from 3 to 6 etc., as well as individual numbers within that range, for example 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than in 50 nucleotides, alternatively, less than in 00 nucleotides, alternatively, less than in 200 nucleotides, alternatively, less than in 500 nucleotides, alternatively, less than in 000 nucleotides, alternatively, less than in 5,000 nucleotides, alternatively, less than in 0,000 nucleotides.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (989); “Current Protocols in Molecular Biology” Volumes I-III Ausubel, R. M., ed. (994); Ausubel et al., “Current Protocols in Molecular Biology”, John Wiley and Sons, Baltimore, Maryland (989); Perbal, “A Practical Guide to Molecular Cloning”, John Wiley & Sons, New York (988); Watson et al., “Recombinant DNA”, Scientific American Books, New York; Birren et al. (eds) “Genome Analysis: A Laboratory Manual Series”, Vols.-4, Cold Spring Harbor Laboratory Press, New York (998); methodologies as set forth in U.S. Pat. Nos. 4,666,828; 4,683,202; 48,053; 592,659 and 5,272,057; “Cell Biology: A Laboratory Handbook”, Volumes I-III Cellis, J. E., ed. (994); “Culture of Animal Cells—A Manual of Basic Technique” by Freshney, Wiley-Liss, N. Y. (994), Third Edition; “Current Protocols in Immunology” Volumes I-III Coligan J. E., ed. (994); Stites et al. (eds), “Basic and Clinical Immunology” (8th Edition), Appleton & Lange, Norwalk, CT (994); Mishell and Shiigi (eds), “Selected Methods in Cellular Immunology”, W. H. Freeman and Co., New York (980); available immunoassays are extensively described in the patent and scientific literature, see, for example, U.S. Pat. Nos. 379,932; 3,839,53; 3,850,752; 3,850,578; 3,853,987; 3,857; 3,879,262; 390,654; 3,935,074; 3,984,533; 3,996,345; 4,034,074; 4,098,876; 487,929; 5,077 and 52,852; “Oligonucleotide Synthesis” Gait, M. J., ed. (984); “Nucleic Acid Hybridization” Hames, B. D., and Higgins S. J., eds. (985); “Transcription and Translation” Hames, B. D., and Higgins S. J., eds. (984); “Animal Cell Culture” Freshney, R. I., ed. (986); “Immobilized Cells and Enzymes” IRL Press, (986); “A Practical Guide to Molecular Cloning” Perbal, B., (984) and “Methods in Enzymology” Vol.-37, Academic Press; “PCR Protocols: A Guide To Methods And Applications”, Academic Press, San Diego, CA (990); Marshak et al., “Strategies for Protein Purification and Characterization—A Laboratory Course Manual” CSHL Press (996); all of which are incorporated by reference as if fully set forth herein. Other general references are provided throughout this document. The procedures therein are believed to be well known in the art and are provided for the convenience of the reader. All the information contained therein is incorporated herein by reference.

### Materials and Methods

### Flow Cytometric Analysis and Single Cell Sorting of Tumor Infiltrating ASCs

Fresh HGSOC primary tumors retrieved from the operating theatre were immediately dissociated to a single cell suspension in growth medium (DMEM, 10% foetal bovine serum, 1× MEM-Eagle non essential amino acids, 2 mM glutamine, 1:100 Pen-Strep solution) and placed on ice. Cells were washed in PBS and stained with the following antibodies: Alexa fluor 700 conjugated anti-human CD19 (Biolegend, clone: HIB19), PerCP/Cy5.5 conjugated anti-human CD38 (Biolegend, Clone: HIT2), APC conjugated anti-human IgG1 FC region (RD Systems, Clone: #97924), PE conjugated anti-human IgM (Biolegend, Clone: MHM-88) and Alexa fluor 488 conjugated anti-human IgK (Biolegend, Clone: MHK-49). Samples were stained on ice for 45 minutes, washed and acquired using a Cytoflex flow cytometer and analysed using FlowJo 10.5.3. For future immunoglobulin sequencing purposes, tumor infiltrating CD19⁺, CD38⁺⁺, IgM⁻, IgG1⁺, IgK⁺ ASCs originating from the primary tumors of 4 HGSOC patients also underwent single cell sorting into 96 well PCR plates (Eppendorf) containing 4 ul per well of mRNA preserving lysis buffer (in RNAse free water, 10% DTT 0.1M v/v, 5% PBS×10 v/v, 7.5% RNasin ribonuclease inhibitor v/v, cat: N2615). Sorted plates were immediately frozen to −80 C in order to preserve mRNA integrity.

### Single Cell Immunoglobulin Sequencing of Tumor Infiltrating ASCs

Single cell sorted tumor infiltrating ACSs were reverse transcribed and underwent nested PCR amplification and sequencing of their heavy and light chain transcripts as previously described (Tiller et al, Efficient generation of monoclonal antibodies from single human B cells by single cell RT-PCR and expression vector cloning. J Immunol Methods, 2008) Upon collection of all transcripts, data analysis was performed as detailed below.

### Determination of Clonality and Reconstruction of Lineage Trees

Ig Fasta sequences were aligned against the IMGT human heavy chain gene database (downloaded at December 2019) and light chain gene database (downloaded at February 2017) using NCBI IgBlast (version 1.14.0) (Ye et al., 2013). Post processing of IgBlast output, and clonal clustering were performed using Change-O v0.4.6 (www(dot)changeo(dot)readthedocs(dot)io) (Gupta et al., 2015), Alakazam v0.3.0 (www(dot)alakazam(dot)readthedocs.), SHazaM v0.2.3 (www(dot)shazam(dot)readthedocs(dot)io), and custom scripts within the R statistical computing environment, as follows. V(D)J sequences were assigned to clonal groups by partitioning sequences based on identity of IGHV gene annotations, IGHJ gene annotations, and junction region lengths. Within these groups, sequences differing from one another by a distance of more than 15 nucleotides between the V genes were defined as separate clones. The clonal distance threshold was determined by manual inspection using heatmaps of V genes hamming distance. Full-length germline sequences were reconstructed for each clonal cluster with D segment and N/P regions masked (replaced with Ns), with any ambiguous gene assignments within clonal groups resolved by the majority rule. Lineage trees were constructed for each clone having at least two unique sequences using PHYLIP (v3.697) (Felsenstein, 2005) and Alakazam. In selected cases in which identical V-J configurations and junction lengths were identical between different sequences for both the heavy and light chains, yet more than 15 nucleotides between the V genes differed, members were manually clustered into single clones albeit a common ancestry could not be ascertained. Such relations appear with a dashed connecting line in lineage trees. Additionally, multiple identical sequences were referred to as a single expanded clone. Selection quantification was calculated using BASELINe's local test (Yaari et al., 2012).

### Selection of Monoclonal Antibody Candidates, Cloning and Expression

ASCs immunoglobulin transcripts were chosen for cloning and expression on the basis of several criteria. These included: relation of the candidate to an expanded clone, occurrence of multiple identical candidates within the clone, candidate harbors a high load of somatic hypermutations. Following selection of antibody candidates, constructs containing the heavy and light chain variable regions together with 30 additional expression vector homologous nucleotides both upstream and downstream were ordered as gBlocks from IDT and cloned into human IgG1 & IgK expression vectors via the restriction free method. Cloning was performed into human IgG1 and IgK expression vectors (AddGene, AbVec2.0-IGHG1, AbVec1.1-IGKC) using Phusion High-Fidelity DNA Polymerase (NEB, cat: M0530L) according to the manufacturer's instructions. The template plasmids were then selectively degraded using the DpnI restriction enzyme (NEB, cat: R0176L) for 16 hours at 37° C. Cloned vectors were transformed into DH5a competent bacteria using the heat shock technique (42 C for 90 s). Plasmid containing bacteria were selected on the basis of vector-acquired ampicillin resistance. After plating, monoclonal bacterial colonies underwent PCR and sequencing validation of their transformed plasmids. Successfully transformed colonies were then expanded and harvested using Qiagen's plasmid purification kit. Purified vectors were transfected into HEK293t cells in 150 mm tissue culture plates (Corning) at 12.5 ug/DNA per chain using linear 25 kDa polyethyleneimine (at a DNA:PEI mass ratio of 1:2) and grown in serum free media for 5 days. Supernatants were filtered through a 0.2 μm strainer and reacted with protein G Sepharose beads (17-0618-05, Cytiva/GE) on a tilt table, overnight in 4° C. Beads were then pulled down, washed in PBS and eluted using IgG elution buffer (21004, ThermoFisher) into TRIS pH 9 1M. The eluate was then dialyzed to PBS overnight and its final concentration measured using Nanodrop.

### Monolayer ELISA/Immunofluorescence Staining

Cell lines of interest were grown on 96 well plates/chamber slides, fixed with 4% PFA, washed with PBS, blocked with 1% BSA for 90 minutes, stained with the antibodies of interest at a concentration of 500 nM overnight in 4° C.—followed by staining with an Alexa Fluor 488 conjugated secondary antibody and DAPI (1:5000). For monolayer ELISA, DAPI normalized Alexa Fluor 488 signal was used to quantify the staining per well using a Synergy HTX plate reader (Biotek). For quantification of immunofluorescence staining, slides were acquired using a Zeiss LSM 880 confocal microscope. The mean fluorescence signal was calculated per cell using QuPath v0.2.0-m9.

### ELISA

ELISA reactions were carried out using flat-bottom MaxiSorp™ 96-well plates (Invitrogen). Antigen coating was performed in PBS at 1001 per well and left overnight at 4° C. For standard dose response ELISA assays, antigens were plated at a concentration of 1 μg/ml. For comparative ELISA assays with multiple antigen targets, antigens were plated at a constant molar concentration of 50 nM. The plates were washed 5 times with washing buffer (1×PBS with 0.05% Tween-20 (Sigma-Aldrich)) and incubated with 1001 blocking buffer (1×PBS with 1% BSA) for 1 h at room temperature. The blocking solution was subsequently replaced by serial dilutions of either mono- or polyclonal antibodies or serum samples for 2.5 h at RT. For standard dose response ELISA assays, antibodies were introduced over a range of dilutions whereas for ELISA screens, antibodies were introduced at 100 nM. Serum samples were assayed at a dilution of 1:100. Plates were washed 5 times with washing buffer and then incubated with anti-human IgG secondary antibody conjugated to horseradish peroxidase (HRP) (Jackson Immuno Research) in PBS at a 1:5,000 dilution. After washing the plates for additional 5 times, the plates were developed using TMB (Thermo Fisher) and absorbance was measured at 630 nm with an ELISA microplate reader (Synergy HTX plate reader, Biotek).

### Polyreactivity and Hep-2 ELISA

ELISA assays for evaluating antibody polyreactivity were performed as previously described (Prigent et al, Scarcity of autoreactive human blood IgA+memory B cells, Eur J Immunol, 2016).

### Biolayer Interferometry

Analysis of the kinetic binding constants of mAb T13 to MMP14 was performed using the Octet QKe platform. Experiments were conducted at 30° C. with shaking at 1,000 rpm. Briefly, biosensors check of the anti-human Fc capture (AHC) biosensors was performed for 1 minute in PBS. Antibody loading of mAb T13 was performed at an optimized pre-calibrated concentration of 12.5 nM for 5 minutes in PBS. Loaded sensors were then exposed to TNC buffer (50 mM Tris pH 8, 150 mM NaCl, 5 mM CaCl₂) for 30 seconds and 1 minute in two consecutive wells. Next, MMP14 association was performed over a range of concentrations (30 nM-2000 nM), in TNC buffer for 30 minutes. Finally, antigen dissociation was performed in TNC buffer for 30 minutes. Data processing, construction of a response model and curve fitting was accomplished using the Fortebio Octet Data analysis software.

### Western Blots

Lysates for western blot experiments were made from cell lines and patient derived tumor specimens. Cell lines grown to confluence and minced tumor specimens were emulsified in 500 ul of RIPA buffer (20 mM Tris pH 7.4, 137 mM NaCl, 10% glycerol, 0.1% SDS, 0.5% deoxycholate, 1% triton X-100, 2 mM EDTA pH 8, 1 mM PMSF, 20 uM Leupeptin, in DDW) and protease inhibitor (1:100). Mixture was vortexed, agitated for 1 hour at 4° C. and centrifuged. Supernatants were separated and flash frozen. Prior to the experiment, the lysates protein concentration was measured in triplicates using a BCA kit (ThemoFisher Scientific). Samples containing 25 ug of protein were mixed with sample buffer in the presence of DTT, heated to 95° C. for 5 minutes and introduced to 15-well gradient gels (Bio-Rad). The gel content was transferred to a nitrocellulose membrane using the rapid transfer method. Membranes were blocked in blocking buffer (5% BSA, 0.1% tween in PBS) for 1 hour, in room temperature on a tilt table. The membranes were exposed to the appropriate primary antibodies at 1 μg/ml in 5% BSA in PBS, overnight in 4° C. on a tilt table. The day after, membranes were washed 3 times in wash buffer (0.1% tween in PBS), exposed to the appropriate isotype targeting horseradish peroxidase (HRP) conjugated secondary antibodies (Jackson ImmunoResearch) at 1:5000 for 1 hour in room temperature, washed 3 times and developed using ECL. Membranes were acquired using the ChemiDoc imaging system (Bio-Rad). Images were analysed using the Image Lab 6.0.1 software (Bio-Rad).

### MMP14 Overexpression Assay

In this assay K562 cells were transfected with an MMP14:Cherry expression vector using the TransIT-X2 transfection reagent (Mirus Bio) according to the manufacturer's protocol. Briefly, 0.5M K562 cells were plated in 6 well plates in 2.5 ml of growth medium (DMEM, 10% foetal bovine serum, 1×MEM-Eagle non essential amino acids, 2 mM glutamine, 1:100 Pen-Strep solution) per well. For each condition, in a separate tube, 2.5 ug of the MMP14:Cherry vector and 7.5 ul of the TransIT-X2 transfection reagent were mixed in 250 ul of Opti-MEM I reduced serum medium (Gibco) and incubated at room temperature for 30 minutes. Following incubation, the mixture was added to the cells in a drop-wise manner. A mock transfection without the MMP14:Cherry construct was performed in parallel as a negative control. Following a 48 hours incubation period, the cells were stained with mAb T13 at a concentration of 500 nM for 45 minutes on ice, washed, stained with an Alexa fluor 488 conjugated anti human IgG secondary antibody (Jackson Immunoresearch) for 30 minutes on ice, washed and analysed using a Cytoflex flow cytometer.

### Phagocytosis Assays (ADCP)

Antibody dependent cell-mediated phagocytosis (ADCP) was assessed by the measurement of the uptake of antibody-opsonized, antigen-coated fluorescent beads by the THP1 monocytic cell line. Briefly, 2 μg of biotinylated MMP14 protein was used to saturate the binding sites of 0.5 mg 1 μm fluorescent NeutrAvidin beads (Invitrogen). Excess antigen was removed by washing the beads, which were then blocked with 1% BSA. Next, the beads were washed and incubated with antibodies at final concentrations of 0.5 μM (for monoclonal antibodies) or 1 μM (for polyclonal antibodies) for 2 h at 37° C. Following opsonization, beads were washed, and unbound antibodies were removed. The beads were then either stained for IgG to confirm IgG coating or incubated with phagocytotic cells. For the phagocytosis assay, THP-1 cells were added, and the cells were incubated for 1 h at 37° C. to allow phagocytosis after which the extent of phagocytosis was measured via flow cytometry (CytoFLEX). For IgG staining purposes, the beads were incubated with anti-human IgG secondary antibody (Jackson Immuno Research) in blocking buffer at a 1:100 dilution for 30 minutes on ice. The beads were then washed, and the IgG was measured using the CytoFLEX flow cytometer.

### Antibody Dependent Cell-Mediated Cytotoxicity (ADCC) Assays

ADCC assays were performed using the xCelligence RTCA DP platform. Briefly, RTCA DP plates were filled with media and measured for background values. Then, OVCAR3 cells were plated at an optimized quantity of 20K cells per well on RTCA DP plates. OVCAR3 cells proliferation during the seeding phase was monitored via their cell index value. 24 hours after seeding, the cells were exposed to monoclonal polyclonal antibodies at 500 nM for 1 hour in PBS and complemented with lymphokine activated donor natural killer cells at various effector to target (E:T) ratios. Upon introduction of the effector cells, the viability of the OVCAR3 tumor cell population was monitored over a 24-72 hour period. Prior to the ADCC assay, isolation of NK cells from the peripheral blood of healthy donors was performed using the EasySep human NK cell enrichment kit (STEMCELL). NK cells were incubated in growth media in the presence of 500 IU/ml of human recombinant IL-2 overnight to achieve their activation.

### Protein Microarray

ProtoArray Human Protein Microarray (ThermoFisher Scientific) were used per the manufacturer's instructions. The array was exposed to the primary antibodies (T13 & T15) at a concentration of 100 nM.

### Phage Display Enrichment Assay and Peptide Validation ELISA

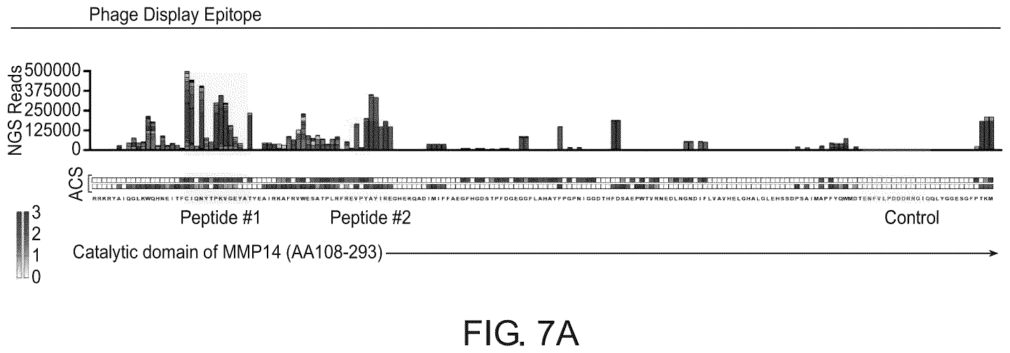

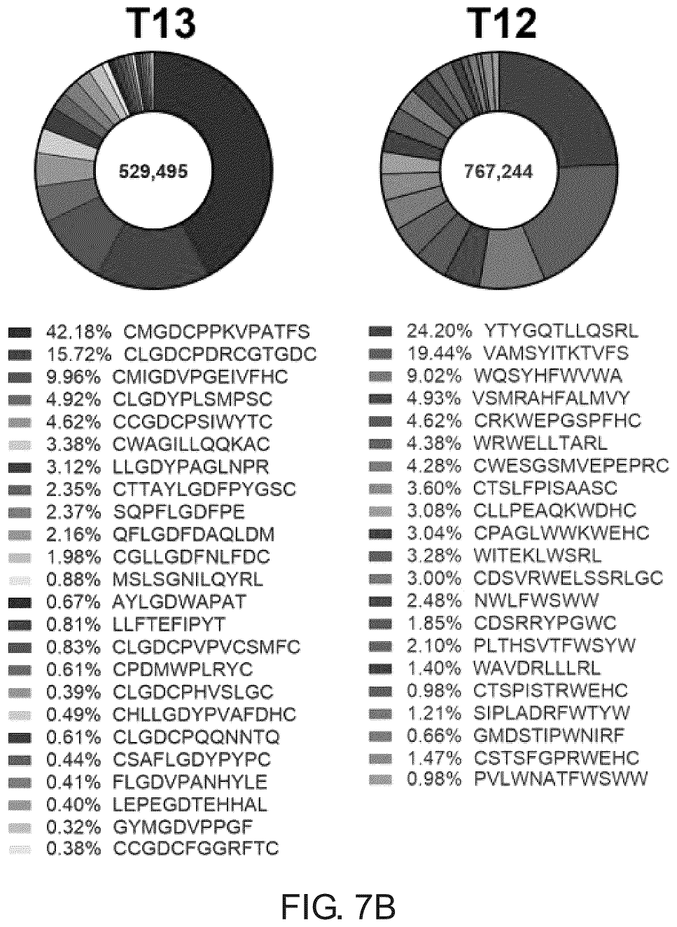

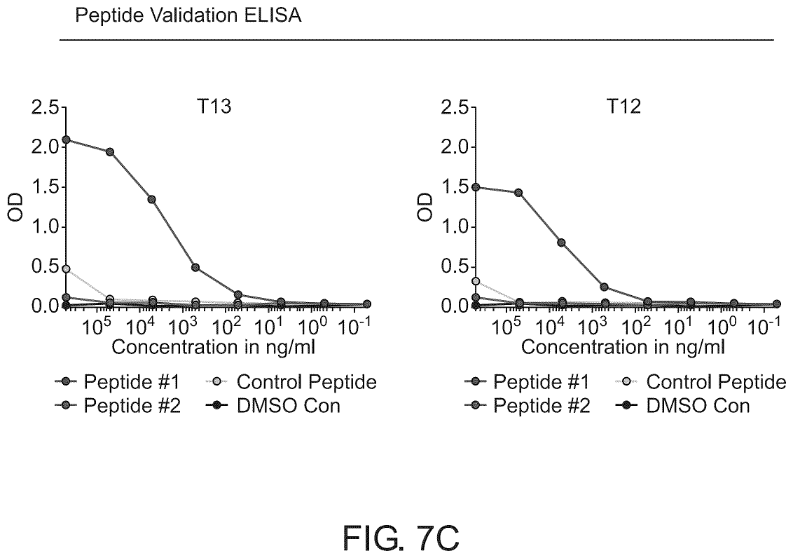

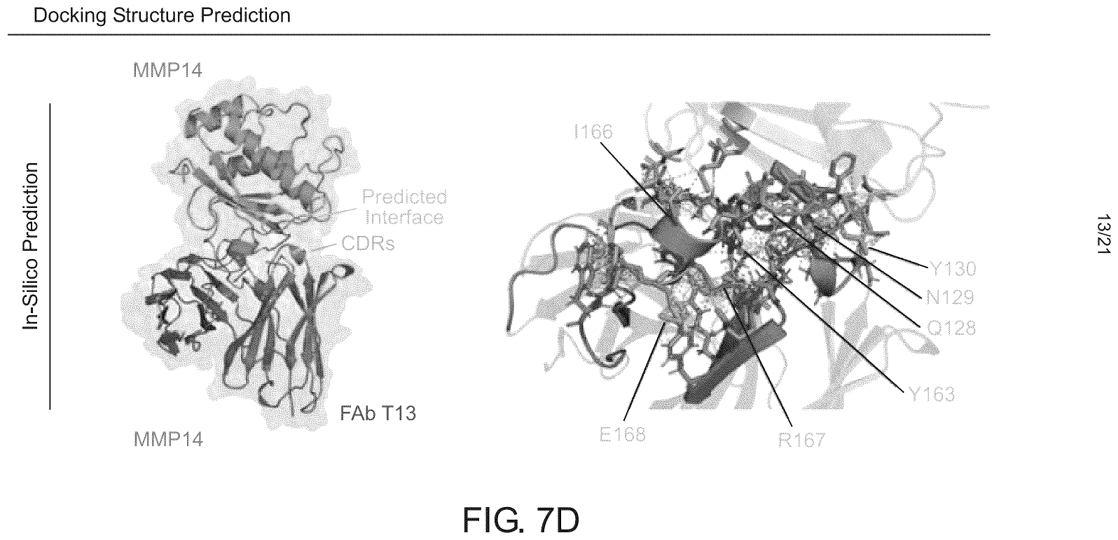

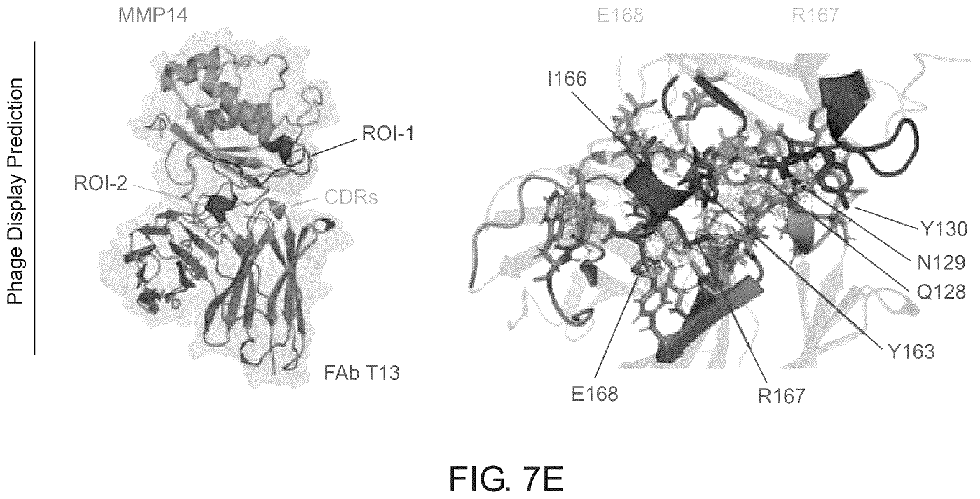

Antibodies were incubated with a phage library which randomly expressed nine order of magnitudes of short 8-14 amino acid peptides. Phages expressing peptides that resembled segments of the original binding motifs were captured by the antibodies, while phages expressing nonreactive peptides were washed out. The enriched phages were then sequenced using next generation sequencing and so the number of NGS reads per a given peptide sequence is proportional to the enrichment of the phage which expressed it. The top 15 peptides derived of three parallel replicate experiments per antibody and their relative share of NGS reads were recorded. Post processing of the data included alignment of each of the top peptide hits to the amino acid sequence of the catalytic domain of MMP14. When plotted (example: FIGS. 7A-E) 11a-e, bars represent a measure of peptide enrichment. The heatmap below the bar plot, labelled “ACS” (Average Conservation Score) represents the average degree of compatibility of all given aligned amino acids to a specific position in the sequence of MMP14. The compatibility score is calculated and binned according to the Gonnet PAM250 matrix and is used to determine amino acid conservation by the Clustal Omega multiple sequence alignment algorithm. Principally, four scores are available: 3—which denotes complete identity between the two aligned amino acids. 2—which represents an overall high degree of similarity between the two amino acids. 1—which indicates a modest degree of similarity—and 0—which states that the amino acids aligned at a given position are completely different. In summary, the ACS score provides a measure of peptide compatibility to the region to which it was aligned to. We selected regions of interest in the sequence of MMP14 to which peptides were both enriched and showed the highest degree of compatibility. Validation ELISA involved plating the antibodies and exposing them to different concentrations of test and control peptides. Peptides were ordered from GenScript, carried an N terminus linker and a biotin, through which the assay was developed using Sterptavidin-HRP.

### Example 1

### HGSOC Infiltrating ASC-Derived Monoclonal Antibodies are Tumor Reactive

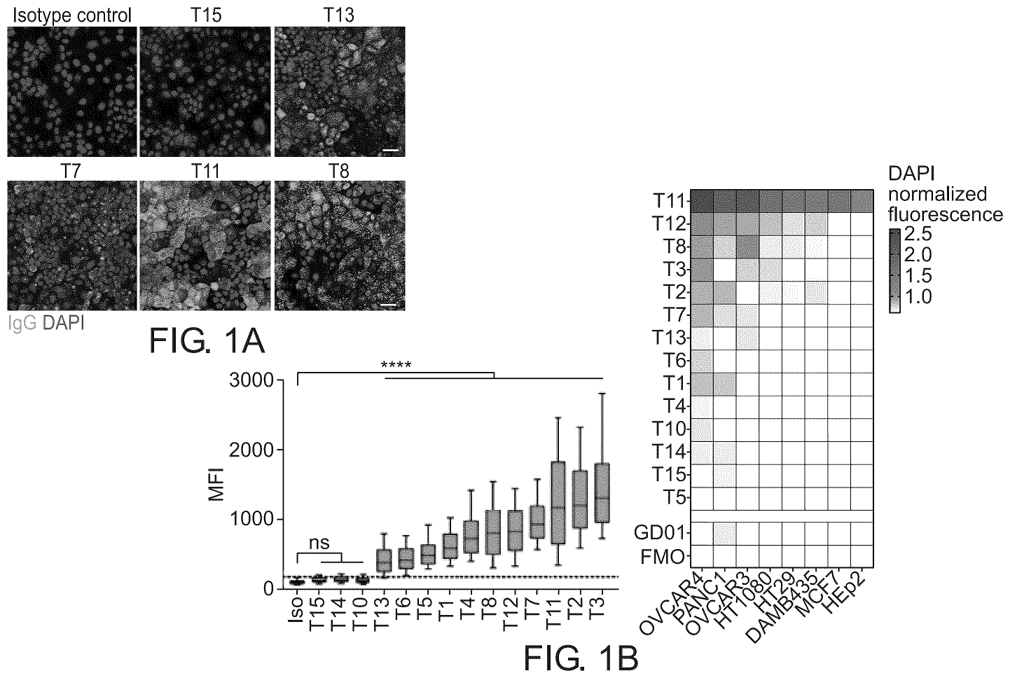

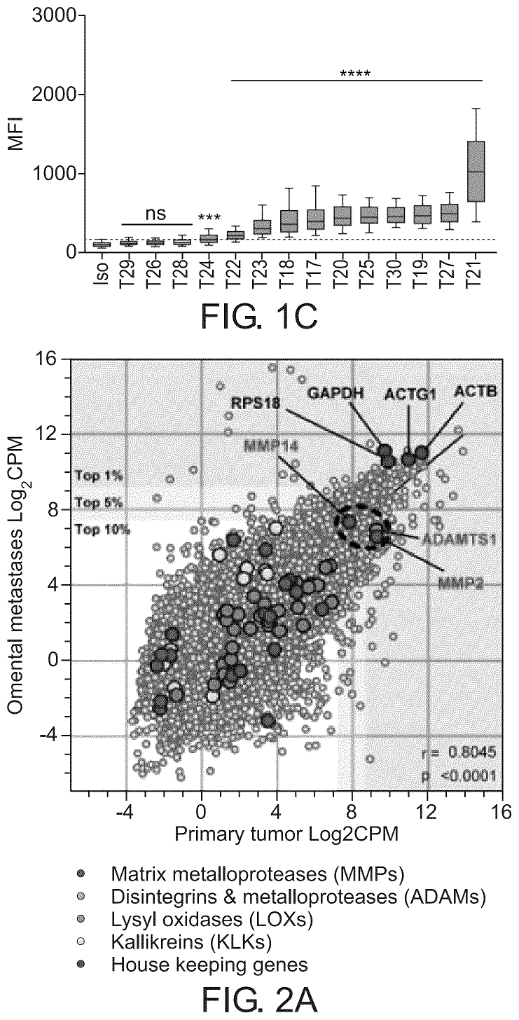

To study the nature of the antibodies found to coat the tumor in HGSOC, antibodies originating from highly mutated and expanded ASC clones were cloned and expressed as monoclonal antibodies. To reveal the surface targets of these patient-derived monoclonal antibodies, their binding capacity to the OVCAR3 cell line was examined. Specifically, the magnitude of antibody binding per single cell was measured within the cultured monolayer using patient-derived monoclonal antibodies. antibodies were found that showed reactivity to OVCAR3 cells, suggesting that they target tumor surface antigens (FIG. 1A, 1C). Furthermore, the monoclonal antibodies primarily interacted with ovarian carcinoma cell lines (OVCAR3 and 4) and a pancreatic cancer cell line, and to a lesser extent with cells derived from other organs, suggesting specificity to both ovarian and pancreatic tumors presumably through shared surface target antigens (FIG. 1B).

### Example 2

### Ascites-Derived Antibodies and Monoclonal Antibodies Derived Therefrom Bind MMP14

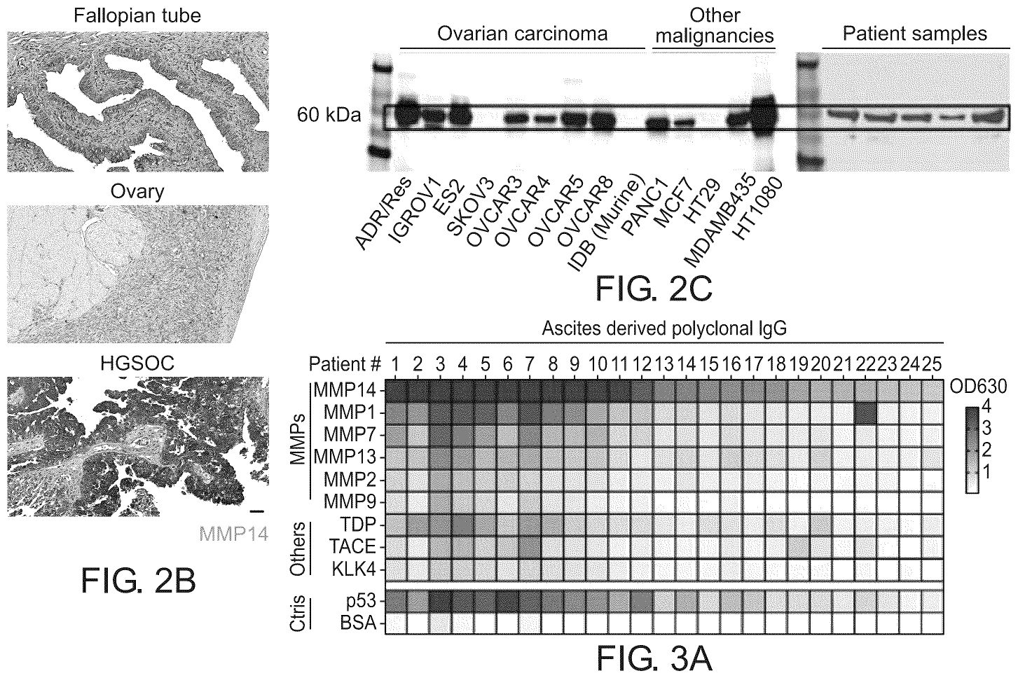

Ovarian and pancreatic cancers are highly desmoplastic (fibrotic) and are constantly subjected to remodeling of their microenvironments by matrix proteases. MMPs were previously demonstrated to trigger the generation of autoreactive antibodies in autoimmune diseases and viral infection suggesting that they may also provoke an immune response in cancer [Wang, E. Y. et al. Diverse Functional Autoantibodies in Patients with COVID-9. medRxiv: the preprint server for health sciences (2020) doi:0.0/2020.2.0.20247205.]. Furthermore, it was suggested that high levels of antigen can lead to a break of tolerance and generation of autoantibodies in cancer, and since MMPs are highly expressed in HGSOC [Cathcart, J. M. et al. Interleukin-6 increases matrix metalloproteinase-14 (MMP-14) levels via down-regulation of p53 to drive cancer progression. Oncotarget 7, 607-620 (206).] they can potentially induce generation of autoantibodies (FIG. 2A-C).

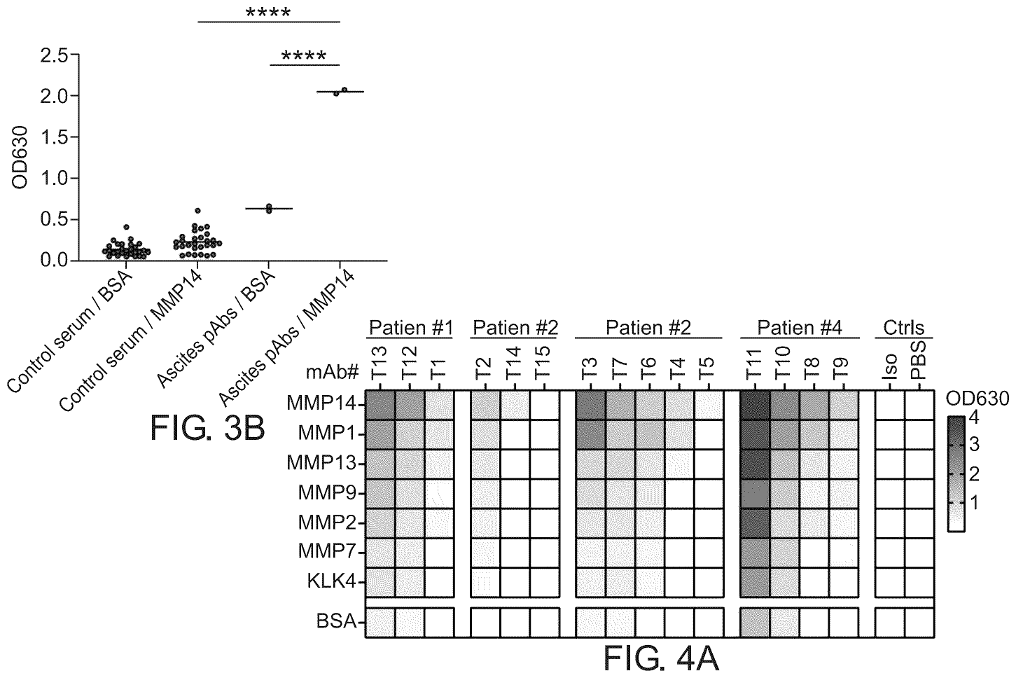

Therefore, the binding capacity of polyclonal IgGs derived from ascites fluids of 25 patients to 6 recombinant MMPs, and additional 3 ECM-associated targets was evaluated. In this setting, BSA was used as a negative control antigen, and p53, which elicits an antibody response in HGSOC, was used as a positive control target. ELISA revealed strong and reproducible antibody reactivity against MMP14 in all of the patients, whereas reactivity to other MMPs was also evident, but to a lesser extent (FIG. 3A). Serum-derived IgGs obtained from healthy individuals did not show binding to MMP14 in this assay (FIG. 3B).

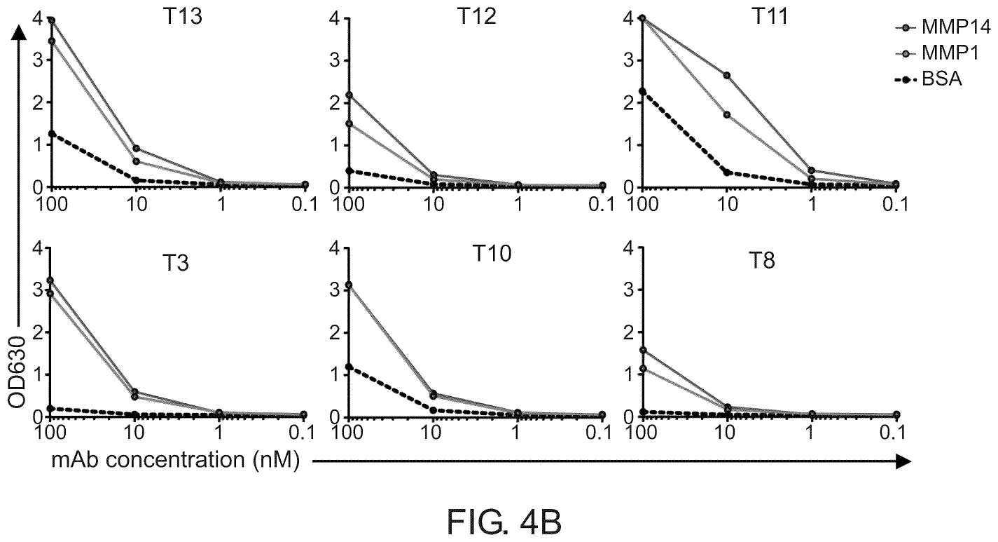

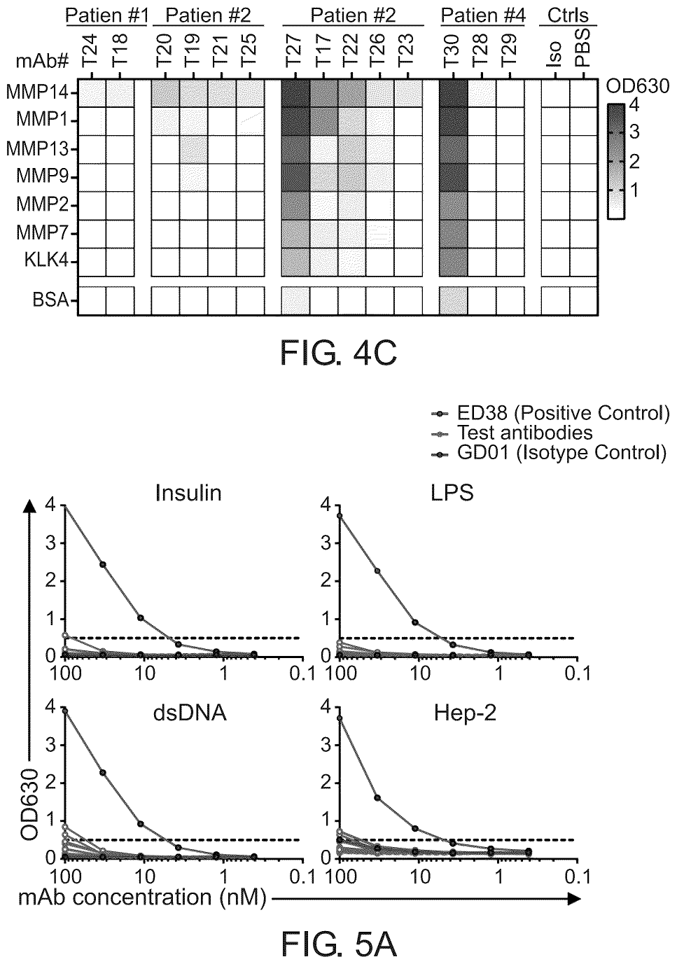

Evaluation of the binding of monoclonal antibodies to ECM-remodeling enzymes by ELISA revealed several mAbs that reproducibly bound to MMP14, with some moderate cross-reactivity with MMP (FIGS. 4A, 4C). Strikingly, in 3/4 patients, monoclonal antibodies that bound MMP14 with various degrees of effectiveness and specificity were detected, confirming the analysis using polyclonal patient-derived antibodies. In order to further examine the specificity of the monoclonal antibodies, the binding activity of the antibodies was tested in different dilutions by ELISA. This analysis showed dose-dependent antibody binding to MMP14 and MMP (FIG. 4B).

### Example 3

### HGSOC Derived Monoclonal Antibodies Bind MMP and MMP14 and are not Poly-Reactive

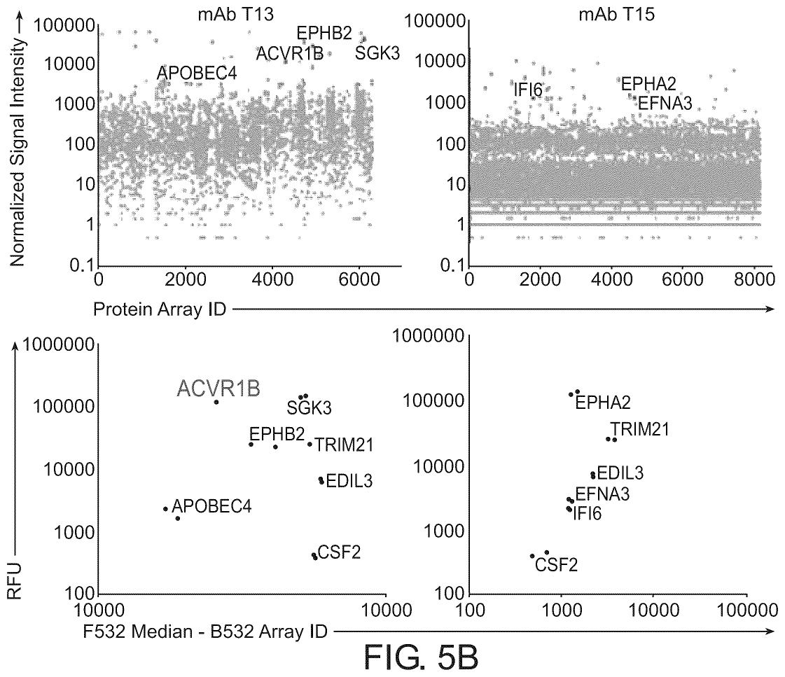

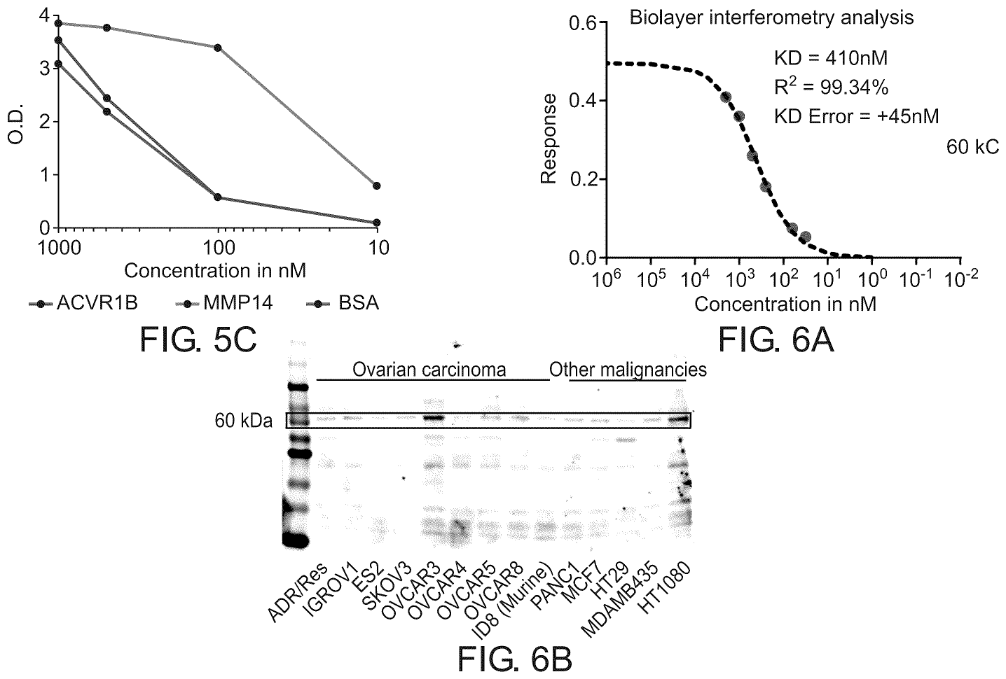

To examine if these antibodies are reactive with many types of antigens (polyreactive) their binding to structurally unrelated antigens was tested, including insulin, double-stranded DNA, and lipopolysaccharide by ELISA. Typically, antibodies that bind at least two members of this defined set of antigens are considered polyreactive⁶. ED38, a well-characterized polyreactive antibody was used as a positive control, and GD0 an antibody that binds an unrelated target (Junin virus GP) was used as a negative control. Minimal binding of the monoclonal antibodies to the unrelated targets was detected even at high antibody concentrations, whereas ED38 was highly reactive in this assay (FIG. 5A). Similar results were obtained by testing binding to human epithelial type 2 (HEp-2) cell lysates, a commonly used assay to determine antibody polyreactivity and for diagnosis of autoimmune diseases (FIG. 5B). To further examine cross-reactivity and to identify additional potential surface targets, the binding of a selected antibody (T3) to a microarray that carries 9000 human protein fragments was tested. This analysis did not reveal additional surface molecules that can serve as a target for the monoclonal antibodies on tumor cells (FIGS. 5B-C).

Hence it can be concluded that tumor-reactive antibodies bind MMP14 and MMP without robust polyreactive binding to unrelated antigens.

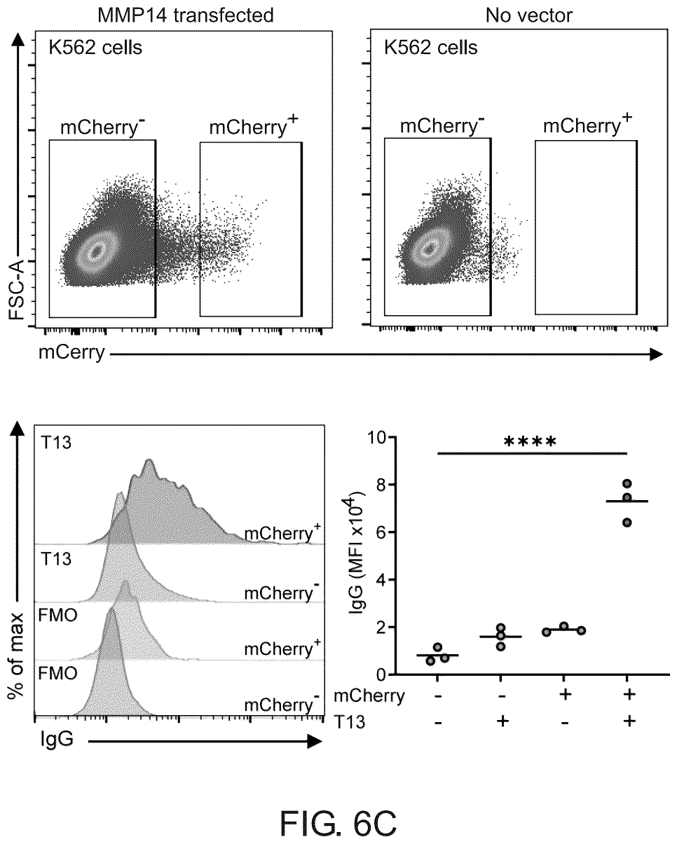

Since MMP is not expressed in HGSOC, subsequent analyses were focused on MMP14. To provide additional evidence for specific binding, T3 was tested in multiple MMP14 binding assays. Using the Octet QKe platform, it was found that the KD value of T3 was 40±45 nM (R2=99.34%) (FIG. 6A). To provide additional evidence for MMP14 binding, a Western blot analysis was performed with T3 antibody on 4 different cell lines of epithelial, mesenchymal and neural crest origin, representing a wide variety of cell types and organs. Bands of approximately 63 kDa were detected in this assay, compatible with the size of MMP14 and an additional ˜35 kDa band appeared as well, which coincides with a known degradation product of MMP14 (FIG. 6B). Finally, to verify that the antibody binds native MMP14, it was expressed together with mCherry in K562 cells that lack endogenous MMP14. Whereas the antibody did not show binding to mCherry-negative cells, mCherry-positive cells showed clear antibody binding by flow cytometric analysis (FIG. 6C). Overall, these results demonstrate that MMP14 is a major target for autoantibodies in HGSOC patients.

### Example 4

### Epitope Mapping of mAbs T2 and T3 Reveals MMP14's I-A Loop as their Target Epitope

Next, the present inventors investigated which epitope in the catalytic domain of MMP14 elicited this immune response. For this purpose, a phage display enrichment assay was utilized with 4 monoclonal antibodies—the MMP14 binding T2 & T3 and the MMP14 nonreactive T4 & T5 as controls. No shared peptides were enriched for both the control and test antibodies. Notably, in the case of T2 & T3, the top 3 peptides alone accounted for 68% and 53% of all the analysed reads for antibodies T3 and T2, respectively, and the majority of enriched peptides for these antibodies aligned to two distinct regions of interest (ROIs) in the catalytic core of MMP14 (FIGS. 7A-B). The first ROI (ROI-) aligned to the I-A loop of MMP14, a region demarcated between the first beta strand and first alpha helix of the catalytic structure. The second ROI (ROI-2) represents a small alpha helix in the II-III loop, a region between the second and third beta strands. This alpha helix is an MMP14 exclusive architecture, as this region does not appear in the majority of the MMP family. Of note, both regions of interest in the sequence of MMP14 are poorly conserved in MMP9, MMP7 and MMP3—targets which fail to bind both antibodies. A consequent peptide ELISA confirmed that both T2 and T3 successfully bound the ROI-peptide, with T3 presenting superior affinity to that of T2, a pattern previously observed with the recombinant form of MMP14. Both antibodies failed to bind the ROI-2 peptide and the control peptide. It was hence suggested that the failure of the ROI-2 to bind the antibodies stem from its linear configuration as opposed to its helical state under native folding conditions (FIG. 7C). Independently, the docking structure of T3 to the catalytic domain of MMP14 was predicted. The structure itself was predicted using the ABpredict algorithm. Multiple iterations of structure prediction demonstrated low structural variance between predictions. Proposed docking structures of T3 prediction to the crystal structure of the catalytic domain of MMP14 were proposed by the PatchDock algorithm. Finally, a lead docking configuration was chosen based on parameters of docking stability (FIG. 7D). Remarkably, the docking epitopes found using the phage display assay and the docking interface determined by the in-silico prediction markedly overlap. This is visible in FIGS. 7D-E. Upon mapping all molecular interactions up to the length of 3.5A between MMP14 and the CDRs, 7 MMP14 residues were identified which are shared by both datasets, and form tight interactions with all the heavy chain CDRs and the light chain CDR3. According to the model, these residues interact with 2 residues located in these CDRs. Out of which, 8 are shared with the germline configuration of T3 whereas 4 residues: heavy chain: S3K, A50R, T53D, and light chain N93P are mutated. It was therefore suggested that reversion of these particular mutations to their germline state would result in a marked loss of affinity towards MMP14.

### Example 5

### Acquisition of Somatic Hypermutations Contributes to Antibody Tumor Reactivity

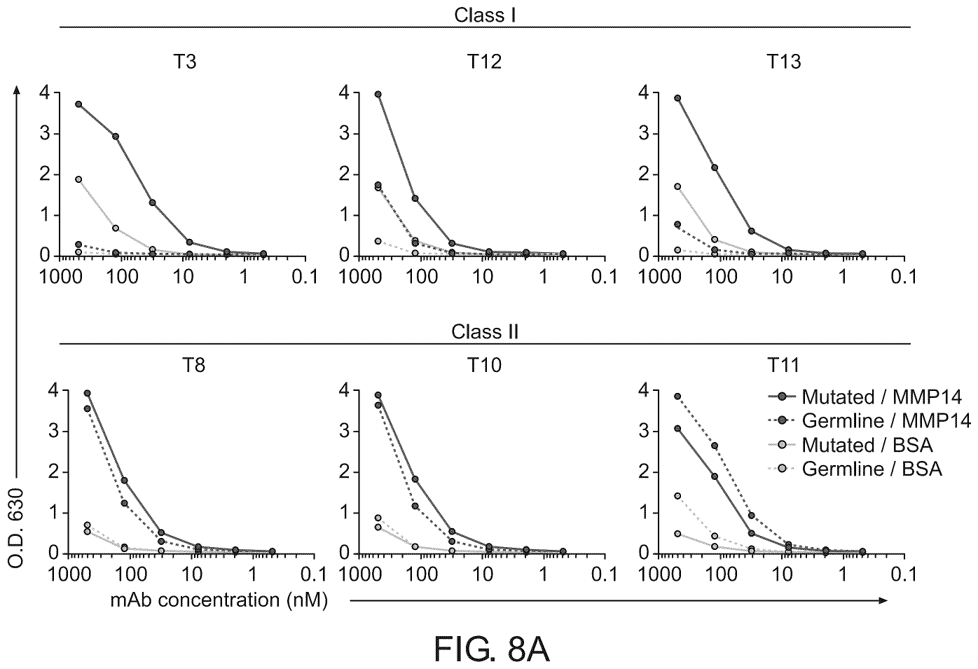

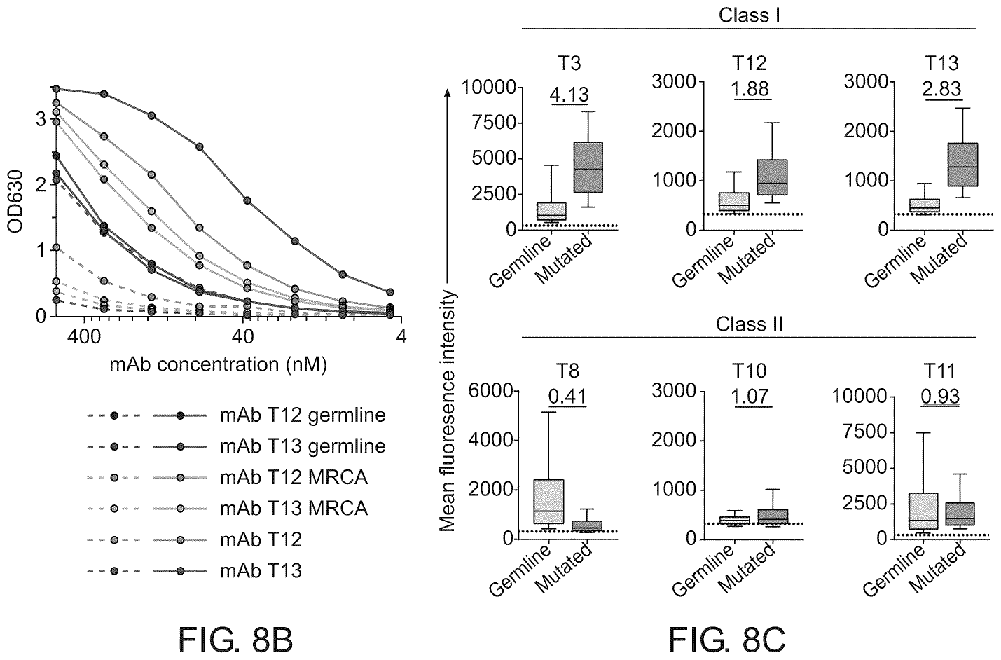

B cell central tolerance is established during development of mature B cells in the bone marrow (BM) where autoreactive clones are eliminated⁶. Nonetheless, 5% of the B cells that emerge from the BM are autoreactive cells that enter the circulation, but typically do not cause an apparent autoimmune disease [Wardemann, H. et al. Predominant autoantibody production by early human B cell precursors. Science 30, 374-377 (2003).]. An additional pathway for the generation of autoreactive antibodies is through insertion of SHM into immunoglobulin genes of antibodies that do not bind a self-target in their original germline version. To examine whether the binding of patient derived anti-MMP14 monoclonal antibodies depended on SHM, parental monoclonal antibody configurations were by reverting their sequence to their germline versions. ELISA revealed three patient-derived monoclonal antibodies (T3, T3, T2) that lost MMP14 binding after removal of SHMs, suggesting that they acquired effective tumor reactivity during the antibody affinity maturation process (FIG. 8A). Acquisition of SHMs appears to confer a stepwise increase in affinity, as the most recent common ancestor (MRCA) versions of T2 and T3, which harbor only the shared mutations between the two antibodies, produce superior binding to the germline versions albeit a lesser affinity compared to T2 and T3 themselves (FIG. 8B). An additional three mAbs (T8, T10, T11) failed to show reduced binding activity when their SHM were removed, suggesting that they were autoreactive before they encountered a cognate antigen (FIG. 8A).

Similarly, T3, T2, and T3 binding to OVCAR3 was significantly reduced in the absence of SHM, whereas mutations did not contribute to tumor binding of T8, T10 and T11 antibodies. (FIG. 8C). Thus, it was concluded that tumor-binding antibodies are divided into two classes: Antibodies were termed class I which depends on the acquisition of SHM for effective tumor reactivity, while the term class II was used to denote antibodies arising from pre-existing autoreactive precursors.

### Example 6

### Tumor Derived Monoclonal Antibodies Exhibit Potential Anti-Tumor Effector Functions

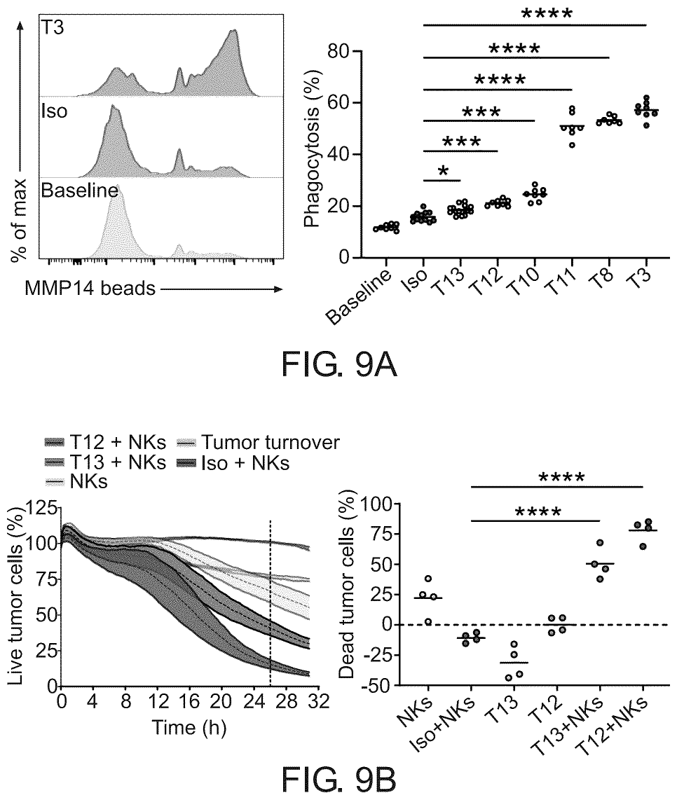

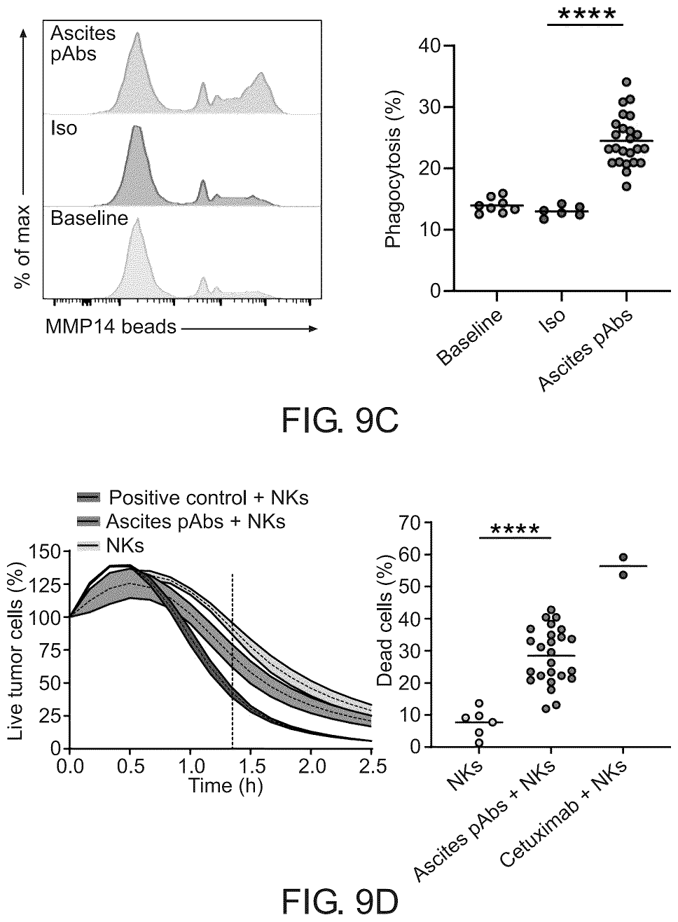

To examine whether the two antibody classes exhibit potential anti-tumor effector activity, their Fc mediated functions were examined. Antibodies can support antibody-dependent cellular phagocytic (ADCP) activity through interaction with Fc receptors expressed on phagocytic cells. Incubation of MMP14-coated beads with some of the monoclonal antibodies, induced their effective uptake by THP-monocytes (3/6 antibodies; T3, T8, T1) (FIG. 9A). Nonetheless, the less functional antibodies, T2 and T3, were able to support antibody-mediated cellular cytotoxicity (ADCC) by NK cells, whereas T10 did not effectively bind the OVCAR3 monolayer (FIG. 9B). Furthermore, incubation of NK cells or THP monocytes in the presence of OVCAR3 cells with polyclonal antibodies derived from 25 patients enhanced their ADCC and ADCP activity, respectively (FIGS. 9C-D). These results demonstrate that endogenous patient-derived antibodies harbor potentially competent Fc mediated antitumor effector functions. Collectively, these findings indicate that both of the newly defined tumor-reactive antibody classes in HGSOC have functional potential.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

It is the intent of the Applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

### Example 7

### HGSOC Infiltrating ASC-Derived Monoclonal Antibodies are Capable of Binding Patient-Derived Primary Cultures

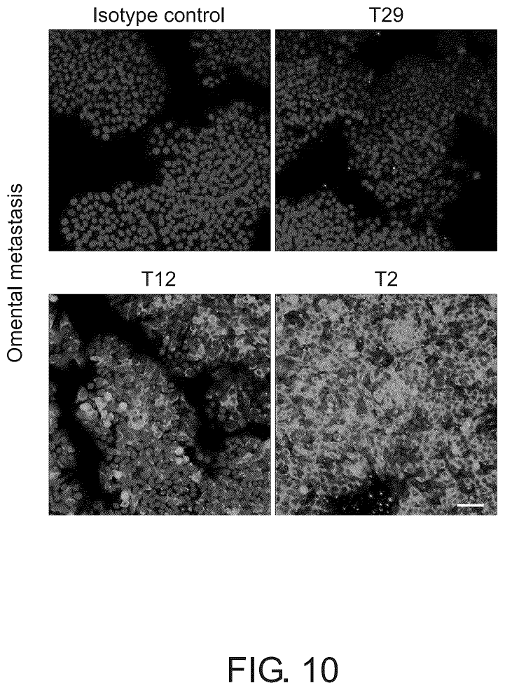

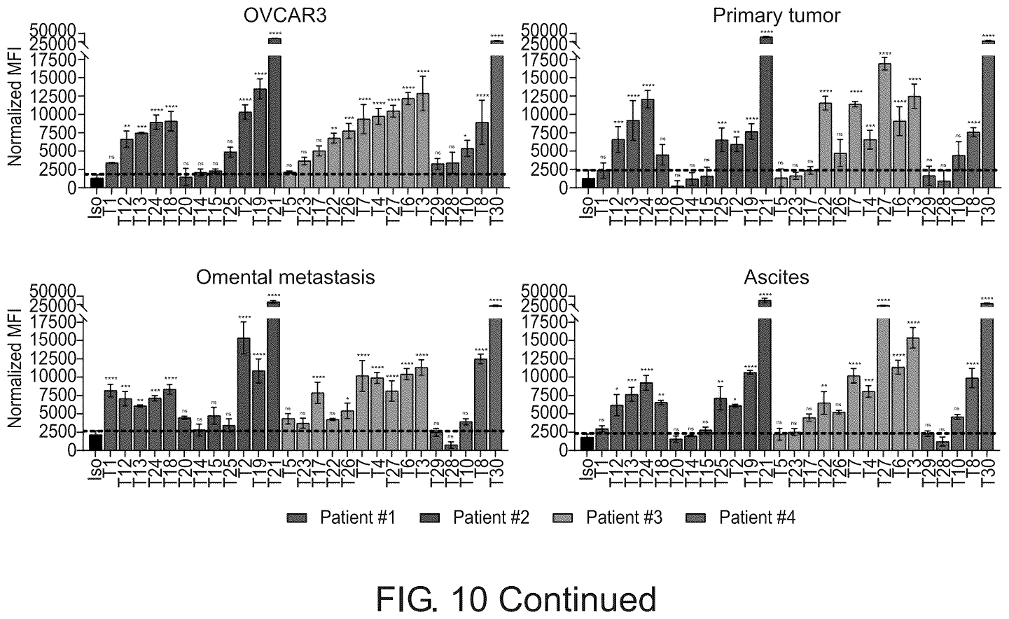

To evaluate whether monoclonal antibodies cloned and expressed from tumor infiltrating ASCs in HGSOC bind original patient derived malignant cells, primary tumor cultures were established and evaluated for monoclonal antibody binding. Primary cultures were established based on primary tumor, omental metastases and ascites borne tumor cells. Monolayers of these primary cultures as well as OVCAR3 cells were reacted with patient-derived monoclonal antibodies and their binding capacity was examined using a Synergy HTX plate reader. Specifically, the magnitude of antibody binding per well was measured using a fluorescently labeled secondary antibody. Various monoclonal antibodies showed reactivity to OVCAR3 cells as well as to patient derived primary cultures, suggesting that antibodies are able to bind patient derived tumor cultures and that target epitopes are shared between the OVCAR3 cell line and these primary cultures (FIG. 10).

### Example 8

### Peritoneal Tumor Implants of ID-8 Murine Ovarian Carcinoma are Preferentially Bound by Monoclonal Antibodies T3 A and T21

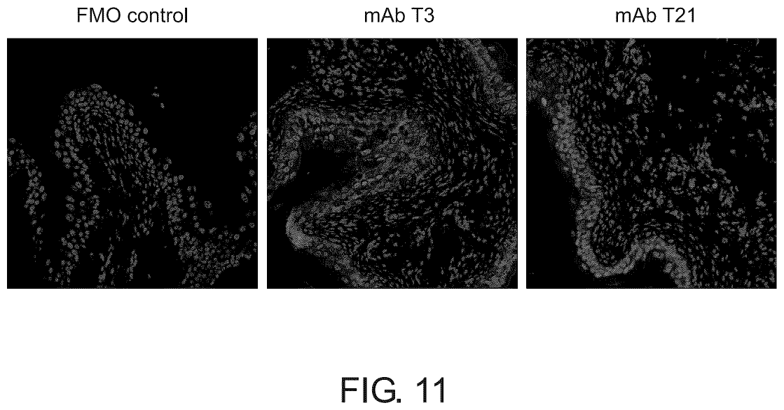

FIG. 11 shows immunofluorescence images of ovarian cancer cells derived from ID8 mouse model of ovarian cancer. This model is the (the best model for ovarian cancer known to date). All cells were stained and acquired back to back.

The middle and right panels depict the ID8 tumor cells stained with antibodies T21 and T3. As is evident from the figure, the antibodies hardly interact with the stroma tissue, below the tumor cells, attesting to their specificity.

In addition, any priority document(s) of this application is/are hereby incorporated herein by reference in its/their entirety.

What is claimed is:

1. A monoclonal antibody comprising an antigen binding domain which comprises the complementarity determining regions (CDRs) CDRH1, CDRH2, CDRH3 of SEQ ID NO: 33 and CDRL1, CDRL2 and CDRL3 of SEQ ID NO: 77.

2. The antibody of claim 1, comprising SEQ ID NO: 33 and SEQ ID NO: 77.

3. An isolated polynucleotide encoding the monoclonal antibody of claim 1.

4. A nucleic acid construct comprising the polynucleotide of claim 3 under a transcriptional control of a cis-acting regulatory element, said element being heterologous to said polynucleotide.

5. The antibody of claim 1, wherein said antibody forms a chimeric antigen receptor (CAR).

6. The antibody of claim 1, wherein said antibody comprises an antibody-dependent cell mediated cytotoxicity (ADCC) or antibody-dependent cellular phagocytosis (ADCP).

7. The antibody of claim 1, being of an IgG serotype.

8. The antibody of claim 1, forms an antibody-drug conjugate (ADC).

9. The antibody of claim 1, wherein said antibody is capable of:
coating a tumor presenting MMP14;
binding the catalytic domain of MMP14;
binding OVCAR3 cells; and/or
recruiting immune cells to a tumor microenvironment.

10. A method of treating cancer in a subject in need thereof, the method comprising administering to the subject a therapeutically effective amount of the antibody of claim 1 or a polyclonal preparation of antibodies from an ascites fluid of an ovarian cancer patient, thereby treating the cancer in the subject.

11. The method of claim 10, wherein said cancer is MMP14+, optionally said cancer is ovarian cancer optionally said ovarian cancer is high grade serous ovarian carcinoma (HGSOC).

12. The method of claim 10, wherein said cancer is pancreatic cancer.

13. The method claim 10, wherein said polyclonal preparation is of the subject.

14. The method of claim 10 further comprising adoptive cell therapy optionally wherein said cells of said adoptive cell therapy comprise ex vivo expanded, lymphokine-activated NK cells or Human activated NK (HaNKs) cells.

15. A method of characterizing an MMP14+ tumor, the method comprising: determining coating the tumor the antibody of claim 1, wherein coating with said anti MMP14 antibodies indicates that the tumor is treatable with adoptive cell therapy.

16. The method of claim 15, further comprising treating the subject with an anti MMP 14 antibody.

17. A method of diagnosing ovarian cancer in a subject in need thereof, the method comprising:
(a) providing a utero-tubal lavage of the subject; and
(b) determining in said utero-tubal lavage a presence or level of MMP14, wherein presence or level above a predetermined threshold is indicative of ovarian cancer in the subject.

18. The method of claim 17, wherein the ovarian cancer is tubal carcinoma in situ.

19. The method of claim 17, wherein said determining is by using anti MMP14 antibodies.

20. A method of treating ovarian cancer in a subject in need thereof, the method comprising:
(a) diagnosing the ovarian cancer according to claim 17;
and
(b) treating the cancer.


SEQUENCE LISTING (541 sequences)

SEQ ID NO: 1 - DNA, 357 bp, synthetic construct
  misc_feature 1..357: note = Monoclonal antibody Heavy chain nucleotide sequence
  source 1..357: mol_type = other DNA
gaggtgcagctggtggagtctgggggaggcttggtacagcctggggggtccctgagactctcctgtgcagcctctggattcatattcagtaagcacgacatgtattgggtccgccaaagaccaggaaagggtctggagtgggtctcacggattggtgatgctggtgacacatactatgcaggctccgtgaagggccgattcaccatcttcagagaaaatgccaagaacacgttgtattttcaaatgaacagactgacagacggggacacggctgtatattattgtggaagaggtatggcagtggctggatttcccctggacgtgtggggcagagggacaagggtcaccgtcacctca

SEQ ID NO: 2 - DNA, 357 bp, synthetic construct
  misc_feature 1..357: note = Monoclonal antibody Heavy chain nucleotide sequence
  source 1..357: mol_type = other DNA
gaggtgcagctggtggagtctgggggaggcttggttcaacctggggggtccctgagactctcctgtgcagcctctggattcactttcaataaccacgacatgcactgggtccgccaagttacagggaaaggtctagaatgggtctcaagtattggtaattttggtgacacatactattcaggctccgtgaagggccgattcaccatctccagacaagatgccaagaactccttgtatcttcaaatgaatagactgagagtcggggacacggctgtctattattgtgcaagaggaagagcagtggctggaaatcccctggacgtctggggcaaagggaccacggtcaccgtctcctca

SEQ ID NO: 3 - DNA, 357 bp, synthetic construct
  misc_feature 1..357: note = Monoclonal antibody Heavy chain nucleotide sequence
  source 1..357: mol_type = other DNA
gaggtgcagctggtggagtctgggggaggcttggtacagcctggggggtccctgagactctcctgtgcagcctctggattcaccttcagtaaccacgacatgcactgggtccgccaagctacaggaaaaggtctggaatgggtctcaggtattggtaatgctggtgacacatactatccaggctccgtgaagggccgattcaccatctccagagaaaatgccaagaactccttgtatcttcaaatgaacagactgagagccggggacacggctgtgtattattgtgcaagaggtatagcagtggctggatttcccctggacgtctggggcaaagggaccacggtcaccgtctcctca

SEQ ID NO: 4 - DNA, 357 bp, synthetic construct
  misc_feature 1..357: note = Monoclonal antibody Heavy chain nucleotide sequence
  source 1..357: mol_type = other DNA
gaggtgcagctggtggagtctgggggaggcttggtacagcctggggggtccctgagactctcctgtgcagcctctggattcaccttcagtaaccacgacatgcactgggtccgccaagctacaggaaaaggtctggaatgggtctcaggtattggtaatgctggtgacacatactatccaggctccgtgaagggccgattcaccatctccagagaaaatgccaagaactccttgtatcttcaaatgaacagactgagagccggggacacggctgtgtattattgtgcaagaggtatagcagtggctggaaatcccctggacgtctggggcaaagggaccacggtcaccgtctcctca

SEQ ID NO: 5 - DNA, 357 bp, synthetic construct
  misc_feature 1..357: note = Monoclonal antibody Heavy chain nucleotide sequence
  source 1..357: mol_type = other DNA
gaggtgcagctggtggagtctgggggaggcttggtacagcctggggggtccctgagactctcctgtgcagcctctggattcaccttcagtagctacgacatgcactgggtccgccaagctacaggaaaaggtctggaatgggtctcagctattggtactgctggtgacacatactatccaggctccgtgaagggccgattcaccatctccagagaaaatgccaagaactccttgtatcttcaaatgaacagcctgagagccggggacacggctgtgtattactgtgcaagaggtatagcagtggctggatttcccctggacgtctggggcaaagggaccacggtcaccgtctcctca

SEQ ID NO: 6 - DNA, 357 bp, synthetic construct
  misc_feature 1..357: note = Monoclonal antibody Heavy chain nucleotide sequence
  source 1..357: mol_type = other DNA
gaggtgcagctggtggagtctgggggaggcttggtacagcctggggggtccctgagactctcctgtgcagcctctggattcaccttcagtagctacgacatgcactgggtccgccaagctacaggaaaaggtctggaatgggtctcagctattggtactgctggtgacacatactatccaggctccgtgaagggccgattcaccatctccagagaaaatgccaagaactccttgtatcttcaaatgaacagcctgagagccggggacacggctgtgtattactgtgcaagaggtatagcagtggctggaaatcccctggacgtctggggcaaagggaccacggtcaccgtctcctca

SEQ ID NO: 7 - DNA, 378 bp, synthetic construct
  misc_feature 1..378: note = Monoclonal antibody Heavy chain nucleotide sequence
  source 1..378: mol_type = other DNA
gaggtgcagctggtggagtctgggggaggcttggtccagcctggggggtccctgagactctcctgtgcagtctctggattcacctttaataactattggatgacctgggtccgccaggctccagggaaggggctggagtgggtggccaacataaagggagatgggagtgagaaaacctatgtggactctgtgaagggccgattcaccgtctccagagacaacgccaacaactcactgcatctgcaaatgaaaagcctgagagccgaggacacggctgtctattactgtgcgagagttgggggcggtgattattatgacagtagtggttactactggctcgatacctggggccagggaaccctggtcaccgtctcctca

SEQ ID NO: 8 - DNA, 378 bp, synthetic construct
  misc_feature 1..378: note = Monoclonal antibody Heavy chain nucleotide sequence
  source 1..378: mol_type = other DNA
gaggtgcagctggtggagtctgggggaggcttggtccagcctggggggtccctgagactctcctgtgcagcctctggattcacctttaataactattggatgacctgggtccgccaggctccagggaaggggctggagtgggtggccaacgtaaaccaagatggcaatgaaaaaaactatgtggactctgtgaggggccgattcaccatctccagagacaacgccaagaactcactgcatctgcaaatgaaaagcctgatatccgacgacacggctgtgtattattgtgcgagagttgggggcggtgattattatgacagtagtggttattactggttcgatacctggggccagggagccctggtcaccgtctcctca

SEQ ID NO: 9 - DNA, 366 bp, synthetic construct
  misc_feature 1..366: note = Monoclonal antibody Heavy chain nucleotide sequence
  source 1..366: mol_type = other DNA
gaggtgcagctggtggagtctgggggaggcctggagcagccagggcggtccctgagactctcctgtgcagcttctggattcagttttggtgataatgctatgacctggttccgccaggctccagggaaggggctggagtgggtcgggatcattagagccaagggttatggtgggacaacagaatacgccgcgtctgtgaaaggcagattcaccatctcaagagatgattccaaaagcatcgcctttctccaaatgaacaacgtgaaagtcgaggacacaggcgtctattattgtgctaaatatgccagtggctgggaggttggattcgacccctggggccagggaaccctggtcaccgtctcgtca

SEQ ID NO: 10 - DNA, 357 bp, synthetic construct
  misc_feature 1..357: note = Monoclonal antibody Heavy chain nucleotide sequence
  source 1..357: mol_type = other DNA
caggtgcagctggtgcagtctggagctgaggtgaagaagcctggggcctcagtgaatgtctcctgcaaggcttctggttacaattttaaggcctatggtgtcggctgggtgcgacaggcccctggacaagggcttgagtggatgggatggatcaccccttacaatggtaaaacaaactatgcacagaagatccagggcagagtcaccatgaccacagacacgtcgacgaccacagcctacatggagctgaggagcctgcaacctgacgacacggccatgtatttctgtgcaagaacccccgctgccctggcaagttttgactactggagccagggaaccctggtcaccgtctcctca

SEQ ID NO: 11 - DNA, 363 bp, synthetic construct
  misc_feature 1..363: note = Monoclonal antibody Heavy chain nucleotide sequence
  source 1..363: mol_type = other DNA
gaggtgcagctggtggagtctgggggaggcttggttcagcctggggggtccctgagactctcctgtgcagcctctggattcacctatttcagctatgccatgacctgggtccgccaggctccagggaaggggctggagtgggtctcatctgttaatgttcgtgttggtacctcatactacgcagactccgtgaagggccggttcaccatctccagtgacatttccaagaacacagtgtttctgcaaatgaacagcctgagagccgacgacacggccatatattactgtgcgacagtgggggctacccaagaccttcgctactttgacttttggggccagggaacgttggtcaccgtctcctca

SEQ ID NO: 12 - DNA, 363 bp, synthetic construct
  misc_feature 1..363: note = Monoclonal antibody Heavy chain nucleotide sequence
  source 1..363: mol_type = other DNA
caggttcagctggtgcagtctggaggtgaggtgaagaagcctggggcctcagcgaaggtctcctgcaagacttctggtttcacctttaccagttatggtatcagctgggtgcgacaggcccctggacaagggcttgagtggatggggtggatcaacacttacaatggtaacacaaagtatgcacagaggctccagggcagagtctccatgaccacagacacatccacgaacacagcctacatggaactgaggagcctgagatctgacgacacggccgtttattactgtgcgcgaggccaaggacggtacggtgactacatttataatcactggggccagggaaccctggtcatcgtctcctca

SEQ ID NO: 13 - DNA, 360 bp, synthetic construct
  misc_feature 1..360: note = Monoclonal antibody Heavy chain nucleotide sequence
  source 1..360: mol_type = other DNA
caggtgcagctggtggagtctgggggaggcgtggtccagcctgggaggtccctgagactctcctgtgcagcctctggattcaccttcagtagccatgctatgcactgggtccgccaggctccaggcaaggggctgcagtgggtggcacttatatcatatgatggatacaataaatactacgcagagtccgtgcagggccgattcaccctctccagagacaattccaagaacacgctgtatctgcaaatgaacagcctgagagctgaggacacggctgtctattactgtgcgagagatcgcgatagtagtggttatatttttgactactggggccagggaaccctggtcaccgtctcctca

SEQ ID NO: 14 - DNA, 372 bp, synthetic construct
  misc_feature 1..372: note = Monoclonal antibody Heavy chain nucleotide sequence
  source 1..372: mol_type = other DNA
caggtacagctgcagcagtcaggtccaggactggtgaagccctcgcagaccctctcactcacctgtgccatctccggggacagtgtctctagcaacagtgctgcttggaactggatcaggcagtccccatcgagaggccttgagtggctgggaaggacatattacaggtccaggtggtatagtgattatgcattctctgtgagaagtcgaataatcgtcaaggcagacacatctaggaacctattctccctgcagttgaactctatgactcccgaggacacggctatatattactgtgcaagagatttgggtatagcagcggctgactggttcgactcgtggggccagggaaccctggtcaccgtctcctca

SEQ ID NO: 15 - DNA, 354 bp, synthetic construct
  misc_feature 1..354: note = Monoclonal antibody Heavy chain nucleotide sequence
  source 1..354: mol_type = other DNA
caggtgcagctggtggagtctgggggaggcgtggtccagcctgggaggtccctgaaactctcctctgtagcctctggattcatcttcaatatgtatggcatgcactgggtccgccaggctccaggcaaggggctggaatgggtggctgtaatatcatatgatggcaccaaagaattctacgcagactccgtgaagggacgattcgctatctccagagacgattccaagaacactctgtctctgcaaatgaacagcctgagacctgaagacacggctgtgtattattctgcgaggagccccagtgggcatgctcttgatctctggggccaagggacagtggtcaccgtctcttca

SEQ ID NO: 16 - DNA, 360 bp, synthetic construct
  misc_feature 1..360: note = Monoclonal antibody Heavy chain nucleotide sequence
  source 1..360: mol_type = other DNA
caggtgcagctgcaggagtcgggcccaggactggtgaagccttcggagaccctgtccctcagctgcgctgtctctggcggctccatcagaggttattcctggagctggatccggcagccccccgggaagggactggagtggattgctgatatgttatatactgggaccaccaccttcaacccatccctcaagagtcgagtcaccatatcggtagacacgtccaagaaccagttctccctgaagctgacctctgtgaccgctgcggacacggccgtgtattactgtgcgaggggaccgactgtgtcagggcccatagtagttgactactggggcccgggaaccctggtcaccgtctcctca

SEQ ID NO: 17 - DNA, 393 bp, synthetic construct
  misc_feature 1..393: note = Monoclonal antibody Heavy chain nucleotide sequence
  source 1..393: mol_type = other DNA
caggttcagctggtgcagtctggagatgaggtgaaggagcctggggcctcagtgaaggtctcctgcaaggcttctggttacagctttaccaggtatggaatcacctgggtgcgactggcccctggacaagggcttgagtggatgggatggatcagcgcttacaatggtgacacaaacttagcacagaagttccagggcagagtcaccatgaccacagacacatccacgaccacagcctacatggagatgaggaacctgagatctgacgacacggccgtttattactgtgggagagatacccgatattgtagtggtgctggctgcccccggccaagttggtactactactacatggacgtctggggcaaagggaccacggtcaccgtctcctca

SEQ ID NO: 18 - DNA, 357 bp, synthetic construct
  misc_feature 1..357: note = Monoclonal antibody Heavy chain nucleotide sequence
  source 1..357: mol_type = other DNA
caggtgcagctggtggagtcggggggaggcgtggtccagcctggggggtccctcagactctcctgtggagcgtccggtttcaccttcagtagatatggcatgcactgggtccgccaggctccaggcaaggggctggagtgggtgtcatttatacggtatgatggaactgaaaaatactatgcggactccgtgaagggccgattcaccatctccagagacaattccaagaacacgctgtatctgcaaatgaatagcctgagagctgacgacgcggctgtatatttctgtgcgaatccttatataacgccgcctactaatgactactggggccagggaaccctggtcaccgtctcctca

SEQ ID NO: 19 - DNA, 363 bp, synthetic construct
  misc_feature 1..363: note = Monoclonal antibody Heavy chain nucleotide sequence
  source 1..363: mol_type = other DNA
gaggtgcagctggtggagtctgggggaggcttggtgcagccgggggggtccctgacactctcctgtacagcctctggattcacctacttcggctttgccatgacctgggtccgccaacctccagggaaggggctggagtggatctcatctattaatcttctttctggtaccacatactatgtggactcggtgaagggccgcttcaccatctccagagacaattccaagaatacagtgtttctgcaaatgaaaggactgacagccgaggacacggccgtttattactgtgcgaaagttggagctacccaggaccttcactactttgacttctggggccagggaaccctggtcaccgtctcctca

SEQ ID NO: 20 - DNA, 363 bp, synthetic construct
  misc_feature 1..363: note = Monoclonal antibody Heavy chain nucleotide sequence
  source 1..363: mol_type = other DNA
gaggtgcagctggtggagtctgggggaggcttggtacagcctggggggtccctgagactctcctgtgcagcctctggattcacctttttcagctatgccatgacctgggtccgccaggctccagggaaggggctggagtgggtctcatctattaatgttcgtggtggtaccacatactacgcagactccgtgaagggccggttcaccatctccagagacaattccaagaacacagtgtttctgcaaatgaacagcctgagagccgaggacacggccgtatattactgtgcgaaagtgggggctacccaagaccttcgctactttgactactggggccagggaaccctggtcaccgtctcctca

SEQ ID NO: 21 - DNA, 363 bp, synthetic construct
  misc_feature 1..363: note = Monoclonal antibody Heavy chain nucleotide sequence
  source 1..363: mol_type = other DNA
gaggtgcagctggtggagtctgggggaggcttggtacagcctggggggtccctgagactctcctgtgcagcctctggattcacctttttcagctatgccatgacctgggtccgccaggctccagggaaggggctggagtgggtctcatctattaatgttcttggtggtaccacatactacgcagactccgtgaagggccggttcaccatctccagagacaattccaagaacacagtgtttctgcaaatgaacagcctgagagccgaggacacggccgtttattactgtgcgaaagtaggggctacccaagaccttcactactttgactactggggccaaggaaccctggtcaccgtctcctca

SEQ ID NO: 22 - DNA, 363 bp, synthetic construct
  misc_feature 1..363: note = Monoclonal antibody Heavy chain nucleotide sequence
  source 1..363: mol_type = other DNA
gaggtgcagctggtggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagtgggagctacccaagaccttcgctactttgactactggggccagggaaccctggtcaccgtctcctca

SEQ ID NO: 23 - protein, 119 aa, synthetic construct
  REGION 1..119: note = Monoclonal antibody Heavy chain amino acid sequence
EVQLVESGGGLVQPGGSLRLSCAASGFIFSKHDMYWVRQRPGKGLEWVSRIGDAGDTYYAGSVKGRFTIFRENAKNTLYFQMNRLTDGDTAVYYCGRGMAVAGFPLDVWGRGTRVTVTS

SEQ ID NO: 24 - protein, 119 aa, synthetic construct
  REGION 1..119: note = Monoclonal antibody Heavy chain amino acid sequence
EVQLVESGGGLVQPGGSLRLSCAASGFTFNNHDMHWVRQVTGKGLEWVSSIGNFGDTYYSGSVKGRFTISRQDAKNSLYLQMNRLRVGDTAVYYCARGRAVAGNPLDVWGKGTTVTVSS

SEQ ID NO: 25 - protein, 119 aa, synthetic construct
  REGION 1..119: note = Monoclonal antibody Heavy chain amino acid sequence
EVQLVESGGGLVQPGGSLRLSCAASGFTFSNHDMHWVRQATGKGLEWVSGIGNAGDTYYPGSVKGRFTISRENAKNSLYLQMNRLRAGDTAVYYCARGIAVAGFPLDVWGKGTTVTVSS

SEQ ID NO: 26 - protein, 119 aa, synthetic construct
  REGION 1..119: note = Monoclonal antibody Heavy chain amino acid sequence
EVQLVESGGGLVQPGGSLRLSCAASGFTFSNHDMHWVRQATGKGLEWVSGIGNAGDTYYPGSVKGRFTISRENAKNSLYLQMNRLRAGDTAVYYCARGIAVAGNPLDVWGKGTTVTVSS

SEQ ID NO: 27 - protein, 119 aa, synthetic construct
  REGION 1..119: note = Monoclonal antibody Heavy chain amino acid sequence
EVQLVESGGGLVQPGGSLRLSCAASGFTFSSYDMHWVRQATGKGLEWVSAIGTAGDTYYPGSVKGRFTISRENAKNSLYLQMNSLRAGDTAVYYCARGIAVAGFPLDVWGKGTTVTVSS

SEQ ID NO: 28 - protein, 119 aa, synthetic construct
  REGION 1..119: note = Monoclonal antibody Heavy chain amino acid sequence
EVQLVESGGGLVQPGGSLRLSCAASGFTFSSYDMHWVRQATGKGLEWVSAIGTAGDTYYPGSVKGRFTISRENAKNSLYLQMNSLRAGDTAVYYCARGIAVAGNPLDVWGKGTTVTVSS

SEQ ID NO: 29 - protein, 126 aa, synthetic construct
  REGION 1..126: note = Monoclonal antibody Heavy chain amino acid sequence
EVQLVESGGGLVQPGGSLRLSCAVSGFTFNNYWMTWVRQAPGKGLEWVANIKGDGSEKTYVDSVKGRFTVSRDNANNSLHLQMKSLRAEDTAVYYCARVGGGDYYDSSGYYWLDTWGQGTLVTVSS

SEQ ID NO: 30 - protein, 126 aa, synthetic construct
  REGION 1..126: note = Monoclonal antibody Heavy chain amino acid sequence
EVQLVESGGGLVQPGGSLRLSCAASGFTFNNYWMTWVRQAPGKGLEWVANVNQDGNEKNYVDSVRGRFTISRDNAKNSLHLQMKSLISDDTAVYYCARVGGGDYYDSSGYYWFDTWGQGALVTVSS

SEQ ID NO: 31 - protein, 122 aa, synthetic construct
  REGION 1..122: note = Monoclonal antibody Heavy chain amino acid sequence
EVQLVESGGGLEQPGRSLRLSCAASGFSFGDNAMTWFRQAPGKGLEWVGIIRAKGYGGTTEYAASVKGRFTISRDDSKSIAFLQMNNVKVEDTGVYYCAKYASGWEVGFDPWGQGTLVTVSS

SEQ ID NO: 32 - protein, 119 aa, synthetic construct
  REGION 1..119: note = Monoclonal antibody Heavy chain amino acid sequence
QVQLVQSGAEVKKPGASVNVSCKASGYNFKAYGVGWVRQAPGQGLEWMGWITPYNGKTNYAQKIQGRVTMTTDTSTTTAYMELRSLQPDDTAMYFCARTPAALASFDYWSQGTLVTVSS

SEQ ID NO: 33 - protein, 121 aa, synthetic construct
  REGION 1..121: note = Monoclonal antibody Heavy chain amino acid sequence
EVQLVESGGGLVQPGGSLRLSCAASGFTYFSYAMTWVRQAPGKGLEWVSSVNVRVGTSYYADSVKGRFTISSDISKNTVFLQMNSLRADDTAIYYCATVGATQDLRYFDFWGQGTLVTVSS

SEQ ID NO: 34 - protein, 121 aa, synthetic construct
  REGION 1..121: note = Monoclonal antibody Heavy chain amino acid sequence
QVQLVQSGGEVKKPGASAKVSCKTSGFTFTSYGISWVRQAPGQGLEWMGWINTYNGNTKYAQRLQGRVSMTTDTSTNTAYMELRSLRSDDTAVYYCARGQGRYGDYIYNHWGQGTLVIVSS

SEQ ID NO: 35 - protein, 120 aa, synthetic construct
  REGION 1..120: note = Monoclonal antibody Heavy chain amino acid sequence
QVQLVESGGGVVQPGRSLRLSCAASGFTFSSHAMHWVRQAPGKGLQWVALISYDGYNKYYAESVQGRFTLSRDNSKNTLYLQMNSLRAEDTAVYYCARDRDSSGYIFDYWGQGTLVTVSS

SEQ ID NO: 36 - protein, 124 aa, synthetic construct
  REGION 1..124: note = Monoclonal antibody Heavy chain amino acid sequence
QVQLQQSGPGLVKPSQTLSLTCAISGDSVSSNSAAWNWIRQSPSRGLEWLGRTYYRSRWYSDYAFSVRSRIIVKADTSRNLFSLQLNSMTPEDTAIYYCARDLGIAAADWFDSWGQGTLVTVSS

SEQ ID NO: 37 - protein, 118 aa, synthetic construct
  REGION 1..118: note = Monoclonal antibody Heavy chain amino acid sequence
QVQLVESGGGVVQPGRSLKLSSVASGFIFNMYGMHWVRQAPGKGLEWVAVISYDGTKEFYADSVKGRFAISRDDSKNTLSLQMNSLRPEDTAVYYSARSPSGHALDLWGQGTVVTVSS

SEQ ID NO: 38 - protein, 120 aa, synthetic construct
  REGION 1..120: note = Monoclonal antibody Heavy chain amino acid sequence
QVQLQESGPGLVKPSETLSLSCAVSGGSIRGYSWSWIRQPPGKGLEWIADMLYTGTTTFNPSLKSRVTISVDTSKNQFSLKLTSVTAADTAVYYCARGPTVSGPIVVDYWGPGTLVTVSS

SEQ ID NO: 39 - protein, 131 aa, synthetic construct
  REGION 1..131: note = Monoclonal antibody Heavy chain amino acid sequence
QVQLVQSGDEVKEPGASVKVSCKASGYSFTRYGITWVRLAPGQGLEWMGWISAYNGDTNLAQKFQGRVTMTTDTSTTTAYMEMRNLRSDDTAVYYCGRDTRYCSGAGCPRPSWYYYYMDVWGKGTTVTVSS

SEQ ID NO: 40 - protein, 119 aa, synthetic construct
  REGION 1..119: note = Monoclonal antibody Heavy chain amino acid sequence
QVQLVESGGGVVQPGGSLRLSCGASGFTFSRYGMHWVRQAPGKGLEWVSFIRYDGTEKYYADSVKGRFTISRDNSKNTLYLQMNSLRADDAAVYFCANPYITPPTNDYWGQGTLVTVSS

SEQ ID NO: 41 - protein, 121 aa, synthetic construct
  REGION 1..121: note = Monoclonal antibody Heavy chain amino acid sequence
EVQLVESGGGLVQPGGSLTLSCTASGFTYFGFAMTWVRQPPGKGLEWISSINLLSGTTYYVDSVKGRFTISRDNSKNTVFLQMKGLTAEDTAVYYCAKVGATQDLHYFDFWGQGTLVTVSS

SEQ ID NO: 42 - protein, 121 aa, synthetic construct
  REGION 1..121: note = Monoclonal antibody Heavy chain amino acid sequence
EVQLVESGGGLVQPGGSLRLSCAASGFTFFSYAMTWVRQAPGKGLEWVSSINVRGGTTYYADSVKGRFTISRDNSKNTVFLQMNSLRAEDTAVYYCAKVGATQDLRYFDYWGQGTLVTVSS

SEQ ID NO: 43 - protein, 121 aa, synthetic construct
  REGION 1..121: note = Monoclonal antibody Heavy chain amino acid sequence
EVQLVESGGGLVQPGGSLRLSCAASGFTFFSYAMTWVRQAPGKGLEWVSSINVLGGTTYYADSVKGRFTISRDNSKNTVFLQMNSLRAEDTAVYYCAKVGATQDLHYFDYWGQGTLVTVSS

SEQ ID NO: 44 - protein, 121 aa, synthetic construct
  REGION 1..121: note = Monoclonal antibody Heavy chain amino acid sequence
EVQLVESGGGLVQPGGSLRLSCAASGFTFSSYAMSWVRQAPGKGLEWVSAISGSGGSTYYADSVKGRFTISRDNSKNTLYLQMNSLRAEDTAVYYCAKVGATQDLRYFDYWGQGTLVTVSS

SEQ ID NO: 45 - DNA, 324 bp, synthetic construct
  misc_feature 1..324: note = Monoclonal antibody Light chain nucleotide sequence
  source 1..324: mol_type = other DNA
gaaatagtgatgacgcagtctccagccaccctgtctgtgtctccaggggaaagggccaccctctcctgcagggccagtcagaatattcacatcaacttagcctggtaccagcagaaacctggccaggctcccaggctcatcatctacgctgcatccaccagggccgcgggtatcccagccaggttcagtgccagtgggtctgggacagagttcactctcactatcagcagtctgcagtctgaagattttgcagtttattactgtcagcagtttaatccctggtctccgtggacgttcggccaagggaccaaggttgaagtcaaa

SEQ ID NO: 46 - DNA, 324 bp, synthetic construct
  misc_feature 1..324: note = Monoclonal antibody Light chain nucleotide sequence
  source 1..324: mol_type = other DNA
gaaatagtgatgacgcagtctccagccaccctgtctgtgtctccaggggagacagccaccctctcctgcagggccaatcagactctttacaacaacttagcctggtaccaacagaaacctggccaggctcccaggctgctcatctatagtggatccaccagggccacgggaatcccagccaggttcagtggcagtgggtctgggacagagttcagtctcaccatcagcagcctgcagtctgaagactttgcagtttattactgtcagcactatactccctggcctccgtatacgttcggccaagggaccaaggtggacttcaaa

SEQ ID NO: 47 - DNA, 324 bp, synthetic construct
  misc_feature 1..324: note = Monoclonal antibody Light chain nucleotide sequence
  source 1..324: mol_type = other DNA
gaaatagtgatgacgcagtctccagccaccctgtctgtgtctccaggggaaagagccaccctctcctgcagggccagtcagagtgttaacagcaacttagcctggtaccagcagaaacctggccaggctcccaggctcctcatctatggtgcatccaccagggccacgggtatcccagccaggttcagtggcagtgggtctgggacagagttcactctcaccatcagcagcctgcagtctgaagattttgcagtttattactgtcagcagtataatccctggcctccgtggacgttcggccaagggaccaaggtggaaatcaaa

SEQ ID NO: 48 - DNA, 324 bp, synthetic construct; identical to SEQ ID NO: 47
  misc_feature 1..324: note = Monoclonal antibody Light chain nucleotide sequence
  source 1..324: mol_type = other DNA

SEQ ID NO: 49 - DNA, 324 bp, synthetic construct
  misc_feature 1..324: note = Monoclonal antibody Light chain nucleotide sequence
  source 1..324: mol_type = other DNA
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggcctccgtggacgttcggccaagggaccaaggtggaaatcaaa

SEQ ID NO: 50 - DNA, 324 bp, synthetic construct; identical to SEQ ID NO: 49
  misc_feature 1..324: note = Monoclonal antibody Light chain nucleotide sequence
  source 1..324: mol_type = other DNA

SEQ ID NO: 51 - DNA, 336 bp, synthetic construct
  misc_feature 1..336: note = Monoclonal antibody Light chain nucleotide sequence
  source 1..336: mol_type = other DNA
gacatcgtgatgacccagtctccagtctccctggctgtgtctctgggcgagagggccaccatcaactgcaagtccagccagagtgttttatacagctccaacaacaagaactatttagcttggtaccagcagaaaccaggacagcctcctaaggtactcatttactgggcttctacccgggaatccggggtccctgaccgattcagtggcagcgggtctggggcagatttcactctcaccatcagcagcctgcaggctgaagatgtggcactttattactgtcagcaatattatgagaatccgacattcggccaagggaccaaggtggaaatcaaa

SEQ ID NO: 52 - DNA, 336 bp, synthetic construct
  misc_feature 1..336: note = Monoclonal antibody Light chain nucleotide sequence
  source 1..336: mol_type = other DNA
gacatcgtgatgacccagtctccagtctccctggctgtgtctctgggcgagagggccaccatcaactgcaagtccagccagagtgttttatacaactccaacaataagaactatttagcttggtaccagcagaaaccaggacagcctcctaaggtgctcatttactgggcatctacccgggaatccggggtccctgaccgattcagtggcagcgggtctgggacagatttcactctcaccatcagcagcctgcaggctgaagatgtggcagtttatttctgtcagcaatattatgatactccgacattcggccaagggaccaaggtggaaatcaaa

SEQ ID NO: 53 - DNA, 321 bp, synthetic construct
  misc_feature 1..321: note = Monoclonal antibody Light chain nucleotide sequence
  source 1..321: mol_type = other DNA
gccatccagatgacccagtctccatcctccctgtctgcatctgtaggagacagggtcaccatcacttgccgggcaagtcagttcattagaaatgatttaggctggtatcagcagaaaccagggagagcccctaagctcctgatctatgctgcatccgggttacacagtggggtcccagcaaggttcagcggcaatggatctgccacagatttcactctcaccatcaccagcctgcagcctgaagattttgcaacttattactgtctacaagattacaatttcccgtggacgttcggccaagggaccaaggtggaaatcaaa

SEQ ID NO: 54 - DNA, 318 bp, synthetic construct
  misc_feature 1..318: note = Monoclonal antibody Light chain nucleotide sequence
  source 1..318: mol_type = other DNA
gacatccagatgacccagtctccttccaccctgtctgcatctgtgggagacagggtcaccatcacttgccgggccagtcagagtataagtagctggttggcctggtatcagcagaaagcagggaaagcccctaagctcctgatctttgatgcctccactttgcaaagtggggtcccatcaaggttcagcggcagtggatctgggacagaattcagtctcaccatcagcagcctgcagcctgacgattttgcaacttattactgccaacagtattatagttattcgacgttcggccaagggaccaaggtggaaatcaga

SEQ ID NO: 55 - DNA, 318 bp, synthetic construct
  misc_feature 1..318: note = Monoclonal antibody Light chain nucleotide sequence
  source 1..318: mol_type = other DNA
gacatccagatgacccagtctccttccaccctgtctgcatctgtaggagacagagtcaccatcacttgccgggccagtcagagtgtttataggttgttggcctggtatcagcagaaaccaggtaaagcccctaaactcctgatctatgatgccttcaaattgaaaagtggggtcccaccaaggttcagcggcagtggatctgggacagaattcactctcaccatcagcagcctgcagactgatgattttgcaagttattactgccaacagtataatagttattcgacgttcggccaagggaccaaggtggaagtcaaa

SEQ ID NO: 56 - DNA, 321 bp, synthetic construct
  misc_feature 1..321: note = Monoclonal antibody Light chain nucleotide sequence
  source 1..321: mol_type = other DNA
gacatccagatgacccagtctccttccaccctgtctgcatctgtgggagaccgagtcaccatcacttgccgggccagtcagattattagtagctgggtggcctggtatcagcagagaccagggaaagcccctaagctcctgatctttgatgcctccattttggaaagtggggtcccatcaaggttcagcggcagtgcaactgggacagaattcagtctcaccatcagcggtctgcagcctgatgattttgcaacttattactgccaacactataatgattttcccctcagtttcggcggagggaccaaggtggagatcaaa

SEQ ID NO: 57 - DNA, 318 bp, synthetic construct
  misc_feature 1..318: note = Monoclonal antibody Light chain nucleotide sequence
  source 1..318: mol_type = other DNA
gacatccagatgacccagtctccatcctccctgtctgcatctgtaggagacagagtcaccatcacttgccaggcgagtcaggacattagtaactatttaaattggtatcagcagaaaccagggaaagcccctaaggtcctgatctacgatgcatccaatttggaaagaggggtcccatcaaggttcagtggaagtggatctgggacagcttttactttcaccatcagcagcctgcagcctgaagatattgcaacatattactgccaacagtatgataatctcccttctttcggcggagggaccaaggtggagatcaaa

SEQ ID NO: 58 - DNA, 318 bp, synthetic construct
  misc_feature 1..318: note = Monoclonal antibody Light chain nucleotide sequence
  source 1..318: mol_type = other DNA
gaaatagtgatgacgcagtctccagccaccctgtctgtgtctccaggggacagagccaccctctcctgcagggccagtcagagcgtaagcaacaggttggcctggtaccagcagaaacctggccaggctcccaggctcctcatctatgatgcatccaccagggccactgatatcccagccagattcagtggcagtgggtctgggacagaattcactctcaccattagcagcctgcagtctgaagactttgcaatttatttctgtcaccagtatcataactggccgggttttggcccggggaccaagctggagatcaaa

SEQ ID NO: 59 - DNA, 336 bp, synthetic construct
  misc_feature 1..336: note = Monoclonal antibody Light chain nucleotide sequence
  source 1..336: mol_type = other DNA
gatattgtgatgactcagtctccactctccctgcccgtcacccctggagagccggcctccatctcctgcaagtctagtcagagcctccttcttagtaatggatacaactatttggattggtacctgcagaagccagggcagtctccacagctcctgatcactctggcctctgatcgggcctccggggtccctgacaggttcagtggcagtggatcaggcacagaattacaactgaaaatcggcagagtggaggctgaggatgttggggtttattactgcatgcaaggtctacaaacccctttcactttcggccctgggaccaaagtggagatcaaa

SEQ ID NO: 60 - DNA, 321 bp, synthetic construct
  misc_feature 1..321: note = Monoclonal antibody Light chain nucleotide sequence
  source 1..321: mol_type = other DNA
gacatccagatgacccagtctccatccaccctgtctgcatctgtaggagacagagtcaccatcacttgccgggccagtcagagtattagtacctggttggcctggtatcagcagaaaccagggaaagcccctaaactcctgatctataaggcgtctagtttagaaagtggggtcccatcaaggttcagcggcagtggatctgggacagaattcactctcaccatcagcagcctgcagcctgatgatcttgcaacctattactgccaacagtattataattacgggatcactttcggccctgggaccaaagtggatatccaa

SEQ ID NO: 61 - DNA, 324 bp, synthetic construct
  misc_feature 1..324: note = Monoclonal antibody Light chain nucleotide sequence
  source 1..324: mol_type = other DNA
gacatccagatgacccagtctccatcctccctgtctgcatctgttggacacagagtcaccatcacttgccgggcaagtcagaccattagcaaccatttaaattggtttcagcagaagccagggaaagcccctaaactcctgatttatgctgcatccaggttgcaaactggggtcccatcaaggttcagtggcagtggatctgggacagatttcactctcaccatcagcagtctgcaacctgaagattttgcgacttacttctgtcaacagacttacgatatccctccatacacttttggccaggggaccaagctggagatcaaa

SEQ ID NO: 62 - DNA, 321 bp, synthetic construct
  misc_feature 1..321: note = Monoclonal antibody Light chain nucleotide sequence
  source 1..321: mol_type = other DNA
gacatccagatgacccagtctccatcctccctgtcggcatctgtaggagacaaagtcaccatcacttgccgggcgagtcaggacattagcaattatttagcctggtttcagcagaaaccagggaaagttcccaagctcctgatctatggtgcatccactttgctgtcaggggtcccatctcggttcagtggcagtcaatctgggacaaatttcactctcatcatcagcagcctgcagcctgaagatgttgcaacttattactgtcaaaaacataacagtgccccgtggacgttcggccaagggaccaaggtggaaatcaaa

SEQ ID NO: 63 - DNA, 318 bp, synthetic construct
  misc_feature 1..318: note = Monoclonal antibody Light chain nucleotide sequence
  source 1..318: mol_type = other DNA
gacatccagatgacccagtctccttccaccctgtctgcttctgtaggagacagagtcaccatcacttgccgggccagtcagagtgtttataggtggttggcctggtatcagcagaaaccaggtgaagcccctaaactcctggtctatggtgccttcagtttgcagagtggggtcccaccgaggttcagcggcagtggatatgggacagaattcactctcaccatcagcagcctgcagcctgatgattttgcaacttattattgccaacaatataatagtcattcgacgttcggccaagggaccaaggtggaagtcaaa

SEQ ID NO: 64 - DNA, 318 bp, synthetic construct
  misc_feature 1..318: note = Monoclonal antibody Light chain nucleotide sequence
  source 1..318: mol_type = other DNA
gacatccagatgacccagtctccttccaccctgtctgcatctgtaggagacagagtcaccatcacttgccgggccagtcagagtgttagtaggtggttggcctggtatcagcagaaaccaggtaaagcccctaaactcctgatctatgatgccttcagtttggaaagtggggtcccaccaaggttcagcggcagtggatctgggacagaattcactctcaccatcagcagcctgcagcctgatgattttgcaacttattactgccaacagtataatagttattcgacgttcggccaagggaccaaggtggaagtcaaa

SEQ ID NO: 65 - DNA, 318 bp, synthetic construct; identical to SEQ ID NO: 64
  misc_feature 1..318: note = Monoclonal antibody Light chain nucleotide sequence
  source 1..318: mol_type = other DNA

SEQ ID NO: 66 - DNA, 318 bp, synthetic construct
  misc_feature 1..318: note = Monoclonal antibody Light chain nucleotide sequence
  source 1..318: mol_type = other DNA
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctatgatgcctccagtttggaaagtggggtcccatcaaggttcagcggcagtggatctgggacagaattcactctcaccatcagcagcctgcagcctgatgattttgcaacttattactgccaacagtataatagttattcgacgttcggccaagggaccaaggtggaaatcaaa

SEQ ID NO: 67 - protein, 108 aa, synthetic construct
  REGION 1..108: note = Monoclonal antibody Light chain amino acid sequence
EIVMTQSPATLSVSPGERATLSCRASQNIHINLAWYQQKPGQAPRLIIYAASTRAAGIPARFSASGSGTEFTLTISSLQSEDFAVYYCQQFNPWSPWTFGQGTKVEVK

SEQ ID NO: 68 - protein, 108 aa, synthetic construct
  REGION 1..108: note = Monoclonal antibody Light chain amino acid sequence
EIVMTQSPATLSVSPGETATLSCRANQTLYNNLAWYQQKPGQAPRLLIYSGSTRATGIPARFSGSGSGTEFSLTISSLQSEDFAVYYCQHYTPWPPYTFGQGTKVDFK

SEQ ID NO: 69 - protein, 108 aa, synthetic construct
  REGION 1..108: note = Monoclonal antibody Light chain amino acid sequence
EIVMTQSPATLSVSPGERATLSCRASQSVNSNLAWYQQKPGQAPRLLIYGASTRATGIPARFSGSGSGTEFTLTISSLQSEDFAVYYCQQYNPWPPWTFGQGTKVEIK

SEQ ID NO: 70 - protein, 108 aa, synthetic construct; identical to SEQ ID NO: 69
  REGION 1..108: note = Monoclonal antibody Light chain amino acid sequence

SEQ ID NO: 71 - protein, 108 aa, synthetic construct
  REGION 1..108: note = Monoclonal antibody Light chain amino acid sequence
EIVMTQSPATLSVSPGERATLSCRASQSVSSNLAWYQQKPGQAPRLLIYGASTRATGIPARFSGSGSGTEFTLTISSLQSEDFAVYYCQQYNNWPPWTFGQGTKVEIK

SEQ ID NO: 72 - protein, 108 aa, synthetic construct; identical to SEQ ID NO: 71
  REGION 1..108: note = Monoclonal antibody Light chain amino acid sequence

SEQ ID NO: 73 - protein, 112 aa, synthetic construct
  REGION 1..112: note = Monoclonal antibody Light chain amino acid sequence
DIVMTQSPVSLAVSLGERATINCKSSQSVLYSSNNKNYLAWYQQKPGQPPKVLIYWASTRESGVPDRFSGSGSGADFTLTISSLQAEDVALYYCQQYYENPTFGQGTKVEIK

SEQ ID NO: 74 - protein, 112 aa, synthetic construct
  REGION 1..112: note = Monoclonal antibody Light chain amino acid sequence
DIVMTQSPVSLAVSLGERATINCKSSQSVLYNSNNKNYLAWYQQKPGQPPKVLIYWASTRESGVPDRFSGSGSGTDFTLTISSLQAEDVAVYFCQQYYDTPTFGQGTKVEIK

SEQ ID NO: 75 - protein, 107 aa, synthetic construct
  REGION 1..107: note = Monoclonal antibody Light chain amino acid sequence
AIQMTQSPSSLSASVGDRVTITCRASQFIRNDLGWYQQKPGRAPKLLIYAASGLHSGVPARFSGNGSATDFTLTITSLQPEDFATYYCLQDYNFPWTFGQGTKVEIK

SEQ ID NO: 76 - protein, 106 aa, synthetic construct
  REGION 1..106: note = Monoclonal antibody Light chain amino acid sequence
DIQMTQSPSTLSASVGDRVTITCRASQSISSWLAWYQQKAGKAPKLLIFDASTLQSGVPSRFSGSGSGTEFSLTISSLQPDDFATYYCQQYYSYSTFGQGTKVEIR

SEQ ID NO: 77 - protein, 106 aa, synthetic construct
  REGION 1..106: note = Monoclonal antibody Light chain amino acid sequence
DIQMTQSPSTLSASVGDRVTITCRASQSVYRLLAWYQQKPGKAPKLLIYDAFKLKSGVPPRFSGSGSGTEFTLTISSLQTDDFASYYCQQYNSYSTFGQGTKVEVK

SEQ ID NO: 78 - protein, 107 aa, synthetic construct
  REGION 1..107: note = Monoclonal antibody Light chain amino acid sequence
DIQMTQSPSTLSASVGDRVTITCRASQIISSWVAWYQQRPGKAPKLLIFDASILESGVPSRFSGSATGTEFSLTISGLQPDDFATYYCQHYNDFPLSFGGGTKVEIK

SEQ ID NO: 79 - protein, 106 aa, synthetic construct
  REGION 1..106: note = Monoclonal antibody Light chain amino acid sequence
DIQMTQSPSSLSASVGDRVTITCQASQDISNYLNWYQQKPGKAPKVLIYDASNLERGVPSRFSGSGSGTAFTFTISSLQPEDIATYYCQQYDNLPSFGGGTKVEIK

SEQ ID NO: 80 - protein, 106 aa, synthetic construct
  REGION 1..106: note = Monoclonal antibody Light chain amino acid sequence
EIVMTQSPATLSVSPGDRATLSCRASQSVSNRLAWYQQKPGQAPRLLIYDASTRATDIPARFSGSGSGTEFTLTISSLQSEDFAIYFCHQYHNWPGFGPGTKLEIK

SEQ ID NO: 81 - protein, 112 aa, synthetic construct
  REGION 1..112: note = Monoclonal antibody Light chain amino acid sequence
DIVMTQSPLSLPVTPGEPASISCKSSQSLLLSNGYNYLDWYLQKPGQSPQLLITLASDRASGVPDRFSGSGSGTELQLKIGRVEAEDVGVYYCMQGLQTPFTFGPGTKVEIK

SEQ ID NO: 82 - protein, 107 aa, synthetic construct
  REGION 1..107: note = Monoclonal antibody Light chain amino acid sequence
DIQMTQSPSTLSASVGDRVTITCRASQSISTWLAWYQQKPGKAPKLLIYKASSLESGVPSRFSGSGSGTEFTLTISSLQPDDLATYYCQQYYNYGITFGPGTKVDIQ

SEQ ID NO: 83 - protein, 108 aa, synthetic construct
  REGION 1..108: note = Monoclonal antibody Light chain amino acid sequence
DIQMTQSPSSLSASVGHRVTITCRASQTISNHLNWFQQKPGKAPKLLIYAASRLQTGVPSRFSGSGSGTDFTLTISSLQPEDFATYFCQQTYDIPPYTFGQGTKLEIK

SEQ ID NO: 84 - protein, 107 aa, synthetic construct
  REGION 1..107: note = Monoclonal antibody Light chain amino acid sequence
DIQMTQSPSSLSASVGDKVTITCRASQDISNYLAWFQQKPGKVPKLLIYGASTLLSGVPSRFSGSQSGTNFTLIISSLQPEDVATYYCQKHNSAPWTFGQGTKVEIK

SEQ ID NO: 85 - protein, 106 aa, synthetic construct
  REGION 1..106: note = Monoclonal antibody Light chain amino acid sequence
DIQMTQSPSTLSASVGDRVTITCRASQSVYRWLAWYQQKPGEAPKLLVYGAFSLQSGVPPRFSGSGYGTEFTLTISSLQPDDFATYYCQQYNSHSTFGQGTKVEVK

SEQ ID NO: 86 - protein, 106 aa, synthetic construct
  REGION 1..106: note = Monoclonal antibody Light chain amino acid sequence
DIQMTQSPSTLSASVGDRVTITCRASQSVSRWLAWYQQKPGKAPKLLIYDAFSLESGVPPRFSGSGSGTEFTLTISSLQPDDFATYYCQQYNSYSTFGQGTKVEVK

SEQ ID NO: 87 - protein, 106 aa, synthetic construct; identical to SEQ ID NO: 86
  REGION 1..106: note = Monoclonal antibody Light chain amino acid sequence

SEQ ID NO: 88 - protein, 106 aa, synthetic construct
  REGION 1..106: note = Monoclonal antibody Light chain amino acid sequence
DIQMTQSPSTLSASVGDRVTITCRASQSISSWLAWYQQKPGKAPKLLIYDASSLESGVPSRFSGSGSGTEFTLTISSLQPDDFATYYCQQYNSYSTFGQGTKVEIK

SEQ ID NO: 89 - protein, 8 aa, synthetic construct
  REGION 1..8: note = Antibody complementarity determining regions (CDRs)
GFIFSKHD

SEQ ID NO: 90 - protein, 7 aa, synthetic construct
  REGION 1..7: note = Antibody complementarity determining regions (CDRs)
IGDAGDT

SEQ ID NO: 91 - protein, 13 aa, synthetic construct
  REGION 1..13: note = Antibody complementarity determining regions (CDRs)
GRGMAVAGFPLDV

SEQ ID NO: 92 - protein, 6 aa, synthetic construct
  REGION 1..6: note = Antibody complementarity determining regions (CDRs)
QNIHIN

SEQ ID NO: 93 - skipped

SEQ ID NO: 94 - protein, 10 aa, synthetic construct
  REGION 1..10: note = Antibody complementarity determining regions (CDRs)
QQFNPWSPWT

SEQ ID NO: 95 - protein, 8 aa, synthetic construct
  REGION 1..8: note = Antibody complementarity determining regions (CDRs)
GFTFNNHD

SEQ ID NO: 96 - protein, 7 aa, synthetic construct
  REGION 1..7: note = Antibody complementarity determining regions (CDRs)
IGNFGDT

SEQ ID NO: 97 - protein, 12 aa, synthetic construct
  REGION 1..12: note = Antibody complementarity determining regions (CDRs)
ARGRAVAGNPLD

SEQ ID NO: 98 - protein, 6 aa, synthetic construct
  REGION 1..6: note = Antibody complementarity determining regions (CDRs)
QTLYNN

SEQ ID NO: 99 - skipped

SEQ ID NO: 100 - protein, 10 aa, synthetic construct
  REGION 1..10: note = Antibody complementarity determining regions (CDRs)
QHYTPWPPYT

SEQ ID NO: 101 - protein, 8 aa, synthetic construct
  REGION 1..8: note = Antibody complementarity determining regions (CDRs)
GFTFSNHD

SEQ ID NO: 102 - protein, 7 aa, synthetic construct
  REGION 1..7: note = Antibody complementarity determining regions (CDRs)
IGNAGDT

SEQ ID NO: 103 - protein, 13 aa, synthetic construct
  REGION 1..13: note = Antibody complementarity determining regions (CDRs)
ARGIAVAGFPLDV

SEQ ID NO: 104 - protein, 6 aa, synthetic construct
  REGION 1..6: note = Antibody complementarity determining regions (CDRs)
QSVNSN

SEQ ID NO: 105 - skipped

SEQ ID NO: 106 - protein, 10 aa, synthetic construct
  REGION 1..10: note = Antibody complementarity determining regions (CDRs)
QQYNPWPPWT

SEQ ID NO: 107 - protein, 8 aa, synthetic construct
  REGION 1..8: note = Antibody complementarity determining regions (CDRs)
GFTFSNHD

SEQ ID NO: 108 - protein, 7 aa, synthetic construct
  REGION 1..7: note = Antibody complementarity determining regions (CDRs)
IGNAGDT

SEQ ID NO: 109 - protein, 13 aa, synthetic construct
  REGION 1..13: note = Antibody complementarity determining regions (CDRs)
ARGIAVAGNPLDV

SEQ ID NO: 110 - protein, 6 aa, synthetic construct
  REGION 1..6: note = Antibody complementarity determining regions (CDRs)
QSVNSN

SEQ ID NO: 111 - skipped

SEQ ID NO: 112 - protein, 10 aa, synthetic construct
  REGION 1..10: note = Antibody complementarity determining regions (CDRs)
QQYNPWPPWT

SEQ ID NO: 113 - protein, 8 aa, synthetic construct
  REGION 1..8: note = Antibody complementarity determining regions (CDRs)
GFTFSSYD

SEQ ID NO: 114 - protein, 7 aa, synthetic construct
  REGION 1..7: note = Antibody complementarity determining regions (CDRs)
IGTAGDT

SEQ ID NO: 115 - protein, 13 aa, synthetic construct
  REGION 1..13: note = Antibody complementarity determining regions (CDRs)
ARGIAVAGFPLDV

SEQ ID NO: 116 - protein, 6 aa, synthetic construct
  REGION 1..6: note = Antibody complementarity determining regions (CDRs)
QSVSSN

SEQ ID NO: 117 - skipped

SEQ ID NO: 118 - protein, 10 aa, synthetic construct [sequence omitted]

SEQ ID NO: 119 - protein, 8 aa, synthetic construct
  REGION 1..8: note = Antibody complementarity determining regions (CDRs)
GFTFSSYD

SEQ ID NO: 120 - protein, 7 aa, synthetic construct
  REGION 1..7: note = Antibody complementarity determining regions (CDRs)
IGTAGDT

SEQ ID NO: 121 - protein, 13 aa, synthetic construct
  REGION 1..13: note = Antibody complementarity determining regions (CDRs)
ARGIAVAGNPLDV

SEQ ID NO: 122 - protein, 6 aa, synthetic construct
  REGION 1..6: note = Antibody complementarity determining regions (CDRs)
QSVSSN

SEQ ID NO: 123 - skipped

SEQ ID NO: 124 - protein, 10 aa, synthetic construct [sequence omitted]

SEQ ID NO: 125 - protein, 8 aa, synthetic construct
  REGION 1..8: note = Antibody complementarity determining regions (CDRs)
GFTFNNYW

SEQ ID NO: 126 - protein, 8 aa, synthetic construct
  REGION 1..8: note = Antibody complementarity determining regions (CDRs)
IKGDGSEK

SEQ ID NO: 127 - protein, 19 aa, synthetic construct
  REGION 1..19: note = Antibody complementarity determining regions (CDRs)
ARVGGGDYYDSSGYYWLDT

SEQ ID NO: 128 - protein, 12 aa, synthetic construct
  REGION 1..12: note = Antibody complementarity determining regions (CDRs)
QSVLYSSNNKNY

SEQ ID NO: 129 - skipped

SEQ ID NO: 130 - protein, 8 aa, synthetic construct
  REGION 1..8: note = Antibody complementarity determining regions (CDRs)
QQYYENPT

SEQ ID NO: 131 - protein, 8 aa, synthetic construct
  REGION 1..8: note = Antibody complementarity determining regions (CDRs)
GFTFNNYW

SEQ ID NO: 132 - protein, 8 aa, synthetic construct
  REGION 1..8: note = Antibody complementarity determining regions (CDRs)
VNQDGNEK

SEQ ID NO: 133 - protein, 19 aa, synthetic construct
  REGION 1..19: note = Antibody complementarity determining regions (CDRs)
ARVGGGDYYDSSGYYWFDT

SEQ ID NO: 134 - protein, 12 aa, synthetic construct
  REGION 1..12: note = Antibody complementarity determining regions (CDRs)
QSVLYNSNNKNY

SEQ ID NO: 135 - skipped

SEQ ID NO: 136 - protein, 8 aa, synthetic construct
  REGION 1..8: note = Antibody complementarity determining regions (CDRs)
QQYYDTPT

SEQ ID NO: 137 - protein, 8 aa, synthetic construct
  REGION 1..8: note = Antibody complementarity determining regions (CDRs)
GFSFGDNA

SEQ ID NO: 138 - protein, 10 aa, synthetic construct
  REGION 1..10: note = Antibody complementarity determining regions (CDRs)
IRAKGYGGTT

SEQ ID NO: 139 - protein, 13 aa, synthetic construct
  REGION 1..13: note = Antibody complementarity determining regions (CDRs)
AKYASGWEVGFDP

SEQ ID NO: 140 - protein, 6 aa, synthetic construct
  REGION 1..6: note = Antibody complementarity determining regions (CDRs)
QFIRND

SEQ ID NO: 141 - skipped

SEQ ID NO: 142 - protein, 9 aa, synthetic construct
  REGION 1..9: note = Antibody complementarity determining regions (CDRs)
LQDYNFPWT

SEQ ID NO: 143 - protein, 8 aa, synthetic construct
  REGION 1..8: note = Antibody complementarity determining regions (CDRs)
GYNFKAYG

SEQ ID NO: 144 - protein, 8 aa, synthetic construct
  REGION 1..8: note = Antibody complementarity determining regions (CDRs)
ITPYNGKT

SEQ ID NO: 145 - protein, 12 aa, synthetic construct
  REGION 1..12: note = Antibody complementarity determining regions (CDRs)
ARTPAALASFDY

SEQ ID NO: 146 - protein, 6 aa, synthetic construct
  REGION 1..6: note = Antibody complementarity determining regions (CDRs)
QSISSW

SEQ ID NO: 147 - skipped

SEQ ID NO: 148 - protein, 8 aa, synthetic construct
  REGION 1..8: note = Antibody complementarity determining regions (CDRs)
QQYYSYST

SEQ ID NO: 149 - protein, 8 aa, synthetic construct
  REGION 1..8: note = Antibody complementarity determining regions (CDRs)
GFTYFSYA

SEQ ID NO: 150 - protein, 8 aa, synthetic construct
  REGION 1..8: note = Antibody complementarity determining regions (CDRs)
VNVRVGTS

SEQ ID NO: 151 - protein, 14 aa, synthetic construct
  REGION 1..14: note = Antibody complementarity determining regions (CDRs)
ATVGATQDLRYFDF

SEQ ID NO: 152 - protein, 6 aa, synthetic construct
  REGION 1..6: note = Antibody complementarity determining regions (CDRs)
QSVYRL

SEQ ID NO: 153 - skipped

SEQ ID NO: 154 - protein, 8 aa, synthetic construct
  REGION 1..8: note = Antibody complementarity determining regions (CDRs)
QQYNSYST

SEQ ID NO: 155 - protein, 8 aa, synthetic construct
  REGION 1..8: note = Antibody complementarity determining regions (CDRs)
GFTFTSYG

SEQ ID NO: 156 - protein, 8 aa, synthetic construct
  REGION 1..8: note = Antibody complementarity determining regions (CDRs)
INTYNGNT

SEQ ID NO: 157 - protein, 14 aa, synthetic construct
  REGION 1..14: note = Antibody complementarity determining regions (CDRs)
ARGQGRYGDYIYNH

SEQ ID NO: 158 - protein, 6 aa, synthetic construct
  REGION 1..6: note = Antibody complementarity determining regions (CDRs)
QIISSW

SEQ ID NO: 159 - skipped

SEQ ID NO: 160 - protein, 9 aa, synthetic construct
  REGION 1..9: note = Antibody complementarity determining regions (CDRs)
QHYNDFPLS

SEQ ID NO: 161 - protein, 8 aa, synthetic construct
  REGION 1..8: note = Antibody complementarity determining regions (CDRs)
GFTFSSHA

SEQ ID NO: 162 - protein, 8 aa, synthetic construct
  REGION 1..8: note = Antibody complementarity determining regions (CDRs)
ISYDGYNK

SEQ ID NO: 163 - protein, 13 aa, synthetic construct
  REGION 1..13: note = Antibody complementarity determining regions (CDRs)
ARDRDSSGYIFDY

SEQ ID NO: 164 - protein, 6 aa, synthetic construct
  REGION 1..6: note = Antibody complementarity determining regions (CDRs)
QDISNY

SEQ ID NO: 165 - skipped

SEQ ID NO: 166 - protein, 8 aa, synthetic construct
  REGION 1..8: note = Antibody complementarity determining regions (CDRs)
QQYDNLPS

SEQ ID NO: 167 - protein, 10 aa, synthetic construct
  REGION 1..10: note = Antibody complementarity determining regions (CDRs)
GDSVSSNSAA

SEQ ID NO: 168 - protein, 9 aa, synthetic construct
  REGION 1..9: note = Antibody complementarity determining regions (CDRs)
TYYRSRWYS

SEQ ID NO: 169 - protein, 14 aa, synthetic construct
  REGION 1..14: note = Antibody complementarity determining regions (CDRs)
ARDLGIAAADWFDS

SEQ ID NO: 170 - protein, 6 aa, synthetic construct
  REGION 1..6: note = Antibody complementarity determining regions (CDRs)
QSVSNR

SEQ ID NO: 171 - skipped

SEQ ID NO: 172 - protein, 8 aa, synthetic construct
  REGION 1..8: note = Antibody complementarity determining regions (CDRs)
HQYHNWPG

SEQ ID NO: 173 - protein, 8 aa, synthetic construct
  REGION 1..8: note = Antibody complementarity determining regions (CDRs)
GFIFNMYG

SEQ ID NO: 174 - protein, 8 aa, synthetic construct
  REGION 1..8: note = Antibody complementarity determining regions (CDRs)
ISYDGTKE

SEQ ID NO: 175 - protein, 11 aa, synthetic construct
  REGION 1..11: note = Antibody complementarity determining regions (CDRs)
ARSPSGHALDL

SEQ ID NO: 176 - protein, 11 aa, synthetic construct
  REGION 1..11: note = Antibody complementarity determining regions (CDRs)
QSLLLSNGYNY

SEQ ID NO: 177 - skipped

SEQ ID NO: 178 - protein, 9 aa, synthetic construct
  REGION 1..9: note = Antibody complementarity determining regions (CDRs)
MQGLQTPFT

SEQ ID NO: 179 - protein, 8 aa, synthetic construct
  REGION 1..8: note = Antibody complementarity determining regions (CDRs)
GGSIRGYS

SEQ ID NO: 180 - protein, 7 aa, synthetic construct
  REGION 1..7: note = Antibody complementarity determining regions (CDRs)
MLYTGTT

SEQ ID NO: 181 - protein, 14 aa, synthetic construct
  REGION 1..14: note = Antibody complementarity determining regions (CDRs)
ARGPTVSGPIVVDY

SEQ ID NO: 182 - protein, 6 aa, synthetic construct
  REGION 1..6: note = Antibody complementarity determining regions (CDRs)
QSISTW

SEQ ID NO: 183 - skipped

SEQ ID NO: 184 - protein, 9 aa, synthetic construct
  REGION 1..9: note = Antibody complementarity determining regions (CDRs)
QQYYNYGIT

SEQ ID NO: 185 - protein, 8 aa, synthetic construct
  REGION 1..8: note = Antibody complementarity determining regions (CDRs)
GYSFTRYG

SEQ ID NO: 186 - protein, 8 aa, synthetic construct
  REGION 1..8: note = Antibody complementarity determining regions (CDRs)
ISAYNGDT

SEQ ID NO: 187 - protein, 24 aa, synthetic construct
  REGION 1..24: note = Antibody complementarity determining regions (CDRs)
GRDTRYCSGAGCPRPSWYYYYMDV

SEQ ID NO: 188 - protein, 6 aa, synthetic construct
  REGION 1..6: note = Antibody complementarity determining regions (CDRs)
QTISNH

SEQ ID NO: 189 - skipped

SEQ ID NO: 190 - protein, 10 aa, synthetic construct
  REGION 1..10: note = Antibody complementarity determining regions (CDRs)
QQTYDIPPYT

SEQ ID NO: 191 - protein, 8 aa, synthetic construct
  REGION 1..8: note = Antibody complementarity determining regions (CDRs)
GFTFSRYG

SEQ ID NO: 192 - protein, 8 aa, synthetic construct
  REGION 1..8: note = Antibody complementarity determining regions (CDRs)
IRYDGTEK

SEQ ID NO: 193 - protein, 12 aa, synthetic construct
  REGION 1..12: note = Antibody complementarity determining regions (CDRs)
ANPYITPPTNDY

SEQ ID NO: 194 - protein, 6 aa, synthetic construct
  REGION 1..6: note = Antibody complementarity determining regions (CDRs)
QDISNY

SEQ ID NO: 195 - skipped

SEQ ID NO: 196 - protein, 9 aa, synthetic construct
  REGION 1..9: note = Antibody complementarity determining regions (CDRs)
QKHNSAPWT

SEQ ID NO: 197 - protein, 8 aa, synthetic construct
  REGION 1..8: note = Antibody complementarity determining regions (CDRs)
GFTYFGFA

SEQ ID NO: 198 - protein, 8 aa, synthetic construct
  REGION 1..8: note = Antibody complementarity determining regions (CDRs)
INLLSGTT

SEQ ID NO: 199 - protein, 14 aa, synthetic construct
  REGION 1..14: note = Antibody complementarity determining regions (CDRs)
AKVGATQDLHYFDF

SEQ ID NO: 200 - protein, 6 aa, synthetic construct
  REGION 1..6: note = Antibody complementarity determining regions (CDRs)
QSVYRW

SEQ ID NO: 201 - skipped

SEQ ID NO: 202 - protein, 8 aa, synthetic construct
  REGION 1..8: note = Antibody complementarity determining regions (CDRs)
QQYNSHST

SEQ ID NO: 203 - protein, 8 aa, synthetic construct
  REGION 1..8: note = Antibody complementarity determining regions (CDRs)
GFTFFSYA

SEQ ID NO: 204 - protein, 8 aa, synthetic construct
  REGION 1..8: note = Antibody complementarity determining regions (CDRs)
INVRGGTT

SEQ ID NO: 205 - protein, 14 aa, synthetic construct
  REGION 1..14: note = Antibody complementarity determining regions (CDRs)
AKVGATQDLRYFDY

SEQ ID NO: 206 - protein, 6 aa, synthetic construct
  REGION 1..6: note = Antibody complementarity determining regions (CDRs)
QSVSRW

SEQ ID NO: 207 - skipped

SEQ ID NO: 208 - protein, 8 aa, synthetic construct
  REGION 1..8: note = Antibody complementarity determining regions (CDRs)
QQYNSYST

SEQ ID NO: 209 - protein, 8 aa, synthetic construct
  REGION 1..8: note = Antibody complementarity determining regions (CDRs)
GFTFFSYA

SEQ ID NO: 210 - protein, 8 aa, synthetic construct
  REGION 1..8: note = Antibody complementarity determining regions (CDRs)
INVLGGTT

SEQ ID NO: 211 - protein, 14 aa, synthetic construct
  REGION 1..14: note = Antibody complementarity determining regions (CDRs)
AKVGATQDLHYFDY

SEQ ID NO: 212 - protein, 6 aa, synthetic construct
  REGION 1..6: note = Antibody complementarity determining regions (CDRs)
QSVSRW

SEQ ID NO: 213 - skipped

SEQ ID NO: 214 - protein, 8 aa, synthetic construct
  REGION 1..8: note = Antibody complementarity determining regions (CDRs)
QQYNSYST

SEQ ID NO: 215 - protein, 8 aa, synthetic construct
  REGION 1..8: note = Antibody complementarity determining regions (CDRs)
GFTFSSYA

SEQ ID NO: 216 - protein, 8 aa, synthetic construct
  REGION 1..8: note = Antibody complementarity determining regions (CDRs)
ISGSGGST

SEQ ID NO: 217 - protein, 14 aa, synthetic construct
  REGION 1..14: note = Antibody complementarity determining regions (CDRs)
AKVGATQDLRYFDY

SEQ ID NO: 218 - protein, 6 aa, synthetic construct
  REGION 1..6: note = Antibody complementarity determining regions (CDRs)
QSISSW

SEQ ID NO: 219 - skipped

SEQ ID NO: 220 - protein, 8 aa, synthetic construct
  REGION 1..8: note = Antibody complementarity determining regions (CDRs)
QQYNSYST

SEQ ID NO: 221 - DNA, 375 bp, synthetic construct
  misc_feature 1..375: note = Monoclonal antibody Heavy chain nucleotide sequence
  source 1..375: mol_type = other DNA
gaggtgcagctggtggagtctgggggaggcttggtccagcctggggggtccctgagactctcctgtgcagcctctggattcacctttagtagctattggatgacctgggtccgccaggctccagggaaggggctggagtgggtggccaatataaagccagatggaagtgagaaaaactatgtggactctgtgaagggccgattcaccatctccagagacaacgccgagaactcactgtatctgcaaatgaacagcctgagagccgaggacacggctgtattttactgtgcgagagacctaagatattgtagtagtactagctgttcaccagctcttgactactggggccagggaaccctgctcaccgtctcctca

SEQ ID NO: 222 - DNA, 363 bp, synthetic construct
  misc_feature 1..363: note = Monoclonal antibody Heavy chain nucleotide sequence
  source 1..363: mol_type = other DNA
gaggtgcagctgttggagtctgggggaggcttggtacagccgggggggtccctgagactctcctgtgcagcctctggattcacctttgacaattatggcatgagttgggtccgccaggctccagggaaggggctggagtgggtctcagttattagcagtggtggtgttggcacatactacgcagactccgtgaagggccggttcatcatctccagagacaatgccaagaacacactgtatctgcaaatgaacagcctgagagccgaggacacggccgtatattattgtgcgaaagatttattacgttatgagagtagtggctatagcccgtggggccagggaaccctggtcaccgtctcctcc

SEQ ID NO: 223 - DNA, 381 bp, synthetic construct
  misc_feature 1..381: note = Monoclonal antibody Heavy chain nucleotide sequence
  source 1..381: mol_type = other DNA
caggtgcagctgcaggagtcgggcccaggactactgaagccttcacagaccctgtctctcacctgcactgtctctggtgccttcctcaacagtggtggttactactggacctggatccgccagcacccaggaaagggcctggagtggattgggtacatctattacagtgggactacctactacaacccgtccctcaagagtcgagttaccatttcagtggacacgtctaagaaccagttctccctcaacgtgaactctgtgattgccgcggacacggccgtgtattactgtgcgagagccccgcggattccttttggggaagttatagggggtgctgctttcgacgtctggggccaagggacaacggtcattgtctcttca

SEQ ID NO: 224 - DNA, 375 bp, synthetic construct
  misc_feature 1..375: note = Monoclonal antibody Heavy chain nucleotide sequence
  source 1..375: mol_type = other DNA
caggtgcagctgcaggagtcgggcccaggactggtgaagccttcacagaccctgtccctcacctgcactgtctctggtggctccttcagtaatagtaattattactggagttggatccggcagcccgccgggaagggactggagtggatagggcatatctatgccagtgcgatcacccactacaatccctcccttaagagtcgagtcaccatatcaaaagacacgtccaagaatcagttctccctgaatctgaggtctgtgaccgccgcagacacggccgtgtatttttgtgcgagaggattccgtttggcagcagaagcctactaccacggcatggacgtctggggccaagggaccaccgtcaccgtctcctca

SEQ ID NO: 225 - DNA, 393 bp, synthetic construct
  misc_feature 1..393: note = Monoclonal antibody Heavy chain nucleotide sequence
  source 1..393: mol_type = other DNA
caggtgcagctggtgcagtctggggctgaggtgaagaagcctgggtcctcggtgaaggtcgcctgcaaggcttctggaggcaccttcagcaggtctgcaatcagctgggtgcgacaggcccctggacaagggcttgagtggatgggagggatcatccgtatttttaatacagcggactacgcacagaagttccagggcagagtcacaatgtccgcggacgaatccacgagcacagcctacatggaattgagcagcctgagatctgaggacacggccgtgtattactgtgcgacgtcctcactcagcgatattgtagtggcggaaggtgcctttgtcgaccactactttggtatggacgtctggggccaagggaccacggtcaccgtctcctca

SEQ ID NO: 226 - DNA, 384 bp, synthetic construct
  misc_feature 1..384: note = Monoclonal antibody Heavy chain nucleotide sequence
  source 1..384: mol_type = other DNA
gaggtgcagctggtggagtctgggggaggcttggtacagcctggagggtccctgagactctcgtgtgtaggctctgcattcaccttgagtagatatgaaataaactgggtccgccaggctccagggaagggactggagtggatttcacacataagtagtagtggggataccataaattacgcagactctgtgaagggccgattcaccatctccagagacaactccaagaactcaaattttctgcaaatgaacaggctgagagccgaggacacggctgtttattactgtgcgacatggggactgggatattgtaatagtaccggctgctatatcactgacggtatggacgtctgggggcaagggaccacggtcaccgtctcctca

SEQ ID NO: 227 - DNA, 363 bp, synthetic construct
  misc_feature 1..363: note = Monoclonal antibody Heavy chain nucleotide sequence
  source 1..363: mol_type = other DNA
cagctgcagctgcaggagtccggctcaggactggtgaagccttcacagaccctgtccctcacctgcgctgtgtctggtggctccatcagcagtagtgattactcctggagctggatccggcagccaccagggaagggcctggagtggattgggtacatctatcatagtgggaacacctactacaatccgtccgtcaagagtcgagtcaccatttcactagacagctccaagaaccacttctccctgaccctgacctctgtgactgccgcagacacggccatatattattgtgccagggatcccggtggtaactccggctggttcgacccctggggccagggagccctggtcaccgtctcctca

SEQ ID NO: 228 - DNA, 357 bp, synthetic construct
  misc_feature 1..357: note = Monoclonal antibody Heavy chain nucleotide sequence
  source 1..357: mol_type = other DNA
gaggtgcagctggtggagtctgggggaggcttggtaaagcctggggggtcccttagactctcctgtgcaccctctggattcattttcagtaatacatggatgagctgggtccgccaggctccagggaaggggctggagtgggttggccacattaaaagcaaagctgatggtgggacaacagactacgctgcacccgtgaaaggcagattcaccatctccagagatgattcaagaaatacgctatatctacaaatggacagcctgaaaaccgaggacacaggcgtgtattactgtaccacaggctggtattccacccttgactactggggccagggaaccctggtcaccgtctcctca

SEQ ID NO: 229 - DNA, 363 bp, synthetic construct
  misc_feature 1..363: note = Monoclonal antibody Heavy chain nucleotide sequence
  source 1..363: mol_type = other DNA
caggttcagctggtgcagtctggatctgaggtgaagaagcctggggcctcagtgaaggtctcctgcaaggcttctggctacacctttaccagtaatggtatcagttgggtgcgacaggcccctggacaagggcttgagtggatgggatggatcagcgcttacaatggtaacacaaactatgcacagaagttccagggcagagtctccttgaccacagacacatccacgagcacagcctacatggagctgcggaacctgacatctgacgacacggccagatattactgtgcgagaagccgaggtcactacggtgactacctttatggctactggggccagggaaccctggtcaccgtctcctca

SEQ ID NO: 230 - DNA, 354 bp, synthetic construct
  misc_feature 1..354: note = Monoclonal antibody Heavy chain nucleotide sequence
  source 1..354: mol_type = other DNA
gaagtgcagctggtggagtctgggggaggcttggtacagcctggcaggtccctgagactctcctgtgcagcctctggattcacatttgaaacttatgccatgcactgggtccgacaagctccagggaagggcctggagtgggtctctggtatcagtttcaatagtcgtagtagaggctatgcggacgctgtgaggggccgattcaccatctccagagacaactccaagaactccctctttctggaaatgaatagtctgagacctgaggacacggccttgtattattgtgcaagagatgagaaatgggggactccgagtgactggggccagggagtcctggtcaccgtctcctca

SEQ ID NO: 231 - DNA, 360 bp, synthetic construct; identical to SEQ ID NO: 16
  misc_feature 1..360: note = Monoclonal antibody Heavy chain nucleotide sequence
  source 1..360: mol_type = other DNA

SEQ ID NO: 232 - DNA, 363 bp, synthetic construct
  misc_feature 1..363: note = Monoclonal antibody Heavy chain nucleotide sequence
  source 1..363: mol_type = other DNA
cagctgcagctgcaggagtcgggcccaggactggtgaagccttcggagaccctgtccctcacctgcactgtctctggtggctccatcagcagtagtagttactactggggctggatccgccagcccccagggaaggggctggagtggattgggagtctctcttatactgggagcacctactacaacccgtccctcaagagtcgagtcaccatatcactagacacgtccaagaaccaattctccctgaagctgagctctgtgaccgccgcggacacggccgtgtattactgtgcgagagagagtggtagtggtggtacccatactgactcctggggccagggaaccctggtctccgtctcctca

SEQ ID NO: 233 - DNA, 387 bp, synthetic construct
  misc_feature 1..387: note = Monoclonal antibody Heavy chain nucleotide sequence
  source 1..387: mol_type = other DNA
gaggtgcagctggtggagtctgggggaggtgtactacggcctggggaatccctgagactctcctgtgtagcctctggattcacctttgaggattatggcatgagttgggtccgccaagttccagggaaggggctggagtgggtctctggaattaattggaatggtggtagcacacgatatgcagactctgtgaagggccgattcaccatctccagagacaacgccaacaattccctgtatctgcaaatgaacagtctgagagccgaagacacggccttgtatcactgtgcgagagataaggccatccagggtgcactaatggtgtatgctatgaggggtcggtggttcgacccctggggccagggaaccctggtcaccgtctcctca

SEQ ID NO: 234 - DNA, 366 bp, synthetic construct
  misc_feature 1..366: note = Monoclonal antibody Heavy chain nucleotide sequence
  source 1..366: mol_type = other DNA
gaggtgcagctggtggagtctgggggaggcttggtacagccagggcggtccctgagactctcctgtacagcttctggattcacctttggtgattatgctatgagctggttccgccaggctccagggaaggggctggagtgggtaggtttcattagaagcaaagcttatggtgggacaacagaatacgccgcgtctgtgaaaggcagattcaccatctcaagagatgattccaaaagcatcgcctatctgcaaatgaacagcctgaaaaccgaggacacagccgtgtattactgtactaaatatgccagtggctgggaggttggattcgacccctggggccagggaaccctggtcaccgtctcctca

SEQ ID NO: 235 - DNA, 357 bp, synthetic construct
  misc_feature 1..357: note = Monoclonal antibody Heavy chain nucleotide sequence
  source 1..357: mol_type = other DNA
caggttcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagaacccccgctgccctggcaagttttgactactggggccagggaaccctggtcaccgtctcctca

SEQ ID NO: 236 - DNA, 363 bp, synthetic construct
  misc_feature 1..363: note = Monoclonal antibody Heavy chain nucleotide sequence
  source 1..363: mol_type = other DNA
caggttcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagaggccaaggacggtacggtgactacatttataatcactggggccagggaaccctggtcaccgtctcctca

SEQ ID NO: 237 - DNA, 360 bp, synthetic construct
  misc_feature 1..360: note = Monoclonal antibody Heavy chain nucleotide sequence
  source 1..360: mol_type = other DNA
caggtgcagctggtggagtctgggggaggcgtggtccagcctgggaggtccctgagactctcctgtgcagcctctggattcaccttcagtagctatgctatgcactgggtccgccaggctccaggcaaggggctggagtgggtggcagttatatcatatgatggaagcaataaatactacgcagactccgtgaagggccgattcaccatctccagagacaattccaagaacacgctgtatctgcaaatgaacagcctgagagctgaggacacggctgtgtattactgtgcgagagatcgcgatagtagtggttatatttttgactactggggccagggaaccctggtcaccgtctcctca

SEQ ID NO: 238 - DNA, 372 bp, synthetic construct
  misc_feature 1..372: note = Monoclonal antibody Heavy chain nucleotide sequence
  source 1..372: mol_type = other DNA
caggtacagctgcagcagtcaggtccaggactggtgaagccctcgcagaccctctcactcacctgtgccatctccggggacagtgtctctagcaacagtgctgcttggaactggatcaggcagtccccatcgagaggccttgagtggctgggaaggacatactacaggtccaagtggtataatgattatgcagtatctgtgaaaagtcgaataaccatcaacccagacacatccaagaaccagttctccctgcagctgaactctgtgactcccgaggacacggctgtgtattactgtgcaagagatttgggtatagcagcggctgactggttcgactcctggggccaaggaaccctggtcaccgtctcctca

SEQ ID NO: 239 - DNA, 354 bp, synthetic construct
  misc_feature 1..354: note = Monoclonal antibody Heavy chain nucleotide sequence
  source 1..354: mol_type = other DNA
caggtgcagctggtggagtctgggggaggcgtggtccagcctgggaggtccctgagactctcctgtgcagcctctggattcaccttcagtagctatggcatgcactgggtccgccaggctccaggcaaggggctggagtgggtggcagttatatcatatgatggaagtaataaatactatgcagactccgtgaagggccgattcaccatctccagagacaattccaagaacacgctgtatctgcaaatgaacagcctgagagctgaggacacggctgtgtattactgtgcgaggagccccagtgggcatgcttttgatgtctggggccaagggacaatggtcaccgtctcttca

SEQ ID NO: 240 - DNA, 360 bp, synthetic construct
  misc_feature 1..360: note = Monoclonal antibody Heavy chain nucleotide sequence
  source 1..360: mol_type = other DNA
caggtgcagctgcaggagtcgggcccaggactggtgaagccttcggagaccctgtccctcacctgcactgtctctggtggctccatcagtagttactactggagctggatccggcagcccccagggaagggactggagtggattgggtatatctattacagtgggagcaccaactacaacccctccctcaagagtcgagtcaccatatcagtagacacgtccaagaaccagttctccctgaagctgagctctgtgaccgctgcggacacggccgtgtattactgtgcgaggggaccgactgtgtcagggcccatagtagttgactactggggccagggaaccctggtcaccgtctcctca

SEQ ID NO: 241 - DNA, 393 bp, synthetic construct
  misc_feature 1..393: note = Monoclonal antibody Heavy chain nucleotide sequence
  source 1..393: mol_type = other DNA
caggttcagctggtgcagtctggagctgaggtgaagaagcctggggcctcagtgaaggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagagatacccgatattgtagtggtggtagctgcccccggccaagttggtactactactacatggacgtctggggcaaagggaccacggtcaccgtctcctca

SEQ ID NO: 242 - DNA, 357 bp, synthetic construct
  misc_feature 1..357: note = Monoclonal antibody Heavy chain nucleotide sequence
  source 1..357: mol_type = other DNA
caggtgcagctggtggagtctgggggaggcgtggtccagcctggggggtccctgagactctcctgtgcagcgtctggattcaccttcagtagctatggcatgcactgggtccgccaggctccaggcaaggggctggagtgggtggcatttatacggtatgatggaagtaataaatactatgcagactccgtgaagggccgattcaccatctccagagacaattccaagaacacgctgtatctgcaaatgaacagcctgagagctgaggacacggctgtgtattactgtgcgaatccttatataacgccgcctactaatgactactggggccagggaaccctggtcaccgtctcctca

SEQ ID NO: 243 - DNA, 375 bp, synthetic construct
  misc_feature 1..375: note = Monoclonal antibody Heavy chain nucleotide sequence
  source 1..375: mol_type = other DNA
gaggtgcagctggtggagtctgggggaggcttggtccagcctggggggtccctgagactctcctgtgcagcctctggattcacctttagtagctattggatgagctgggtccgccaggctccagggaaggggctggagtgggtggccaacataaagcaagatggaagtgagaaatactatgtggactctgtgaagggccgattcaccatctccagagacaacgccaagaactcactgtatctgcaaatgaacagcctgagagccgaggacacggctgtgtattactgtgcgagagacctaagatattgtagtagtaccagctgttcaccagctcttgactactggggccagggaaccctggtcaccgtctcctca

SEQ ID NO: 244 - DNA, 363 bp, synthetic construct
  misc_feature 1..363: note = Monoclonal antibody Heavy chain nucleotide sequence
  source 1..363: mol_type = other DNA
gaggtgcagctgttggagtctgggggaggcttggtacagcctggggggtccctgagactctcctgtgcagcctctggattcacctttagcagctatgccatgagctgggtccgccaggctccagggaaggggctggagtgggtctcagctattagtggtagtggtggtagcacatactacgcagactccgtgaagggccggttcaccatctccagagacaattccaagaacacgctgtatctgcaaatgaacagcctgagagccgaggacacggccgtatattactgtgcgaaagatttattacgttatgatagtagtggttatagcccctggggccagggaaccctggtcaccgtctcctcc

SEQ ID NO: 245 - DNA, 381 bp, synthetic construct
  misc_feature 1..381: note = Monoclonal antibody Heavy chain nucleotide sequence
  source 1..381: mol_type = other DNA
caggtgcagctgcaggagtcgggcccaggactggtgaagccttcacagaccctgtccctcacctgcactgtctctggtggctccatcagcagtggtggttactactggagctggatccgccagcacccagggaagggcctggagtggattgggtacatctattacagtgggagcacctactacaacccgtccctcaagagtcgagttaccatatcagtagacacgtctaagaaccagttctccctgaagctgagctctgtgactgccgcggacacggccgtgtattactgtgcgagagccccgcggattacgtttgggggagttatagggggtgctgcttttgatgtctggggccaagggacaatggtcaccgtctcttca

SEQ ID NO: 246 - DNA, 375 bp, synthetic construct
  misc_feature 1..375: note = Monoclonal antibody Heavy chain nucleotide sequence
  source 1..375: mol_type = other DNA
caggtgcagctgcaggagtcgggcccaggactggtgaagccttcacagaccctgtccctcacctgcactgtctctggtggctccatcagcagtggtagttactactggagctggatccggcagcccgccgggaagggactggagtggattgggcgtatctataccagtgggagcaccaactacaacccctccctcaagagtcgagtcaccatatcagtagacacgtccaagaaccagttctccctgaagctgagctctgtgaccgccgcagacacggccgtgtattactgtgcgagaggattacgtttggcagcagaagcctactactacggtatggacgtctggggccaagggaccacggtcaccgtctcctca

SEQ ID NO: 247 - DNA, 393 bp, synthetic construct
  misc_feature 1..393: note = Monoclonal antibody Heavy chain nucleotide sequence
  source 1..393: mol_type = other DNA
caggtgcagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggagggatcatccctatctttggtacagcaaactacgcacagaagttccagggcagagtcacgattaccgcggacgaatccacgagcacagcctacatggagctgagcagcctgagatctgaggacacggccgtgtattactgtgcgacgtcctcactcagcgatattgtagtggtggtagctgcctttgtcgaccactactacggtatggacgtctggggccaagggaccacggtcaccgtctcctca

SEQ ID NO: 248 - DNA, 384 bp, synthetic construct
  misc_feature 1..384: note = Monoclonal antibody Heavy chain nucleotide sequence
  source 1..384: mol_type = other DNA
gaggtgcagctggtggagtctgggggaggcttggtacagcctggagggtccctgagactctcctgtgcagcctctggattcaccttcagtagttatgaaatgaactgggtccgccaggctccagggaaggggctggagtgggtttcatacattagtagtagtggtagtaccatatactacgcagactctgtgaagggccgattcaccatctccagagacaacgccaagaactcactgtatctgcaaatgaacagcctgagagccgaggacacggctgtttattactgtgcgacatggggactgggatattgtagtagtaccagctgctatatcactgacggtatggacgtctgggggcaagggaccacggtcaccgtctcctca

SEQ ID NO: 249 - DNA, 357 bp, synthetic construct
  misc_feature 1..357: note = Monoclonal antibody Heavy chain nucleotide sequence
  source 1..357: mol_type = other DNA
gaggtgcagctggtggagtctgggggaggcttggtaaagcctggggggtcccttagactctcctgtgcagcctctggattcactttcagtaacgcctggatgagctgggtccgccaggctccagggaaggggctggagtgggttggccgtattaaaagcaaaactgatggtgggacaacagactacgctgcacccgtgaaaggcagattcaccatctcaagagatgattcaaaaaacacgctgtatctgcaaatgaacagcctgaaaaccgaggacacagccgtgtattactgtaccacaggctgctattccacccttgactactggggccagggaaccctggtcaccgtctcctca

SEQ ID NO: 250 - DNA, 354 bp, synthetic construct
  misc_feature 1..354: note = Monoclonal antibody Heavy chain nucleotide sequence
  source 1..354: mol_type = other DNA
gaagtgcagctggtggagtctgggggaggcttggtacagcctggcaggtccctgagactctcctgtgcagcctctggattcacctttgatgattatgccatgcactgggtccggcaagctccagggaagggcctggagtgggtctcaggtattagttggaatagtggtagcataggctatgcggactctgtgaagggccgattcaccatctccagagacaacgccaagaactccctgtatctgcaaatgaacagtctgagagctgaggacacggccttgtattactgtgcaaaagatgagaaatgggggactccgagtgactggggccagggaaccctggtcaccgtctcctca

SEQ ID NO: 251 - DNA, 360 bp, synthetic construct; identical to SEQ ID NO: 240
  misc_feature 1..360: note = Monoclonal antibody Heavy chain nucleotide sequence
  source 1..360: mol_type = other DNA

SEQ ID NO: 252 - DNA, 387 bp, synthetic construct
  misc_feature 1..387: note = Monoclonal antibody Heavy chain nucleotide sequence
  source 1..387: mol_type = other DNA
gaggtgcagctggtggagtctgggggaggtgtggtacggcctggggggtccctgagactctcctgtgcagcctctggattcacctttgatgattatggcatgagctgggtccgccaagctccagggaaggggctggagtgggtctctggtattaattggaatggtggtagcacaggttatgcagactctgtgaagggccgattcaccatctccagagacaacgccaagaactccctgtatctgcaaatgaacagtctgagagccgaggacacggccttgtatcactgtgcgagagataaggccatccagggtgcactaatggtgtatgctatgaggggtcggtggttcgacccctggggccagggaaccctggtcaccgtctcctca

SEQ ID NO: 253 - protein, 125 aa, synthetic construct
  REGION 1..125: note = Monoclonal antibody Heavy chain amino acid sequence
EVQLVESGGGLVQPGGSLRLSCAASGFTFSSYWMTWVRQAPGKGLEWVANIKPDGSEKNYVDSVKGRFTISRDNAENSLYLQMNSLRAEDTAVFYCARDLRYCSSTSCSPALDYWGQGTLLTVSS

SEQ ID NO: 254 - protein, 121 aa, synthetic construct
  REGION 1..121: note = Monoclonal antibody Heavy chain amino acid sequence
EVQLLESGGGLVQPGGSLRLSCAASGFTFDNYGMSWVRQAPGKGLEWVSVISSGGVGTYYADSVKGRFIISRDNAKNTLYLQMNSLRAEDTAVYYCAKDLLRYESSGYSPWGQGTLVTVSS

SEQ ID NO: 255 - protein, 127 aa, synthetic construct
  REGION 1..127: note = Monoclonal antibody Heavy chain amino acid sequence
QVQLQESGPGLLKPSQTLSLTCTVSGAFLNSGGYYWTWIRQHPGKGLEWIGYIYYSGTTYYNPSLKSRVTISVDTSKNQFSLNVNSVIAADTAVYYCARAPRIPFGEVIGGAAFDVWGQGTTVIVSS

SEQ ID NO: 256 - protein, 125 aa, synthetic construct
  REGION 1..125: note = Monoclonal antibody Heavy chain amino acid sequence
QVQLQESGPGLVKPSQTLSLTCTVSGGSFSNSNYYWSWIRQPAGKGLEWIGHIYASAITHYNPSLKSRVTISKDTSKNQFSLNLRSVTAADTAVYFCARGFRLAAEAYYHGMDVWGQGTTVTVSS

SEQ ID NO: 257 - protein, 131 aa, synthetic construct
  REGION 1..131: note = Monoclonal antibody Heavy chain amino acid sequence
QVQLVQSGAEVKKPGSSVKVACKASGGTFSRSAISWVRQAPGQGLEWMGGIIRIFNTADYAQKFQGRVTMSADESTSTAYMELSSLRSEDTAVYYCATSSLSDIVVAEGAFVDHYFGMDVWGQGTTVTVSS

SEQ ID NO: 258 - protein, 128 aa, synthetic construct
  REGION 1..128: note = Monoclonal antibody Heavy chain amino acid sequence
EVQLVESGGGLVQPGGSLRLSCVGSAFTLSRYEINWVRQAPGKGLEWISHISSSGDTINYADSVKGRFTISRDNSKNSNFLQMNRLRAEDTAVYYCATWGLGYCNSTGCYITDGMDVWGQGTTVTVSS

SEQ ID NO: 259 - protein, 121 aa, synthetic construct
  REGION 1..121: note = Monoclonal antibody Heavy chain amino acid sequence
QLQLQESGSGLVKPSQTLSLTCAVSGGSISSSDYSWSWIRQPPGKGLEWIGYIYHSGNTYYNPSVKSRVTISLDSSKNHFSLTLTSVTAADTAIYYCARDPGGNSGWFDPWGQGALVTVSS

SEQ ID NO: 260 - protein, 119 aa, synthetic construct
  REGION 1..119: note = Monoclonal antibody Heavy chain amino acid sequence
EVQLVESGGGLVKPGGSLRLSCAPSGFIFSNTWMSWVRQAPGKGLEWVGHIKSKADGGTTDYAAPVKGRFTISRDDSRNTLYLQMDSLKTEDTGVYYCTTGWYSTLDYWGQGTLVTVSS

SEQ ID NO: 261 - protein, 121 aa, synthetic construct
  REGION 1..121: note = Monoclonal antibody Heavy chain amino acid sequence
QVQLVQSGSEVKKPGASVKVSCKASGYTFTSNGISWVRQAPGQGLEWMGWISAYNGNTNYAQKFQGRVSLTTDTSTSTAYMELRNLTSDDTARYYCARSRGHYGDYLYGYWGQGTLVTVSS

SEQ ID NO: 262 - protein, 118 aa, synthetic construct
  REGION 1..118: note = Monoclonal antibody Heavy chain amino acid sequence
EVQLVESGGGLVQPGRSLRLSCAASGFTFETYAMHWVRQAPGKGLEWVSGISFNSRSRGYADAVRGRFTISRDNSKNSLFLEMNSLRPEDTALYYCARDEKWGTPSDWGQGVLVTVSS

SEQ ID NO: 263 - protein, 120 aa, synthetic construct; identical to SEQ ID NO: 38
  REGION 1..120: note = Monoclonal antibody Heavy chain amino acid sequence

SEQ ID NO: 264 - protein, 121 aa, synthetic construct
  REGION 1..121: note = Monoclonal antibody Heavy chain amino acid sequence
QLQLQESGPGLVKPSETLSLTCTVSGGSISSSSYYWGWIRQPPGKGLEWIGSLSYTGSTYYNPSLKSRVTISLDTSKNQFSLKLSSVTAADTAVYYCARESGSGGTHTDSWGQGTLVSVSS

SEQ ID NO: 265 - protein, 129 aa, synthetic construct
  REGION 1..129: note = Monoclonal antibody Heavy chain amino acid sequence
EVQLVESGGGVLRPGESLRLSCVASGFTFEDYGMSWVRQVPGKGLEWVSGINWNGGSTRYADSVKGRFTISRDNANNSLYLQMNSLRAEDTALYHCARDKAIQGALMVYAMRGRWFDPWGQGTLVTVSS

SEQ ID NO: 266 - protein, 122 aa, synthetic construct
  REGION 1..122: note = Monoclonal antibody Heavy chain amino acid sequence
EVQLVESGGGLVQPGRSLRLSCTASGFTFGDYAMSWFRQAPGKGLEWVGFIRSKAYGGTTEYAASVKGRFTISRDDSKSIAYLQMNSLKTEDTAVYYCTKYASGWEVGFDPWGQGTLVTVSS

SEQ ID NO: 267 - protein, 119 aa, synthetic construct
  REGION 1..119: note = Monoclonal antibody Heavy chain amino acid sequence
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARTPAALASFDYWGQGTLVTVSS

SEQ ID NO: 268 - protein, 121 aa, synthetic construct
  REGION 1..121: note = Monoclonal antibody Heavy chain amino acid sequence
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARGQGRYGDYIYNHWGQGTLVTVSS

SEQ ID NO: 269 - protein, 120 aa, synthetic construct
  REGION 1..120: note = Monoclonal antibody Heavy chain amino acid sequence
QVQLVESGGGVVQPGRSLRLSCAASGFTFSSYAMHWVRQAPGKGLEWVAVISYDGSNKYYADSVKGRFTISRDNSKNTLYLQMNSLRAEDTAVYYCARDRDSSGYIFDYWGQGTLVTVSS

SEQ ID NO: 270 - protein, 124 aa, synthetic construct
  REGION 1..124: note = Monoclonal antibody Heavy chain amino acid sequence
QVQLQQSGPGLVKPSQTLSLTCAISGDSVSSNSAAWNWIRQSPSRGLEWLGRTYYRSKWYNDYAVSVKSRITINPDTSKNQFSLQLNSVTPEDTAVYYCARDLGIAAADWFDSWGQGTLVTVSS

SEQ ID NO: 271 - protein, 118 aa, synthetic construct
  REGION 1..118: note = Monoclonal antibody Heavy chain amino acid sequence
QVQLVESGGGVVQPGRSLRLSCAASGFTFSSYGMHWVRQAPGKGLEWVAVISYDGSNKYYADSVKGRFTISRDNSKNTLYLQMNSLRAEDTAVYYCARSPSGHAFDVWGQGTMVTVSS

SEQ ID NO: 272 - protein, 120 aa, synthetic construct
  REGION 1..120: note = Monoclonal antibody Heavy chain amino acid sequence
QVQLQESGPGLVKPSETLSLTCTVSGGSISSYYWSWIRQPPGKGLEWIGYIYYSGSTNYNPSLKSRVTISVDTSKNQFSLKLSSVTAADTAVYYCARGPTVSGPIVVDYWGQGTLVTVSS

SEQ ID NO: 273 - protein, 131 aa, synthetic construct
  REGION 1..131: note = Monoclonal antibody Heavy chain amino acid sequence
QVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARDTRYCSGGSCPRPSWYYYYMDVWGKGTTVTVSS

SEQ ID NO: 274 - protein, 119 aa, synthetic construct
  REGION 1..119: note = Monoclonal antibody Heavy chain amino acid sequence
QVQLVESGGGVVQPGGSLRLSCAASGFTFSSYGMHWVRQAPGKGLEWVAFIRYDGSNKYYADSVKGRFTISRDNSKNTLYLQMNSLRAEDTAVYYCANPYITPPTNDYWGQGTLVTVSS

SEQ ID NO: 275 - protein, 125 aa, synthetic construct
  REGION 1..125: note = Monoclonal antibody Heavy chain amino acid sequence
EVQLVESGGGLVQPGGSLRLSCAASGFTFSSYWMSWVRQAPGKGLEWVANIKQDGSEKYYVDSVKGRFTISRDNAKNSLYLQMNSLRAEDTAVYYCARDLRYCSSTSCSPALDYWGQGTLVTVSS

SEQ ID NO: 276 - protein, 121 aa, synthetic construct
  REGION 1..121: note = Monoclonal antibody Heavy chain amino acid sequence
EVQLLESGGGLVQPGGSLRLSCAASGFTFSSYAMSWVRQAPGKGLEWVSAISGSGGSTYYADSVKGRFTISRDNSKNTLYLQMNSLRAEDTAVYYCAKDLLRYDSSGYSPWGQGTLVTVSS

SEQ ID NO: 277 - protein, 127 aa, synthetic construct
  REGION 1..127: note = Monoclonal antibody Heavy chain amino acid sequence
QVQLQESGPGLVKPSQTLSLTCTVSGGSISSGGYYWSWIRQHPGKGLEWIGYIYYSGSTYYNPSLKSRVTISVDTSKNQFSLKLSSVTAADTAVYYCARAPRITFGGVIGGAAFDVWGQGTMVTVSS

SEQ ID NO: 278 - protein, 125 aa, synthetic construct
  REGION 1..125: note = Monoclonal antibody Heavy chain amino acid sequence
QVQLQESGPGLVKPSQTLSLTCTVSGGSISSGSYYWSWIRQPAGKGLEWIGRIYTSGSTNYNPSLKSRVTISVDTSKNQFSLKLSSVTAADTAVYYCARGLRLAAEAYYYGMDVWGQGTTVTVSS

SEQ ID NO: 279 - protein, 131 aa, synthetic construct
  REGION 1..131: note = Monoclonal antibody Heavy chain amino acid sequence
QVQLVQSGAEVKKPGSSVKVSCKASGGTFSSYAISWVRQAPGQGLEWMGGIIPIFGTANYAQKFQGRVTITADESTSTAYMELSSLRSEDTAVYYCATSSLSDIVVVVAAFVDHYYGMDVWGQGTTVTVSS

SEQ ID NO: 280 - protein, 128 aa, synthetic construct
  REGION 1..128: note = Monoclonal antibody Heavy chain amino acid sequence
EVQLVESGGGLVQPGGSLRLSCAASGFTFSSYEMNWVRQAPGKGLEWVSYISSSGSTIYYADSVKGRFTISRDNAKNSLYLQMNSLRAEDTAVYYCATWGLGYCSSTSCYITDGMDVWGQGTTVTVSS

SEQ ID NO: 281 - protein, 119 aa, synthetic construct
  REGION 1..119: note = Monoclonal antibody Heavy chain amino acid sequence
EVQLVESGGGLVKPGGSLRLSCAASGFTFSNAWMSWVRQAPGKGLEWVGRIKSKTDGGTTDYAAPVKGRFTISRDDSKNTLYLQMNSLKTEDTAVYYCTTGCYSTLDYWGQGTLVTVSS

SEQ ID NO: 282 - protein, 118 aa, synthetic construct
  REGION 1..118: note = Monoclonal antibody Heavy chain amino acid sequence
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDEKWGTPSDWGQGTLVTVSS

SEQ ID NO: 283 - protein, 120 aa, synthetic construct; identical to SEQ ID NO: 272
  REGION 1..120: note = Monoclonal antibody Heavy chain amino acid sequence

SEQ ID NO: 284 - protein, 129 aa, synthetic construct
  REGION 1..129: note = Monoclonal antibody Heavy chain amino acid sequence
EVQLVESGGGVVRPGGSLRLSCAASGFTFDDYGMSWVRQAPGKGLEWVSGINWNGGSTGYADSVKGRFTISRDNAKNSLYLQMNSLRAEDTALYHCARDKAIQGALMVYAMRGRWFDPWGQGTLVTVSS

SEQ ID NO: 285 - DNA, 315 bp, synthetic construct
  misc_feature 1..315: note = Monoclonal antibody Light chain nucleotide sequence
  source 1..315: mol_type = other DNA
gacatccagatgacccagtctccatcctccctgtctgcatctgtaggagacagagtcaccatcacttgccgggctagtcagagcattgccagctatttaaattggtatcagcagaaaccagggagagcccctaagctcctgatctatgctgcatccacaagtggggtcccatcaaggttcagtggcagtggctctggtacagatttcactctcaccatcagcagtctgcaacctgaagattttgcaacttactactgtcaacagagttacactaccccccggacgttcggccaagggaccaaggtggaaatcaaa

SEQ ID NO: 286 - DNA, 324 bp, synthetic construct
  misc_feature 1..324: note = Monoclonal antibody Light chain nucleotide sequence
  source 1..324: mol_type = other DNA
gaaattgtgttgacacagtctccagccaccctgtctttgtctccaggggacagagccaccctctcctgcagggccagtcagagtgttaacacctacttagcctggtaccaacacaaacctggccaggctcccaggctcctcatctatgatgcatccaagagggccactggcatcccagccaggttcagtggcagtgggtctgggacagacttcactctcaccatcagcagcctagagcctgaagattttgcagtttattactgtcagcagcgtgccaacaggcctccgctcactttcggcggagggacgaaggtggagatcaaa

SEQ ID NO: 287 - DNA, 324 bp, synthetic construct
  misc_feature 1..324: note = Monoclonal antibody Light chain nucleotide sequence
  source 1..324: mol_type = other DNA
gaaattgtgttgacgcagtctccaggcaccctgtctttgtctccaggggaaagagccaccctctcctgcagggccagtcagagtgttactagcaacttcgtaggctggtatcagcagaaacctggccaggctcccaggctcctcatctatgctgcatccagcaggcccactggcatcccagagagattcagtggcagtgggtctgggacagacttcactctcaccatcagcagactggagcctgaagattttgcagtgtattactgtgaacaatatggtagctcacctcggacgttcggcccagggaccaaggtggaaatcaaa

SEQ ID NO: 288 - DNA, 321 bp, synthetic construct
  misc_feature 1..321: note = Monoclonal antibody Light chain nucleotide sequence
  source 1..321: mol_type = other DNA
gacatccagatgacccagtctccatcctccctgtctgcatctgtaggggacagagtcaccatcacttgccgggcaagtcagagcattaacaactttttaaattggtatcaacagaagccagggaaagcccctaagctcctgatctatggagcatccagtttgcagagtggggtcccatcacggttcagtgccagtgggtctgggacagatttcagtctcaccatcagcggtctgattcctgaggattttgcaatttactactgtcaacagagtgacactaccccgtggacgttcggccaagggaccaaggtggacatcaag

SEQ ID NO: 289 - DNA, 339 bp, synthetic construct
  misc_feature 1..339: note = Monoclonal antibody Light chain nucleotide sequence
  source 1..339: mol_type = other DNA
gacatcgtgatgacccagtctccagactccctggctgtgtctctgggcgagagggccaccatcaagtgcaagtccagccagactattttatacagctccaacaataacaactacttagcttggtaccagcagaaaccaggacagcctcctaagctgctcatttactgggcgtctacccgggaatccggggtccctgaccgattcagtggcagtgggtctgggacagatttcactctcaccatcagcagcctgcaggctgaagatgtggcagtttattactgtcaacaatattatagttctcccctcactttcggcggagggaccaaagtggagatcaaa

SEQ ID NO: 290 - DNA, 321 bp, synthetic construct
  misc_feature 1..321: note = Monoclonal antibody Light chain nucleotide sequence
  source 1..321: mol_type = other DNA
gacatccagatgacccagtctccatcctccctgtctgcatctgtaggagacagagtcaccatcacttgccgggcaagtcagagcattagcagttatttaaattggtatcagcagaaaccagggaaagcccctaaactcctgatctatgctgcatctactttgcaaagtggggtcccatcaaggttcagtggcagtggatctgggacagatttcactctcaccatcagcagtctacaatctgaagattttgctacctactactgtcaacagagcttcaatacccctcggacgttcggccaagggaccaaggtggaaatcaaa

SEQ ID NO: 291 - DNA, 321 bp, synthetic construct
  misc_feature 1..321: note = Monoclonal antibody Light chain nucleotide sequence
  source 1..321: mol_type = other DNA
tcctatgagctgacacagccaacctcggtgtcagtgtccccaggacagacggcgaggatcacctgctctggagatgcattgccgagccaatatgcttattggtaccagcagaagccaggccaggcccctatcttaataatatataaagacaataagaggccctcagggatccctgagcgattctctggctccagctcagggacaagagtcaccttgaccatcagtggagtccagccagaggacgaggctgactattactgtcaatcagcagaccgcagtggtcgttatgtcttcgggactggaaccagggtccccgtccta

SEQ ID NO: 292 - DNA, 330 bp, synthetic construct
  misc_feature 1..330: note = Monoclonal antibody Light chain nucleotide sequence
  source 1..330: mol_type = other DNA
cagtctgtgctgactcagccaccctcagcgtctgggacccccgggcagagggtcaccatctcttgttctggaagcacctccaacgtcggaactaattatgtgtactggtgccagcagttcccaggagcggcccccaaactcctcatgtataggaatgatcgccggccctcaggggtccctgaccgattctctggctccaagtctggcacctcagcctccctggccatcagtgggctccggtccgaggatgaggctgattattattgtgcagcatgggatgacagcctgagtagttgggtgttcggcggagggaccaagctgaccgtccta

SEQ ID NO: 293 - DNA, 336 bp, synthetic construct
  misc_feature 1..336: note = Monoclonal antibody Light chain nucleotide sequence
  source 1..336: mol_type = other DNA
cagtctgtgctgacgcagccgccctcagtgtctggggccccagggcagagggtcaccatctcctgcactgggagtagttccaacatcggggcaggttatgatgtacactggtaccagcaacttccagggacagcccccaaactcctcatccatggtaacagcaatcggccctcaggggtccctgaccgattttctggctccaagtctggcacctcagcctccctggccatcactgggctccaggctgaggatgaggctgattattactgccagtcctttgacagcagcctgagtggttctgtggtgttcggcggagggaccaaactgaccgtccta

SEQ ID NO: 294 - DNA, 330 bp, synthetic construct
  misc_feature 1..330: note = Monoclonal antibody Light chain nucleotide sequence
  source 1..330: mol_type = other DNA
cagtctgccctgactcagcctgcctccgtgtctcggtctcccggacagtcgatcaccatctcctgcacgggaaccaaaaatgatgttggaagttataaccatgtctcctggtaccaacagcacccaggcaagggccccaaactcataatttatgaggtcaataagcggccctcaggagtttctgatcgcttctctggctccaagtctggcaacacggcctccctgacaatctctgggctccaggctgaggacgaggcttattattcctgctcctcatatgcacctatgaccactttagtgttcggcggagggaccaagctgaccgtccta

SEQ ID NO: 295 - DNA, 333 bp, synthetic construct
  misc_feature 1..333: note = Monoclonal antibody Light chain nucleotide sequence
  source 1..333: mol_type = other DNA
aattttatgctgactcagccccactctgtgtcggagtctccggggaagacggtaaccatctcctgcaccggcagcagtggcagcattgccagcaactatgtgcagtggtaccagcagcgcccgggcagtgcccccaccactttgatctatgaggataagcaaagaccctctggggtccctgatcggttctctggctccatcgacagctccaccaactctgcctccctcaccatctctggactgaagactgaggacgaggctgactactactgtcagtcttttgatagcagtaatcgttgggtgttcggcggagggaccaaagtgaccgtccta

SEQ ID NO: 296 - DNA, 330 bp, synthetic construct
  misc_feature 1..330: note = Monoclonal antibody Light chain nucleotide sequence
  source 1..330: mol_type = other DNA
cagtctgtgttgacgcagccgccctcagtgtctgcggccccaggacagaaggtcaccatctcctgctctggaagcagctccaacattgggaataattatgtatcctggtaccagcagctcccaggaacagcccccaaactcctcatttatgacaataataagcgaccctcagggattcctgaccgattctctggctccaagtctggcacgtcagccaccctgggcatcagcggactccagactggggacgaggccgattattactgcggaacatgggatagcagcctgagtgcttgggttttcggcggagggaccaagctgaccgtccta

SEQ ID NO: 297 - DNA, 327 bp, synthetic construct
  misc_feature 1..327: note = Monoclonal antibody Light chain nucleotide sequence
  source 1..327: mol_type = other DNA
caggctgtggtgactcaggagccctcactgactgtgtccccaggagggacagtcactctcacctgtggctccagcactggagctgtcaccagtggtcattatccctactggttccagcagaagcctggccaagccccccggacactgatttatgataccagcgacagacactcctggacacctgcccggttttcaggctcccttcgtgggggcaaagcagccctgaccctttcgggtgcgcagcctgaagatgaggctgactattattgcttcctctcctataatggtgctcgggtattcggcggagggaccaagctgaccgtcctg

SEQ ID NO: 298 - DNA, 321 bp, synthetic construct
  misc_feature 1..321: note = Monoclonal antibody Light chain nucleotide sequence
  source 1..321: mol_type = other DNA
gccatccagatgacccagtctccatcctccctgtctgcatctgtaggagacagagtcaccatcacttgccgggcaagtcagggcattagaaatgatttaggctggtatcagcagaaaccagggaaagcccctaagctcctgatctatgctgcatccagtttacaaagtggggtcccatcaaggttcagcggcagtggatctggcacagatttcactctcaccatcagcagcctgcagcctgaagattttgcaacttattactgtctacaagattacaattacccgtggacgttcggccaagggaccaaggtggaaatcaaa

SEQ ID NO: 299 - DNA, 318 bp, synthetic construct
  misc_feature 1..318: note = Monoclonal antibody Light chain nucleotide sequence
  source 1..318: mol_type = other DNA
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctatgatgcctccagtttggaaagtggggtcccatcaaggttcagcggcagtggatctgggacagaattcactctcaccatcagcagcctgcagcctgatgattttgcaacttattactgccaacagtataatagttattcgacgttcggccaagggaccaaggtggaaatcaga

SEQ ID NO: 300 - DNA, 321 bp, synthetic construct
  misc_feature 1..321: note = Monoclonal antibody Light chain nucleotide sequence
  source 1..321: mol_type = other DNA
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctatgatgcctccagtttggaaagtggggtcccatcaaggttcagcggcagtggatctgggacagaattcactctcaccatcagcagcctgcagcctgatgattttgcaacttattactgccaacagtataatgattttcccctcactttcggcggagggaccaaggtggagatcaaa

SEQ ID NO: 301 - DNA, 318 bp, synthetic construct
  misc_feature 1..318: note = Monoclonal antibody Light chain nucleotide sequence
  source 1..318: mol_type = other DNA
gacatccagatgacccagtctccatcctccctgtctgcatctgtaggagacagagtcaccatcacttgccaggcgagtcaggacattagcaactatttaaattggtatcagcagaaaccagggaaagcccctaagctcctgatctacgatgcatccaatttggaaacaggggtcccatcaaggttcagtggaagtggatctgggacagattttactttcaccatcagcagcctgcagcctgaagatattgcaacatattactgtcaacagtatgataatctcccttctttcggcggagggaccaaggtggagatcaaa

SEQ ID NO: 302 - DNA, 318 bp, synthetic construct
  misc_feature 1..318: note = Monoclonal antibody Light chain nucleotide sequence
  source 1..318: mol_type = other DNA
gaaatagtgatgacgcagtctccagccaccctgtctgtgtctccaggggaaagagccaccctctcctgcagggccagtcagagtgttagcagcaacttagcctggtaccagcagaaacctggccaggctcccaggctcctcatctatggtgcatccaccagggccactggtatcccagccaggttcagtggcagtgggtctgggacagagttcactctcaccatcagcagcctgcagtctgaagattttgcagtttattactgtcagcagtataataactggccgggttttggccaggggaccaagctggagatcaaa

SEQ ID NO: 303 - DNA, 336 bp, synthetic construct
  misc_feature 1..336: note = Monoclonal antibody Light chain nucleotide sequence
  source 1..336: mol_type = other DNA
gatattgtgatgactcagtctccactctccctgcccgtcacccctggagagccggcctccatctcctgcaggtctagtcagagcctcctgcatagtaatggatacaactatttggattggtacctgcagaagccagggcagtctccacagctcctgatctatttgggttctaatcgggcctccggggtccctgacaggttcagtggcagtggatcaggcacagattttacactgaaaatcagcagagtggaggctgaggatgttggggtttattactgcatgcaagctctacaaactcctttcactttcggccctgggaccaaagtggatatcaaa

SEQ ID NO: 304 - DNA, 321 bp, synthetic construct
  misc_feature 1..321: note = Monoclonal antibody Light chain nucleotide sequence
  source 1..321: mol_type = other DNA
gacatccagatgacccagtctccttccaccctgtctgcatctgtaggagacagagtcaccatcacttgccgggccagtcagagtattagtagctggttggcctggtatcagcagaaaccagggaaagcccctaagctcctgatctataaggcgtctagtttagaaagtggggtcccatcaaggttcagcggcagtggatctgggacagaattcactctcaccatcagcagcctgcagcctgatgattttgcaacttattactgccaacagtataatagttacgggatcactttcggccctgggaccaaagtggatatcaaa

SEQ ID NO: 305 - DNA, 324 bp, synthetic construct
  misc_feature 1..324: note = Monoclonal antibody Light chain nucleotide sequence
  source 1..324: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctccatacacttttggccaggggaccaagctggagatcaaa

SEQ ID NO: 306 - DNA, 321 bp, synthetic construct
  misc_feature 1..321: note = Monoclonal antibody Light chain nucleotide sequence
  source 1..321: mol_type = other DNA
gacatccagatgacccagtctccatcctccctgtctgcatctgtaggagacagagtcaccatcacttgccgggcgagtcagggcattagcaattatttagcctggtatcagcagaaaccagggaaagttcctaagctcctgatctatgctgcatccactttgcaatcaggggtcccatctcggttcagtggcagtggatctgggacagatttcactctcaccatcagcagcctgcagcctgaagatgttgcaacttattactgtcaaaagtataacagtgccccgtggacgttcggccaagggaccaaggtggaaatcaaa

SEQ ID NO: 307 - DNA, 321 bp, synthetic construct
  misc_feature 1..321: note = Monoclonal antibody Light chain nucleotide sequence
  source 1..321: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccccccggacgttcggccaagggaccaaggtggaaatcaaa

SEQ ID NO: 308 - DNA, 324 bp, synthetic construct
  misc_feature 1..324: note = Monoclonal antibody Light chain nucleotide sequence
  source 1..324: mol_type = other DNA
gaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccgctcactttcggcggagggaccaaggtggagatcaaa

SEQ ID NO: 309 - DNA, 324 bp, synthetic construct
  misc_feature 1..324: note = Monoclonal antibody Light chain nucleotide sequence
  source 1..324: mol_type = other DNA
gaaattgtgttgacgcagtctccaggcaccctgtctttgtctccaggggaaagagccaccctctcctgcagggccagtcagagtgttagcagcagctacttagcctggtaccagcagaaacctggccaggctcccaggctcctcatctatggtgcatccagcagggccactggcatcccagacaggttcagtggcagtgggtctgggacagacttcactctcaccatcagcagactggagcctgaagattttgcagtgtattactgtcagcagtatggtagctcacctcggacgttcggccaagggaccaaggtggaaatcaaa

SEQ ID NO: 310 - DNA, 321 bp, synthetic construct
  misc_feature 1..321: note = Monoclonal antibody Light chain nucleotide sequence
  source 1..321: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtaccccgtggacgttcggccaagggaccaaggtggaaatcaag

SEQ ID NO: 311 - DNA, 339 bp, synthetic construct
  misc_feature 1..339: note = Monoclonal antibody Light chain nucleotide sequence
  source 1..339: mol_type = other DNA
gacatcgtgatgacccagtctccagactccctggctgtgtctctgggcgagagggccaccatcaactgcaagtccagccagagtgttttatacagctccaacaataagaactacttagcttggtaccagcagaaaccaggacagcctcctaagctgctcatttactgggcatctacccgggaatccggggtccctgaccgattcagtggcagcgggtctgggacagatttcactctcaccatcagcagcctgcaggctgaagatgtggcagtttattactgtcagcaatattatagtactcccctcactttcggcggagggaccaaggtggagatcaaa

SEQ ID NO: 312 - DNA, 321 bp, synthetic construct
  misc_feature 1..321: note = Monoclonal antibody Light chain nucleotide sequence
  source 1..321: mol_type = other DNA
gacatccagatgacccagtctccatcctccctgtctgcatctgtaggagacagagtcaccatcacttgccgggcaagtcagagcattagcagctatttaaattggtatcagcagaaaccagggaaagcccctaagctcctgatctatgctgcatccagtttgcaaagtggggtcccatcaaggttcagtggcagtggatctgggacagatttcactctcaccatcagcagtctgcaacctgaagattttgcaacttactactgtcaacagagttacagtacccctcggacgttcggccaagggaccaaggtggaaatcaaa

SEQ ID NO: 313 - DNA, 330 bp, synthetic construct
  misc_feature 1..330: note = Monoclonal antibody Light chain nucleotide sequence
  source 1..330: mol_type = other DNA
cagtctgtgctgactcagccaccctcagcgtctgggacccccgggcagagggtcaccatctcttgttctggaagcagctccaacatcggaagtaattatgtatactggtaccagcagctcccaggaacggcccccaaactcctcatctataggaataatcagcggccctcaggggtccctgaccgattctctggctccaagtctggcacctcagcctccctggccatcagtgggctccggtccgaggatgaggctgattattactgtgcagcatgggatgacagcctgagtggttgggtgttcggcggagggaccaagctgaccgtccta

SEQ ID NO: 314 - DNA, 330 bp, synthetic construct
  misc_feature 1..330: note = Monoclonal antibody Light chain nucleotide sequence
  source 1..330: mol_type = other DNA
cagtctgccctgactcagcctgcctccgtgtctgggtctcctggacagtcgatcaccatctcctgcactggaaccagcagtgatgttgggagttataaccttgtctcctggtaccaacagcacccaggcaaagcccccaaactcatgatttatgaggtcagtaagcggccctcaggggtttctaatcgcttctctggctccaagtctggcaacacggcctccctgacaatctctgggctccaggctgaggacgaggctgattattactgctgctcatatgcaggtagtagcactttagtgttcggcggagggaccaagctgaccgtccta

SEQ ID NO: 315 - DNA, 333 bp, synthetic construct
  misc_feature 1..333: note = Monoclonal antibody Light chain nucleotide sequence
  source 1..333: mol_type = other DNA
aattttatgctgactcagccccactctgtgtcggagtctccggggaagacggtaaccatctcctgcaccggcagcagtggcagcattgccagcaactatgtgcagtggtaccagcagcgcccgggcagtgcccccaccactgtgatctatgaggataaccaaagaccctctggggtccctgatcggttctctggctccatcgacagctcctccaactctgcctccctcaccatctctggactgaagactgaggacgaggctgactactactgtcagtcttatgatagcagcaatcgttgggtgttcggcggagggaccaagctgaccgtccta

SEQ ID NO: 316 - DNA, 327 bp, synthetic construct
  misc_feature 1..327: note = Monoclonal antibody Light chain nucleotide sequence
  source 1..327: mol_type = other DNA
caggctgtggtgactcaggagccctcactgactgtgtccccaggagggacagtcactctcacctgtggctccagcactggagctgtcaccagtggtcattatccctactggttccagcagaagcctggccaagcccccaggacactgatttatgatacaagcaacaaacactcctggacacctgcccggttctcaggctccctccttgggggcaaagctgccctgaccctttcgggtgcgcagcctgaggatgaggctgagtattactgcttgctctcctatagtggtgctcgggtattcggcggagggaccaagctgaccgtcctg

SEQ ID NO: 317 - protein, 105 aa, synthetic construct
  REGION 1..105: note = Monoclonal antibody Light chain amino acid sequence
DIQMTQSPSSLSASVGDRVTITCRASQSIASYLNWYQQKPGRAPKLLIYAASTSGVPSRFSGSGSGTDFTLTISSLQPEDFATYYCQQSYTTPRTFGQGTKVEIK

SEQ ID NO: 318 - protein, 108 aa, synthetic construct
  REGION 1..108: note = Monoclonal antibody Light chain amino acid sequence
EIVLTQSPATLSLSPGDRATLSCRASQSVNTYLAWYQHKPGQAPRLLIYDASKRATGIPARFSGSGSGTDFTLTISSLEPEDFAVYYCQQRANRPPLTFGGGTKVEIK

SEQ ID NO: 319 - protein, 108 aa, synthetic construct
  REGION 1..108: note = Monoclonal antibody Light chain amino acid sequence
EIVLTQSPGTLSLSPGERATLSCRASQSVTSNFVGWYQQKPGQAPRLLIYAASSRPTGIPERFSGSGSGTDFTLTISRLEPEDFAVYYCEQYGSSPRTFGPGTKVEIK

SEQ ID NO: 320 - protein, 107 aa, synthetic construct
  REGION 1..107: note = Monoclonal antibody Light chain amino acid sequence
DIQMTQSPSSLSASVGDRVTITCRASQSINNFLNWYQQKPGKAPKLLIYGASSLQSGVPSRFSASGSGTDFSLTISGLIPEDFAIYYCQQSDTTPWTFGQGTKVDIK

SEQ ID NO: 321 - protein, 113 aa, synthetic construct
  REGION 1..113: note = Monoclonal antibody Light chain amino acid sequence
DIVMTQSPDSLAVSLGERATIKCKSSQTILYSSNNNNYLAWYQQKPGQPPKLLIYWASTRESGVPDRFSGSGSGTDFTLTISSLQAEDVAVYYCQQYYSSPLTFGGGTKVEIK

SEQ ID NO: 322 - protein, 107 aa, synthetic construct
  REGION 1..107: note = Monoclonal antibody Light chain amino acid sequence
DIQMTQSPSSLSASVGDRVTITCRASQSISSYLNWYQQKPGKAPKLLIYAASTLQSGVPSRFSGSGSGTDFTLTISSLQSEDFATYYCQQSFNTPRTFGQGTKVEIK

SEQ ID NO: 323 - protein, 107 aa, synthetic construct
  REGION 1..107: note = Monoclonal antibody Light chain amino acid sequence
SYELTQPTSVSVSPGQTARITCSGDALPSQYAYWYQQKPGQAPILIIYKDNKRPSGIPERFSGSSSGTRVTLTISGVQPEDEADYYCQSADRSGRYVFGTGTRVPVL

SEQ ID NO: 324 - protein, 110 aa, synthetic construct
  REGION 1..110: note = Monoclonal antibody Light chain amino acid sequence
QSVLTQPPSASGTPGQRVTISCSGSTSNVGTNYVYWCQQFPGAAPKLLMYRNDRRPSGVPDRFSGSKSGTSASLAISGLRSEDEADYYCAAWDDSLSSWVFGGGTKLTVL

SEQ ID NO: 325 - protein, 112 aa, synthetic construct
  REGION 1..112: note = Monoclonal antibody Light chain amino acid sequence
QSVLTQPPSVSGAPGQRVTISCTGSSSNIGAGYDVHWYQQLPGTAPKLLIHGNSNRPSGVPDRFSGSKSGTSASLAITGLQAEDEADYYCQSFDSSLSGSVVFGGGTKLTVL

SEQ ID NO: 326 - protein, 110 aa, synthetic construct
  REGION 1..110: note = Monoclonal antibody Light chain amino acid sequence
QSALTQPASVSRSPGQSITISCTGTKNDVGSYNHVSWYQQHPGKGPKLIIYEVNKRPSGVSDRFSGSKSGNTASLTISGLQAEDEAYYSCSSYAPMTTLVFGGGTKLTVL

SEQ ID NO: 327 - protein, 111 aa, synthetic construct
  REGION 1..111: note = Monoclonal antibody Light chain amino acid sequence
NFMLTQPHSVSESPGKTVTISCTGSSGSIASNYVQWYQQRPGSAPTTLIYEDKQRPSGVPDRFSGSIDSSTNSASLTISGLKTEDEADYYCQSFDSSNRWVFGGGTKVTVL

SEQ ID NO: 328 - protein, 110 aa, synthetic construct
  REGION 1..110: note = Monoclonal antibody Light chain amino acid sequence
QSVLTQPPSVSAAPGQKVTISCSGSSSNIGNNYVSWYQQLPGTAPKLLIYDNNKRPSGIPDRFSGSKSGTSATLGISGLQTGDEADYYCGTWDSSLSAWVFGGGTKLTVL

SEQ ID NO: 329 - protein, 109 aa, synthetic construct
  REGION 1..109: note = Monoclonal antibody Light chain amino acid sequence
QAVVTQEPSLTVSPGGTVTLTCGSSTGAVTSGHYPYWFQQKPGQAPRTLIYDTSDRHSWTPARFSGSLRGGKAALTLSGAQPEDEADYYCFLSYNGARVFGGGTKLTVL

SEQ ID NO: 330 - protein, 107 aa, synthetic construct
  REGION 1..107: note = Monoclonal antibody Light chain amino acid sequence
AIQMTQSPSSLSASVGDRVTITCRASQGIRNDLGWYQQKPGKAPKLLIYAASSLQSGVPSRFSGSGSGTDFTLTISSLQPEDFATYYCLQDYNYPWTFGQGTKVEIK

SEQ ID NO: 331 - protein, 106 aa, synthetic construct
  REGION 1..106: note = Monoclonal antibody Light chain amino acid sequence
DIQMTQSPSTLSASVGDRVTITCRASQSISSWLAWYQQKPGKAPKLLIYDASSLESGVPSRFSGSGSGTEFTLTISSLQPDDFATYYCQQYNSYSTFGQGTKVEIR

SEQ ID NO: 332 - protein, 107 aa, synthetic construct
  REGION 1..107: note = Monoclonal antibody Light chain amino acid sequence
DIQMTQSPSTLSASVGDRVTITCRASQSISSWLAWYQQKPGKAPKLLIYDASSLESGVPSRFSGSGSGTEFTLTISSLQPDDFATYYCQQYNDFPLTFGGGTKVEIK

SEQ ID NO: 333 - protein, 106 aa, synthetic construct
  REGION 1..106: note = Monoclonal antibody Light chain amino acid sequence
DIQMTQSPSSLSASVGDRVTITCQASQDISNYLNWYQQKPGKAPKLLIYDASNLETGVPSRFSGSGSGTDFTFTISSLQPEDIATYYCQQYDNLPSFGGGTKVEIK

SEQ ID NO: 334 - protein, 106 aa, synthetic construct
  REGION 1..106: note = Monoclonal antibody Light chain amino acid sequence
EIVMTQSPATLSVSPGERATLSCRASQSVSSNLAWYQQKPGQAPRLLIYGASTRATGIPARFSGSGSGTEFTLTISSLQSEDFAVYYCQQYNNWPGFGQGTKLEIK

SEQ ID NO: 335 - protein, 112 aa, synthetic construct
  REGION 1..112: note = Monoclonal antibody Light chain amino acid sequence
DIVMTQSPLSLPVTPGEPASISCRSSQSLLHSNGYNYLDWYLQKPGQSPQLLIYLGSNRASGVPDRFSGSGSGTDFTLKISRVEAEDVGVYYCMQALQTPFTFGPGTKVDIK

SEQ ID NO: 336 - protein, 107 aa, synthetic construct
  REGION 1..107: note = Monoclonal antibody Light chain amino acid sequence
DIQMTQSPSTLSASVGDRVTITCRASQSISSWLAWYQQKPGKAPKLLIYKASSLESGVPSRFSGSGSGTEFTLTISSLQPDDFATYYCQQYNSYGITFGPGTKVDIK

SEQ ID NO: 337 - protein, 108 aa, synthetic construct
  REGION 1..108: note = Monoclonal antibody Light chain amino acid sequence
DIQMTQSPSSLSASVGDRVTITCRASQSISSYLNWYQQKPGKAPKLLIYAASSLQSGVPSRFSGSGSGTDFTLTISSLQPEDFATYYCQQSYSTPPYTFGQGTKLEIK

SEQ ID NO: 338 - protein, 107 aa, synthetic construct
  REGION 1..107: note = Monoclonal antibody Light chain amino acid sequence
DIQMTQSPSSLSASVGDRVTITCRASQGISNYLAWYQQKPGKVPKLLIYAASTLQSGVPSRFSGSGSGTDFTLTISSLQPEDVATYYCQKYNSAPWTFGQGTKVEIK

SEQ ID NO: 339 - protein, 107 aa, synthetic construct
  REGION 1..107: note = Monoclonal antibody Light chain amino acid sequence
DIQMTQSPSSLSASVGDRVTITCRASQSISSYLNWYQQKPGKAPKLLIYAASSLQSGVPSRFSGSGSGTDFTLTISSLQPEDFATYYCQQSYSTPRTFGQGTKVEIK

SEQ ID NO: 340 - protein, 108 aa, synthetic construct [sequence omitted]

SEQ ID NO: 341 - protein, 108 aa, synthetic construct
  REGION 1..108: note = Monoclonal antibody Light chain amino acid sequence
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPRTFGQGTKVEIK

SEQ ID NO: 342 - protein, 107 aa, synthetic construct
  REGION 1..107: note = Monoclonal antibody Light chain amino acid sequence
DIQMTQSPSSLSASVGDRVTITCRASQSISSYLNWYQQKPGKAPKLLIYAASSLQSGVPSRFSGSGSGTDFTLTISSLQPEDFATYYCQQSYSTPWTFGQGTKVEIK

SEQ ID NO: 343 - protein, 113 aa, synthetic construct
  REGION 1..113: note = Monoclonal antibody Light chain amino acid sequence
DIVMTQSPDSLAVSLGERATINCKSSQSVLYSSNNKNYLAWYQQKPGQPPKLLIYWASTRESGVPDRFSGSGSGTDFTLTISSLQAEDVAVYYCQQYYSTPLTFGGGTKVEIK

SEQ ID NO: 344 - protein, 107 aa, synthetic construct; identical to SEQ ID NO: 339
  REGION 1..107: note = Monoclonal antibody Light chain amino acid sequence

SEQ ID NO: 345 - protein, 110 aa, synthetic construct
  REGION 1..110: note = Monoclonal antibody Light chain amino acid sequence
QSVLTQPPSASGTPGQRVTISCSGSSSNIGSNYVYWYQQLPGTAPKLLIYRNNQRPSGVPDRFSGSKSGTSASLAISGLRSEDEADYYCAAWDDSLSGWVFGGGTKLTVL

SEQ ID NO: 346 - protein, 110 aa, synthetic construct
  REGION 1..110: note = Monoclonal antibody Light chain amino acid sequence
QSALTQPASVSGSPGQSITISCTGTSSDVGSYNLVSWYQQHPGKAPKLMIYEVSKRPSGVSNRFSGSKSGNTASLTISGLQAEDEADYYCCSYAGSSTLVFGGGTKLTVL

SEQ ID NO: 347 - protein, 111 aa, synthetic construct
  REGION 1..111: note = Monoclonal antibody Light chain amino acid sequence
NFMLTQPHSVSESPGKTVTISCTGSSGSIASNYVQWYQQRPGSAPTTVIYEDNQRPSGVPDRFSGSIDSSSNSASLTISGLKTEDEADYYCQSYDSSNRWVFGGGTKLTVL

SEQ ID NO: 348 - protein, 109 aa, synthetic construct
  REGION 1..109: note = Monoclonal antibody Light chain amino acid sequence
QAVVTQEPSLTVSPGGTVTLTCGSSTGAVTSGHYPYWFQQKPGQAPRTLIYDTSNKHSWTPARFSGSLLGGKAALTLSGAQPEDEAEYYCLLSYSGARVFGGGTKLTVL

SEQ ID NO: 349 - protein, 8 aa, synthetic construct
  REGION 1..8: note = Antibody complementarity determining regions (CDRs)
GFTFSSYW

SEQ ID NO: 350 - protein, 8 aa, synthetic construct
  REGION 1..8: note = Antibody complementarity determining regions (CDRs)
IKPDGSEK

SEQ ID NO: 351 - protein, 18 aa, synthetic construct
  REGION 1..18: note = Antibody complementarity determining regions (CDRs)
ARDLRYCSSTSCSPALDY

SEQ ID NO: 352 - protein, 6 aa, synthetic construct
  REGION 1..6: note = Antibody complementarity determining regions (CDRs)
QSIASY

SEQ ID NO: 353 - skipped

SEQ ID NO: 354 - protein, 9 aa, synthetic construct
  REGION 1..9: note = Antibody complementarity determining regions (CDRs)
QQSYTTPRT

SEQ ID NO: 355 - protein, 8 aa, synthetic construct
  REGION 1..8: note = Antibody complementarity determining regions (CDRs)
GFTFDNYG

SEQ ID NO: 356 - protein, 8 aa, synthetic construct
  REGION 1..8: note = Antibody complementarity determining regions (CDRs)
ISSGGVGT

SEQ ID NO: 357 - protein, 14 aa, synthetic construct
  REGION 1..14: note = Antibody complementarity determining regions (CDRs)
AKDLLRYESSGYSP

SEQ ID NO: 358 - protein, 6 aa, synthetic construct
  REGION 1..6: note = Antibody complementarity determining regions (CDRs)
QSVNTY

SEQ ID NO: 359 - skipped

SEQ ID NO: 360 - protein, 10 aa, synthetic construct
  REGION 1..10: note = Antibody complementarity determining regions (CDRs)
QQRANRPPLT

SEQ ID NO: 361 - protein, 10 aa, synthetic construct
  REGION 1..10: note = Antibody complementarity determining regions (CDRs)
GAFLNSGGYY

SEQ ID NO: 362 - protein, 7 aa, synthetic construct
  REGION 1..7: note = Antibody complementarity determining regions (CDRs)
IYYSGTT

SEQ ID NO: 363 - protein, 18 aa, synthetic construct
  REGION 1..18: note = Antibody complementarity determining regions (CDRs)
RAPRIPFGEVIGGAAFDV

SEQ ID NO: 364 - protein, 7 aa, synthetic construct
  REGION 1..7: note = Antibody complementarity determining regions (CDRs)
QSVTSNF

SEQ ID NO: 365 - skipped

SEQ ID NO: 366 - protein, 9 aa, synthetic construct
  REGION 1..9: note = Antibody complementarity determining regions (CDRs)
EQYGSSPRT

SEQ ID NO: 367 - protein, 10 aa, synthetic construct
  REGION 1..10: note = Antibody complementarity determining regions (CDRs)
GAFLNSGGYY

SEQ ID NO: 368 - protein, 10 aa, synthetic construct
  REGION 1..10: note = Antibody complementarity determining regions (CDRs)
GGSFSNSNYY

SEQ ID NO: 369 - protein, 7 aa, synthetic construct
  REGION 1..7: note = Antibody complementarity determining regions (CDRs)
IYASAIT

SEQ ID NO: 370 - protein, 17 aa, synthetic construct
  REGION 1..17: note = Antibody complementarity determining regions (CDRs)
ARGFRLAAEAYYHGMDV

SEQ ID NO: 371 - protein, 6 aa, synthetic construct
  REGION 1..6: note = Antibody complementarity determining regions (CDRs)
QSINNF

SEQ ID NO: 372 - skipped

SEQ ID NO: 373 - protein, 9 aa, synthetic construct
  REGION 1..9: note = Antibody complementarity determining regions (CDRs)
QQSDTTPWT

SEQ ID NO: 374 - protein, 8 aa, synthetic construct
  REGION 1..8: note = Antibody complementarity determining regions (CDRs)
GGTFSRSA

SEQ ID NO: 375 - protein, 8 aa, synthetic construct
  REGION 1..8: note = Antibody complementarity determining regions (CDRs)
IIRIFNTA

SEQ ID NO: 376 - protein, 24 aa, synthetic construct
  REGION 1..24: note = Antibody complementarity determining regions (CDRs)
ATSSLSDIVVAEGAFVDHYFGMDV

SEQ ID NO: 377 - protein, 12 aa, synthetic construct
  REGION 1..12: note = Antibody complementarity determining regions (CDRs)
QTILYSSNNNNY

SEQ ID NO: 378 - skipped

SEQ ID NO: 379 - protein, 9 aa, synthetic construct
  REGION 1..9: note = Antibody complementarity determining regions (CDRs)
QQYYSSPLT

SEQ ID NO: 380 - protein, 8 aa, synthetic construct
  REGION 1..8: note = Antibody complementarity determining regions (CDRs)
AFTLSRYE

SEQ ID NO: 381 - protein, 8 aa, synthetic construct
  REGION 1..8: note = Antibody complementarity determining regions (CDRs)
ISSSGDTI

SEQ ID NO: 382 - protein, 21 aa, synthetic construct
  REGION 1..21: note = Antibody complementarity determining regions (CDRs)
ATWGLGYCNSTGCYITDGMDV

SEQ ID NO: 383 - protein, 6 aa, synthetic construct
  REGION 1..6: note = Antibody complementarity determining regions (CDRs)
QSISSY

SEQ ID NO: 384 - skipped

SEQ ID NO: 385 - protein, 8 aa, synthetic construct
  REGION 1..8: note = Antibody complementarity determining regions (CDRs)
QQSFNTPR

SEQ ID NO: 386 - protein, 10 aa, synthetic construct
  REGION 1..10: note = Antibody complementarity determining regions (CDRs)
GGSISSSDYS

SEQ ID NO: 387 - protein, 7 aa, synthetic construct
  REGION 1..7: note = Antibody complementarity determining regions (CDRs)
IYHSGNT

SEQ ID NO: 388 - protein, 13 aa, synthetic construct
  REGION 1..13: note = Antibody complementarity determining regions (CDRs)
ARDPGGNSGWFDP

SEQ ID NO: 389 - protein, 6 aa, synthetic construct
  REGION 1..6: note = Antibody complementarity determining regions (CDRs)
ALPSQY

SEQ ID NO: 390 - skipped

SEQ ID NO: 391 - protein, 10 aa, synthetic construct
  REGION 1..10: note = Antibody complementarity determining regions (CDRs)
QSADRSGRYV

SEQ ID NO: 392 - protein, 8 aa, synthetic construct
  REGION 1..8: note = Antibody complementarity determining regions (CDRs)
GFIFSNTW

SEQ ID NO: 393 - protein, 10 aa, synthetic construct
  REGION 1..10: note = Antibody complementarity determining regions (CDRs)
IKSKADGGTT

SEQ ID NO: 394 - protein, 10 aa, synthetic construct
  REGION 1..10: note = Antibody complementarity determining regions (CDRs)
TTGWYSTLDY

SEQ ID NO: 395 - protein, 8 aa, synthetic construct
  REGION 1..8: note = Antibody complementarity determining regions (CDRs)
TSNVGTNY

SEQ ID NO: 396 - skipped

SEQ ID NO: 397 - protein, 11 aa, synthetic construct
  REGION 1..11: note = Antibody complementarity determining regions (CDRs)
AAWDDSLSSWV

SEQ ID NO: 398 - protein, 8 aa, synthetic construct
  REGION 1..8: note = Antibody complementarity determining regions (CDRs)
GYTFTSNG

SEQ ID NO: 399 - protein, 8 aa, synthetic construct
  REGION 1..8: note = Antibody complementarity determining regions (CDRs)
ISAYNGNT

SEQ ID NO: 400 - protein, 14 aa, synthetic construct
  REGION 1..14: note = Antibody complementarity determining regions (CDRs)
ARSRGHYGDYLYGY

SEQ ID NO: 401 - protein, 9 aa, synthetic construct
  REGION 1..9: note = Antibody complementarity determining regions (CDRs)
SSNIGAGYD

SEQ ID NO: 402 - skipped

SEQ ID NO: 403 - protein, 12 aa, synthetic construct
  REGION 1..12: note = Antibody complementarity determining regions (CDRs)
QSFDSSLSGSVV

SEQ ID NO: 404 - protein, 8 aa, synthetic construct
  REGION 1..8: note = Antibody complementarity determining regions (CDRs)
GFTFETYA

SEQ ID NO: 405 - protein, 8 aa, synthetic construct
  REGION 1..8: note = Antibody complementarity determining regions (CDRs)
ISFNSRSR

SEQ ID NO: 406 - protein, 11 aa, synthetic construct
  REGION 1..11: note = Antibody complementarity determining regions (CDRs)
ARDEKWGTPSD

SEQ ID NO: 407 - protein, 9 aa, synthetic construct
  REGION 1..9: note = Antibody complementarity determining regions (CDRs)
KNDVGSYNH

SEQ ID NO: 408 - skipped

SEQ ID NO: 409 - protein, 10 aa, synthetic construct
  REGION 1..10: note = Antibody complementarity determining regions (CDRs)
SSYAPMTTLV

SEQ ID NO: 410 - protein, 8 aa, synthetic construct
  REGION 1..8: note = Antibody complementarity determining regions (CDRs)
GGSIRGYS

SEQ ID NO: 411 - protein, 7 aa, synthetic construct
  REGION 1..7: note = Antibody complementarity determining regions (CDRs)
MLYTGTT

SEQ ID NO: 412 - protein, 14 aa, synthetic construct
  REGION 1..14: note = Antibody complementarity determining regions (CDRs)
ARGPTVSGPIVVDY

SEQ ID NO: 413 - protein, 8 aa, synthetic construct
  REGION 1..8: note = Antibody complementarity determining regions (CDRs)
SGSIASNY

SEQ ID NO: 414 - skipped

SEQ ID NO: 415 - protein, 10 aa, synthetic construct
  REGION 1..10: note = Antibody complementarity determining regions (CDRs)
QSFDSSNRWV

SEQ ID NO: 416 - protein, 10 aa, synthetic construct
  REGION 1..10: note = Antibody complementarity determining regions (CDRs)
GGSISSSSYY

SEQ ID NO: 417 - protein, 7 aa, synthetic construct
  REGION 1..7: note = Antibody complementarity determining regions (CDRs)
LSYTGST

SEQ ID NO: 418 - protein, 13 aa, synthetic construct
  REGION 1..13: note = Antibody complementarity determining regions (CDRs)
ARESGSGGTHTDS

SEQ ID NO: 419 - protein, 8 aa, synthetic construct
  REGION 1..8: note = Antibody complementarity determining regions (CDRs)
SSNIGNNY

SEQ ID NO: 420 - skipped

SEQ ID NO: 421 - protein, 11 aa, synthetic construct
  REGION 1..11: note = Antibody complementarity determining regions (CDRs)
GTWDSSLSAWV

SEQ ID NO: 422 - protein, 8 aa, synthetic construct
  REGION 1..8: note = Antibody complementarity determining regions (CDRs)
GFTFEDYG

SEQ ID NO: 423 - protein, 7 aa, synthetic construct
  REGION 1..7: note = Antibody complementarity determining regions (CDRs)
NWNGGST

SEQ ID NO: 424 - protein, 22 aa, synthetic construct
  REGION 1..22: note = Antibody complementarity determining regions (CDRs)
ARDKAIQGALMVYAMRGRWFDP

SEQ ID NO: 425 - protein, 9 aa, synthetic construct
  REGION 1..9: note = Antibody complementarity determining regions (CDRs)
TGAVTSGHY

SEQ ID NO: 426 - skipped

SEQ ID NO: 427 - protein, 9 aa, synthetic construct
  REGION 1..9: note = Antibody complementarity determining regions (CDRs)
FLSYNGARV

SEQ ID NO: 428 - protein, 8 aa, synthetic construct
  REGION 1..8: note = Antibody complementarity determining regions (CDRs)
GFTFGDYA

SEQ ID NO: 429 - protein, 10 aa, synthetic construct
  REGION 1..10: note = Antibody complementarity determining regions (CDRs)
IRSKAYGGTT

SEQ ID NO: 430 - protein, 13 aa, synthetic construct
  REGION 1..13: note = Antibody complementarity determining regions (CDRs)
TKYASGWEVGFDP

SEQ ID NO: 431 - protein, 6 aa, synthetic construct
  REGION 1..6: note = Antibody complementarity determining regions (CDRs)
QGIRND

SEQ ID NO: 432 - skipped

SEQ ID NO: 433 - protein, 9 aa, synthetic construct
  REGION 1..9: note = Antibody complementarity determining regions (CDRs)
LQDYNYPWT

SEQ ID NO: 434 - protein, 8 aa, synthetic construct
  REGION 1..8: note = Antibody complementarity determining regions (CDRs)
GYTFTSYG

SEQ ID NO: 435 - protein, 8 aa, synthetic construct
  REGION 1..8: note = Antibody complementarity determining regions (CDRs)
ISAYNGNT

SEQ ID NO: 436 - protein, 12 aa, synthetic construct
  REGION 1..12: note = Antibody complementarity determining regions (CDRs)
ARTPAALASFDY

SEQ ID NO: 437 - protein, 6 aa, synthetic construct
  REGION 1..6: note = Antibody complementarity determining regions (CDRs)
QSISSW

SEQ ID NO: 438 - skipped

SEQ ID NO: 439 - protein, 8 aa, synthetic construct
  REGION 1..8: note = Antibody complementarity determining regions (CDRs)
QQYNSYST

SEQ ID NO: 440 - protein, 8 aa, synthetic construct
  REGION 1..8: note = Antibody complementarity determining regions (CDRs)
GYTFTSYG

SEQ ID NO: 441 - protein, 8 aa, synthetic construct
  REGION 1..8: note = Antibody complementarity determining regions (CDRs)
ISAYNGNT

SEQ ID NO: 442 - protein, 14 aa, synthetic construct
  REGION 1..14: note = Antibody complementarity determining regions (CDRs)
ARGQGRYGDYIYNH

SEQ ID NO: 443 - protein, 6 aa, synthetic construct
  REGION 1..6: note = Antibody complementarity determining regions (CDRs)
QSISSW

SEQ ID NO: 444 - skipped

SEQ ID NO: 445 - protein, 9 aa, synthetic construct
  REGION 1..9: note = Antibody complementarity determining regions (CDRs)
QQYNDFPLT

SEQ ID NO: 446 - protein, 8 aa, synthetic construct
  REGION 1..8: note = Antibody complementarity determining regions (CDRs)
GFTFSSYA

SEQ ID NO: 447 - protein, 8 aa, synthetic construct
  REGION 1..8: note = Antibody complementarity determining regions (CDRs)
ISYDGSNK

SEQ ID NO: 448 - protein, 13 aa, synthetic construct
  REGION 1..13: note = Antibody complementarity determining regions (CDRs)
ARDRDSSGYIFDY

SEQ ID NO: 449 - protein, 6 aa, synthetic construct
  REGION 1..6: note = Antibody complementarity determining regions (CDRs)
QDISNY

SEQ ID NO: 450 - skipped

SEQ ID NO: 451 - protein, 8 aa, synthetic construct
  REGION 1..8: note = Antibody complementarity determining regions (CDRs)
QQYDNLPS

SEQ ID NO: 452 - protein, 10 aa, synthetic construct
  REGION 1..10: note = Antibody complementarity determining regions (CDRs)
GDSVSSNSAA

SEQ ID NO: 453 - protein, 9 aa, synthetic construct
  REGION 1..9: note = Antibody complementarity determining regions (CDRs)
TYYRSKWYN

SEQ ID NO: 454 - protein, 14 aa, synthetic construct
  REGION 1..14: note = Antibody complementarity determining regions (CDRs)
ARDLGIAAADWFDS

SEQ ID NO: 455 - protein, 6 aa, synthetic construct
  REGION 1..6: note = Antibody complementarity determining regions (CDRs)
QSVSSN

SEQ ID NO: 456 - skipped

SEQ ID NO: 457 - protein, 8 aa, synthetic construct
  REGION 1..8: note = Antibody complementarity determining regions (CDRs)
QQYNNWPG

SEQ ID NO: 458 - protein, 8 aa, synthetic construct
  REGION 1..8: note = Antibody complementarity determining regions (CDRs)
GFTFSSYG

SEQ ID NO: 459 - protein, 8 aa, synthetic construct
  REGION 1..8: note = Antibody complementarity determining regions (CDRs)
ISYDGSNK

SEQ ID NO: 460 - protein, 11 aa, synthetic construct
  REGION 1..11: note = Antibody complementarity determining regions (CDRs)
ARSPSGHAFDV

SEQ ID NO: 461 - protein, 11 aa, synthetic construct
  REGION 1..11: note = Antibody complementarity determining regions (CDRs)
QSLLHSNGYNY

SEQ ID NO: 462 - skipped

SEQ ID NO: 463 - protein, 9 aa, synthetic construct
  REGION 1..9: note = Antibody complementarity determining regions (CDRs)
MQALQTPFT

SEQ ID NO: 464 - protein, 8 aa, synthetic construct
  REGION 1..8: note = Antibody complementarity determining regions (CDRs)
GGSISSYY

SEQ ID NO: 465 - protein, 7 aa, synthetic construct
  REGION 1..7: note = Antibody complementarity determining regions (CDRs)
IYYSGST

SEQ ID NO: 466 - protein, 14 aa, synthetic construct
  REGION 1..14: note = Antibody complementarity determining regions (CDRs)
ARGPTVSGPIVVDY

SEQ ID NO: 467 - protein, 6 aa, synthetic construct
  REGION 1..6: note = Antibody complementarity determining regions (CDRs)
QSISSW

SEQ ID NO: 468 - skipped

SEQ ID NO: 469 - protein, 9 aa, synthetic construct
  REGION 1..9: note = Antibody complementarity determining regions (CDRs)
QQYNSYGIT

SEQ ID NO: 470 - protein, 8 aa, synthetic construct
  REGION 1..8: note = Antibody complementarity determining regions (CDRs)
GYTFTSYG

SEQ ID NO: 471 - protein, 8 aa, synthetic construct
  REGION 1..8: note = Antibody complementarity determining regions (CDRs)
ISAYNGNT

SEQ ID NO: 472 - protein, 24 aa, synthetic construct
  REGION 1..24: note = Antibody complementarity determining regions (CDRs)
ARDTRYCSGGSCPRPSWYYYYMDV

SEQ ID NO: 473 - protein, 6 aa, synthetic construct
  REGION 1..6: note = Antibody complementarity determining regions (CDRs)
QSISSY

SEQ ID NO: 474 - skipped

SEQ ID NO: 475 - protein, 10 aa, synthetic construct
  REGION 1..10: note = Antibody complementarity determining regions (CDRs)
QQSYSTPPYT

SEQ ID NO: 476 - protein, 8 aa, synthetic construct
  REGION 1..8: note = Antibody complementarity determining regions (CDRs)
GFTFSSYG

SEQ ID NO: 477 - protein, 8 aa, synthetic construct
  REGION 1..8: note = Antibody complementarity determining regions (CDRs)
IRYDGSNK

SEQ ID NO: 478 - protein, 12 aa, synthetic construct
  REGION 1..12: note = Antibody complementarity determining regions (CDRs)
ANPYITPPTNDY

SEQ ID NO: 479 - protein, 6 aa, synthetic construct
  REGION 1..6: note = Antibody complementarity determining regions (CDRs)
QGISNY

SEQ ID NO: 480 - skipped

SEQ ID NO: 481 - protein, 9 aa, synthetic construct
  REGION 1..9: note = Antibody complementarity determining regions (CDRs)
QKYNSAPWT

SEQ ID NO: 482 - protein, 8 aa, synthetic construct
  REGION 1..8: note = Antibody complementarity determining regions (CDRs)
GFTFSSYW

SEQ ID NO: 483 - protein, 8 aa, synthetic construct
  REGION 1..8: note = Antibody complementarity determining regions (CDRs)
IKQDGSEK

SEQ ID NO: 484 - protein, 18 aa, synthetic construct
  REGION 1..18: note = Antibody complementarity determining regions (CDRs)
ARDLRYCSSTSCSPALDY

SEQ ID NO: 485 - protein, 6 aa, synthetic construct
  REGION 1..6: note = Antibody complementarity determining regions (CDRs)
QSISSY

SEQ ID NO: 486 - skipped

SEQ ID NO: 487 - protein, 9 aa, synthetic construct
  REGION 1..9: note = Antibody complementarity determining regions (CDRs)
QQSYSTPRT

SEQ ID NO: 488 - protein, 8 aa, synthetic construct
  REGION 1..8: note = Antibody complementarity determining regions (CDRs)
GFTFSSYA

SEQ ID NO: 489 - protein, 8 aa, synthetic construct
  REGION 1..8: note = Antibody complementarity determining regions (CDRs)
ISGSGGST

SEQ ID NO: 490 - protein, 14 aa, synthetic construct
  REGION 1..14: note = Antibody complementarity determining regions (CDRs)
AKDLLRYDSSGYSP

SEQ ID NO: 491 - protein, 6 aa, synthetic construct
  REGION 1..6: note = Antibody complementarity determining regions (CDRs)
QSVSSY

SEQ ID NO: 492 - skipped

SEQ ID NO: 493 - protein, 10 aa, synthetic construct
  REGION 1..10: note = Antibody complementarity determining regions (CDRs)
QQRSNWPPLT

SEQ ID NO: 494 - protein, 10 aa, synthetic construct
  REGION 1..10: note = Antibody complementarity determining regions (CDRs)
GGSISSGGYY

SEQ ID NO: 495 - protein, 7 aa, synthetic construct
  REGION 1..7: note = Antibody complementarity determining regions (CDRs)
IYYSGST

SEQ ID NO: 496 - protein, 19 aa, synthetic construct
  REGION 1..19: note = Antibody complementarity determining regions (CDRs)
ARAPRITFGGVIGGAAFDV

SEQ ID NO: 497 - protein, 7 aa, synthetic construct
  REGION 1..7: note = Antibody complementarity determining regions (CDRs)
QSVSSSY

SEQ ID NO: 498 - skipped

SEQ ID NO: 499 - protein, 9 aa, synthetic construct
  REGION 1..9: note = Antibody complementarity determining regions (CDRs)
QQYGSSPRT

SEQ ID NO: 500 - protein, 10 aa, synthetic construct
  REGION 1..10: note = Antibody complementarity determining regions (CDRs)
GGSISSGSYY

SEQ ID NO: 501 - protein, 7 aa, synthetic construct
  REGION 1..7: note = Antibody complementarity determining regions (CDRs)
IYTSGST

SEQ ID NO: 502 - protein, 17 aa, synthetic construct
  REGION 1..17: note = Antibody complementarity determining regions (CDRs)
ARGLRLAAEAYYYGMDV

SEQ ID NO: 503 - protein, 6 aa, synthetic construct
  REGION 1..6: note = Antibody complementarity determining regions (CDRs)
QSISSY

SEQ ID NO: 504 - skipped

SEQ ID NO: 505 - protein, 9 aa, synthetic construct
  REGION 1..9: note = Antibody complementarity determining regions (CDRs)
QQSYSTPWT

SEQ ID NO: 506 - protein, 8 aa, synthetic construct
  REGION 1..8: note = Antibody complementarity determining regions (CDRs)
GGTFSSYA

SEQ ID NO: 507 - protein, 8 aa, synthetic construct
  REGION 1..8: note = Antibody complementarity determining regions (CDRs)
IIPIFGTA

SEQ ID NO: 508 - protein, 24 aa, synthetic construct
  REGION 1..24: note = Antibody complementarity determining regions (CDRs)
ATSSLSDIVVVVAAFVDHYYGMDV

SEQ ID NO: 509 - protein, 12 aa, synthetic construct
  REGION 1..12: note = Antibody complementarity determining regions (CDRs)
QSVLYSSNNKNY

SEQ ID NO: 510 - skipped

SEQ ID NO: 511 - protein, 9 aa, synthetic construct
  REGION 1..9: note = Antibody complementarity determining regions (CDRs)
QQYYSTPLT

SEQ ID NO: 512 - protein, 8 aa, synthetic construct
  REGION 1..8: note = Antibody complementarity determining regions (CDRs)
GFTFSSYE

SEQ ID NO: 513 - protein, 8 aa, synthetic construct
  REGION 1..8: note = Antibody complementarity determining regions (CDRs)
ISSSGSTI

SEQ ID NO: 514 - protein, 21 aa, synthetic construct
  REGION 1..21: note = Antibody complementarity determining regions (CDRs)
ATWGLGYCSSTSCYITDGMDV

SEQ ID NO: 515 - protein, 6 aa, synthetic construct
  REGION 1..6: note = Antibody complementarity determining regions (CDRs)
QSISSY

SEQ ID NO: 516 - skipped

SEQ ID NO: 517 - protein, 9 aa, synthetic construct
  REGION 1..9: note = Antibody complementarity determining regions (CDRs)
QQSYSTPRT

SEQ ID NO: 518 - protein, 8 aa, synthetic construct
  REGION 1..8: note = Antibody complementarity determining regions (CDRs)
GFTFSNAW

SEQ ID NO: 519 - protein, 9 aa, synthetic construct
  REGION 1..9: note = Antibody complementarity determining regions (CDRs)
IKSKTDGGT

SEQ ID NO: 520 - protein, 10 aa, synthetic construct
  REGION 1..10: note = Antibody complementarity determining regions (CDRs)
TTGCYSTLDY

SEQ ID NO: 521 - protein, 8 aa, synthetic construct
  REGION 1..8: note = Antibody complementarity determining regions (CDRs)
SSNIGSNY

SEQ ID NO: 522 - skipped

SEQ ID NO: 523 - protein, 11 aa, synthetic construct
  REGION 1..11: note = Antibody complementarity determining regions (CDRs)
AAWDDSLSGWV

SEQ ID NO: 524 - protein, 8 aa, synthetic construct
  REGION 1..8: note = Antibody complementarity determining regions (CDRs)
GFTFDDYA

SEQ ID NO: 525 - protein, 8 aa, synthetic construct
  REGION 1..8: note = Antibody complementarity determining regions (CDRs)
ISWNSGSI

SEQ ID NO: 526 - protein, 11 aa, synthetic construct
  REGION 1..11: note = Antibody complementarity determining regions (CDRs)
AKDEKWGTPSD

SEQ ID NO: 527 - protein, 9 aa, synthetic construct
  REGION 1..9: note = Antibody complementarity determining regions (CDRs)
SSDVGSYNL

SEQ ID NO: 528 - skipped

SEQ ID NO: 529 - protein, 10 aa, synthetic construct
  REGION 1..10: note = Antibody complementarity determining regions (CDRs)
CSYAGSSTLV

SEQ ID NO: 530 - protein, 8 aa, synthetic construct
  REGION 1..8: note = Antibody complementarity determining regions (CDRs)
GGSISSYY

SEQ ID NO: 531 - protein, 7 aa, synthetic construct
  REGION 1..7: note = Antibody complementarity determining regions (CDRs)
IYYSGST

SEQ ID NO: 532 - protein, 14 aa, synthetic construct
  REGION 1..14: note = Antibody complementarity determining regions (CDRs)
ARGPTVSGPIVVDY

SEQ ID NO: 533 - protein, 8 aa, synthetic construct
  REGION 1..8: note = Antibody complementarity determining regions (CDRs)
SGSIASNY

SEQ ID NO: 534 - skipped

SEQ ID NO: 535 - protein, 10 aa, synthetic construct
  REGION 1..10: note = Antibody complementarity determining regions (CDRs)
QSYDSSNRWV

SEQ ID NO: 536 - protein, 8 aa, synthetic construct
  REGION 1..8: note = Antibody complementarity determining regions (CDRs)
GFTFDDYG

SEQ ID NO: 537 - protein, 8 aa, synthetic construct
  REGION 1..8: note = Antibody complementarity determining regions (CDRs)
INWNGGST

SEQ ID NO: 538 - protein, 22 aa, synthetic construct
  REGION 1..22: note = Antibody complementarity determining regions (CDRs)
ARDKAIQGALMVYAMRGRWFDP

SEQ ID NO: 539 - protein, 9 aa, synthetic construct
  REGION 1..9: note = Antibody complementarity determining regions (CDRs)
TGAVTSGHY

SEQ ID NO: 540 - skipped

SEQ ID NO: 541 - protein, 9 aa, synthetic construct
  REGION 1..9: note = Antibody complementarity determining regions (CDRs)
LLSYSGARV